(19) United States
(12) Patent Application Publication
(10) Pub. No.: US 2021/0236476 A1
(43) Pub. Date: Aug. 5, 2021

(54) METHODS OF TREATING CANCER WITH SMALL MOLECULE PD-L1 INHIBITORS

(71) Applicant: CHEMOCENTRYX, INC., Mountain View, CA (US)

(72) Inventors: Shijie LI, Los Altos, CA (US); Marta VILALTA-COLOMER, Mountain View, CA (US); Sreenivas PUNNA, Sunnyvale, CA (US); Viengkham MALATHONG, Mountain View, CA (US); Rajinder SINGH, Belmont, CA (US); Penglie ZHANG, Foster City, CA (US)

(21) Appl. No.: 17/267,937

(22) PCT Filed: Aug. 28, 2019
(86) PCT No.: PCT/US19/48466

§ 371 Date: Feb. 11, 2021

Related U.S. Application Data
(60) Provisional application No. 62/724,435, filed on Aug. 29, 2018.
(60) Provisional application No. 62/772,226, filed on Nov. 28, 2018.

(51) Int. Cl. A61K 31/4545; A61K 9/00; A61P 35/00; A61K 31/4427; A61K 31/4025; A61K 31/445; A61K 31/40; A61K 31/4439; A61K 31/44; A61K 31/397; A61K 31/5377; A61K 31/496; A61K 31/454; A61K 31/453; A61K 31/55; A61K 31/551; A61K 31/41; A61K 31/69

(52) U.S. Cl. CPC A61K 31/4545; A61K 31/69; A61P 35/00; A61K 31/4427; A61K 31/4025; A61K 31/445; A61K 31/40; A61K 31/4439; A61K 31/44; A61K 31/397; A61K 31/5377; A61K 31/496; A61K 31/454; A61K 31/453; A61K 31/55; A61K 31/551; A61K 31/41; A61K 9/0053

(57) ABSTRACT

The present disclosure provides, inter alia, methods of treating cancer by administering an effective amount of a com-pound of Formula (I). In some embodiments, the cancer is a solid cancer. In some embodiments, the cancer is melanoma. In some aspects, the present disclosure provides methods of increasing the CD8+ T cell/CD4+ T cell ratio in a solid tumor microenvironment by administering an effective amount of a compound of Formula (I).

### CROSS-REFERENCES TO RELATED APPLICATIONS

This application claims the benefit of priority under 35 U.S.C § 119(e) to U.S. Provisional Application Ser. Nos. 62/724,435 filed 29 Aug. 2018 and 62/772,226 filed 28 Nov. 2018, the disclosures of each are incorporated herein by reference in their entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER

PROGRAM LISTING APPENDIX SUBMITTED ON A COMPACT DISK

NOT APPLICABLE

### BACKGROUND

FDA approved antibody-based therapies targeting the Programmed cell Death-1/Programmed Death-Ligand 1 (PD-1/PD-L1) immune checkpoint axis have gained considerable attention and success in cancer immunotherapy recently. Despite the improvements in treatment options provided by these antibodies, immune related adverse effects and the costs to produce viable antibodies are remaining issues in the field.

Small molecules targeting PD-L1 would provide more cost effective options and reduce immune related adverse effects, thereby providing improved cancer treatment options.

The present disclosure addresses these needs and provides related advantages as well.

BRIEF SUMMARY

The present disclosure provides methods of preventing and/or treating cancer in an individual in need thereof, said method comprising administering effective amount of a small molecule programmed death ligand 1 (PD-L1) inhibitor.

In some embodiments, the small molecule PD-L1 inhibitor is a compound of Formula (I)

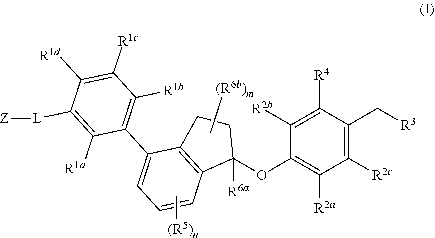

where each variable is described below.

In some embodiments, the small molecule PD-L1 inhibitor has the formula of Compound 1.004

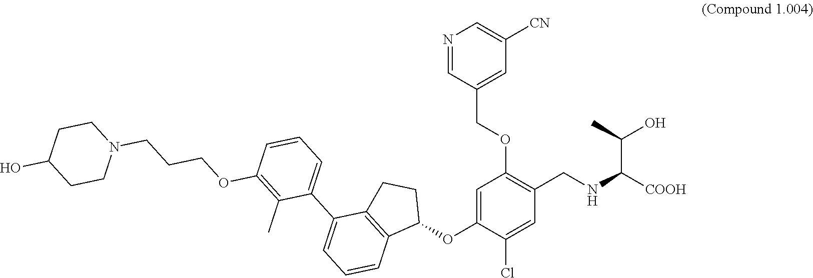 [SMILES: Cc1c(OCCCN2CCC(O)CC2)cccc1-c1cccc2c1CC[C@@H]2Oc1cc(OCc2cncc(C#N)c2)c(CN[C@H](C(=O)O)[C@@H](C)O)cc1Cl]

or a pharmaceutically acceptable salt thereof.

In some embodiments, the small molecule PD-L1 inhibitor has the formula of Compound 1.041

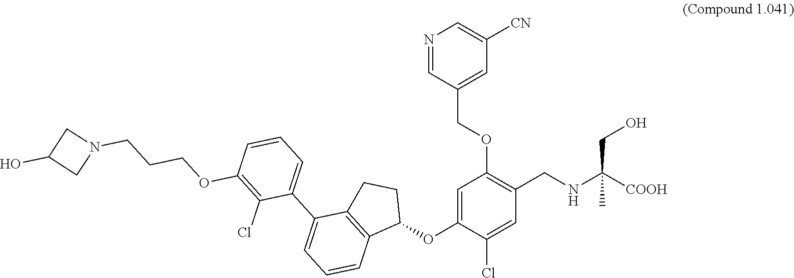

or a pharmaceutically acceptable salt thereof.

In some embodiments, the small molecule PD-L1 inhibitor has the formula of Compound 1.227

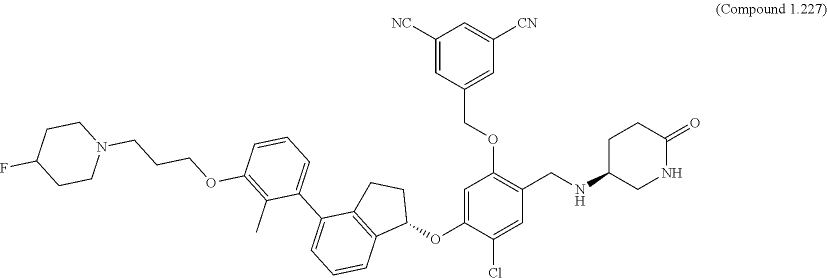 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC(=O)NC2)cc1Cl]

or a pharmaceutically acceptable salt thereof.

In some embodiments, the small molecule PD-L1 inhibitor has the formula of Compound 1.347

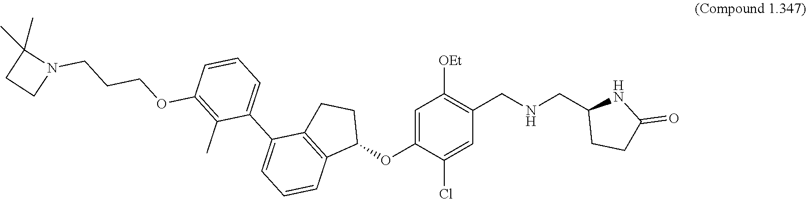 [SMILES: CCOc1cc(O[C@H]2CCc3c(-c4cccc(OCCCN5CCC5(C)C)c4C)cccc32)c(Cl)cc1CNC[C@@H]1CCC(=O)N1]

or a pharmaceutically acceptable salt thereof.

In some embodiments, the cancer is melanoma.

In some aspects, provided herein are methods of preventing and/or treating melanoma in an individual in need thereof, said method including the step of administering an effective amount of a small molecule programmed death ligand 1 (PD-L1) inhibitor selected from the group consisting of

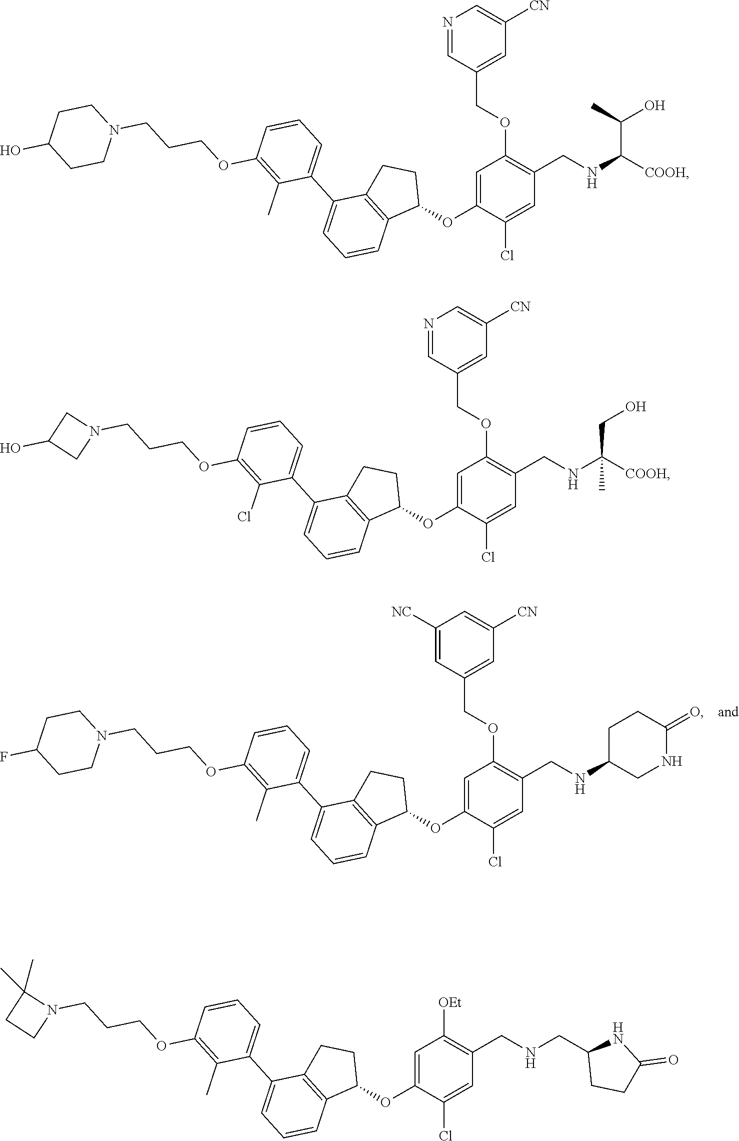

or a pharmaceutically acceptable salt thereof.

In some aspects, provided herein are methods of increasing the CD8+ T cell/CD4+ T cell ratio in a solid tumor microenvironment, said method comprising administering an effective amount of a small molecule programmed death ligand 1 (PD-L1) inhibitor of Formula (I)

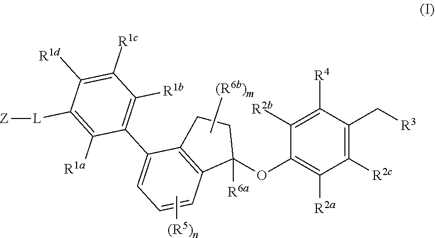

where each variable is described below.

### BRIEF DESCRIPTION OF THE DRAWINGS

FIG. 1A-D reports the relative activity of of Compound 1.041 in various assays: an ELISA Assay (Panel A); a dimerization ELISA assay (Panel B); a Cell-based assay (Panel C); and a mixed lymphocyte reaction assay (Panel D).

FIG. 2A-C demonstrates that Compound 1.041 reverses T-Cell exhaustion phenotype induced by Staphylococcal enterotoxin B. Panel A reports data from a T-Cell exhaustion assay; Panel B reports data from a Proliferation Assay (CellTiter-Glo assay); and Panel C reports data from a PD-L1 Surface Expression Assay. ****p<0.0001, ***p<0.001, **p<0.01, n=3, (x3).

FIG. 3A-B reports data from a flow cytometry assay determining PD-L1 expression levels on break tumor cells (MDA-MB-231). Panel A shows that PD-L1 expression decreases when Compound 1.041 is provided. Panel B shows that a similar effect is not seen when an anti-PD-L1 antibody is provided.

FIG. 4 illustrates the study design used for a prophylatic dosing of Compound 1.041 mouse model.

FIG. 5A-H plots the tumor volume measured for each mouse in the prophylactic dosing study, each panel represents a different treatment cohort: A375 cells alone with vehicle treatment (Panel A); A375 cells+hPBMCs with vehicle treatment (Panel B); A375 cells alone with Compound 1.041 treatment (Panel C); A375 cells+hPBMCs with Compound 1.041 treatment (Panel D); A375 cells alone with isotype treatment (Panel E); A375 cells+hPBMCs with isotype treatment (Panel F); A375 cells alone with anti-PD-L1 treatment (Panel G); A375 cells+hPBMCs with anti-PD-L1 treatment (Panel H). “TF” refers to “tumor free.”

FIG. 6 illustrates the study design used for a therapeutic dosing of Compound 1.041 mouse model.

FIG. 7 plots the relative tumor growth for each cohort in the therapeutic dosing study. **p<0.01

FIG. 8A-D plos the growth percentage for each tumor in the therapeutic dosing study, each panel represents a different treatment cohort: Vehicle (Panel A); Compound 1.041 (Panel B); Isotype antibody control (Panel C); anti-PD-L1 (Panel D).

FIG. 9A-C provide flow cytometry data demonstrating that Compound 1.041 increases the percentage of hCH8⁺ T-cells relative to hCH4⁺ T-cells as compared to vehicle control. Panel A reports the percent of hCD4⁺/hCD45⁺ cells in vehicle and Compound 1.041 treatment groups; Panel B reports the percent of hCD8⁺/hCD45⁺ cells in vehicle and Compound 1.041 treatment groups; Panel C reports the ration of hCH8⁺/hCD4⁺ T-Cells in vehicle and Compound 1.041 treatment groups.

FIG. 10 plots the mean fluorescence intensity of PD-1 of vehicle and Compound 1.041 treatment groups. Compound 1.041 treatment groups show a reduction in PD-1 expression suggesting these cells are less exhausted compared to cells from the vehicle treatment group.

### DETAILED DESCRIPTION OF THE INVENTION

### I. GENERAL

The present disclosure is drawn, in part, to the finding that a compound of Formula (I) (a PD-L1 inhibitor) can be used to effectively treat cancers in vivo. In particular, the compounds of the present disclosure reduce tumor growth to a similar extent as an anti-human PD-L1 antibody. Thus, the current disclosure provides the advantageous property of PD-L1 inhibition and the associated T cell modulation without the immune complications or costs associated with biologic therapeutics.

### II. ABBREVIATIONS AND DEFINITIONS

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “alkyl”, by itself or as part of another substituent, means, unless otherwise stated, a straight or branched chain hydrocarbon group, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of alkenyl groups include vinyl, 2-propenyl, crotyl, 2-isopentenyl, 2-(butadienyl), 2,4-pentadienyl and 3-(1,4-pentadienyl). Examples of alkynyl groups include ethynyl, 1- and 3-propynyl, 3-butynyl, and the higher homologs and isomers. The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc. The bicyclic or polycyclic rings may be fused, bridged, spiro or a combination thereof. The term “heterocycloalkyl” or “heterocyclyl” refers to a cycloalkyl group that contain from one to five heteroatoms selected from N, O, and S, wherein the nitrogen and sulfur atoms are optionally oxidized, and the nitrogen atom(s) are optionally quaternized. The heterocycloalkyl may be a monocyclic, a bicyclic or a polycylic ring system. The bicyclic or polycyclic rings may be fused, bridged, spiro or a combination thereof. It is understood that the recitation for C₄₋₁₂ heterocyclyl, refers to a group having from 4 to 12 ring members where at least one of the ring members is a heteroatom. Non limiting examples of heterocycloalkyl groups include pyrrolidine, imidazolidine, pyrazolidine, butyrolactam, valerolactam, imidazolidinone, tetrazolone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, and the like. A heterocycloalkyl group can be attached to the remainder of the molecule through a ring carbon or a heteroatom.

The term “alkylene” by itself or as part of another substituent means a divalent group derived from an alkane, as exemplified by —CH₂CH₂CH₂CH₂—. Typically, an alkyl (or alkylene) group will have from 1 to 12 carbon atoms, with those groups having 8 or fewer carbon atoms being preferred in the present disclosure. Similarly, “alkenylene” and “alkynylene” refer to the unsaturated forms of “alkylene” having double or triple bonds, respectively.

The term “heteroalkyl,” by itself or in combination with another term, means, unless otherwise stated, a stable straight or branched chain, or cyclic hydrocarbon group, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the terms “heteroalkenyl” and “heteroalkynyl” by itself or in combination with another term, means, unless otherwise stated, an alkenyl group or alkynyl group, respectively, that contains the stated number of carbons and having from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group.

The term “heteroalkylene” by itself or as part of another substituent means a divalent group, saturated or unsaturated or polyunsaturated, derived from heteroalkyl, as exemplified by —CH₂—CH₂—S—CH₂CH₂— and —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄haloalkyl” is mean to include trifluoromethyl, 2,2,2-trifluoroethyl, 4-chlorobutyl, 3-bromopropyl, and the like.

The term “hydroxyalkyl” or “alkyl-OH” refers to an alkyl group, as defined above, where at least one (and up to three) of the hydrogen atoms is replaced with a hydroxy group. As for the alkyl group, hydroxyalkyl groups can have any suitable number of carbon atoms, such as C₁₋₆. Exemplary hydroxyalkyl groups include, but are not limited to, hydroxymethyl, hydroxyethyl (where the hydroxy is in the 1- or 2-position), hydroxypropyl (where the hydroxy is in the 1,- 2- or 3-position), and 2,3-dihydroxypropyl.

The term “C₁₋₃ alkyl-guanidinyl” refers to a C₁₋₃ alkyl group, as defined above, where at least one of the hydrogen atoms is replaced with a guanidinyl group (—NHC(NH)NH₂).

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. It is understood that the recitation for C₅₋₁₀ heteroaryl, refers to a heteroaryl moiety having from 5 to 10 ring members where at least one of the ring members is a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for each of the above noted aryl and heteroaryl ring systems are selected from the group of acceptable substituents described below.

The term “carbocyclic ring,” “carbocyclic” or “carbocyclyl” refers to cyclic moieties with only carbon atoms as ring vertices. Carbocyclic ring moieties are saturated or unsaturated and can be aromatic. Generally, carbocyclic moieties have from 3 to 10 ring members. Carbocylic moieties with multiple ring structure (e.g. bicyclic) can include a cycloalkyl ring fused to an aromatic ring (e.g. 1,2,3,4-tetrahydronaphthalene). Thus, carbocyclic rings include cyclopentyl, cyclohexenyl, naphthyl, and 1,2,3,4-tetrahydronaphthyl. The term “heterocyclic ring” refers to both “heterocycloalkyl” and “heteroaryl” moieties. Thus, heterocyclic rings are saturated or unsaturated and can be aromatic. Generally, heterocyclic rings are 4 to 10 ring members and include piperidinyl, tetrazinyl, pyrazolyl and indolyl.

When any of the above terms (e.g., “alkyl,” “aryl” and “heteroaryl”) are referred to as ‘substituted’ without further notation on the substituents, the substituted forms of the indicated group will be as provided below.

Substituents for the alkyl groups (including those groups often referred to as alkylene, alkenyl, alkynyl and cycloalkyl) can be a variety of groups selected from: —halogen, —OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂ in a number ranging from zero to (2 m′+1), where m′ is the total number of carbon atoms in such group. R′, R″ and R″′ each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. The term “acyl” as used by itself or as part of another group refers to an alkyl group wherein two substitutents on the carbon that is closest to the point of attachment for the group is replaced with the substitutent ═O (e.g., —C(O)CH₃, —C(O)CH₂CH₂OR′ and the like).

Similarly, substituents for the aryl and heteroaryl groups are varied and are generally selected from: —halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R″′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —N_{3,} perfluoro(C₁-C₄)alkoxy, and perfluoro(C₁-C₄)alkyl, in a number ranging from zero to the total number of open valences on the aromatic ring system; and where R′, R″ and R″′ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula —T-C(O)—(CH₂)_{q}—U—, wherein T and U are independently —NH—, —O—, —CH ₂— or a single bond, and q is an integer of from 0 to 2. Alternatively, two of the substituents on adjacent atoms of the aryl or heteroaryl ring may optionally be replaced with a substituent of the formula —A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

The disclosure herein further relates to prodrugs and bioisosteres thereof. Suitable bioisosteres, for example, will include carboxylate replacements (phosphonic acids, phosphinic acids, sulfonic acids, sulfinic acids, and acidic heterocyclic groups such as tetrazoles). Suitable prodrugs will include those conventional groups known to hydrolyze and/or oxidize under physiological conditions to provide a compound of Formula I.

The terms “patient” and “subject” include primates (especially humans), domesticated companion animals (such as dogs, cats, horses, and the like) and livestock (such as cattle, pigs, sheep, and the like).

As used herein, the term “treating” or “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms).

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. When a stereochemical depiction is shown, it is meant to refer to the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are intended to be encompassed within the scope of the present disclosure. For example, the compounds may be prepared such that any number of hydrogen atoms are replaced with a deuterium (²H) isotope. The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. All isotopic variations of the compounds of the present disclosure, whether radioactive or not, are intended to be encompassed within the scope of the present disclosure.

As used herein, the term “solid tumor” refers to a malignant neoplasm. A solid tumors is generally localized mass of tissue; however, solid tumors are able to invade surrounding tissue and metastasize to new body sides. Solid tumors may be benign (not cancer), or malignant (cancer). Different types of solid tumors are named for the type of cells that form them. Examples of solid tumors are sarcomas, carcinomas, and lymphomas. The term “solid tumor” does not include leukemia. (cancers of the blood). “Sarcomas” are cancers arising from connective or supporting tissues such as bone or muscle. “Carcinomas” are cancers arising from glandular cells and epithelial cells, which line body tissues. “Lymphomas” are cancers of the lymphoid organs such as the lymph nodes, spleen, and thymus. As these cells occur in most tissues of the body, lymphomas may develop in a wide variety of organs. Exemplary solid tumors include but are not limited to sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioblastoma multiforme, astrocytoma, medulloblastoma, craniopharyngioma, ependymoma, pinealoma, hemangioblastoma, acoustic neuroma, oligodendroglioma, cutaneous T cell lymphoma (CTCL), melanoma, neuroblastoma, and retinoblastoma.

### III. DETAILED DESCRIPTION OF EMBODIMENTS

A. Methods

In one aspect, the present disclosure provides methods of preventing and/or treating cancer in an individual in need thereof, said method comprising administering an effective amount of a small molecule programmed death ligand 1 (PD-L1) inhibitor.

The PD-L1 inhibitors of the present disclosure are useful antagonists of the PD-1/PD-L1 protein protein interaction. In some embodiments, the compounds of the disclosure may be used to inhibit VISTA and/or TIM-3. In some embodiments, the PD-L1 inhibitors of the disclosure may be inhibitors of the PD-1/PD-L1 protein protein interaction and inhibitors of VISTA and/or TIM-3. In some embodiments, in addition to being inhibitors of the PD-1/PD-L1 protein protein interaction, the compounds of the disclosure may be inhibitors of CTLA-4 and/or BTLA and/or LAG-3 and/or KLRG-1 and/or 2B4 and/or CD160 and/or HVEM and/or CD48 and/or E-cadherin and/or MHC-II and/or galectin-9 and/or CD86 and/or PD-L2 and/or VISTA and/or TIM-3 and/or CD80.

In some embodiments, the small molecule PD-L1 inhibitor is a compound of Formula (I)

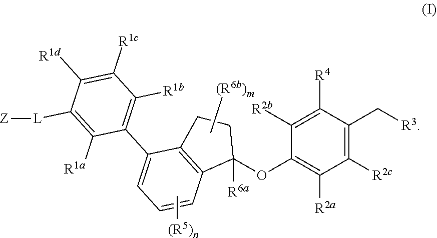

Embodiments of the small molecule PD-L1 inhibitors provided herein are further discussed in subsection B of this disclosure.

PD-L1 is a ligand of programed cell death protein-1 (PD-1), an immune checkpoint protein. The PD-1/PD-L1 immune checkpoint axis is a key component in modulating T-cell regulation. When PD-1 is associated with PD-L1, the anticancer activity of T cells are down regulated. By providing an inhibitor targeting PD-L1, this association is blocked, allowing for the upregulation of T cell anticancer activity. Accordingly, small molecules effectively targeting PD-L1 and altering PD-1/PD-L1 association in vivo provide an excellent means for improving cancer treatments in a number of patients.

In some embodiments the cancer is a solid tumor. In some embodiments the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendo-theliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioblastoma multiforme, astrocytoma, medulloblastoma, craniopharyngioma, ependymoma, pinealoma, hemangioblastoma, acoustic neuroma, oligodendroglioma, melanoma, neuroblastoma, and retinoblastoma.

In some embodiments the solid tumor is a melanoma.

As shown herein, upon treatment with a small molecule PD-L1 inhibitor of the present disclosure to individuals with cancer, tumor growth is reduced to a similar extent as an anti-human PD-L1 antibody.

In a second aspect, the present disclosure provides methods of preventing and/or treating melanoma in an individual in need thereof, said method comprising administering an effective amount of a small molecule programmed death ligand 1 (PD-L1) inhibitor selected from the group consisting of

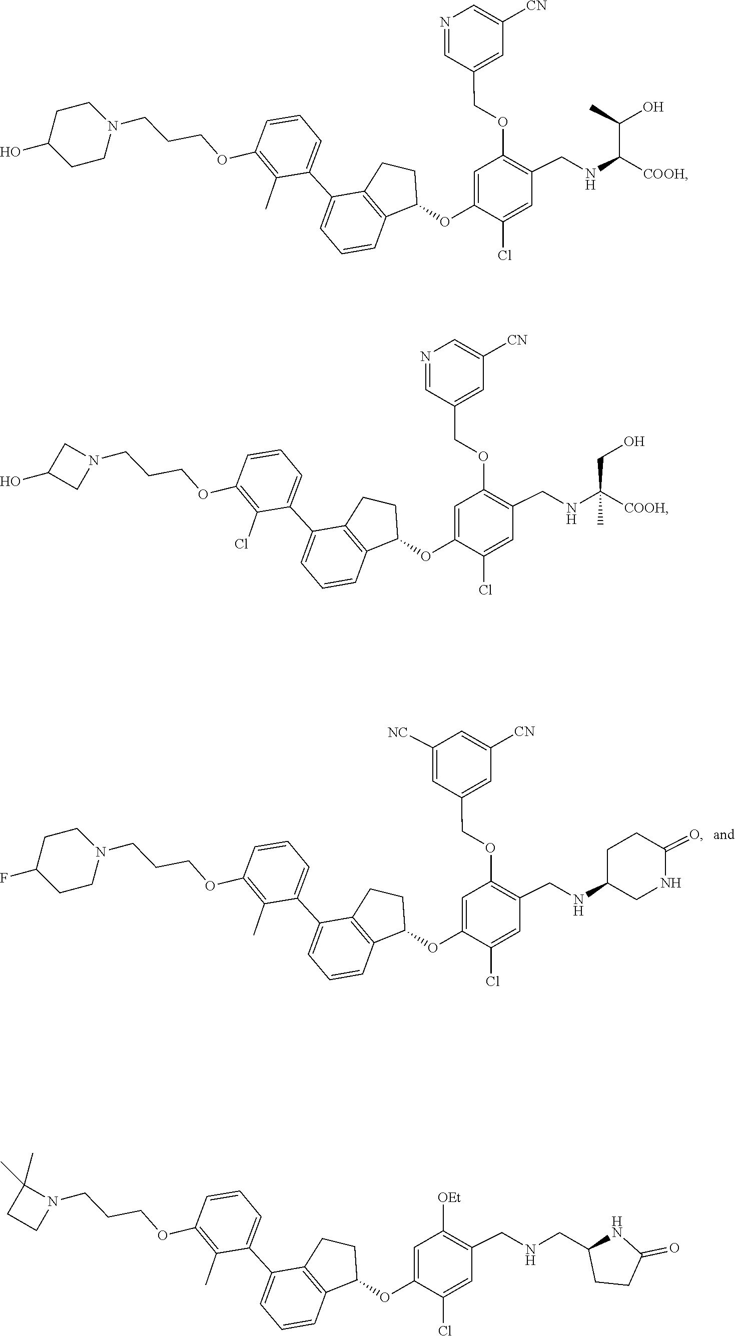

or a pharmaceutically acceptable salt thereof.

In a third aspect, the present disclosure provides methods of increasing the CD8+ T cell/CD4+ T cell ratio in a solid tumor microenvironment, said method comprising administering effective amount of a small molecule PD-L1 inhibitor.

B. Small Molecule PD-L1 Inhibitors

In some embodiments, the small molecule PD-L1 inhibitors are compounds having the formula (I):

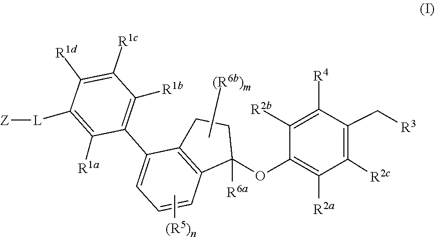

or a pharmaceutically acceptable salt thereof, or a prodrug or bioisostere thereof;

wherein:
each of R^{1a}, R^{1b} R^{1c} and R^{1d} is independently selected from the group consisting of H, halogen, CF₃, CN, C₁₋₄ alkyl and —O—Ci₁₋₄ alkyl, wherein the C₁₋₄ alkyl and —O—C₁₋₄ alkyl are optionally further substituted with halogen, hydroxyl, methoxy or ethoxy;
   L is a linking group selected from the group consisting of:

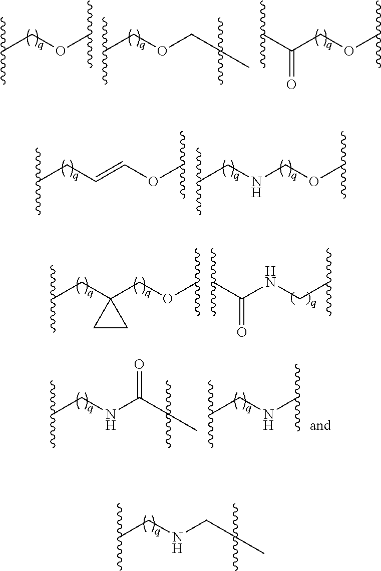

wherein each of the subscripts q is independently 1, 2, 3 or 4, and L is optionally further substituted with one or two members selected from the group consisting of halogen, hydroxy, C₁₋₃ alkyl, —O—C₁₋₃ alkyl, C₁₋₃ hydroxyalkyl, C₁₋₃ haloalkyl and —CO₂H;
Z is selected from the group consisting of azetidinyl, pyrollidinyl, piperidinyl, morpholinyl, pyridyl, pyrimidinyl, guanidinyl, quinuclidine, and 8-azabicyclo[3.2.1]octane, each of which is optionally substituted with from 1 to 3 groups independently selected from halogen, hydroxy, C₁₋₃ alkyl, —NH₂, —NHC₁₋₃alkyl, —N(C₁₋₃alkyl)₂, —O—C₁₋₃ alkyl, C₁₋₃ hydroxyalkyl, C₁₋₃ haloalkyl and —CO₂H;
or
Z is selected from the group consisting of —CO₂R^{a} and —NR^{a}R^{b}; wherein R^{a} is selected from the group consisting of H, C₁₋₈ alkyl, C₁₋₈ haloalkyl and C₁₋₈ hydroxyalkyl; and R^{b} is selected from —C₁₋₈ alkyl, C₁₋₈ haloalkyl, C₁₋₈ alkyl-COOH, C₁₋₈ alkyl-OH, C₁₋₈ alkyl-CONH₂, C₁₋₈ alkyl-SO₂NH₂, C₁₋₈alkyl-PO₃H₂, C₁₋₈ alkyl-C(O)NHOH, —C(O)—C₁₋₈alkyl—OH, —C(O)—C₁₋₈alkyl-COOH, C₃₋₁₀ cycloalkyl, —C₃₋₁₀ cycloalkyl-COOH, —C₃₋₁₀ cycloalkyl-OH, C₄₋₈ heterocyclyl, —C₄₋₈ heterocyclyl-COOH, —C₄₋₈heterocyclyl-OH, —C₁₋₈ alkyl-C₄₋₈ heterocyclyl, —C₁₋₈ alkyl-C₃₋₁₀ cycloalkyl, C₅₋₁₀ heteroaryl and —C₁₋₈alkyl-C₅₋₁₀ heteroaryl;
each R^{2a}, R^{2b} and R^{2c} is independently selected from the group consisting of H, halogen, —CN, —R^{d}, —CO₂R^{e}, —CONR^{e}R^{f}, —OC(O)NR^{e}R^{f}, —NR^{f}C(O)R^{e}, —NR^{f}C(O)₂R^{d}, —NR^{e}—C(O)NR^{e}R^{f}, —NR^{e}—OR^{e}, —X²—OR³, —X²—NR^{e}R^{f}, —X²—CO₂R^{e}, —SF₅, and —S(O)₂NR^{e}R^{f}, wherein each X² is a C₁₋₄ alkylene; each R^{e} and R^{f} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O and S, and optionally substituted with oxo; each R^{d} is independently selected from the group consisting of C₁₋₈ alkyl, C₂₋₈ alkenyl, and C₁₋₈ haloalkyl;
R³ is selected from the group consisting of —NR^{g}R^{h} and C₄₋₁₂ heterocyclyl, wherein the C₄₋₁₂ heterocyclyl is optionally substituted with 1 to 6 R^{3a};
each R^{3a} is independently selected from the group consisting of halogen, —CN, —Rⁱ, —CO₂R^{j}, —CONR^{j}R^{k}, —CONHC₁₋₆alkyl-OH, —C(O)R^{j}, —OC(O)NR^{j}R^{k}, —NR^{j}C(O)R^{k}, -NR^{j}C(O)₂R^{k}, —CONHOH, PO₃H₂, —NR^{j}X³—C(O)₂R^{k}, —NR^{j}C(O)NR^{j}R^{k}, —NR^{j}R^{k}, —OR^{j}, —S(O)₂NR^{j}R^{k}, —O—X³—OR^{j}, —O—X³—NR^{j}R^{k}, —O—X³—CO₂R^{j}, —O—X³—CONR^{j}R^{k}, —X³—OR^{j}, —X³—NR^{j}R^{k}, —X³—CO₂R^{j}, —X³—CONR^{j}R^{k}, and SF₅; wherein X³ is C₁₋₆ alkylene and is optionally further substituted with OH, SO₂NH₂, CONH₂, C(O)NHOH, PO₃H₂, COO—C₁₋₈alkyl or CO₂H, wherein each R^{j} and R^{k} is independently selected from hydrogen, C₁₋₈ alkyl optionally substituted with 1 to 2 substituents selected from OH, SO₂NH₂, CONH₂, C(O)NHOH, PO₃H₂, COO—C₁₋₈alkyl or CO₂H, and C₁₋₈ haloalkyl optionally substituted with 1 to 2 substituents selected from OH, SO₂NH₂, CONH₂, C(O)NHOH, PO₃H₂, COO—C₁₋₈alkyl or CO₂H, or when attached to the same nitrogen atom R^{j} and R^{k} can be combined with the nitrogen atom to form a five or six-membered ring having from 0 to 2 additional heteroatoms as ring members selected from N, O or S, and optionally substituted with oxo; each Rⁱ is independently selected from the group consisting of —OH, C₁₋₈ alkyl, C₂₋₈ alkenyl, and C₁₋₈haloalkyl each of which may be optionally substituted with OH, SO₂NH₂, CONH₂, C(O)NHOH, PO₃H₂, COO—C₁₋₈alkyl or CO₂H;
R^{g} is selected from the group consisting of H, C₁₋₈ haloalkyl and C₁₋₈ alkyl;
R^{h} is selected from —C₁₋₈alkyl, C₁₋₈haloalkyl, C₁₋₈hydroxyalkyl, C₁₋₈alkyl-CO₂R^{j}, C₁₋₈alkyl-CONR^{j}R^{k}, and C₁₋₈alkyl-CONHSO₂R^{j}, C₁₋₈alkyl-SO₂NR^{j}R^{k}, C₁₋₈alkyl-PO₃H₂, C₁₋₈alkyl-C(O)NHOH, C₁₋₈ alkyl-NR^{h1}R^{h2}, —C(O)R^{j}, C₃₋₁₀ cycloalkyl, —C₃₋₁₀ cycloalkyl-COOR^{j}, —C₃₋₁₀ cycloalkyl-OR^{j}, C₄₋₈ heterocyclyl, —C₄₋₈ heterocyclyl-COOR^{j}, —C₄₋₈ heterocyclyl-OR^{j}, —C₁₋₈ alkyl-C₄₋₈ heterocyclyl, —C(═O)OC₁₋₈ alkyl-C₄₋₈ heterocyclyl, —C₁₋₈ alkyl-C₃₋₁₀ cycloalkyl, C₅₋₁₀ heteroaryl, —C₁₋₈alkyl-C₅₋₁₀ heteroaryl, —C₁₋₈ alkyl-C₆₋₁₀ aryl, —C₁₋₈ alkyl—(C═O)—C₆₋₁₀ aryl, —CO₂-13 C₁₋₈ alkyl—O₂C—C₁₋₈ alkyl, —C₁₋₈ alkyl-NH(C═O)—C₂₋₈ alkenyl , —C₁₋₈ alkyl-NH(C═O)—C₁₋₈ alkyl, —C₁₋₈ alkyl-NH(C═O )—C₂₋₈ alkynyl, —C₁₋₈ alkyl-(C═O)—NH—C₁₋₈ alkyl-COOR^{j}, and —C₁₋₈alkyl-(C═O)—NH—C₁₋₈ alkyl-OR^{j} optionally substituted with CO2H; or

R^{h} combined with the N to which it is attached is a mono-, di- or tri-peptide comprising 1-3 natural amino acids and 0-2 non-natural amino acids, wherein

the non-natural aminoacids have an alpha carbon substituent selected from the group consisting of C₂₋₄ hydroxyalkyl, C₁₋₃ alkyl-guanidinyl, and C₁₋₄ alkyl-heteroaryl,

the alpha carbon of each natural or non-natural amino acids are optionally further substituted with a methyl group, and

the terminal moiety of the mono-, di-, or tri-peptide is selected from the group consisting of C(O)OH, C(O)O—C₁₋₆ alkyl, and PO₃H₂, wherein

R^{h1} and R^{h2} are each independently selected from the group consisting of H, C₁₋₆ alkyl, and C₁₋₄ hydroxyalkyl;

the C₁₋₈ alkyl portions of R^{h} are optionally further substituted with from 1 to 3 substituents independently selected from OH, COOH, SO₂NH₂, CONH₂, C(O)NHOH, COO—C₁₋₈ alkyl, PO₃H₂ and C₅₋₆ heteroaryl optionally substituted with 1 to 2 C₁₋₃ alkyl substituents,

the C₅₋₁₀ heteroaryl and the C₆₋₁₀ aryl portions of R^{h} are optionally substituted with 1 to 3 substituents independently selected from OH, B(OH)₂, COOH, SO₂NH₂, CONH₂, C(O)NHOH, PO₃H₂, COO—C₁₋₈alkyl, C₁₋₄alkyl, C₁₋₄alkyl-OH, C₁₋₄alkyl-SO₂NH₂, C₁₋₄alkyl-CONH₂, C₁₋₄alkyl-C(O)NHOH, C₁₋₄alkyl- PO₃H₂, C₁₋₄alkyl-COOH, and phenyl and the C₄₋₈ heterocyclyl and C₃₋₁₀ cycloalkyl portions of R^{h} are optionally substituted with 1 to 4 R^{w} substituents;
each R^{w} substituent is independently selected from C₁₋₄ alkyl, C₁₋₄ alkyl-OH, C₁₋₄ alkyl-COOH, C₁₋₄ alkyl-SO₂NH₂, C₁₋₄ alkyl CONH₂, C₁₋₄ alkyl-C(O)NHOH, C₁₋₄ alkyl-PO₃H, OH, COO—C₁₋₈ alkyl, COOH, SO₂NH₂, CONH₂, C(O)NHOH, PO₃H₂ and oxo;
R⁴ is selected from the group consisting of O—C₁₋₈ alkyl, O—C₁₋₈ haloalkyl, C₆₋₁₀ aryl, C₅₋₁₀ heteroaryl , —O—C₁₋₄ alkyl-C₄₋₇ heterocycloalkyl, —O—C₁₋₄ alkyl-C₆₋₁₀aryl and —O—C₁₋₄ alkyl-C₅₋₁₀ heteroaryl, each of which is optionally substituted with 1 to 5 R^{4a};
each R^{4a} is independently selected from the group consisting of halogen, —CN, —R^{m}, —CO₂Rⁿ, —CONRⁿR^{p}, —C(O)Rⁿ, —OC(O)NRⁿR^{p}, —NRⁿC(O)R^{p}, —NRⁿC(O)₂R^{m}, —NRⁿ—C(O)NRⁿR^{p}, —NRⁿR^{p}, —ORⁿ, —O—X⁴—ORⁿ, —O—X⁴—NRⁿR^{p}, —O—X⁴—CO₂Rⁿ, —O—X⁴-CONRⁿR^{p}, —X⁴—ORⁿ, —X⁴—NRⁿR^{p}, —X⁴—CO₂Rⁿ, —X⁴—CONRⁿR^{p}, —SF₅, —S(O)₂RⁿR^{p}, —S(O)₂NRⁿR^{p}, C₃₋₇ cycloalkyl and C₄₋₇ heterocycloalkyl, wherein the cycloalkyl and heterocycloalkyl rings are optionally substituted with 1 to 5 R^{t}, wherein each R^{t} is independently selected from the group consisting of C₁₋₈ alkyl, C₁₋₈haloalkyl, —CO₂Rⁿ, —CONRⁿR^{p}, —C(O)Rⁿ, —OC(O)NRⁿR^{p}, —NRⁿC(O)R^{p}, —NRⁿC(O )₂R^{m}, —NRⁿ—C(O)NRⁿR^{p}, —NRⁿR^{p}, —ORₙ, —O—X⁴—ORⁿ, —O—X⁴—NRⁿR^{p},—O—X⁴—CO₂Rⁿ, —O—X⁴—CONRⁿR^{p}, —X⁴—ORⁿ, —X⁴—NRⁿR^{p}

, —X⁴—CO₂Rⁿ, —X⁴—CONRⁿR^{p}, —SF₅, and —S(O)₂NRⁿR^{p};
wherein each X⁴ is a C₁₋₆ alkylene; each Rⁿ and R^{p} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each R^{m} is independently selected from the group consisting of C₁₋₈ alkyl, C₂₋₈ alkenyl, and C₁₋₈haloalkyl; and optionally when two R^{4a} substituents are on adjacent atoms, they are combined to form a fused five or six-membered carbocyclic or heterocyclic ring optionally substituted with oxo;
n is 0, 1, 2 or 3;
each R⁵ is independently selected from the group consisting of halogen, —CN, —R^{q}, —CO₂R^{r}, —CONR^{r}R^{s}, —C(O)R^{r}, —OC(O )NR^{r}R^{s}, —NR^{r}C(O)R^{s}, —NR^{r}C(O)₂R^{q}, —NR^{r}—C(O)NR^{r}R^{s}, —NR^{r}R^{s}, —OR^{r}, —O—X⁵—OR^{r}, —O—⁵—NR^{r}R^{s}, —O—X⁵—CO₂R^{r}—O—X⁵—CONR^{r}R^{s}, —X⁵—OR^{r}, —X⁵—NR^{r}R^{s}, —X⁵-CO₂Rᵣ, —X⁵—CONR^{r}R^{s}, —SF₅, —S(O)₂NR^{r}R^{s}, wherein each X⁵ is a C₁₋₄ alkylene; each R^{r} and R^{s} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each R^{q} is independently selected from the group consisting of C₁₋₈ alkyl, and C₁₋₈ haloalkyl;
R^{6a} is selected from the group consisting of H, C₁₋₄ alkyl and C₁₋₄ haloalkyl;
m is 0, 1, 2, 3 or 4;
each R^{6b} is independently selected from the group consisting of F, C₁₋₄ alkyl, O—R^{u}, C¹⁻⁴ haloalkyl, NR^{u}R^{v}, wherein each R^{u} and R^{v} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo.

In some embodiments, the small molecule PD-L1 inhibitors have a formula (Ia) or (Ib):

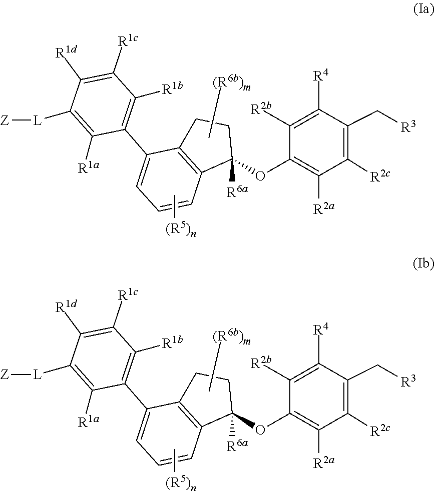

In some embodiments, the small molecule PD-L1 inhibitors have a formula (Ia1) or (Ia2):

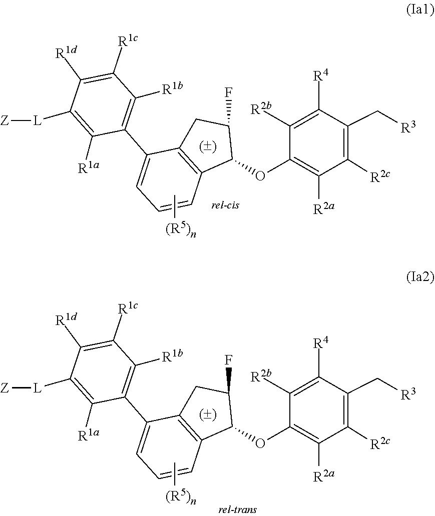

In some embodiments, for each of formula (I), (Ia), (Ia1), (Ia2) and (lb), or a pharmaceutically acceptable salt thereof, each of R^{2a}, R^{2b} and R^{2c} is independently selected from the group consisting of hydrogen, halogen, CN, C₁₋₄ alkyl, and C₁₋₄ haloalkyl.

In some embodiments, for each of formula (I), (Ia), (Ia1), (Ia2) and (lb), or a pharmaceutically acceptable salt thereof, R^{2b} and R²C are both H and R^{2a} is selected from the group consisting of halogen, C₁₋₄ alkyl, C₂₋₄ alkenyl, C₁₋₃haloalkyl, —CN, —OMe and OEt. In some embodiments, R^{2b} and R^{2c}are both H and R^{2a} is halogen. In some embodiments, R^{2b} and R^{2c} are both H and R^{2a} is Cl.

In some embodiments, for each of formula (I), (Ia), (Ia1), (Ia2) and (Ib), or a pharmaceutically acceptable salt thereof, R³ is NR^{g}R^{h}. In some embodiments, R³ is selected from the group consisting of:

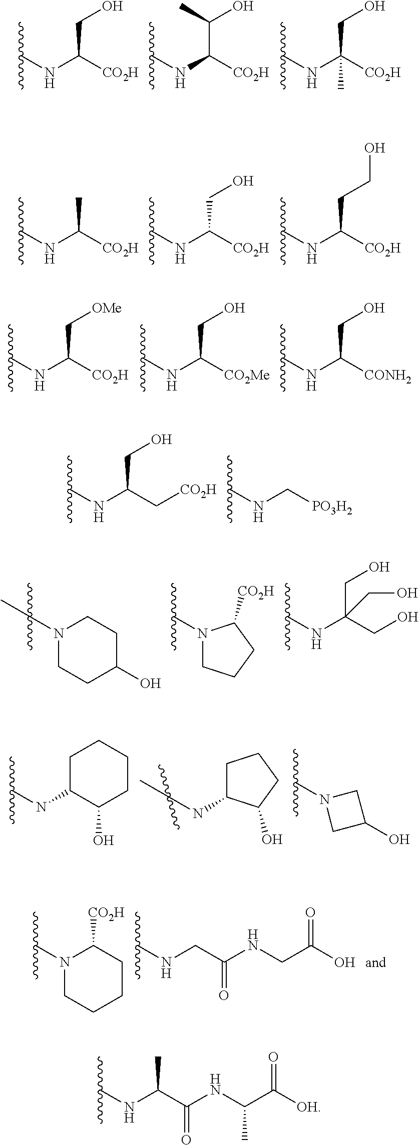

In some embodiments, for each of formula (I), (Ia), (Ia1), (Ia2) and (Ib), or a pharmaceutically acceptable salt thereof, R³ is —NR^{g}R^{h}, and is selected from the group consisting of

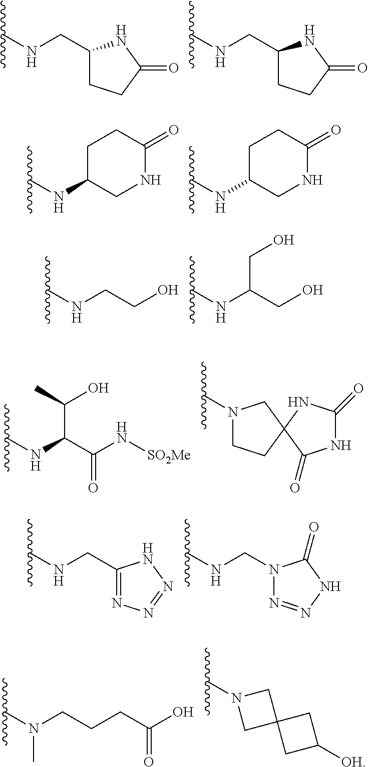

In some embodiments, for each of formula (I), (Ia), (Ia1), (Ia2) and (Ib), or a pharmaceutically acceptable salt thereof, R³ is —NR^{g}R^{h}, and R^{h} combined with the N to which it is attached is a mono-, di- or tri-peptide comprising 1-3 natural amino acids and 0-2 non-natural amino acids, wherein
the non-natural aminoacids have an alpha carbon substituent selected from the group consisting of C₂₋₄ hydroxyalkyl, C₁₋₃ alkyl-guanidinyl, and C₁₋₄ alkly-heteroaryl,
the alpha carbon of each natural or non-natural amino acids are optionally further substituted with a methyl group, and
the terminal moiety of the mono-, di-, or tri-peptide is selected from the group consisting of C(O)OH, C(O)O—C₁₋₆ alkyl, and PO₃H_{2.}

In some embodiments, for each of formula (I), (Ia), (Ia1), (Ia2) and (lb), or a pharmaceutically acceptable salt thereof, each natural amino acid of R^{h} is independently selected from the group consisting of serine, alanine, glycine, lysine, argining, threonine, phenylalanine, tyrosine, asparatate, asparagine, histidine, and leucine.

In some embodiments, for each of formula (I), (Ia), (Ia1), (Ia2) and (Ib), or a pharmaceutically acceptable salt thereof, R⁴ is selected from the group consisting of:

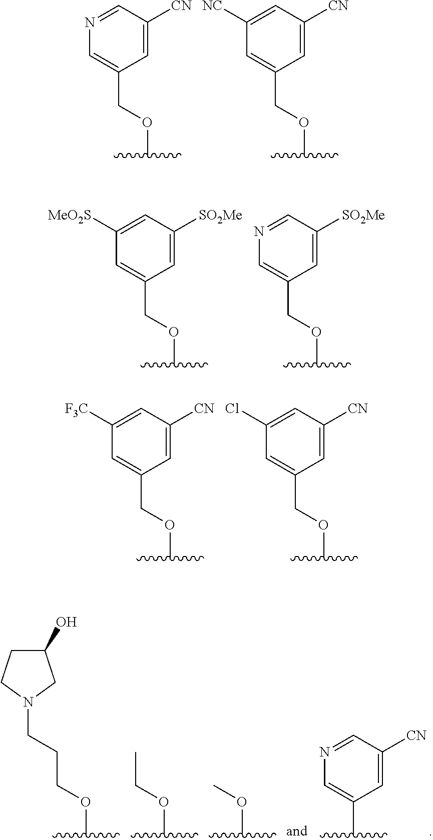

In selected embodiments, R⁴ is selected from the group consisting of:

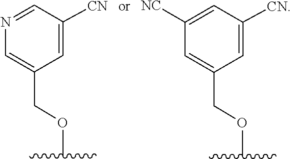 [SMILES: *OCc1cncc(C#N)c1 ; "or" ; *OCc1cc(C#N)cc(C#N)c1]

In some embodiments, for each of formula (I), (Ia), (Ia1), (Ia2) and (Ib), or a pharmaceutically acceptable salt thereof, n is 0.

In some embodiments, for each of formula (I), (Ia), (Ia1), (Ia2) and (Ib), or a pharmaceutically acceptable salt thereof, R^{6a} and R^{6a} are each independently selected from the group consisting of hydrogen, halogen, C₁₋₄ alkyl and C₁₋₄haloalkyl.

In some embodiments, for each of formula (I), (Ia), (Ia1), (Ia2) and (Ib), or a pharmaceutically acceptable salt thereof, the group Z-L- is selected from the group consisting of:

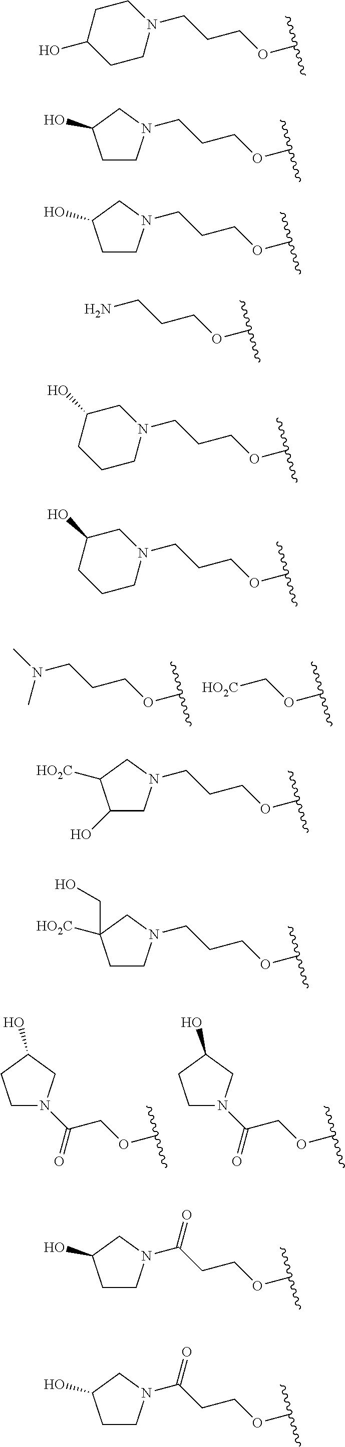

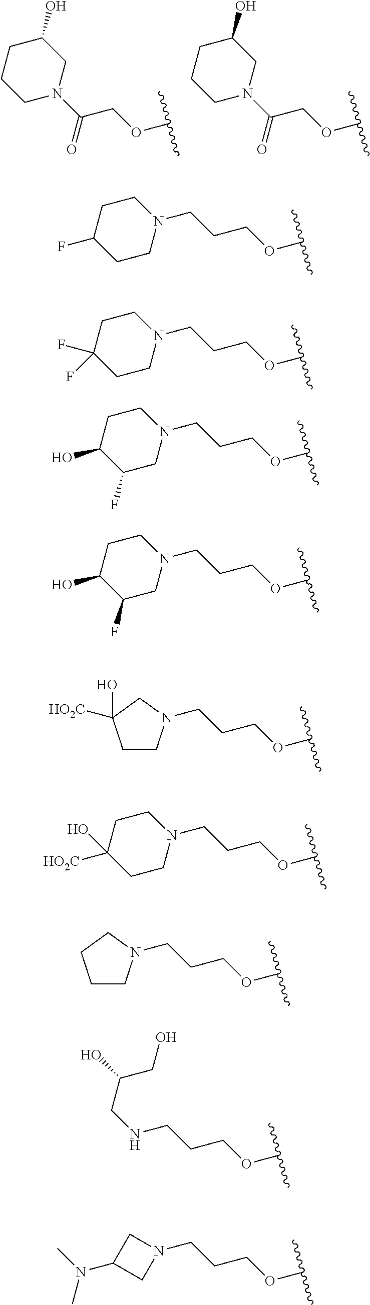

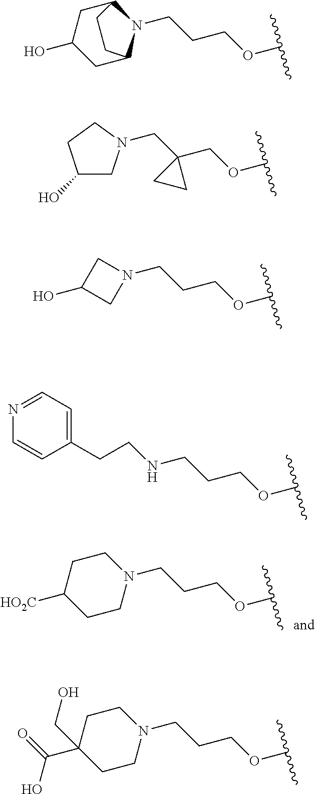

In some embodiments, for each of formula (I), (Ia), (Ia1), (Ia2) and (Ib), or a pharmaceutically acceptable salt thereof, the group Z-L- is selected from the group consisting of:

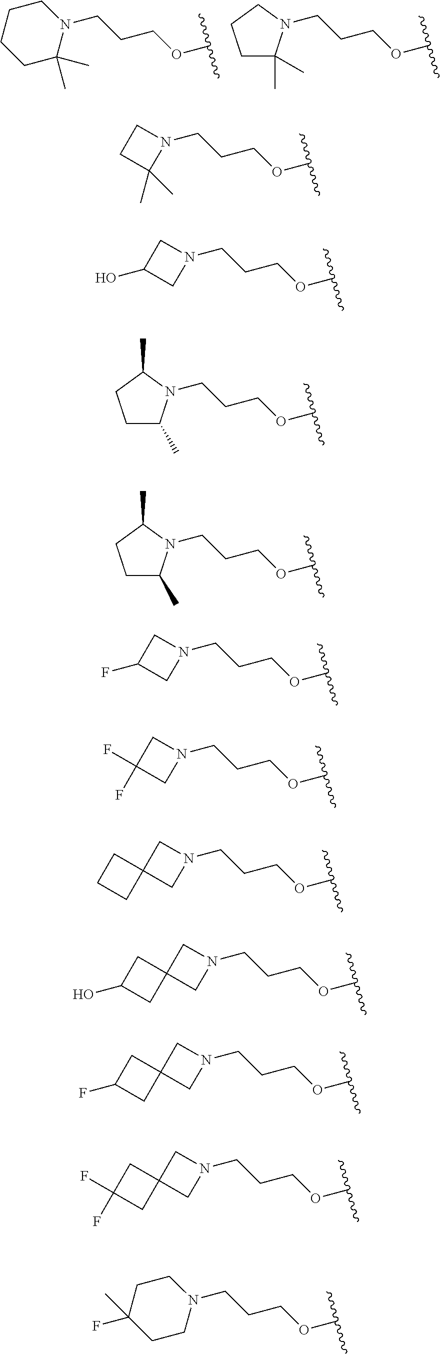

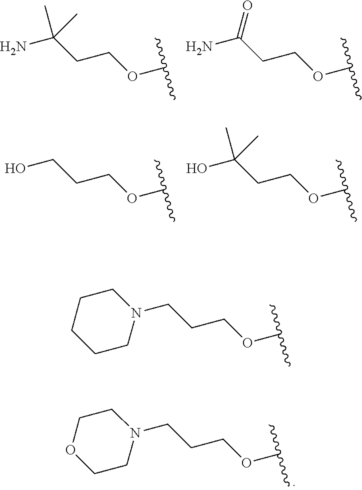

In some embodiments, for each of formula (I), (Ia), (Ia1), (Ia2) and (Ib), or a pharmaceutically acceptable salt thereof, R^{h}a is H.

In some embodiments, for each of formula (I), (Ia) and (Ib), or a pharmaceutically acceptable salt thereof, m is 0.

In some embodiments, for each of formula (I), (Ia) and (Ib), or a pharmaceutically acceptable salt thereof, m is 1 and R^{6b} is selected from the group consisting of F, C₁₋₄ alkyl, O—R^{u}, C₁₋₄haloalkyl and NR^{u}R^{v}, wherein each R^{u} and R^{v} is independently selected from hydrogen, C₁₋₈ alkyl, and C₁₋₈ shaloalkyl.

In some embodiments, for each of formula (I), (Ia) and (Ib), or a pharmaceutically acceptable salt thereof, m is 1 and R^{6b} is F.

In addition to the compounds provided above, pharmaceutically acceptable salts of those compounds are also provided. In some embodiments, the pharmaceutically acceptable salts are selected from ammonium, calcium, magnesium, potassium, sodium, zinc, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, procaine, purines, theobromine, triethylamine, trimethylamine, tripropylamine, tromethamine, hydrochloric, carbonic, monohydrogencarbonic, phosphoric, monohydrogenphosphoric, dihydrogenphosphoric, acetic, propionic, isobutyric, malonic, benzoic, succinic, suberic, fumaric, mandelic, phthalic, benzenesulfonic, p-tolylsulfonic, citric, tartaric, methanesulfonic, arginate, glucuronic acid and galactunoric acids. In some embodiments, the pharmaceutically acceptable salts are selected from ammonium, calcium, magnesium, potassium, sodium, hydrochloric, carbonic, monohydrogencarbonic, phosphoric, monohydrogenphosphoric, dihydrogenphosphoric, acetic, propionic, isobutyric, malonic, benzoic, succinic, suberic, fumaric, mandelic, phthalic, benzenesulfonic, p-tolylsulfonic, citric, tartaric, methanesulfonic, arginate, glucuronic acid and galactunoric acids. In some embodiments, the pharmaceutically acceptable salts are sodium or hydrochloric.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

An ester may be used as a prodrug for the corresponding carboxylic acid. A C₁₋₁₀ alkyl ester or a C₁₋₁₀ haloalkyl ester may be used as a prodrug for the corresponding carboxylic acid. The following esters may be used: ter-butyl ester, methyl ester, ethyl ester, isopropyl ester. More specifically, ester prodrugs may be used as R³ groups such as threonine or serine prodrug esters which are linked to the rest of the molecule through their nitrogen. More specifically, the following prodrugs may be used for R³:

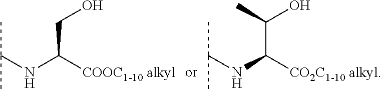

More specifically, the following prodrugs may be used for R³:

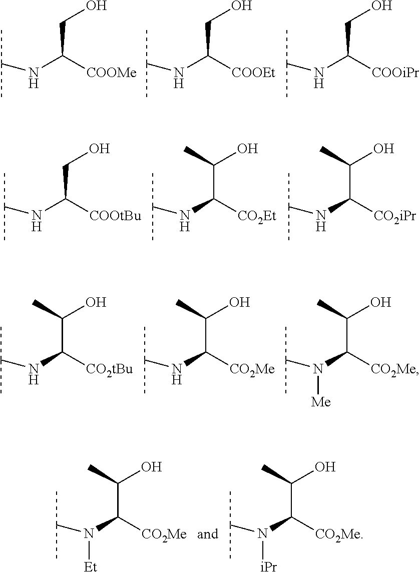

In some embodiments, the small molecule PD-L1 inhibitors are a compound listed in Table 1.

In some embodiments, the small molecule PD-L1 inhibitors are compound having the formula

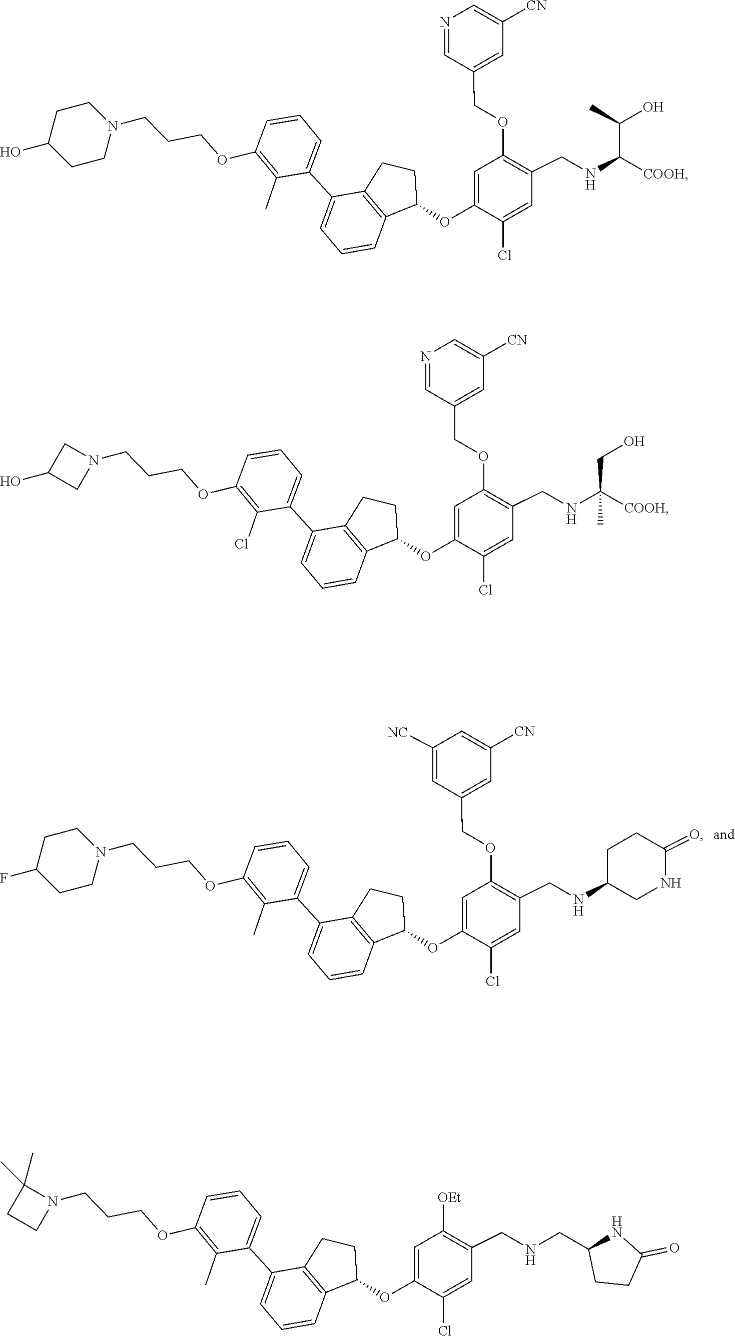

In some embodiments, the small molecule PD-L1 inhibitor has the formula of Compound 1.004

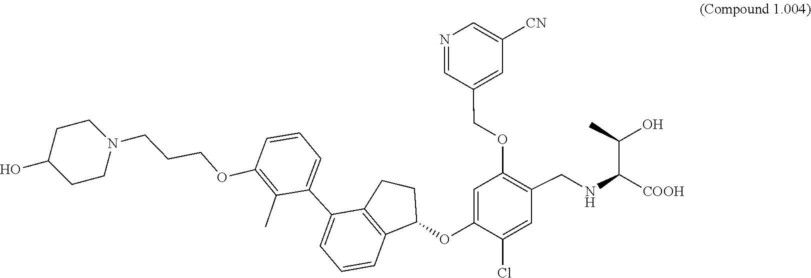 [SMILES: Cc1c(OCCCN2CCC(O)CC2)cccc1-c1cccc2c1CC[C@@H]2Oc1cc(OCc2cncc(C#N)c2)c(CN[C@H](C(=O)O)[C@@H](C)O)cc1Cl]

or a pharmaceutically acceptable salt thereof.

In some embodiments, the small molecule PD-L1 inhibitor has the formula 1.041

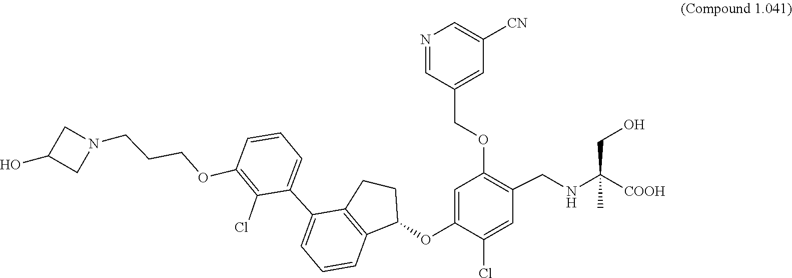

or a pharmaceutically acceptable salt thereof.

In some embodiments, the small molecule PD-L1 inhibitor has the formula 1.227

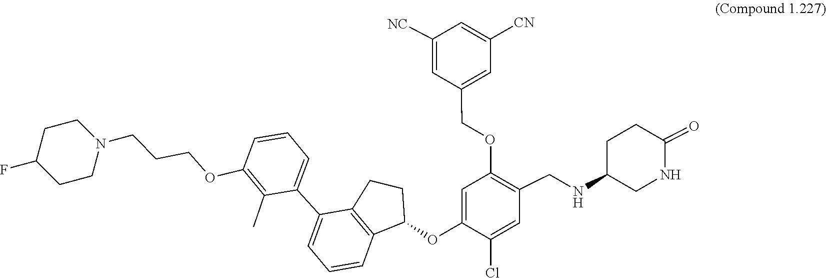 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC(=O)NC2)cc1Cl]

or a pharmaceutically acceptable salt thereof.

In some embodiments, the small molecule PD-L1 inhibitor has the formula of Compound 1.347

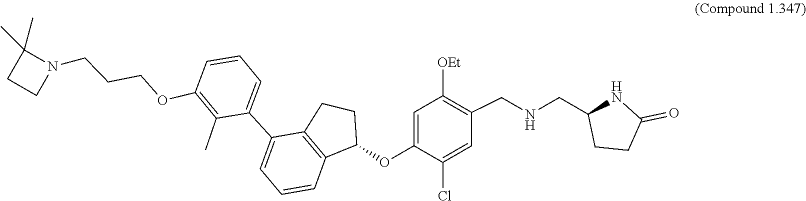 [SMILES: CCOc1cc(O[C@H]2CCc3c(-c4cccc(OCCCN5CCC5(C)C)c4C)cccc32)c(Cl)cc1CNC[C@@H]1CCC(=O)N1]

or a pharmaceutically acceptable salt thereof.

In some embodiments, the small molecule PD-L1 inhibitor is selected from the compounds or pharmaceutical compositions disclosed in WO2019/023575 filed by ChemoCentryx on Jul. 27, 2018. The contents of which is incorporated herein for all purposes.

C. Methods of Administration

The term “therapeutically effective amount” means the amount of the subject compound that will elicit the biological or medical response of a cell, tissue, system, or animal, such as a human, that is being sought by the researcher, veterinarian, medical doctor or other treatment provider.

In general, treatment methods provided herein comprise administering to a patient an effective amount of a compound one or more compounds provided herein. In a preferred embodiment, the compound(s) of the invention are preferably administered to a patient (e.g., a human) orally. Treatment regimens may vary depending on the compound used and the particular condition to be treated; for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, rate of excretion, drug combination (i.e., other drugs being administered to the patient) and the severity of the particular disease undergoing therapy, as well as the judgment of the prescribing medical practitioner. In general, the use of the minimum dose sufficient to provide effective therapy is preferred. Patients may generally be monitored for therapeutic effectiveness using medical or veterinary criteria suitable for the condition being treated or prevented.

Depending on the disease to be treated and the subject's condition, the compounds and compositions of the present invention may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical routes of administration and may be formulated, alone or together, in suitable dosage unit formulations containing conventional nontoxic pharmaceutically acceptable carriers, adjuvants and vehicles appropriate for each rouse of administration. The present invention also contemplates administration of the compounds and compositions of the present invention in a depot formulation.

Dosage levels of the order of from about 0.1 mg to about 140 mg per kilogram of body weight per day are useful (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. A sufficient amount of compounds should be administered to achieve a serum concentration of 50 ng/ml-200 ng/ml.

D. Kits

In some aspects, provided herein are kits containing a small molecule PD-L1 inhibitor described herein that are useful for treating a cancer. In some embodiments, the cancer is melanoma. A kit can contain a pharmaceutical composition containing a small molecule PD-L1 inhibitor, e.g., a compound of Formula (I). In some instances, the kit includes written materials e.g., instructions for use of the compound or pharmaceutical compositions thereof. Without limitation, the kit may include buffers, diluents, filters, needles, syringes, and package inserts with instructions for performing any methods.

E. Combination Therapy

In treating, preventing, ameliorating, controlling or reducing tumour growth and metastases, the compounds of the present invention may be used in conjunction with the following: (1) cancer vaccination strategies, (2) other immune-checkpoint modulators such as antagonistic antibodies against immune-checkpoint inhibitors (anti-PD1, anti-CTLA4, anti-Tim3, anti-VISTA, anti-KIR) or agonistic antibodies against immune-accelators (anti-Lag3, anti-OX40, anti-ICOS, anti-4-1BB, (3) blocking or depleting antibodies against cell surface proteins commonly up-regulated in transformed cells (CEACAM1, Syndecan-2, GRP78), (4) anti-angiogenic therapies (anti-VEGF, anti-VEGFR, VEGFR small molecule inhibitors), (5) anti-lymphangiogenesis (blocking antibodies or inhibitors against VEGF, FDF2, PDGF as well as its respective receptors), (6) standard chemotherapeutic therapies (Gemcitabine, Paclitaxel, FOLFORINOX), (7) irradiation therapy, (8) chemokine antagonists (CCR1, CCR2, CCR4, CCR6, CXCR4, CXCR2, CXCR7 small molecule inhibitors, blocking antibodies, or depleting antibodies), (9) depleting antibodies against chemokines that activate the aforementioned chemokine receptors, (10) inhibitors targeting common somatic mutations in cancer such as those specifically targeting the following genes (BRAF, KRAS, NRAS, EGFR, CTNNB1, NOTCH1, PIK3CA, PTEN, APC, FLT3, IDH1, IDH2, KIT, TP53, JAK2).

F. General Synthetic Procedures

The small molecules PD-L1 compounds described herein can be prepared a described herein.

Exemplary chemical entities will now be described by reference to illustrative synthetic schemes for their general preparation herein and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

Representative syntheses of compounds of the present disclosure are described in the scheme below, and the particular examples that follow. Schemes 1 and 2 are provided as further embodiments of the disclosure and illustrate general methods which were used to prepare compounds of the present disclosure including compounds of Formula (I), (Ia), or (Ib), and which can be used to prepare additional compounds having the Formula (I), (Ia), or (Ib). The methodology is compatible with a wide variety of functionalities.

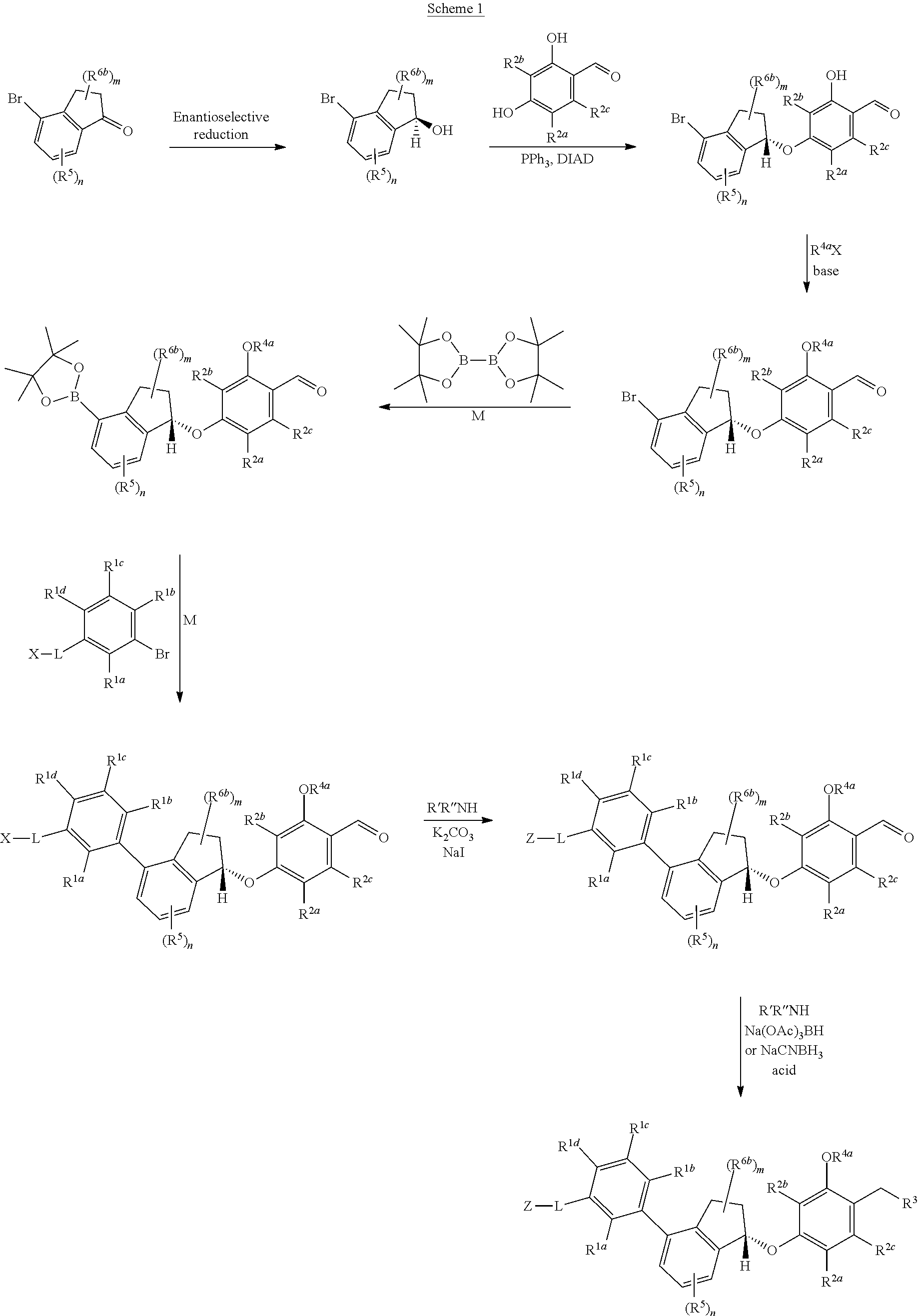

The 4-Bromoindanone compound can be enantioselectively reduced to its optically pure 4-bromoindanol derivative using a chiral reducing agent containing boron. In the subsequent step, the ether bond can be formed using reagents such as triphenyl phosphine and diisopropyl or diethyl azodicarboxylate (in this case, the reaction leads to an inversion of configuration, however, some racemization was observed). Alkylation of the phenol intermediate can be achieved using the appropriate alkyl halide or mesylate reagent. Introduction of the boronate at the 4-position of the indane ring can be accomplished via transition metal mediated coupling using bis(pinacalato)diboron. Coupling at the 4-position of the indane ring can be accomplished via transition metal mediated coupling using the appropriate aryl halide. Displacement of the halide X with appropriate amine can be achieved using potassium or cesium carbonate in presence of metal bromide or metal iodide. The reductive amination can be accomplished using the appropriate primary or secondary amine and a reducing agent such as sodium cyanoborohydride or sodium triacetoxyborohydride in presence of a mild acid such as acetic acid. The amine group added in the reductive amination is shown as R³ in the diagram above. The transformations shown in Scheme 1 may be performed in any order that is compatible with the functionality of the particular pendant groups.

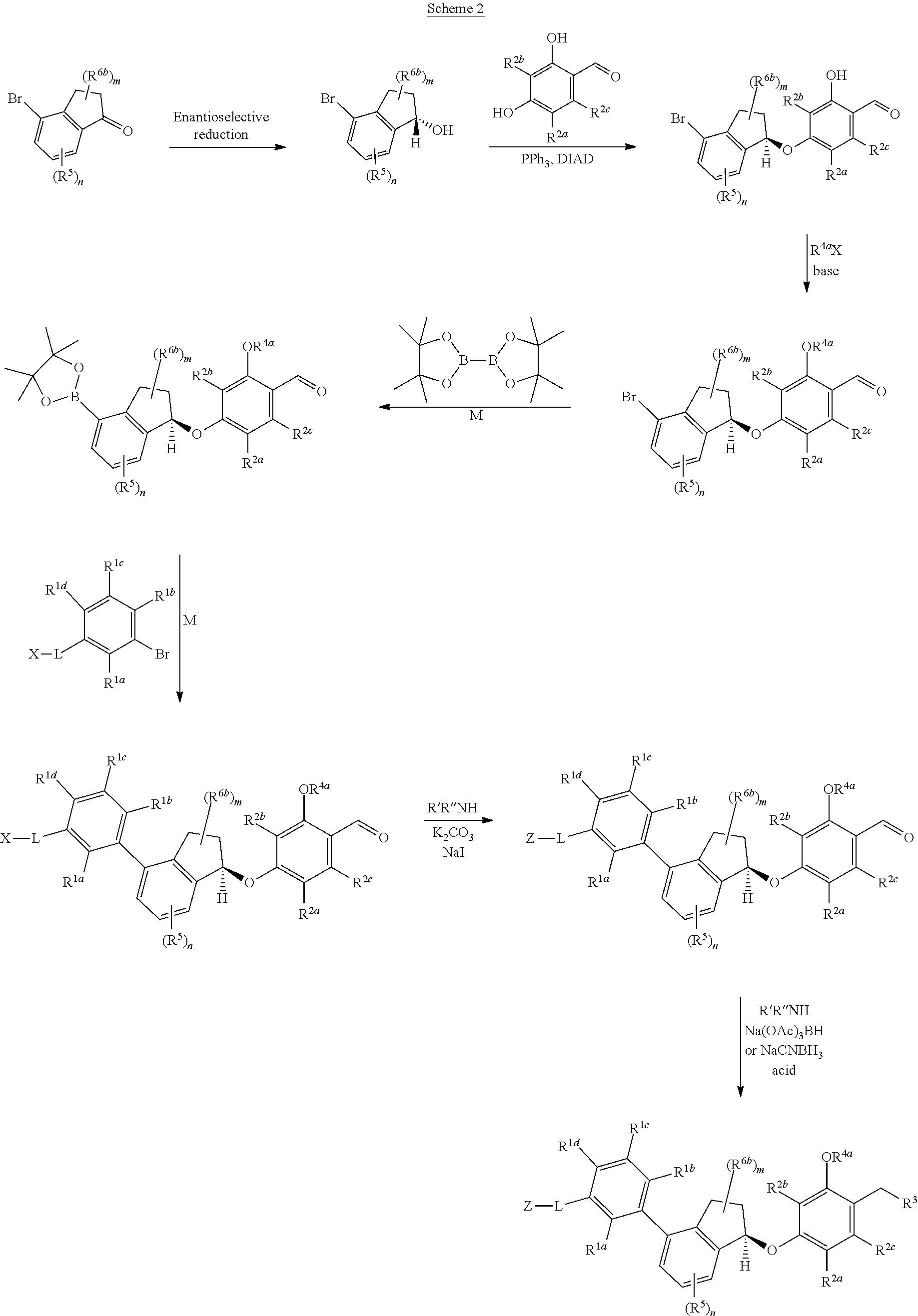

The 4-Bromoindanone compound can be enantioselectively reduced to its optically pure 4-bromoindanol derivative using a chiral reducing agent containing boron. In the subsequent step, the ether bond can be formed using reagents such as triphenyl phosphine and diisopropyl or diethyl azodicarboxylate (in this case, the reaction leads to an inversion of configuration, however, some racemization was observed). Alkylation of the phenol intermediate can be achieved using the appropriate alkyl halide or mesylate reagent. Introduction of the boronate at the 4-position of the indane ring can be accomplished via transition metal mediated coupling using bis(pinacalato)diboron. Coupling at the 4-position of the indane ring can be accomplished via transition metal mediated coupling using the appropriate aryl halide. Displacement of the halide X with appropriate amine can be achieved using potassium or cesium carbonate in presence of metal bromide or metal iodide. The reductive amination can be accomplished using the appropriate primary or secondary amine and a reducing agent such as sodium cyanoborohydride or sodium triacetoxyborohydride in presence of a mild acid such as acetic acid. The amine group added in the reductive amination is shown as R³ in the diagram above. The transformations shown in Scheme 2 may be performed in any order that is compatible with the functionality of the particular pendant groups.

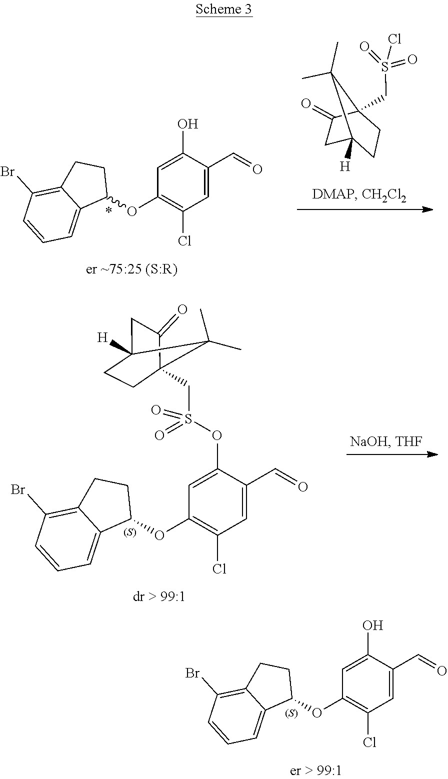

As an example, enrichment of optical purity of chiral intermediates can be achieved as described in Scheme 3.

### IV. EXAMPLES

### Example 1

### Tumor Reduction by a Small Molecule Human PD-1/PD-L1 Inhibitor in a Melanoma/PBMC Co-implantation Model (Summary)

Small molecule Programmed Death-1 (PD-1)/ Programmed Death-Ligand 1 (PD-L1) checkpoint inhibitors may provide the potential for increased tumor penetration, shorter half-life (to better manage immune related adverse events), and lower cost of goods. We embarked on an effort to identify and develop small molecules capable of targeting the immune checkpoint molecules PD-1/PD-L1, with an aim to improve anticancer immune responses in vivo.

### Methods

Co-crystallized human PD-1/PD-L1 provided structural information from which we developed a number of small molecule checkpoint inhibitors. Active compounds were first profiled by an ELISA assay measuring inhibition of the PD-1/PD-L1 interaction, followed by functional cell-based reporter and mixed lymphocyte reaction (MLR) assays. PD-1/PD-L1 inhibitory compounds thus identified were further selected for in vivo model testing. Since our human-specific PD-1/PD-L1 inhibitors did not cross react with murine PD-1/PD-L1, we co-implanted A375 human melanoma cells along with human peripheral blood mononuclear cells (PBMCs) into immunodeficient NOD/SCID mice to test their efficacy in vivo.

### Results

The optimized human PD-1/PD-L1 inhibitors exhibited marked activities in both the cell-based reporter and MLR assays. Compound 1.041, reduced tumor growth in vivo to a similar extent as the positive control anti-human PD-L1 antibody when dosed either prophylactically or therapeutically. Anti-tumor activity was completely dependent on the presence of human PBMCs. The tumor microenvironment analysis by flow cytometry indicated that the anti-tumor activity of Compound 1.041was accompanied by a significantly higher CD8⁺ T-Cell/CD4⁺ T-cell ratio. An X-ray structure of Compound 1.041 co-crystallized with PD-L1 revealed several vital interactions within the PD-1-binding-region of PD-L1, providing information about the structural basis by which the compound disrupts the PD-1/PD-L1 immune checkpoint interaction.

### Example 2

### Compound 1.041 is a Highly Potent Inhibitor of Human PD-1/PD-L1 Interaction that can reverse T-Cell Exhaustion

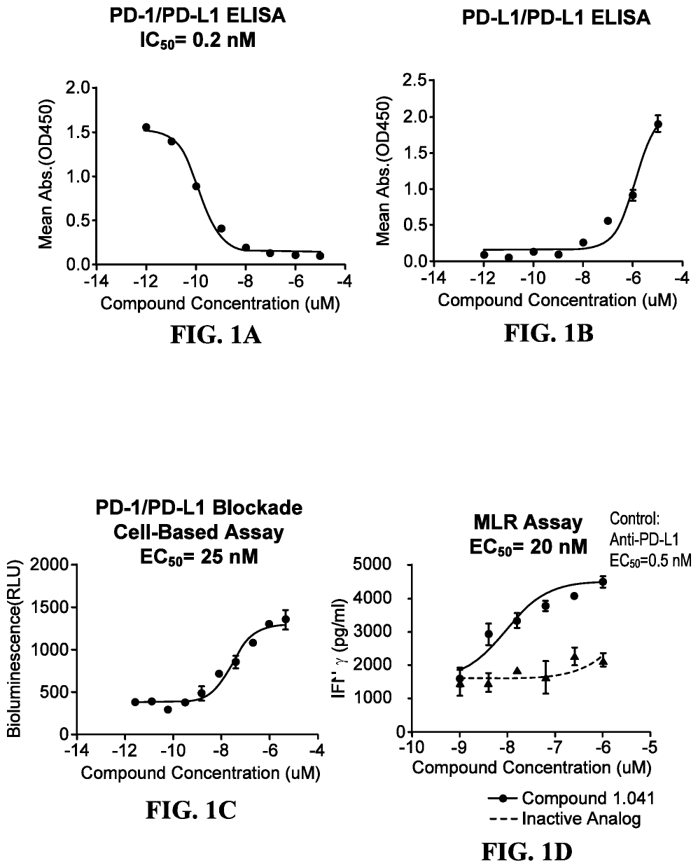

The activity of Compound 1.041 was assessed with an ELISA assay assessing the direct binding of recombinant PD-1 and PD-L1 proteins (FIG. 1A), a dimerization ELISA assay assessing the direct binding/dimerization of recombinant PD-L1 proteins (FIG. 1B), a cell based assay measuring the downstream signaling following the interaction of PD-1 and PD-L1 using a Luciferase reporter assay with cell lines overexpressing PD-1 and PD-L1 (FIG. 1C), and a mixed lymphocyte reaction assay measuring the functional outcome of upregulation of IFNg secretion, using allogenic primary human immune cells (FIG. 1D). Briefly, the mixed lymophocyte reaction assay was performed as follows: Dendritic cells and CD4+T Cells from unmatched donors were cultured together for 5 days in 96 well-flat bottom plates. Test compound was added as indicated at starting concentration of 1 μM with 1:4 dilutions with DMSO. Supernatants were harvested after 5 days of incubation and detection of human IFNg was performed by ELISA. Collectively, these assays show that Compound 1.041 is highly potent and blocks PD-1/PD-L1 interaction through induction of hPD-L1 dimerization.

Next, assays were prefored looking at the role of Compound 1.041 in reversing T-Cell

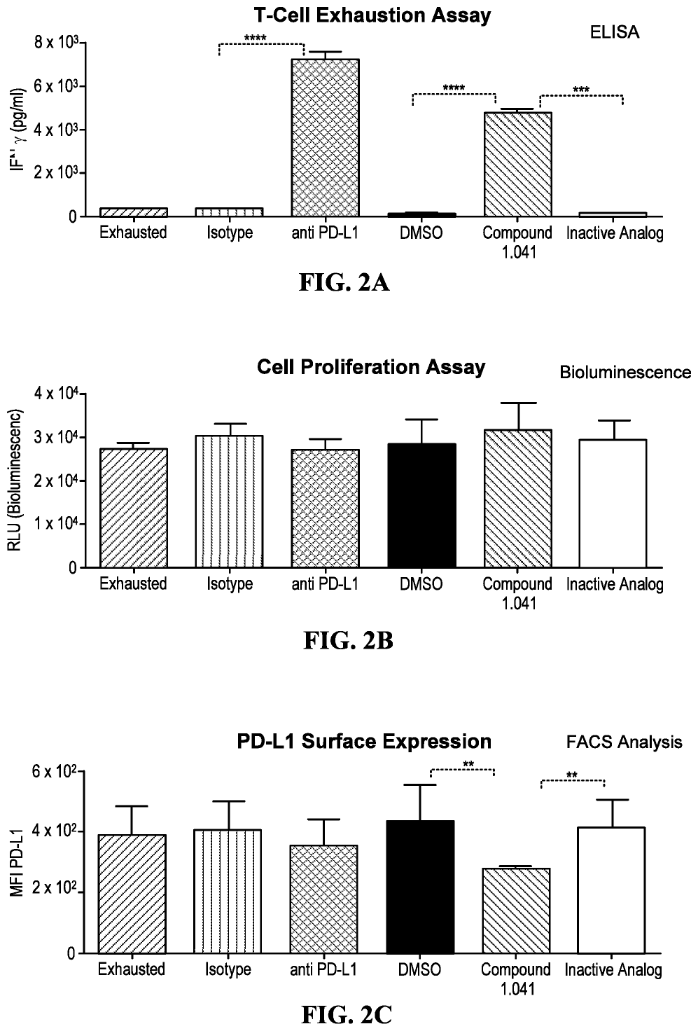

Exhaustion. A T-Cell Exhaustion assay was performed: freshly isolated human PBMCs were stimulated with 100ng/mL of Staphylococcal Enterotoxin B SEB for three days. Cells were washed and treated with an anti-PD-L1 antibody, an isopyte control of this antibody, DMSO, Compound 1.041, and an inactive analogue. As shown in FIG. 2A, Compound 1.041 reversed T cell exhaustion phenotype (as measured by IFN-g induction by SEB) similar to an anti-PD-L1 antibody. An inactive compound did not reverse T cell exhcuastion.

A cell proliferation assay (CellTiter-Glo assay) was performed using the same treatments as in FIG. 2A. This assay confirmed that the differences in IFN-γ detection reported in FIG. 2A were not due to changes in cell proliferation (FIG. 2B).

Further tests looked at PD-L1 surface expression on hCD11C⁺hCD3⁻ cells when treatetd with an anti-PD-L1 antibody, an isopyte control of this antibody, DMSO, Compound 1.041, and an inactive analogue. Briefly, this assay was performed as follows: Cells were seeded in 12 well plates. Test compounds or antibodies were added for 16 hours at different concentrations. Cells were washed and stained with anti-human PD-L1, anti-human CD11C and anti-human CD3 for 20 minutes at RT before performing FACS analysis. Matching isotype was used as a control. As shown in FIG. 2C, Compound 1.041 significantly reduced PD-L1 surface expression in hCD11C^{h}hCD3⁻ cells. A similar effect was not seen in the anti-PD-L1 or inactive analogue treatment.

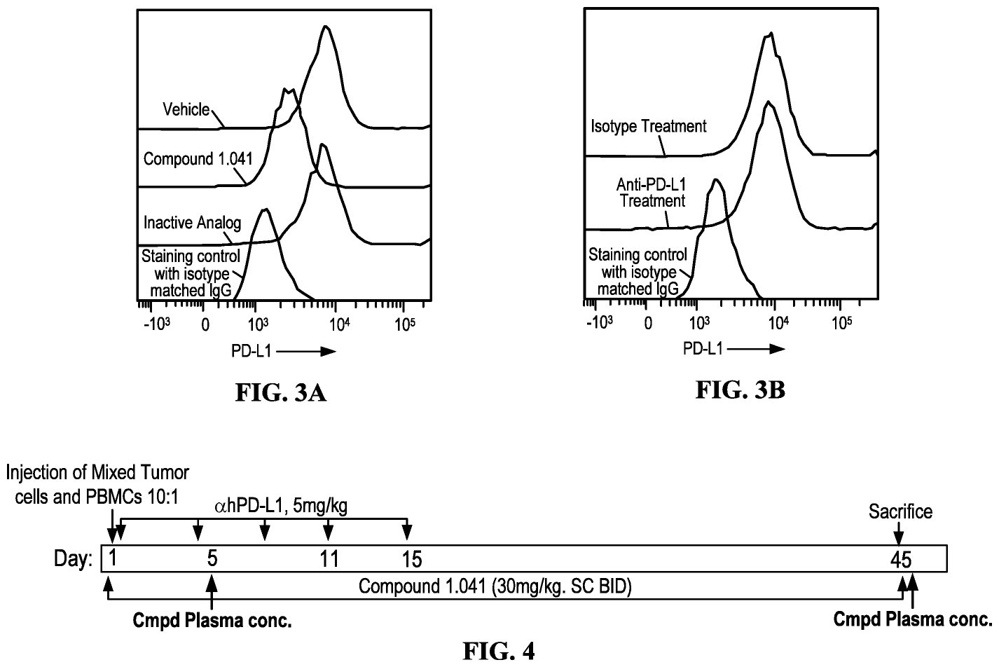

To further investigate the possibility that Compound 1.041 reduces PD-L1 surface expression, flow cytometry analysis was formed on MDA-MB-231 cells after PD-L1 surface staining. An acid-wash protocol (cells were washed 3 times (5 minutes with shaking) with cold acid wash buffer (DMEM containing 0.2% BSA and adjusted to a 3.5 pH) followed by 3 washes (5 minutes with shaking) with ice cold PBS 1X) was used to confirm that differences in PD-L1 cell surface staining was not due to tested article interfering with detection antibody binding. FIG. 3A shows treatments with vehicle, Compound 1.041, and a staining control. FIG. 3B shows treatments with anti-PD-L1 antibody treatment, an isotype control, and a staining control. As seen in FIG. 3A and FIG. 3B, Compound 1.041 reduced PD-L1 surface staining intensive, while PD-L1 antibody, isotype antibody, and vehicle did not.

Example 3: Prophylactic Dosing of Compound 1.041 Inhibits tumor Growth in the Presence of Human PBMCs

As briefly described in Example 1, the prophylactic activity of Compound 1.041 was tested using immunodeficientp NOD/SCID mice (NOD.CB17-Prkdc^{scid}/NCrHsd) where A375 human melanoma cells were co-implanted along with human peripheral blood mononuclear cells (PBMCs). The study design is shown in FIG. 4. The mice were broken into 8 groups (n =10 each group): +/−PBMCs+Vehicle; +/−PBMCs+Compound 1.041; +/−PBMCs+Isotype antibody; and +/−PBMCs+anti-hPD-L1. Anti-hPD-L1 and isotype matched control antibody were injected i.p. at 5 mg/kg on on day 1, 5, 9, 13, and 17. Compound 1.041 (30mg/kg) and vehicle dose (1% HPMC) p.i. twice daily (bid) starting on day 1.

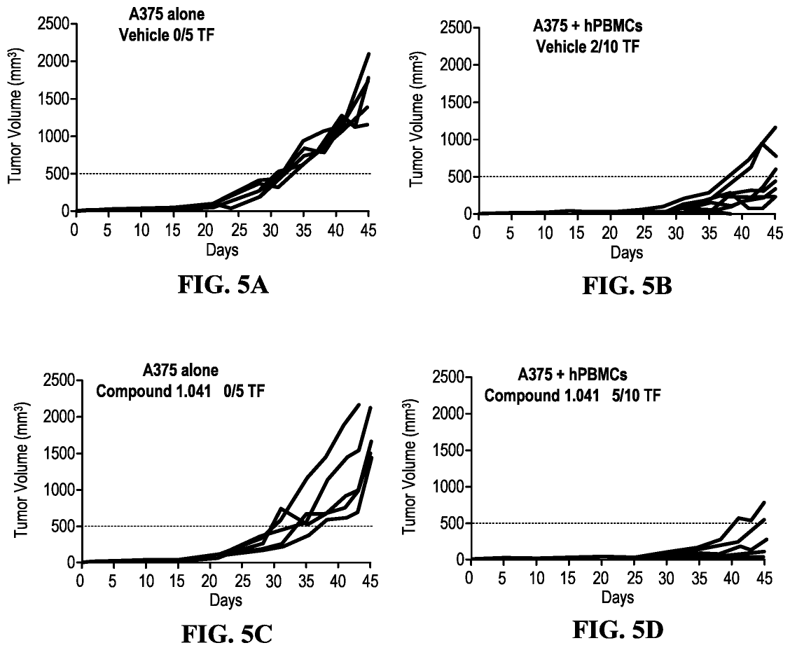

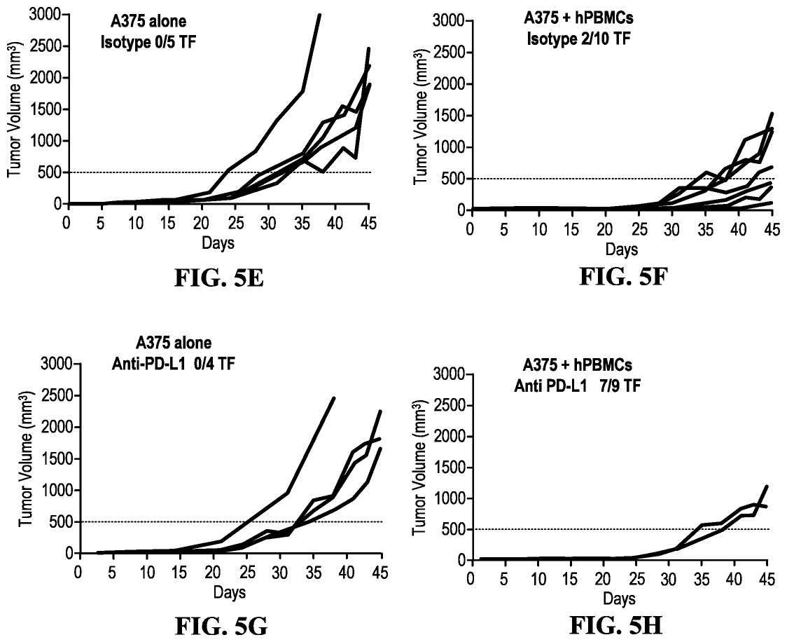

Results from this experiment are displayed in FIG. 5A-H. As seen in FIG. 5C and FIG. 5G, administration of Compound 1.041 or anti-PD-L1 had no effect on A375 tumor growth (compare to FIG. 5A and 5B—vehicle and isotype administration respectively). However, as seen in FIG. 5D and FIG. 5H, when Compound 1.041 and anti-PD-L1 was administered in mice with co-injected hPBMCs, both agents inhibited tumor growth. This suggests, that the presence of PD-1 expressing PBMCs are necessary for Compound 1.041 and anti-PD-L1 function.

### Example 4

### Therapeutic Dosing of Compound 1.041 Inhibits tumor Growth in the Presence of Human PBMCs

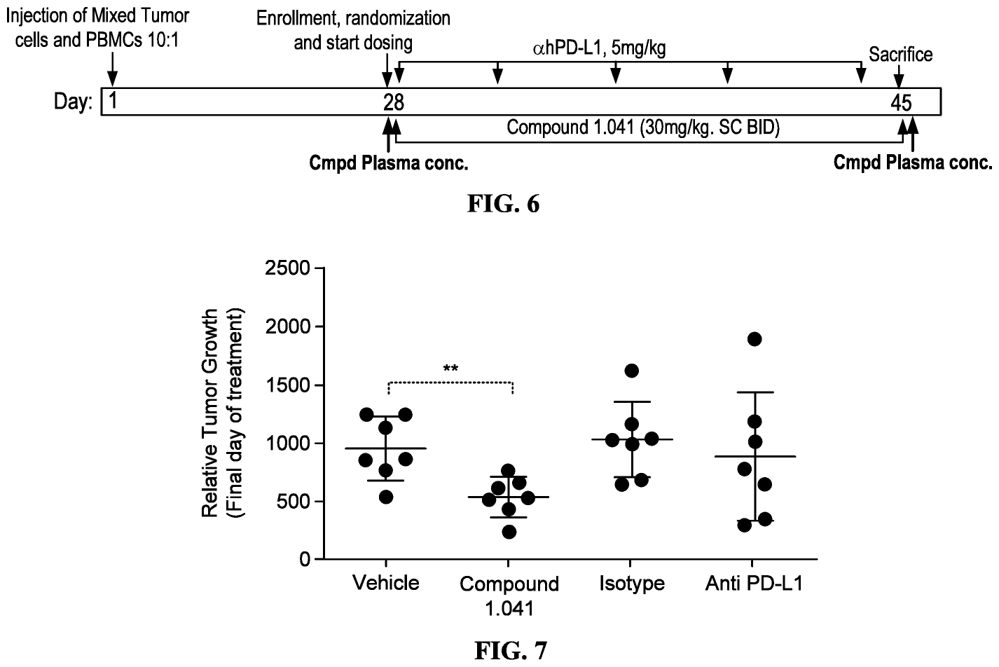

As briefly described in Example 1 the therapeutic activity of Compound 1.041 was tested using immunodeficient NOD/SCID mice (NOD.CB17-Prkdc^{scid}/NCrHsd) where A375 human melanoma cells were co-implanted along with human peripheral blood mononuclear cells (PBMCs). The study design is shown in FIG. 6. The mice were broken into 4 groups (n=7 each group): +PBMCs+Vehicle; +PBMCs+Compound 1.041; +PBMCs +Isotype antibody; and +PBMCs+anti-hPD-L1. Anti-hPD-L1 and isotype matched control antibody were injected i.p. at 5 mg/kg on on day 28, 32, 36, 40, and 44. Compound 1.041 (30mg/kg) and vehicle dose (1% HPMC) p.i. twice daily (bid) starting on day 28.

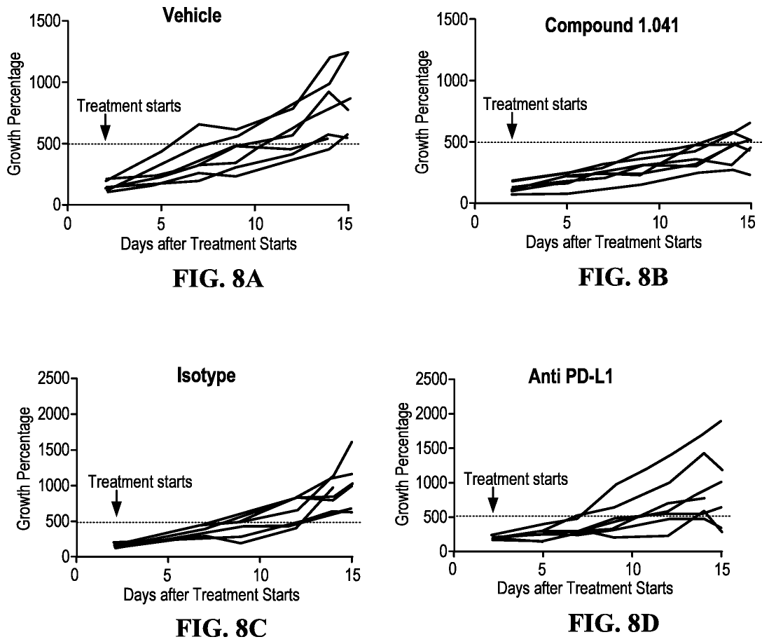

Results from this experiment are displayed in FIG. 7, reporting relative tumor growth. As seen in this figure, Compound 1.041 shows a significant inhibitory effect on established A375 tumors co-injected with human PBMCs. In contrast, anti-PD-L1 shows no effect in percentage of tumor growth. Plots of the percent growth per day for the various treatment groups are shown in FIG. 8A-D.

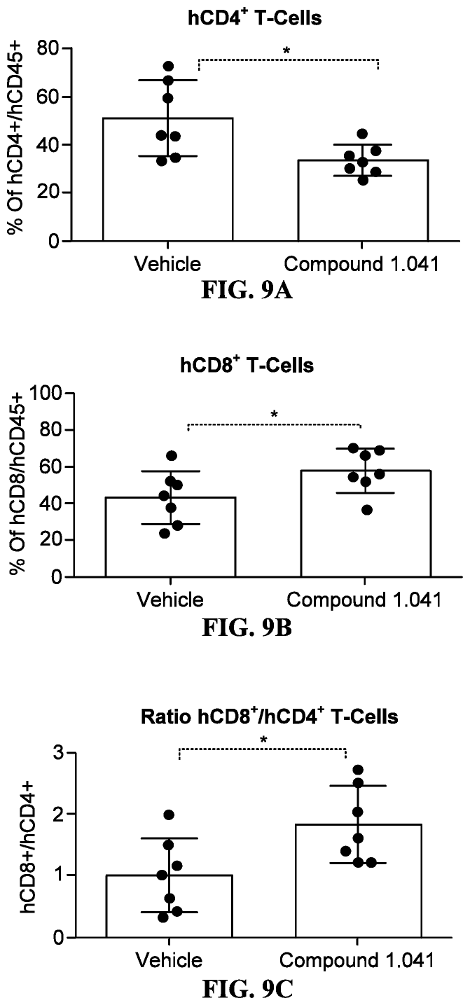

The tumor microenvironement of vehicle and Compound 1.041 treatments from the above experiment were further explored by dissecting, mincing and digested with collagenase D the tumors from relevant mice for analysis by flow cytometry. Briefly, this assay was performed as follows: Excised tumors were finely chopped with a blade and digested for 60 minutes at 37° C. in MSS 1X containing 2mg/ml of collagenase D and 1U/ml of DNAse I. Single cells were washed and resuspended in FACS buffer (PBS 1X with 10% FBS and 0.1% azide) containing the following monoclonal antibodies (hCD4 in FITC, PD-L1 in PE, CD3 in PercP-Cy5.5, CD45 in PE-Cy7, PD-1 in APC, CD8a in APC-Cy7, CD69 in Pacific Blue and LIVE/DEAD fixable Aqua stain). Isotype-matched controls were used as negative controls. Flow cytometry data were acquired with a FACSCanto II (BD Biosciences, San Jose, Calif.) cytometer and analyzed using FlowJO. Results looking at expression levels of hCD4⁺ T-Cells and hCD8⁺ T-Cells are shown in FIG. 9A-C. As seen in this figure, tumors from mice treated therapeutically with Compound 1 showed a higher percentage of hCD8⁺ T-Cells in contrast to hCD4⁺ T-Cells as compared to vehicle treated tumors (compare Panel A and Panel B). The ratio of of hCD8⁺/hCD4⁺ T-cells was significantly higher in tumors treated with Compound 1.041 compared to vehicle control (Panel C). The flow cytometer was gated on live cells singlets, hCD45⁺ and hCD3⁺ cells.

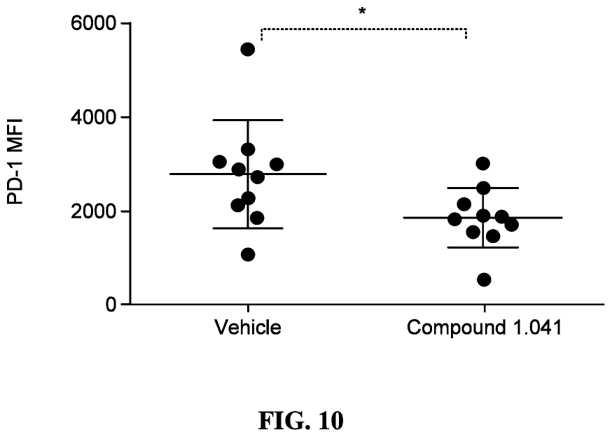

Further analyzing the flow cytometry data between vehicle and Compound 1.041 treated mice reveals that human CD8⁺ T-cells from mice treated with Compound 1.041 exhibit a reduction in PD-1 expression suggesting these cells are less exhausted compared to cells from vehicle treatment (as calculated by mean fluorescence intensity). See, FIG. 10.

### Example 5

### Synthesis of (5-chloro-2-((5-cyanopyridin-3-yl)methoxy)-4-4(((S)-4-(3-(3-(4-hydroxypiperidin-1-yl)propoxy)-2-methylphenyl)-2,3-dihydro-1H-inden-1-yl)oxy)benzyl)-L-threonine (Compound 1.004)

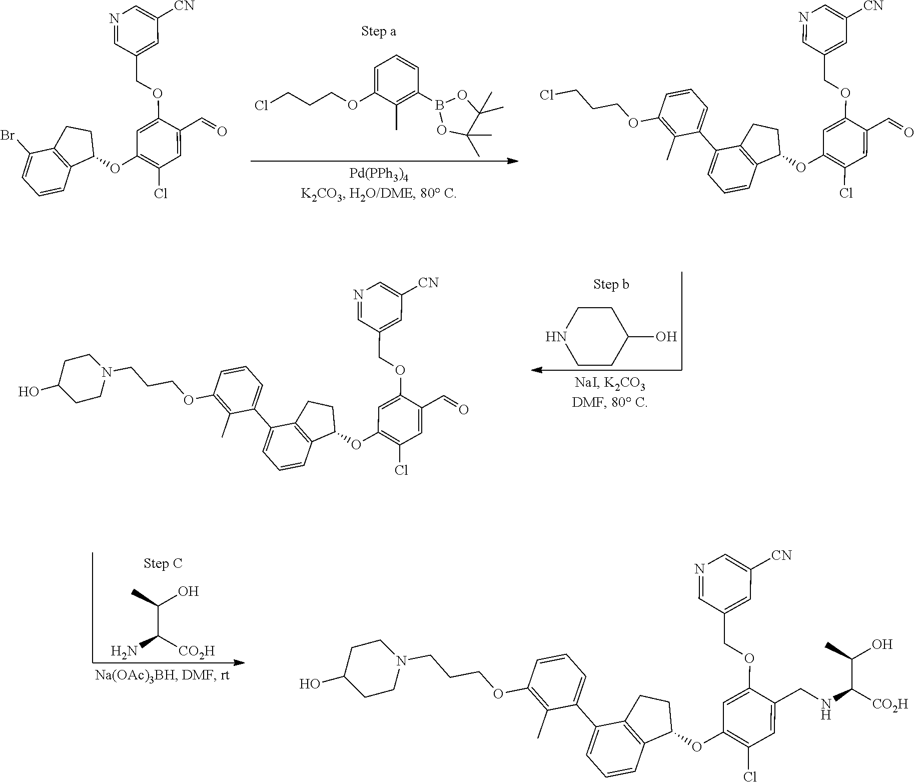

Step a: A biphasic solution of 5[[5-[(1S)-4-bromoindan-1-yl]oxy-4-chloro-2-formyl-phenoxy]methyl]pyridine-3-carbonitrile (7.7 g, 16 mmol), 2-(3-(3-chloropropoxy)-2-methylphenyl)-4,4,5,5-tetramethyl-1,3,2-dioxaborolane (6.0 g, 19 mmol), and Pd(PPh₃)₄ (3.7 g, 32 mmol) in aqueous 2 M K₂CO₃ (24 mL, 48 mmol) and 1,2-dimethoxyethane (240 mL) was degassed with nitrogen for 20 min. The mixture was then heated to 80° C. for 8 h before it was cooled to rt and water (150 mL) was added. The organic layer was separated and the aqueous layer was re-extracted with EtOAc (75 mL×2). The organics were combined, dried over MgSO₄, filtered, and concentrated in vacuo. Purification of the crude material by flash chromatography (SiO2, 100% hexane to 50% EtOAc in hexane) gave (S)-5-((4-chloro-5-((4-(3-(3-chloropropoxy)-2-methylphenyl)-2,3-dihydro-1H-inden-l-yl)oxy)-2-formylphenoxy)methyl)nicotinonitrile.

Step b: A slurry of (S)-5-((4-chloro-5-((4-(3-(3-chloropropoxy)-2-methylphenyl)-2,3-dihydro-1H-inden-1-yl)oxy)-2-formylphenoxy)methyl)nicotinonitrile (7.3 g, 12.4 mmol), 4-hydroxypiperidine (1.9 g, 18.6 mmol), sodium iodide (0.56 g, 3.72 mmol), and K₂CO₃ (3.4 g, 24.8 mmol) was heated to 80° C. and allowed to stir at this temperature for 8 h. After cooling to rt, the reaction mixture was poured into a separatory funnel containing water (100 mL). The mixture was extracted with 2:1 CHCl_{3:}isopropanol (60 mL×3). The organics were combined, dried over MgSO₄, filtered, and concentrated in vacuo. Purification of the crude material by flash chromatography (SiO₂, 100% DCM to 15% MeOH in DCM) gave (S)-5-((4-chloro-2-formyl-5-((4-(3 -(3 -(4-hy droxypiperidin-1-yl)propoxy)-2-methylphenyl)-2,3 -dihydro-1H-inden-1-yl)oxy)phenoxy)methyl)nicotinonitrile.

Step c: A solution of (S)-5-((4-chloro-2-formyl-5-((4-(3-(3-(4-hydroxypiperidin-1-yl)propoxy)-2-methylphenyl)-2,3-dihydro-1H-inden-1-yl)oxy)phenoxy)methyl)nicotinonitrile (3.2 g, 4.9 mmol) and L-threonine (1.5 g, 12.2 mmol) was stirred in DMF (48 mL) for 3 h before sodium triacetoxyborohydride (3.1 g, 14.6 mmol) was added in portions over 10 min. The reaction mixture was left to stir overnight at room temperature. The majority of DMF was removed in vacuo, and the crude material was re-diluted in MeO H and filtered. The filtrate was purified by reverse phase preparative HPLC (CH3CN—H₂O with 0.1% NH₄HCO₃) to obtain (5-chloro-2-((5-cyanopyri din-3 -yl)methoxy)-4-(((S)-4-(3 -(3 -(4-hydroxypiperidin-1-yl)propoxy)-2-methylphenyl)-2,3 -dihydro-1H-inden-1-yl)oxy)benzyl)-L-threonine. MS: (ES) m/z calculated for C₄₂H₄₇ClN4O₇ [M+H]⁺ 755.3, found 755.2. ^{N} NMR (400 MHz, Methanol-d₄) δ 8.97 (d, J=2.1 Hz, 1H), 8.87 (d, J=1.9 Hz, 1H), 8.41 (t, J=2.1 Hz, 1H), 7.38 (s, 1H), 7.35-7.22 (m, 2H), 7.21-7.06 (m, 2H), 6.97-6.87 (m, 2H), 6.73 (dd, J=24.8, 7.5 Hz, 1H), 5.97-5.80 (m, 1H), 5.30 (s, 2H), 4.07 (t, J=6.0 Hz, 2H), 3.85-3.77 (m, 1H), 3.77-3.55 (m, 3H), 2.95 (d, J=6.3 Hz, 1H), 2.92-2.74 (m, 2H), 2.69-2.57 (m, 3H), 2.55-2.36 (m, 1H), 2.31-1.99 (m, 4H), 1.96 (d, J=18.4 Hz, 2H), 1.91-1.81 (m, 2H), 1.65-1.53 (m, 2H), 1.44 (s, 1H), 1.28 (d, J=1.5 Hz, 2H), 1.20 (d, J=6.3 Hz, 3H).

### Example 6

### Synthesis of (S)-2-((5-chloro-4-(((S)-4(2-chloro-3-(3-(3-hydroxyazetidin-1-yl)propoxy)phenyl)-2,3-dihydro-1H-inden-1-yl)oxy)-2-((5-cyanopyridin-3-yl)methoxy)benzyl)amino)-3-hydroxy-2-methylpropanoic acid (Compound 1.041)

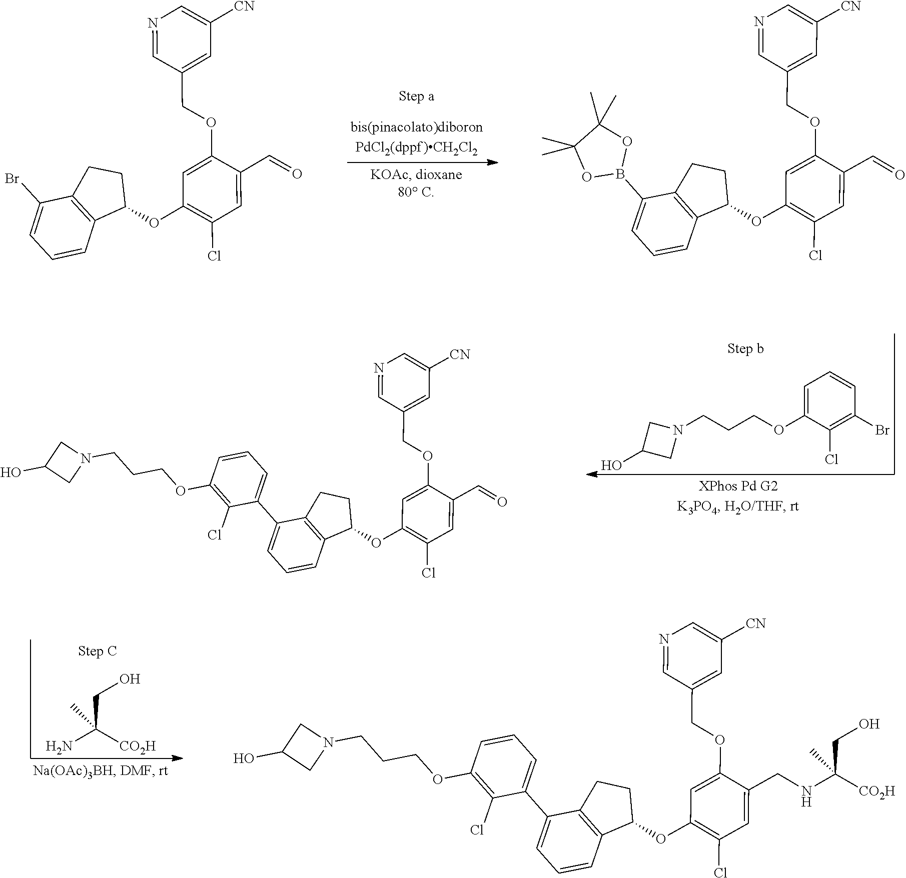

Step a: A solution of 5-[[5-[(1S)-4-bromoindan-1-yl]oxy-4-chloro-2-formyl-phenoxy]methyl]pyridine-3-carbonitrile (3.0 g, 6.2 mmol), bis(pinacolato)diboron (2.37 g, 9.3 mmol), and potassium acetate (1.83 g, 18.6 mmol) in dioxane (100 mL) was degassed with nitrogen for 15 min before the addition of 1,1′-bis(diphenylphosphino)ferrocene]-dichloropalladium(II) complexed with dichloromethane. The mixture was degassed further for 5 min and then heated to 80° C. After 11 h, the solution was cooled to room temperature and water was added (50 mL). The reaction mixture was extracted with EtOAc (30 mL×3) and the combined organics were dried over MgSO₄, filtered, and concentrated in vacuo. Purification of the crude material by flash chromatography (SiO₂, 100% hexane to 50% EtOAc in hexane) gave (S)-5-((4-chloro-2-formyl-5-((4-(4,4,5,5-tetramethyl-1,3 ,2-dioxaborolan-2-yl)-2,3 -dihydro-1H-inden-1-yl)oxy)phenoxy)methyl)nicotinonitrile.

Step b: A solution of (S)-5-((4-chloro-2-formyl-5-((4-(4,4,5,5-tetramethyl-1,3,2-dioxaborolan-2-yl)-2,3-dihydro-1H-inden-1-yl)oxy)phenoxy)methyl)nicotinonitrile (2.5 g, 4.68 mmol), 1-(3-(3-bromo-2-chlorophenoxy)propyl)azetidin-3-ol (1.5 g, 4.68 mmol), and aqueous 0.5 M K₃PO₄ (28 mL, 14 mmol) in THF (30 mL) was degassed with nitrogen for 25 min before XPhos Pd G2 (0.74 g, 0.94 mmol) was added. After degassing for an additional 10 min, the solution was allowed to stir at room temperature for 20 h. Water (30 mL) was then added to the reaction mixture, and the mixture was extracted with 2:1 chloroform: isopropanol (40 mL x 3). The combined organics were dried over MgSO₄, filtered, and concentrated in vacuo. Purification of the crude material by flash chromatography (SiO₂, 100% DCM to 15% MeOH in DCM) gave (S)-5 -((4-(2-chloro-3-(3-(3-hydroxyazetidin-1-yl)propoxy)phenyl)-2,3 -dihydro-1H-inden-1-yl)oxy)-2-formylphenoxy)methyl)nicotinonitrile.

Step c: A solution of (S)-5-((4-chloro-5-((4-(2-chloro-3-(3-(3-hydroxyazetidin-1-yl)propoxy)phenyl)-2,3-dihydro-1H-inden-1-yl)oxy)-2-formylphenoxy)methyl)nicotinonitrile (0.73 g, 1.1 mmol) and 2-Me-L-serine (0.40 g, 3.4 mmol) was stirred in DMF (36 mL) for 1 h before sodium triacetoxyborohydride (0.72 g, 3.4 mmol) was added in small portions over 1 h. The reaction mixture was left to stir overnight at room temperature. The majority of DMF was removed in vacuo and the crude material was purified by reverse phase preparative HPLC (CH₃CN—H₂O with 0.1% NH₄HCO₃) to obtain (S)-2-((4-chloro-4-(((S)-4-(2-chloro-3-(3-(3-hydroxyazetidin-1-yl)propoxy)phenyl)-2,3-dihydro-1H-inden-1-yl)oxy)-2-((5-cyanopyridin-3-yl)methoxy)benzyl)amino)-3-hydroxy-2-methylpropanoic acid. MS: (ES) m/z calculated for C₃₉H₄₀Cl₂N₄O₇ [M+H]⁺ 747.2, found 747.2. ¹H NMR (400 MHz, Methanol-d₄) δ 8.98 (s, 1H), 8.87 (d, J=1.9 Hz, 1H), 8.43 (s, 1H), 7.45 (s, 1H), 7.39-7.21 (m, 3H), 7.16 (d, J=7.4 Hz, 1H), 7.08 (d, J=9.2 Hz, 1H), 7.02-6.79 (m, 2H), 6.04-5.81 (m, 1H), 5.32 (s, 2H), 4.34 (q, J=6.4 Hz, 1H), 4.12 (t, J=6.0 Hz, 2H), 3.84 (s, 2H), 3.69 (td, J=6.3, 2.3 Hz, 3H), 3.62 (d, J=11.2 Hz, 1H), 3.01-2.79 (m, 1H), 2.92 (td, J=6.5, 2.1 Hz, 2H), 2.75 (t, J=7.4 Hz, 2H), 2.70-2.54 (m, 1H), 2.53-2.39 (m, 1H), 2.18-2.01 (m, 1H), 1.91 (q, J=6.5 Hz, 2H), 1.29 (s, 3H).

Synthesis of 1-(3-(3-bromo-2-chlorophenoxy)propyl)azetidin-3-ol

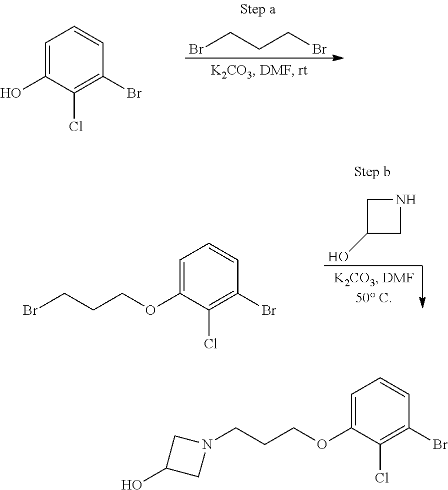 [SMILES: Oc1cccc(Br)c1Cl ; BrCCCBr ; Clc1c(Br)cccc1OCCCBr ; OC1CNC1 ; OC1CN(CCCOc2cccc(Br)c2Cl)C1]

Step a: To a slurry of 3-bromo-2-chlorophenol (9.82 g, 47.3 mmol) and potassium carbonate (13.7 g, 94.6 mmol) in DMF (20 mL) was slowly added 1,3-dibromopropane (28.7 g, 142 mmol and the mixture was stirred at room temperature for 18 h. Water (30 mL) and DCM (50 mL) were added to the reaction mixture and after stirring for a few minutes, the biphasic solution was poured into a separatory funnel. The organic layer was separated and the aqueous layer was re-extracted with DCM (2×50 mL). The combined organics were dried over MgSO₄, filtered, and concentrated in vacuo. The crude material was purified by flash chromatography (SiO₂, 100% hexane to 5% Et₂O in hexane) to obtain 1-bromo-3-(3-bromopropoxy)-2-chlorobenzene.

Step b: To a slurry of 1-bromo-3-(3-bromopropoxy)-2-chlorobenzene (3.7 g, 11.3 mmol) and potassium carbonate (3.12 g, 22.6 mmol) in DMF (10 mL) at 50° C. was added a pre-heated (50° C.) solution of finely suspended 3-hydroxyazetidine (1.07 g, 14.6 mmol) in DMF (25 mL). After 1 h, the reaction mixture was allowed to cool to room temperature and filtered through Celite. The filtrate was concentrated in vacuo and the crude material was purified by flash chromatography (SiO₂, 10% to 20% Et₂O in hexane then 10% MeOH in DCM) to obtain 1-(3-(3 -bromo-2-chlorophenoxy)propyl)azetidin-3 -ol.

### Example 7

### Synthesis of (S)-2-((5-chloro-2-((3,5-dicyanobenzyl)oxy)-4-(((S)-4-(3-(3-(4-hydroxypiperidin-1-yl)propoxy)-2-methylphenyl)-2,3-dihydro-1H-inden-1-yl)oxy)benzyl)amino)-3-hydroxy-2-methylpropanoic acid

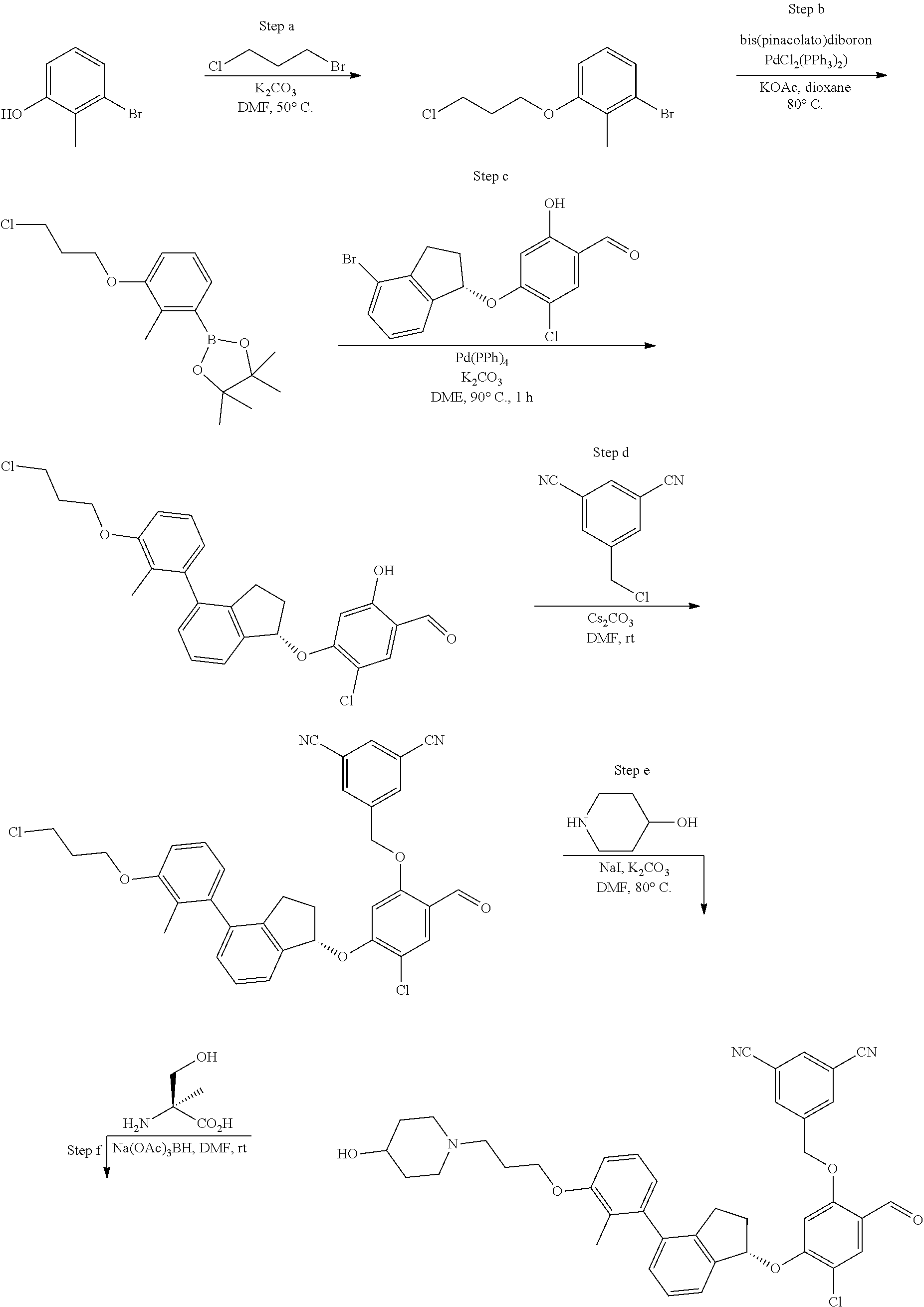

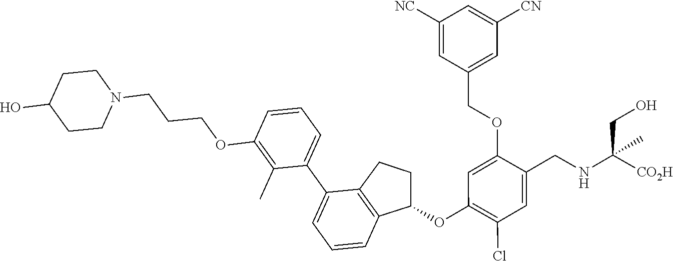 [SMILES: Cc1c(OCCCN2CCC(O)CC2)cccc1-c1cccc2c1CC[C@@H]2Oc1cc(OCc2cc(C#N)cc(C#N)c2)c(CN[C@@](C)(CO)C(=O)O)cc1Cl]

Step a: To a solution of 3-bromo-2-methylphenol (10.0 g, 53.5 mmol) in DMF (50 mL) was added 1-bromo-3-chloropropane (8.42 g, 53.5 mmol) and potassium carbonate (8.87 g, 64.2 mmol). The reaction mixture was heated up to 50° C. and stirred at 50° C. for 16 h. Then it was cooled down to room temperature. Water (50 mL) and DCM (100 mL) were added to the reaction mixture and after stirring for a few minutes, the biphasic solution was poured into a separatory funnel. The aqueous layer was extracted with DCM (2×50 mL). The combined organics was dried over MgSO₄, filtered, and concentrated in vacuo. The crude product was purified by flash chromatography (SiO₂, 100% hexane to 20% EtOAc in hexane) to obtain 1-bromo-3-(3-chloropropoxy)-2-methylbenzene.

Step b: To a slurry of 1-bromo-3-(3-chloropropoxy)-2-methylbenzene (2.40 g, 9.10 mmol), bis(pinacolato)diboron (3.00 g, 11.83 mmol), and potassium acetate (2.68 g, 27.30 mmol) in dioxane (40 mL) was degassed with nitrogen for 15 min before the addition of bis(triphenylphosphino)dichloropalladium. The mixture was degassed further for 5 min and the reaction mixture was heated to 80° C. After 11 h, the solution was cooled to room temperature and water was added (20 mL). The reaction mixture was extracted with EtOAc (30 mL×3). The combined organics was dried over MgSO4, filtered, and concentrated in vacuo. The crude product was purified by flash chromatography (SiO₂, 100% hexane to 10% EtOAc in hexane) to obtain a colorless oil 2-(3-(3-chloropropoxy)-2-methylphenyl)-4,4,5,5-tetramethyl-1,3,2-dioxaborolane.

Step c: To a slurry of (S)-4-((4-bromo-2,3-dihydro-1H-inden-1-yl)oxy)-5-chloro-2-hydroxybenzaldehyde (370.0 mg, 1.0 mmol), 2-(3-(3-chloropropoxy)-2-methylphenyl)-4,4,5,5-tetramethyl-1,3,2-dioxaborolane (370.0 mg, 1.20 mmol), and 2 M potassium carbonate (1.50 mL, 3.0 mmol) in DME (10 mL) was degassed with nitrogen for 15 min before the addition of tetra(triphenylphosphino)palladium (120.0 mg, 0.10 mmol). The mixture was degassed further for 5 min and the reaction mixture was heated to 90° C. After 1 h, the solution was cooled to room temperature and water was added (10 mL). The reaction mixture was extracted with EtOAc (10 mL×3). The combined organics was dried over MgSO₄, filtered, and concentrated in vacuo. The crude product was purified by flash chromatography (SiO₂, 100% hexane to 5% EtOAc in hexane) to obtain a brown oil To a slurry of (S)-5-chloro-4-((4-(3-(3-chloropropoxy)-2-methylphenyl)-2,3 -dihydro-1H-inden-1 -yl)oxy)-2-hydroxybenzaldehyde.

Step d: To a slurry of (S)-4-((4-bromo-2,3-dihydro-1H-inden-l-yl)oxy)-5-chloro-2-hydroxybenzaldehyde(S)-5 -chloro-4-((4-(3 -(3 -chloropropoxy)-2-methylphenyl)-2,3 -dihydro-1H-inden-1-yl)oxy)-2-hydroxybenzaldehyde (410.0 mg, 0.88 mmol) and cesium carbonate (860.0 mg, 2.64 mmol) in DMF (3 mL) was added 5-(chloromethyl)isophthalonitrile (310.0 mg, 1.75 mmol). The mixture was stirred at room temperature for 1 h and water was added (3 mL). The reaction mixture was extracted with EtOAc (10 mL×3). The combined organics was dried over MgSO₄, filtered, and concentrated in vacuo. The crude product was purified by flash chromatography (SiO₂, 100% hexane to 30% EtOAc in hexane) to obtain white solid (9-5-((4-chloro-5-((4-(3-(chloropropoxy)-2-methylphenyl)-2,3 -dihydro-1H-inden-1 -yl)oxy)-2-formylphenoxy)methyl)isophthalonitrile.

Step e: A slurry of (S)-5-((4-chloro-5-((4-(3-(3-chloropropoxy)-2-methylphenyl)-2,3-dihydro-1H-inden-1-yl)oxy)-2-formylphenoxy)methyl)isophthalonitrile (259.0 mg, 0.42 mmol), piperidin-4-ol (51.4 mg, 0.50 mmol), potassium carbonate (70.0 mg, 0.50 mmol) and sodium iodide (63.0 mg, 0.42 mmol) in DMF (2 mL) was warmed up to 80° C. and stirred for 12 h. The reaction mixture was cooled to room temperature and water was added (2 mL). The reaction mixture was extracted with EtOAc (5 mL×3). The combined organics was dried over Na₂SO₄, filtered, and concentrated in vacuo. The crude product was purified by flash chromatography (SiO₂, 100% dichloromethane to 20% methanol in dichloromethane) to obtain white solid (S)-5-((4-chloro-2-formyl-5-((4-(3 -(3 -(4-hydroxypiperidin-1-yl)propoxy)-2-methylphenyl)-2,3-dihydro-1H-inden-1 -yl)oxy)phenoxy)methyl)isophthalonitrile.

Step f: A mixture of (S)-5-((4-chloro-2-formyl-5-((4-(3-(3-(4-hydroxypiperidin-1-yl)propoxy)-2-methylphenyl)-2,3-dihydro-1H-inden-1-yl)oxy)phenoxy)methyl)isophthalonitrile (100 mg, 0.15 mmol) and α-Me-L-serine (119.1 mg, 0.74 mmol) was stirred in DMF (2 mL) for 1 h before sodium triacetoxyborohydride (127.0 mg, 0.6 mmol) was added in small portions over 1 h. The reaction mixture was left to stir overnight at room temperature. The majority of DMF was removed in vacuo and the crude material was purified by reverse phase preparative HPLC (CH₃CN—H₂O with 0.1% TFA) to obtain (S)-2-((5-chloro-2-((3,5-dicyanobenzyl)oxy)-4-(((S)-4-(3-(3-(4-hydroxypiperidin-1-yl)propoxy)-2-methylphenyl)-2,3-dihydro-1H-inden-1-yl)oxy)benzyl)amino)-3-hydroxy-2-methylpropanoic acid as TFA salt form and later converted to trifluoro ammonium salt. MS: 779.3 [M+H]; ¹H NMR (400 MHz, Methanol-d₄) ¹H NMR (400 MHz, Methanol-d₄) δ 8.26 (d, J=4.6 Hz, 2H), 8.17 (t, J=1.5 Hz, 1H), 7.55 (s, 1H), 7.26 (s, 2H), 7.20 (s, 1H), 7.14-7.07 (m, 1H), 6.96 (t, J=9.9 Hz, 2H), 6.78 (dd, J=20.6, 7.6 Hz, 1H), 5.98 (s, 1H), 5.37 (d, J=16.3 Hz, 2H), 4.33-4.23 (m, 2H), 4.15 (s, 3H), 3.96 (d, J=12.0 Hz, 1H), 3.76 (d, J=12.0 Hz, 1H), 3.37 (s, 1H), 3.23 (s, 5H), 2.59 (s, 1H), 2.46 (s, 1H), 2.29 (s, 2H), 2.13 (s, 2H), 1.98 (d, J=6.8 Hz, 4H), 1.48 (s, 3H).

### Example 8

### Synthesis of 5-((4-chloro-5-(((S)-4-(3-(3-(4-fluoropiperidin-1-yl)propoxy)-2-methylphenyl) methylphenyl)-2,3-dihydro-1H-inden-1-yl)oxy)-2-((((S)-6-oxopiperidin-3-yl)amino)methyl)phenoxy)methyl)isophthalonitrile (Compound 1.227)

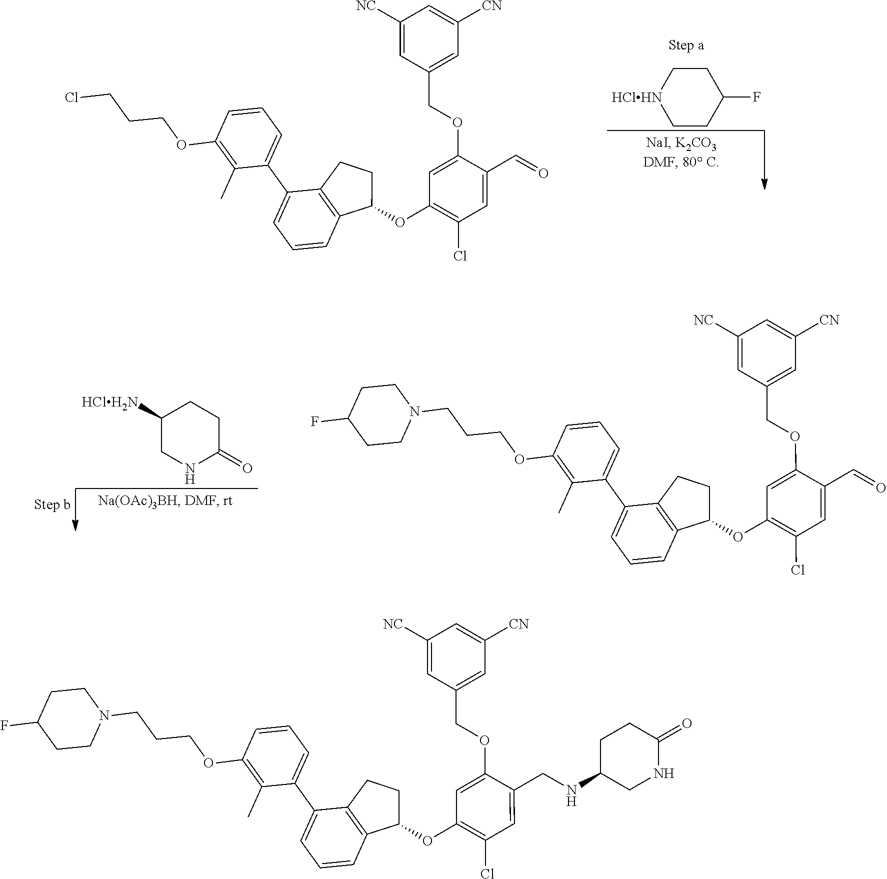

Step a: A slurry of (S)-5-((4-chloro-5-((4-(3-(3-chloropropoxy)-2-methylphenyl)-2,3-dihydro-1H-inden-1-yl)oxy)-2-formylphenoxy)methyl)isophthalonitrile (728.0 mg, 1.19 mmol), 4-fluoropiperidine hydrochloride (200.0 mg, 1.43 mmol), potassium carbonate (411.0 mg, 2.98 mmol) and sodium iodide (179.0 mg, 1.19 mmol) in DMF (5 mL) was warmed up 80° C. and stirred for 12 h. The reaction mixture was cooled to room temperature and water was added (2 mL). The reaction mixture was extracted with EtOAc (5 mL×3). The combined organics were dried over Na₂SO₄, filtered, and concentrated in vacuo. The crude product was purified by flash chromatography (SiO₂, 100% dichloromethane to 20% methanol in dichloromethane) to obtain yellow oil (S)-5-((4-chloro-5-((4-(3-(3-(4-fluoropiperidin-1-yl)propoxy)-2-methylphenyl)-2,3-dihydro-1H-inden-1-yl)oxy)-2-formylphenoxy)methyl)isophthalonitrile.

Step b: A mixture of (S)-5-((4-chloro-5-((4-(3-(3-(4-fluoropiperidin-1-yl)propoxy)-2-methylphenyl)-2,3-dihydro-1H-inden-1-yl)oxy)-2-formylphenoxy)methyl)isophthalonitrile (50.0 mg, 0.074 mmol) and (S)-5-aminopiperidin-2-one hydrochloride (33.0 mg, 0.22 mmol) was stirred in DMF (2 mL) for 1 h before sodium triacetoxyborohydride (47.0 mg, 0.22 mmol) was added. The reaction mixture was left to stir overnight at room temperature. The majority of DMF was removed in vacuo and the crude material was purified by reverse phase preparative HPLC (CH₃CN-H₂O with 0.1% TFA) to obtain 5-((4-chloro-5-(((S)-4-(3-(3-(4-fluoropiperidin-1-yl)propoxy)-2-methylphenyl)-2,3-dihydro-1H-inden-1-yl)oxy)-2-((((S)-6-oxopiperidin-3-yl)amino)methyl)phenoxy)methyl)isophthalonitrile as TFA salt form which was then passed through basic cartridge to convert it to freeform. MS: 776.2 [M+H]; ¹H NMR (400 MHz, Methanol-d₄) δ 8.23-8.15 (m, 2 H), 8.02-7.95 (m, 1H), 7.47-7.38 (m, 2H), 7.30-7.14 (m, 2H), 7.11 (s, 1H), 6.96 (dd, J=17.7, 8.6 Hz, 2H), 6.75 (dd, J=19.1, 7.6 Hz, 1H), 5.92 (s, 1H), 5.36-5.27 (m, 2H), 4.75 (d, J=12.2 Hz, 1H), 4.13 (t, J=5.9 Hz, 2H), 3.97 (d, J=13.2 Hz, 1H), 3.92-3.82 (m, 1H), 3.52 (dd, J=17.5, 8.9 Hz, 1H), 3.30 (dt, J=3.3, 1.7 Hz, 6H), 3.05 (s, 6H), 2.83 (m, 1H), 2.43 (dt, J=11.5, 6.5 Hz, 1H), 2.32 (dd, J=15.5, 8.6 Hz, 1H), 2.19 (m, 2H), 2.12 (m, 1H), 2.06 (m, 4H), 1.97 (d, J=16.2 Hz, 3H).

Compounds in Table 1 were prepared by methods as described in the Examples, and evaluated according to the assay below. The IC₅₀ of the compounds are presented in Table 1 as follows:
+, 20000 nM≥IC₅₀≥500 nM;
++, 500 nM>IC₅₀≥5 nM:
+++, 5 nM>IC₅₀.

### Characterization Conditions

Reverse phase HPLC conditions used for determination of retention times in Table 1:
Column: ZORBAX (SB-C18 2.1×50 mm, 5 μm)
Mobile phase A: 95% H₂O, 5% MeCN (with 0.1% Formic Acid)
Mobile phase B: 5% H₂O, 95% MeCN (with 0.1% Formic Acid)
Flow rate: 1.0 mL/min
Gradient: 20 to 100% B in 3.5 min (for Rₜ without *) or 20 to 100% B in 5.5 min (for Rₜ with *).
   In vitro Biological Example: Enzyme-Linked Immunosorbent Assay—ELISA

96 Well plates were coated with 1 μg/mL of human PD-L1 (obtained from R&D) in

PBS overnight at 4° C. The wells were then blocked with 2% BSA in PBS (WN) with 0.05% TWEEN-20 for 1 hour at 37° C. The plates were washed 3 times with PBS/0.05% TWEEN-20 and the compounds were serial diluted (1:5) in dilution medium and added to the ELISA plates. Human PD-1 and biotin 0.3μg/mL (ACRO Biosystems) were added and incubated for 1 hour at 37° C. then washed 3 times with PBS/0.05% TWEEN-20. A second block was performed with 2% BSA in PBS (WN)/0.05% TWEEN-20 for 10 min at 37° C. and was washed 3 times with PBS/0.05% TWEEN-20. Streptavidin—HRP was added for 1 hour at 37° C. then washed 3 times with PBS/0.05% TWEEN-20. TMB substrate was added and reacted for 20 min at 37° C. A stop solution (2 N aqueous H₂SO₄) was added. The absorbance was read at 450 nm using a micro-plate spectrophotometer. The results are shown in Table 1.

TABLE 1

| | | MS: | RP |
|---|---|---|---|
| | ELISA | (ES) | HPLC |
| | IC₅₀ | m/z | Rₜ |
| Compound Structure | (nM) | (M + H) | (min) |
| 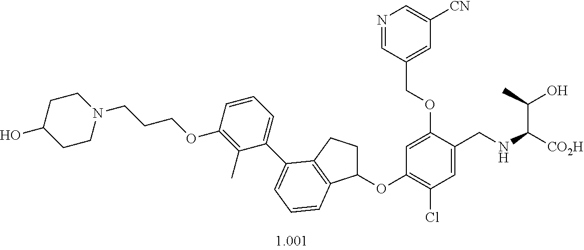 [SMILES: Cc1c(OCCCN2CCC(O)CC2)cccc1-c1cccc2c1CCC2Oc1cc(OCc2cncc(C#N)c2)c(CN[C@H](C(=O)O)[C@@H](C)O)cc1Cl] | +++ | 755.30 | 1.76 |
| 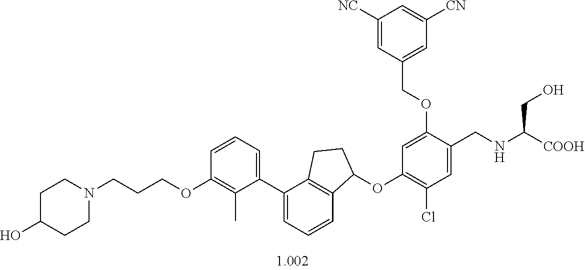 [SMILES: Cc1c(OCCCN2CCC(O)CC2)cccc1-c1cccc2c1CCC2Oc1cc(OCc2cc(C#N)cc(C#N)c2)c(CN[C@@H](CO)C(=O)O)cc1Cl] | +++ | 765.20 | 1.73 |
| 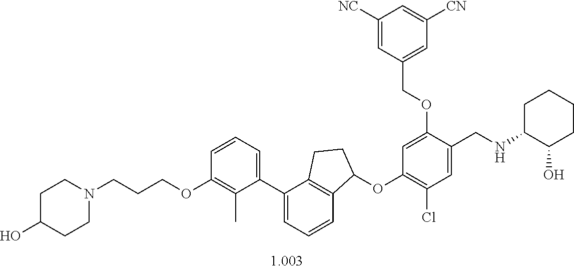 [SMILES: Cc1c(OCCCN2CCC(O)CC2)cccc1-c1cccc2c1CCC2Oc1cc(OCc2cc(C#N)cc(C#N)c2)c(CN[C@@H]2CCCC[C@@H]2O)cc1Cl] | +++ | 775.30 | 1.99 |
| 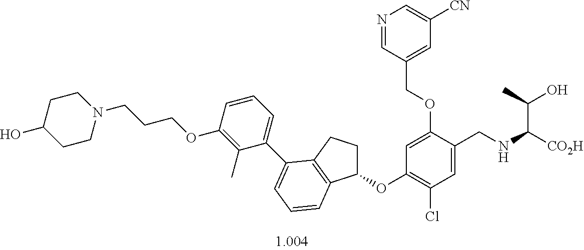 [SMILES: Cc1c(OCCCN2CCC(O)CC2)cccc1-c1cccc2c1CC[C@@H]2Oc1cc(OCc2cncc(C#N)c2)c(CN[C@H](C(=O)O)[C@@H](C)O)cc1Cl] | +++ | 755.20 | 2.00 |
| 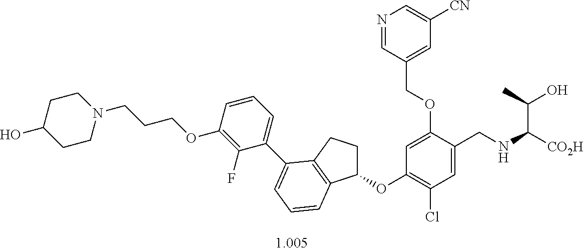 [SMILES: C[C@@H](O)[C@H](NCc1cc(Cl)c(O[C@H]2CCc3c(-c4cccc(OCCCN5CCC(O)CC5)c4F)cccc32)cc1OCc1cncc(C#N)c1)C(=O)O] | +++ | 759.20 | 1.55 |
| 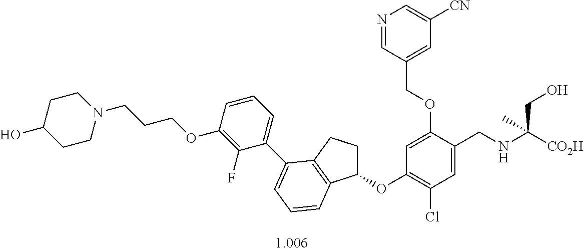 [SMILES: C[C@@](CO)(NCc1cc(Cl)c(O[C@H]2CCc3c(-c4cccc(OCCCN5CCC(O)CC5)c4F)cccc32)cc1OCc1cncc(C#N)c1)C(=O)O] | +++ | 759.20 | 1.57 |
| 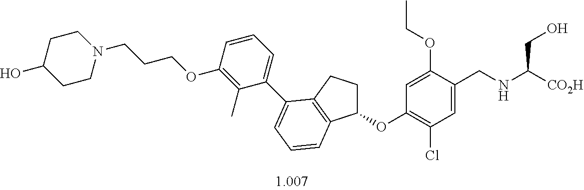 [SMILES: CCOc1cc(O[C@H]2CCc3c(-c4cccc(OCCCN5CCC(O)CC5)c4C)cccc32)c(Cl)cc1CN[C@@H](CO)C(=O)O] | +++ | 653.30 | 1.82 |
| 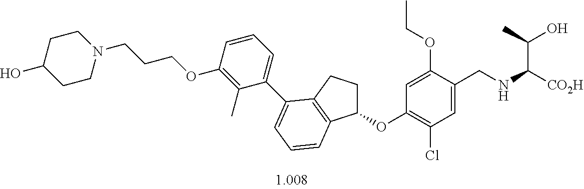 [SMILES: CCOc1cc(O[C@H]2CCc3c(-c4cccc(OCCCN5CCC(O)CC5)c4C)cccc32)c(Cl)cc1CN[C@H](C(=O)O)[C@@H](C)O] | +++ | 667.20 | 1.5* |
| 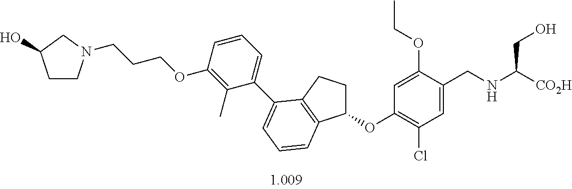 [SMILES: CCOc1cc(O[C@H]2CCc3c(-c4cccc(OCCCN5CC[C@@H](O)C5)c4C)cccc32)c(Cl)cc1CN[C@@H](CO)C(=O)O] | +++ | 639.20 | 1.75 |
| 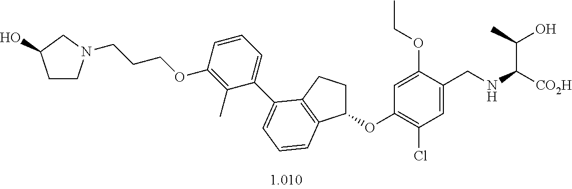 [SMILES: CCOc1cc(O[C@H]2CCc3c(-c4cccc(OCCCN5CC[C@@H](O)C5)c4C)cccc32)c(Cl)cc1CN[C@H](C(=O)O)[C@@H](C)O] | +++ | 653.30 | 1.91 |
| 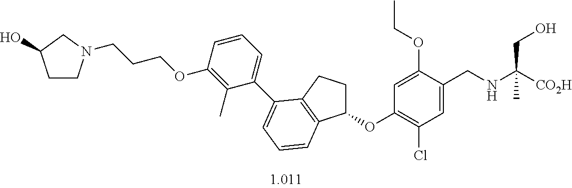 | +++ | 653.30 | 1.52 |
| 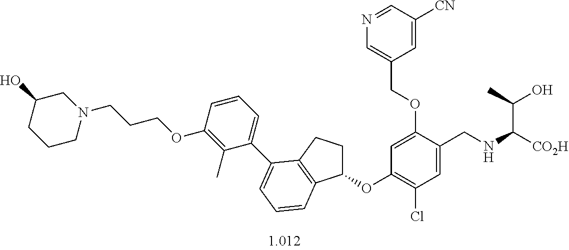 [SMILES: Cc1c(OCCCN2CCC[C@@H](O)C2)cccc1-c1cccc2c1CC[C@@H]2Oc1cc(OCc2cncc(C#N)c2)c(CN[C@H](C(=O)O)[C@@H](C)O)cc1Cl] | +++ | 755.20 | 1.73 |
| 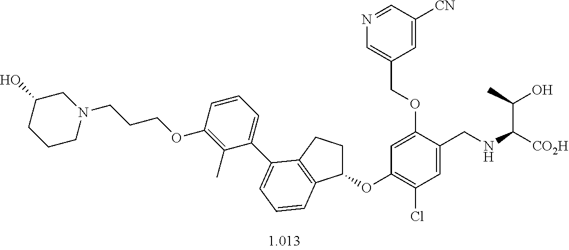 [SMILES: Cc1c(OCCCN2CCC[C@H](O)C2)cccc1-c1cccc2c1CC[C@@H]2Oc1cc(OCc2cncc(C#N)c2)c(CN[C@H](C(=O)O)[C@@H](C)O)cc1Cl] | +++ | 755.20 | 1.71 |
| 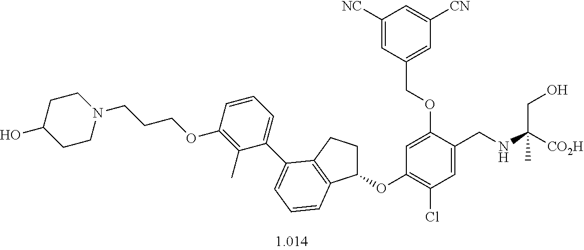 [SMILES: Cc1c(OCCCN2CCC(O)CC2)cccc1-c1cccc2c1CC[C@@H]2Oc1cc(OCc2cc(C#N)cc(C#N)c2)c(CN[C@@](C)(CO)C(=O)O)cc1Cl] | +++ | 779.30 | 1.98 |
| 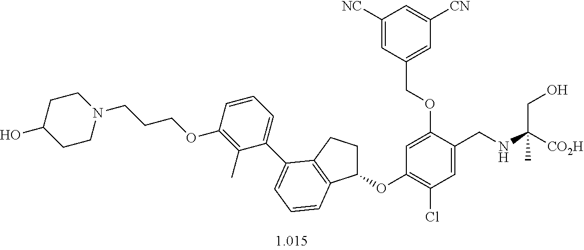 [SMILES: Cc1c(OCCCN2CCC(O)CC2)cccc1-c1cccc2c1CC[C@@H]2Oc1cc(OCc2cc(C#N)cc(C#N)c2)c(CN[C@@](C)(CO)C(=O)O)cc1Cl] | +++ | 779.30 | 2.10 |
| 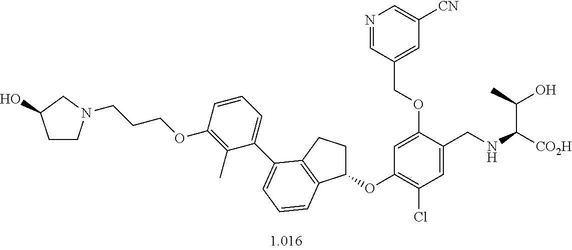 [SMILES: Cc1c(OCCCN2CC[C@@H](O)C2)cccc1-c1cccc2c1CC[C@@H]2Oc1cc(OCc2cncc(C#N)c2)c(CN[C@H](C(=O)O)[C@@H](C)O)cc1Cl] | +++ | 741.30 | 1.77 |
| 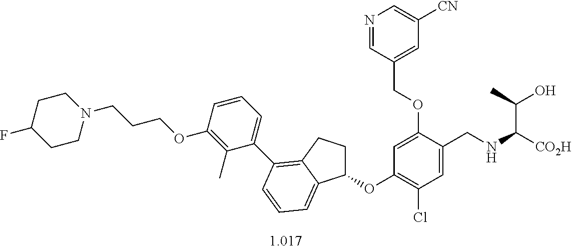 [SMILES: Cc1c(OCCCN2CCC(F)CC2)cccc1-c1cccc2c1CC[C@@H]2Oc1cc(OCc2cncc(C#N)c2)c(CN[C@H](C(=O)O)[C@@H](C)O)cc1Cl] | +++ | 757.20 | 1.90 |
| 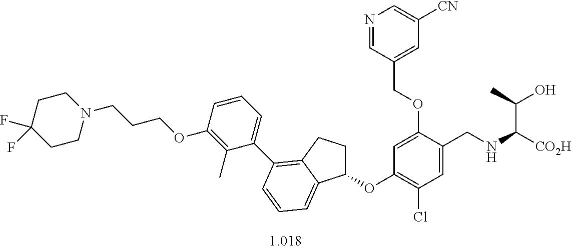 [SMILES: Cc1c(OCCCN2CCC(F)(F)CC2)cccc1-c1cccc2c1CC[C@@H]2Oc1cc(OCc2cncc(C#N)c2)c(CN[C@H](C(=O)O)[C@@H](C)O)cc1Cl] | ++ | 776.30 | 1.90 |
| 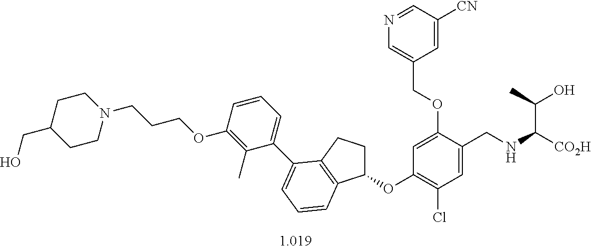 [SMILES: Cc1c(OCCCN2CCC(CO)CC2)cccc1-c1cccc2c1CC[C@@H]2Oc1cc(OCc2cncc(C#N)c2)c(CN[C@H](C(=O)O)[C@@H](C)O)cc1Cl] | +++ | 769.20 | 2.00 |
| 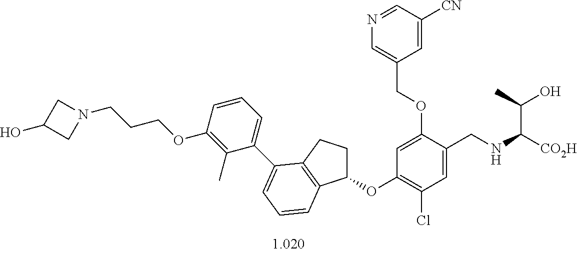 [SMILES: Cc1c(OCCCN2CC(O)C2)cccc1-c1cccc2c1CC[C@@H]2Oc1cc(OCc2cncc(C#N)c2)c(CN[C@H](C(=O)O)[C@@H](C)O)cc1Cl] | +++ | 727.20 | 2.01 |
| 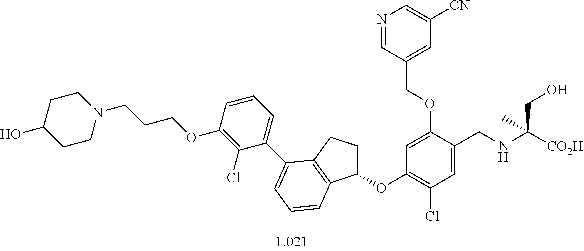 [SMILES: C[C@@](CO)(NCc1cc(Cl)c(O[C@H]2CCc3c(-c4cccc(OCCCN5CCC(O)CC5)c4Cl)cccc32)cc1OCc1cncc(C#N)c1)C(=O)O] | +++ | 775.20 | 1.75 |
| 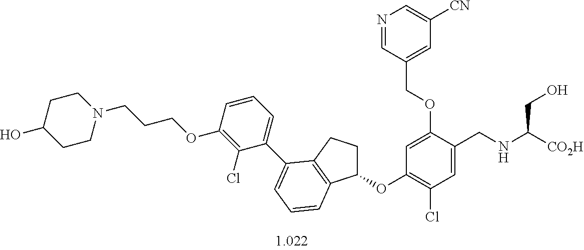 [SMILES: N#Cc1cncc(COc2cc(O[C@H]3CCc4c(-c5cccc(OCCCN6CCC(O)CC6)c5Cl)cccc43)c(Cl)cc2CN[C@@H](CO)C(=O)O)c1] | +++ | 761.10 | 1.67 |
| 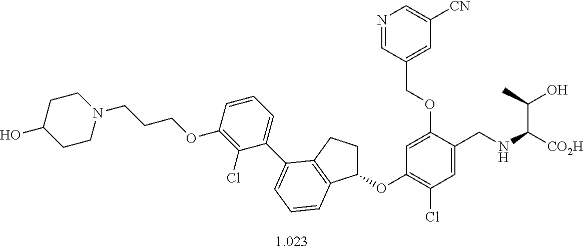 [SMILES: C[C@@H](O)[C@H](NCc1cc(Cl)c(O[C@H]2CCc3c(-c4cccc(OCCCN5CCC(O)CC5)c4Cl)cccc32)cc1OCc1cncc(C#N)c1)C(=O)O] | +++ | 775.20 | 1.68 |
| 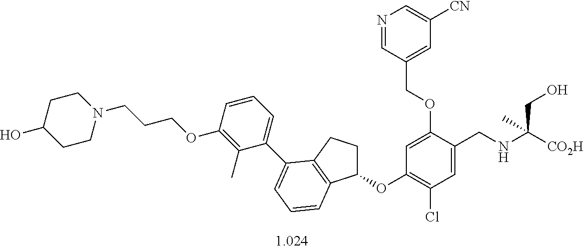 [SMILES: Cc1c(OCCCN2CCC(O)CC2)cccc1-c1cccc2c1CC[C@@H]2Oc1cc(OCc2cncc(C#N)c2)c(CN[C@@](C)(CO)C(=O)O)cc1Cl] | +++ | 755.20 | 1.80 |
| 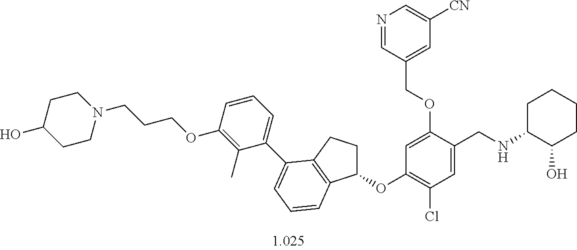 [SMILES: Cc1c(OCCCN2CCC(O)CC2)cccc1-c1cccc2c1CC[C@@H]2Oc1cc(OCc2cncc(C#N)c2)c(CN[C@@H]2CCCC[C@@H]2O)cc1Cl] | +++ | 751.20 | 2.07 |
| 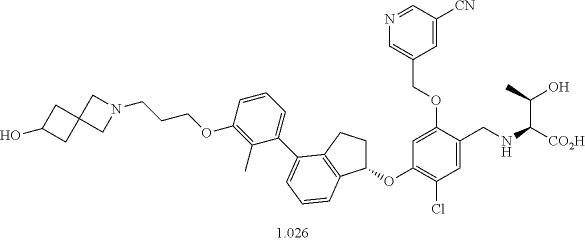 [SMILES: Cc1c(OCCCN2CC3(CC(O)C3)C2)cccc1-c1cccc2c1CC[C@@H]2Oc1cc(OCc2cncc(C#N)c2)c(CN[C@H](C(=O)O)[C@@H](C)O)cc1Cl] | +++ | 767.20 | 1.84 |
| 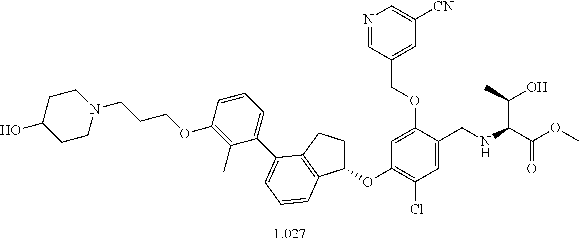 [SMILES: COC(=O)[C@@H](NCc1cc(Cl)c(O[C@H]2CCc3c(-c4cccc(OCCCN5CCC(O)CC5)c4C)cccc32)cc1OCc1cncc(C#N)c1)[C@@H](C)O] | ++ | 769.30 | 3.26* |
| 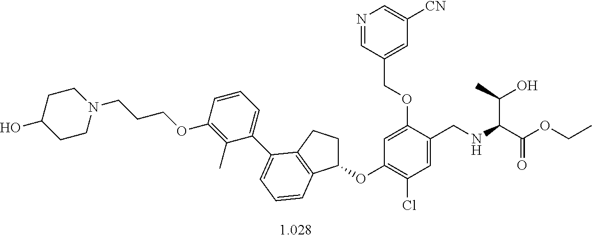 [SMILES: CCOC(=O)[C@@H](NCc1cc(Cl)c(O[C@H]2CCc3c(-c4cccc(OCCCN5CCC(O)CC5)c4C)cccc32)cc1OCc1cncc(C#N)c1)[C@@H](C)O] | ++ | 783.20 | 3.58* |
| 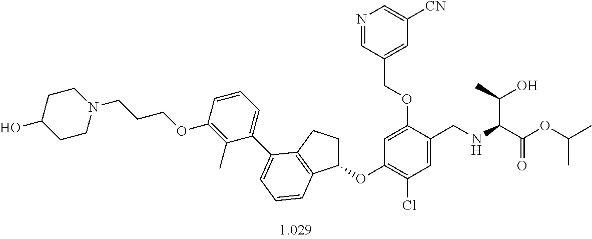 [SMILES: Cc1c(OCCCN2CCC(O)CC2)cccc1-c1cccc2c1CC[C@@H]2Oc1cc(OCc2cncc(C#N)c2)c(CN[C@H](C(=O)OC(C)C)[C@@H](C)O)cc1Cl] | ++ | 783.30 | 2.24 |
| 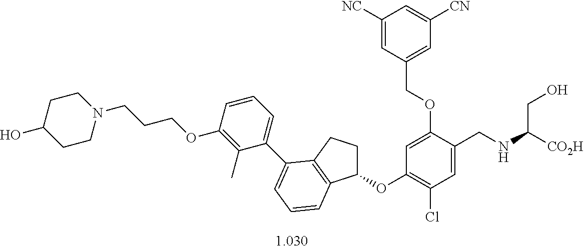 [SMILES: Cc1c(OCCCN2CCC(O)CC2)cccc1-c1cccc2c1CC[C@@H]2Oc1cc(OCc2cc(C#N)cc(C#N)c2)c(CN[C@@H](CO)C(=O)O)cc1Cl] | +++ | 765.20 | 1.96 |
| 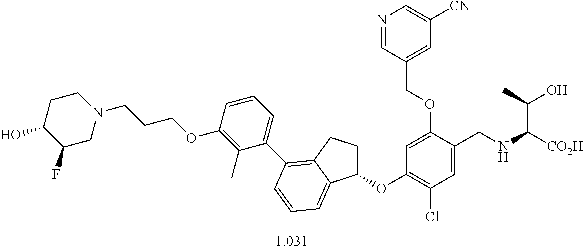 [SMILES: Cc1c(OCCCN2CC[C@@H](O)[C@H](F)C2)cccc1-c1cccc2c1CC[C@@H]2Oc1cc(OCc2cncc(C#N)c2)c(CN[C@H](C(=O)O)[C@@H](C)O)cc1Cl] | ++ | 774.30 | 1.70 |
| 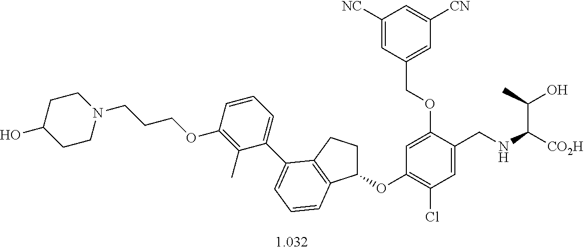 [SMILES: Cc1c(OCCCN2CCC(O)CC2)cccc1-c1cccc2c1CC[C@@H]2Oc1cc(OCc2cc(C#N)cc(C#N)c2)c(CN[C@H](C(=O)O)[C@@H](C)O)cc1Cl] | +++ | 779.30 | 2.02 |
| 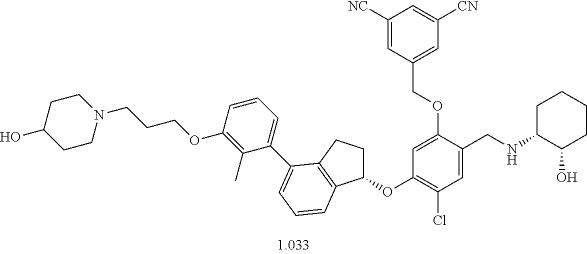 [SMILES: Cc1c(OCCCN2CCC(O)CC2)cccc1-c1cccc2c1CC[C@@H]2Oc1cc(OCc2cc(C#N)cc(C#N)c2)c(CN[C@@H]2CCCC[C@@H]2O)cc1Cl] | +++ | 775.30 | 2.30 |
| 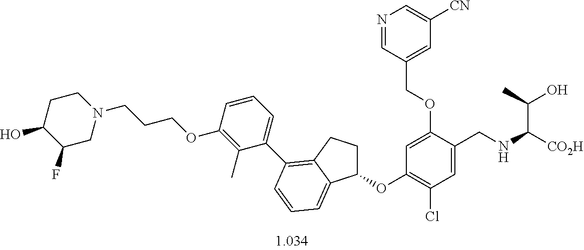 [SMILES: Cc1c(OCCCN2CC[C@H](O)[C@H](F)C2)cccc1-c1cccc2c1CC[C@@H]2Oc1cc(OCc2cncc(C#N)c2)c(CN[C@H](C(=O)O)[C@@H](C)O)cc1Cl] | +++ | 774.30 | 1.70 |
| 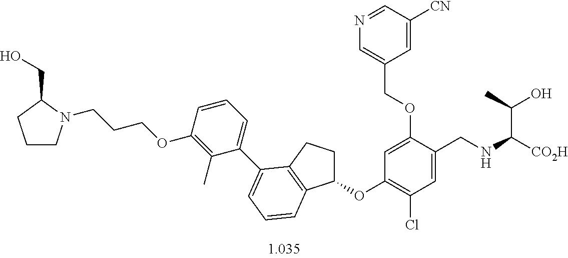 [SMILES: Cc1c(OCCCN2CCC[C@H]2CO)cccc1-c1cccc2c1CC[C@@H]2Oc1cc(OCc2cncc(C#N)c2)c(CN[C@H](C(=O)O)[C@@H](C)O)cc1Cl] | +++ | 755.20 | 1.90 |
| 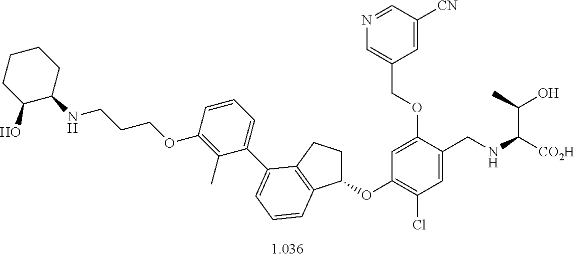 [SMILES: Cc1c(OCCCN[C@@H]2CCCC[C@@H]2O)cccc1-c1cccc2c1CC[C@@H]2Oc1cc(OCc2cncc(C#N)c2)c(CN[C@H](C(=O)O)[C@@H](C)O)cc1Cl] | +++ | 769.20 | 2.07 |
| 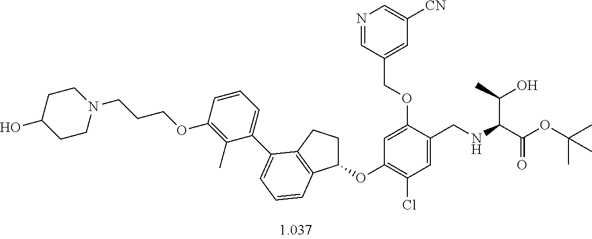 [SMILES: Cc1c(OCCCN2CCC(O)CC2)cccc1-c1cccc2c1CC[C@@H]2Oc1cc(OCc2cncc(C#N)c2)c(CN[C@H](C(=O)OC(C)(C)C)[C@@H](C)O)cc1Cl] | ++ | 811.30 | 2.32 |
| 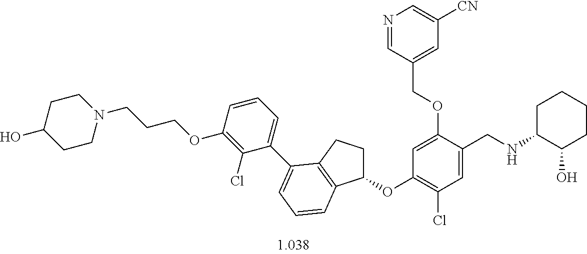 [SMILES: N#Cc1cncc(COc2cc(O[C@H]3CCc4c(-c5cccc(OCCCN6CCC(O)CC6)c5Cl)cccc43)c(Cl)cc2CN[C@@H]2CCCC[C@@H]2O)c1] | +++ | 771.20 | 1.99 |
| 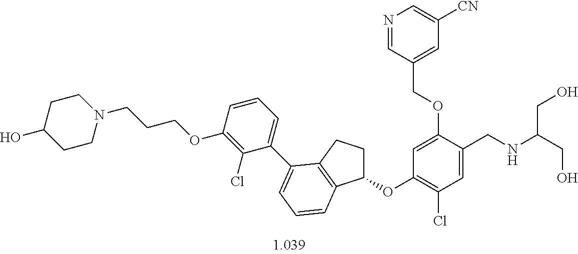 [SMILES: N#Cc1cncc(COc2cc(O[C@H]3CCc4c(-c5cccc(OCCCN6CCC(O)CC6)c5Cl)cccc43)c(Cl)cc2CNC(CO)CO)c1] | +++ | 747.20 | 1.74 |
| 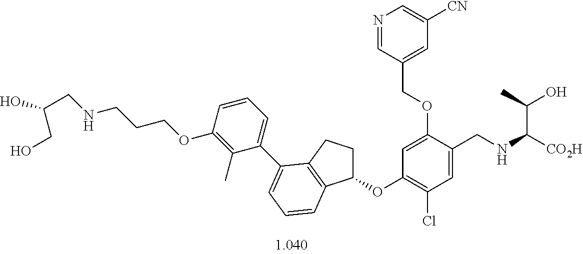 [SMILES: Cc1c(OCCCNC[C@@H](O)CO)cccc1-c1cccc2c1CC[C@@H]2Oc1cc(OCc2cncc(C#N)c2)c(CN[C@H](C(=O)O)[C@@H](C)O)cc1Cl] | +++ | 745.30 | 1.70 |
| 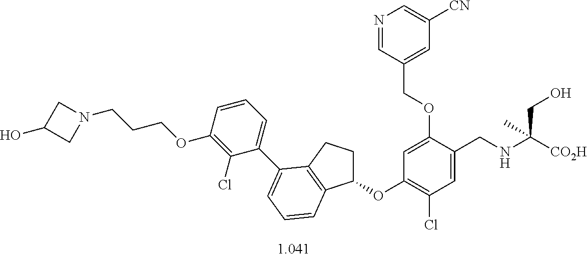 [SMILES: C[C@@](CO)(NCc1cc(Cl)c(O[C@H]2CCc3c(-c4cccc(OCCCN5CC(O)C5)c4Cl)cccc32)cc1OCc1cncc(C#N)c1)C(=O)O] | +++ | 747.20 | 1.90 |
| 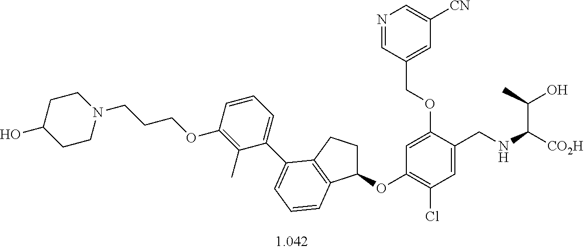 [SMILES: Cc1c(OCCCN2CCC(O)CC2)cccc1-c1cccc2c1CC[C@H]2Oc1cc(OCc2cncc(C#N)c2)c(CN[C@H](C(=O)O)[C@@H](C)O)cc1Cl] | +++ | 755.20 | 1.68 |
| 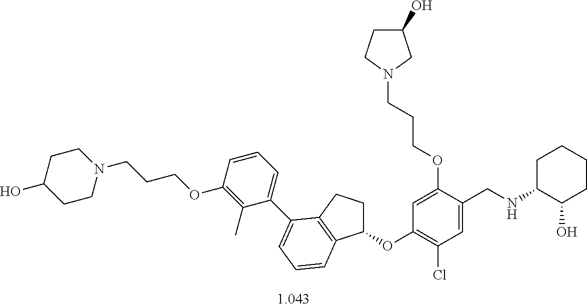 [SMILES: Cc1c(OCCCN2CCC(O)CC2)cccc1-c1cccc2c1CC[C@@H]2Oc1cc(OCCCN2CC[C@@H](O)C2)c(CN[C@@H]2CCCC[C@@H]2O)cc1Cl] | +++ | 762.30 | 1.20 |
| 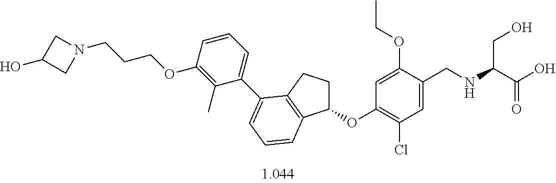 [SMILES: CCOc1cc(O[C@H]2CCc3c(-c4cccc(OCCCN5CC(O)C5)c4C)cccc32)c(Cl)cc1CN[C@@H](CO)C(=O)O] | +++ | 625.20 | 1.88 |
| 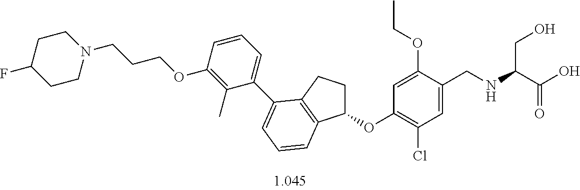 [SMILES: CCOc1cc(O[C@H]2CCc3c(-c4cccc(OCCCN5CCC(F)CC5)c4C)cccc32)c(Cl)cc1CN[C@@H](CO)C(=O)O] | +++ | 655.20 | 1.76 |
| 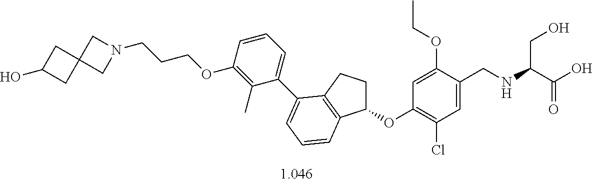 [SMILES: CCOc1cc(O[C@H]2CCc3c(-c4cccc(OCCCN5CC6(CC(O)C6)C5)c4C)cccc32)c(Cl)cc1CN[C@@H](CO)C(=O)O] | +++ | 665.20 | 1.76 |
| 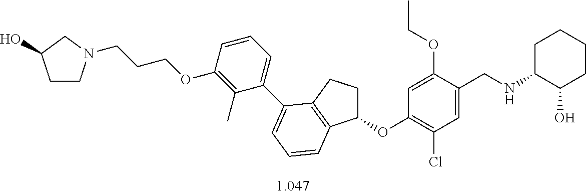 [SMILES: CCOc1cc(O[C@H]2CCc3c(-c4cccc(OCCCN5CC[C@@H](O)C5)c4C)cccc32)c(Cl)cc1CN[C@@H]1CCCC[C@@H]1O] | ++ | 649.20 | 1.76 |
| 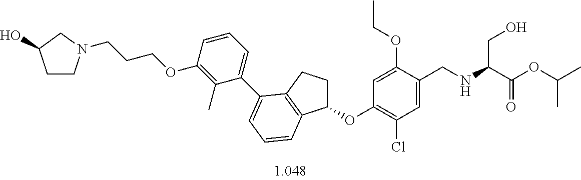 [SMILES: CCOc1cc(O[C@H]2CCc3c(-c4cccc(OCCCN5CC[C@@H](O)C5)c4C)cccc32)c(Cl)cc1CN[C@@H](CO)C(=O)OC(C)C] | ++ | 681.30 | 2.31 |
| 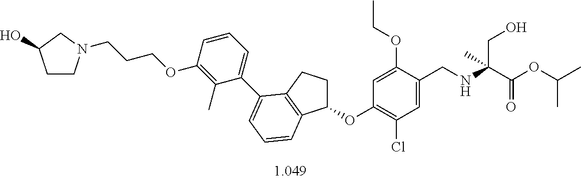 [SMILES: CCOc1cc(O[C@H]2CCc3c(-c4cccc(OCCCN5CC[C@@H](O)C5)c4C)cccc32)c(Cl)cc1CN[C@@](C)(CO)C(=O)OC(C)C] | ++ | 695.30 | 2.05 |
| 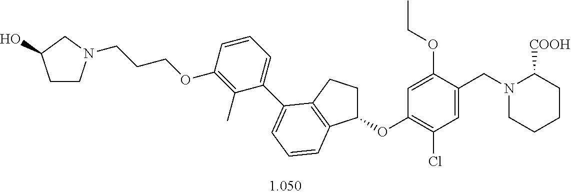 [SMILES: CCOc1cc(O[C@H]2CCc3c(-c4cccc(OCCCN5CC[C@@H](O)C5)c4C)cccc32)c(Cl)cc1CN1CCCC[C@H]1C(=O)O] | ++ | 663.30 | 1.86 |
| 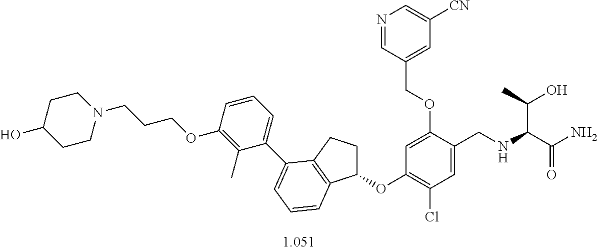 [SMILES: Cc1c(OCCCN2CCC(O)CC2)cccc1-c1cccc2c1CC[C@@H]2Oc1cc(OCc2cncc(C#N)c2)c(CN[C@H](C(N)=O)[C@@H](C)O)cc1Cl] | +++ | 754.20 | 1.69 |
| 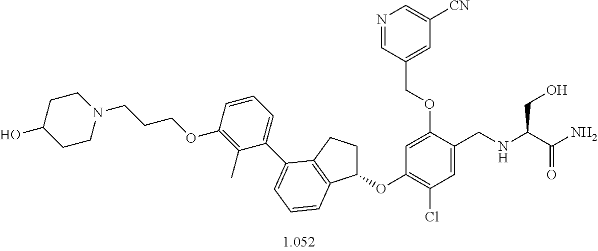 [SMILES: Cc1c(OCCCN2CCC(O)CC2)cccc1-c1cccc2c1CC[C@@H]2Oc1cc(OCc2cncc(C#N)c2)c(CN[C@@H](CO)C(N)=O)cc1Cl] | +++ | 740.30 | 1.55 |
| 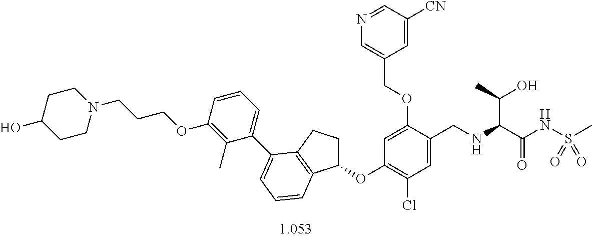 [SMILES: Cc1c(OCCCN2CCC(O)CC2)cccc1-c1cccc2c1CC[C@@H]2Oc1cc(OCc2cncc(C#N)c2)c(CN[C@H](C(=O)NS(C)(=O)=O)[C@@H](C)O)cc1Cl] | +++ | 832.70 | 2.03 |
| 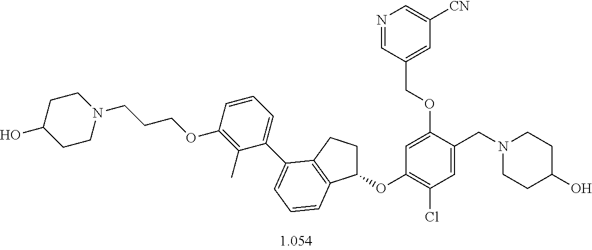 [SMILES: Cc1c(OCCCN2CCC(O)CC2)cccc1-c1cccc2c1CC[C@@H]2Oc1cc(OCc2cncc(C#N)c2)c(CN2CCC(O)CC2)cc1Cl] | +++ | 737.20 | 1.70 |
| 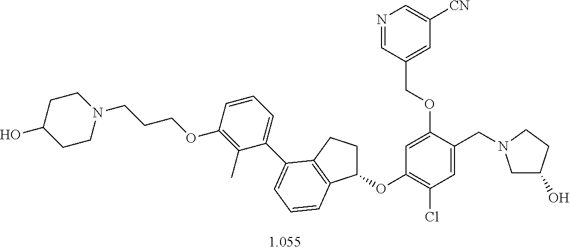 [SMILES: Cc1c(OCCCN2CCC(O)CC2)cccc1-c1cccc2c1CC[C@@H]2Oc1cc(OCc2cncc(C#N)c2)c(CN2CC[C@H](O)C2)cc1Cl] | +++ | 723.20 | 1.56 |
| 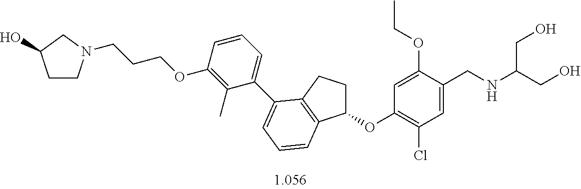 [SMILES: CCOc1cc(O[C@H]2CCc3c(-c4cccc(OCCCN5CC[C@@H](O)C5)c4C)cccc32)c(Cl)cc1CNC(CO)CO] | +++ | 625.20 | 1.64 |
| 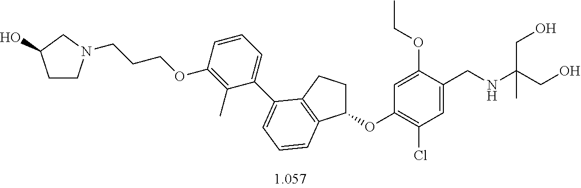 [SMILES: CCOc1cc(O[C@H]2CCc3c(-c4cccc(OCCCN5CC[C@@H](O)C5)c4C)cccc32)c(Cl)cc1CNC(C)(CO)CO] | +++ | 639.30 | 1.85 |
| 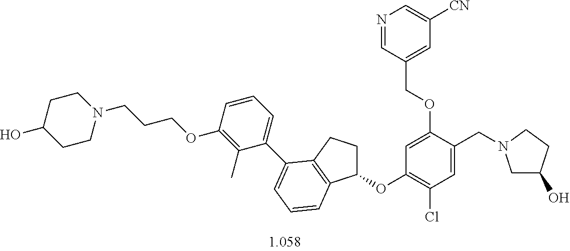 [SMILES: Cc1c(OCCCN2CCC(O)CC2)cccc1-c1cccc2c1CC[C@@H]2Oc1cc(OCc2cncc(C#N)c2)c(CN2CC[C@@H](O)C2)cc1Cl] | +++ | 723.20 | 1.77 |
| 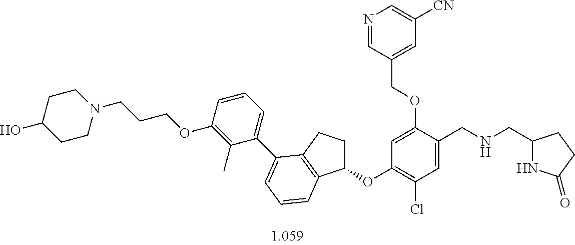 [SMILES: Cc1c(OCCCN2CCC(O)CC2)cccc1-c1cccc2c1CC[C@@H]2Oc1cc(OCc2cncc(C#N)c2)c(CNCC2CCC(=O)N2)cc1Cl] | +++ | 750.30 | 1.55 |
| 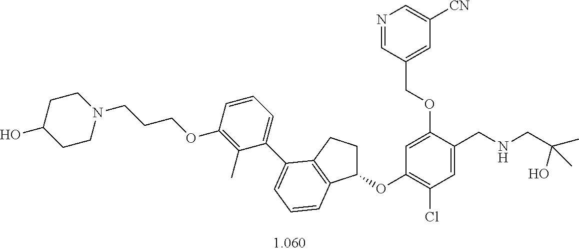 [SMILES: Cc1c(OCCCN2CCC(O)CC2)cccc1-c1cccc2c1CC[C@@H]2Oc1cc(OCc2cncc(C#N)c2)c(CNCC(C)(C)O)cc1Cl] | +++ | 725.30 | 1.79 |
| 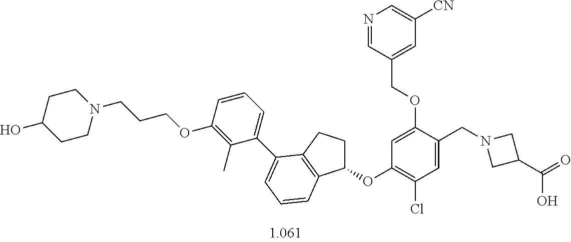 [SMILES: Cc1c(OCCCN2CCC(O)CC2)cccc1-c1cccc2c1CC[C@@H]2Oc1cc(OCc2cncc(C#N)c2)c(CN2CC(C(=O)O)C2)cc1Cl] | +++ | 737.20 | 2.51 |
| 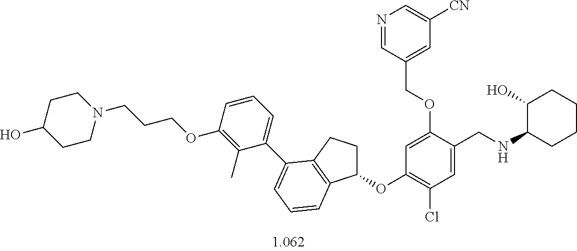 [SMILES: Cc1c(OCCCN2CCC(O)CC2)cccc1-c1cccc2c1CC[C@@H]2Oc1cc(OCc2cncc(C#N)c2)c(CN[C@@H]2CCCC[C@H]2O)cc1Cl] | +++ | 751.20 | 1.91 |
| 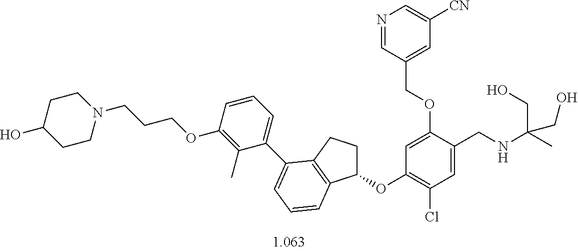 [SMILES: Cc1c(OCCCN2CCC(O)CC2)cccc1-c1cccc2c1CC[C@@H]2Oc1cc(OCc2cncc(C#N)c2)c(CNC(C)(CO)CO)cc1Cl] | ++ | 741.30 | 1.54 |
| 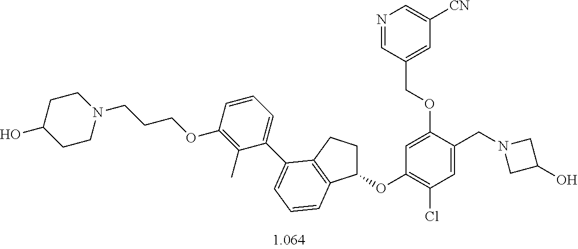 [SMILES: Cc1c(OCCCN2CCC(O)CC2)cccc1-c1cccc2c1CC[C@@H]2Oc1cc(OCc2cncc(C#N)c2)c(CN2CC(O)C2)cc1Cl] | +++ | 709.20 | 2.06 |
| 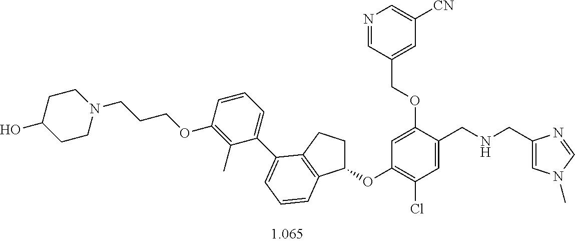 [SMILES: Cc1c(OCCCN2CCC(O)CC2)cccc1-c1cccc2c1CC[C@@H]2Oc1cc(OCc2cncc(C#N)c2)c(CNCc2cn(C)cn2)cc1Cl] | +++ | 747.20 | 1.65 |
| 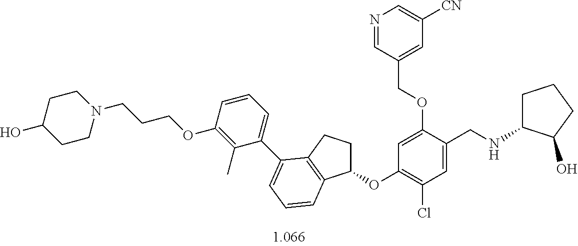 [SMILES: Cc1c(OCCCN2CCC(O)CC2)cccc1-c1cccc2c1CC[C@@H]2Oc1cc(OCc2cncc(C#N)c2)c(CN[C@@H]2CCC[C@H]2O)cc1Cl] | +++ | 737.20 | 1.83 |
| 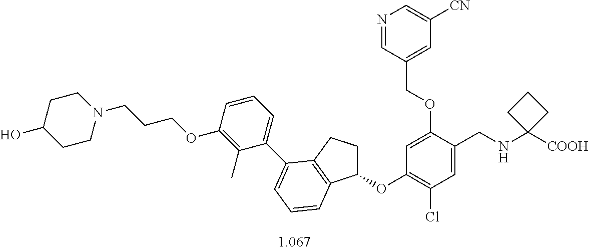 [SMILES: Cc1c(OCCCN2CCC(O)CC2)cccc1-c1cccc2c1CC[C@@H]2Oc1cc(OCc2cncc(C#N)c2)c(CNC2(C(=O)O)CCC2)cc1Cl] | +++ | 751.20 | 1.88 |
| 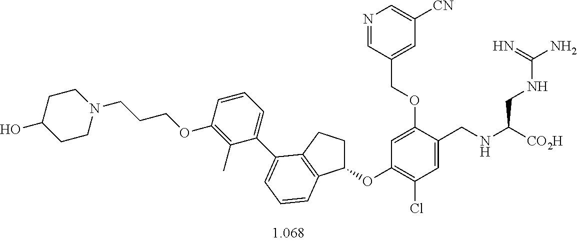 [SMILES: Cc1c(OCCCN2CCC(O)CC2)cccc1-c1cccc2c1CC[C@@H]2Oc1cc(OCc2cncc(C#N)c2)c(CN[C@@H](CNC(=N)N)C(=O)O)cc1Cl] | +++ | 782.30 | 1.76 |
| 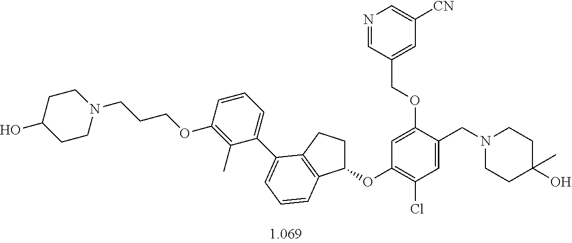 [SMILES: Cc1c(OCCCN2CCC(O)CC2)cccc1-c1cccc2c1CC[C@@H]2Oc1cc(OCc2cncc(C#N)c2)c(CN2CCC(C)(O)CC2)cc1Cl] | +++ | 751.20 | 1.64 |
| 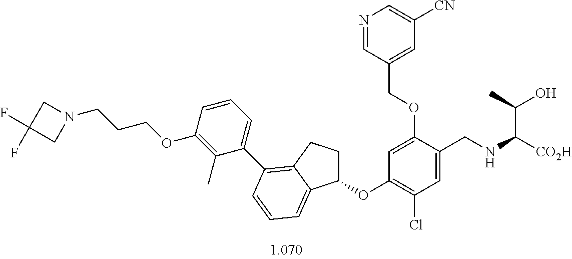 [SMILES: Cc1c(OCCCN2CC(F)(F)C2)cccc1-c1cccc2c1CC[C@@H]2Oc1cc(OCc2cncc(C#N)c2)c(CN[C@H](C(=O)O)[C@@H](C)O)cc1Cl] | +++ | 755.20 | 1.70 |
| 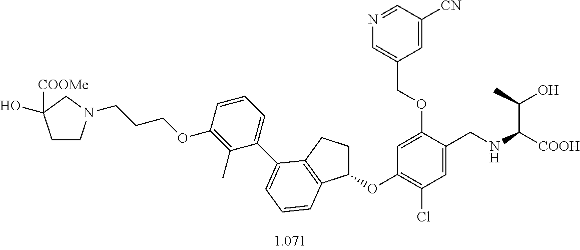 [SMILES: COC(=O)C1(O)CCN(CCCOc2cccc(-c3cccc4c3CC[C@@H]4Oc3cc(OCc4cncc(C#N)c4)c(CN[C@H](C(=O)O)[C@@H](C)O)cc3Cl)c2C)C1] | +++ | 799.70 | 1.89 |
| 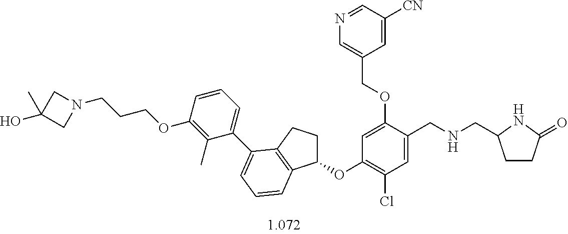 [SMILES: Cc1c(OCCCN2CC(C)(O)C2)cccc1-c1cccc2c1CC[C@@H]2Oc1cc(OCc2cncc(C#N)c2)c(CNCC2CCC(=O)N2)cc1Cl] | +++ | 736.20 | 1.65 |
| 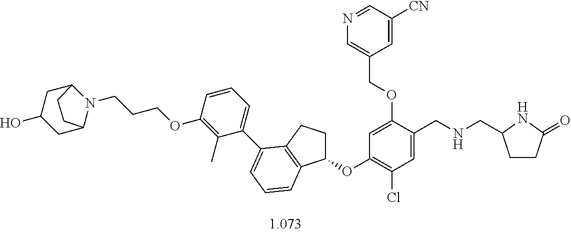 [SMILES: Cc1c(OCCCN2C3CCC2CC(O)C3)cccc1-c1cccc2c1CC[C@@H]2Oc1cc(OCc2cncc(C#N)c2)c(CNCC2CCC(=O)N2)cc1Cl] | +++ | 776.20 | 1.63 |
| 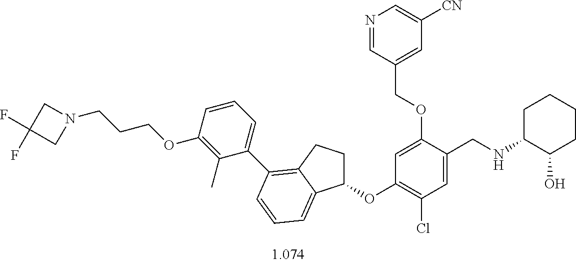 [SMILES: Cc1c(OCCCN2CC(F)(F)C2)cccc1-c1cccc2c1CC[C@@H]2Oc1cc(OCc2cncc(C#N)c2)c(CN[C@@H]2CCCC[C@@H]2O)cc1Cl] | +++ | 751.20 | 2.20 |
| 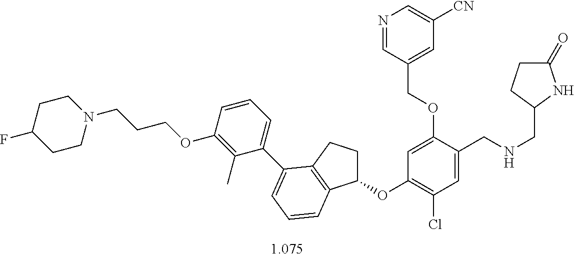 [SMILES: Cc1c(OCCCN2CCC(F)CC2)cccc1-c1cccc2c1CC[C@@H]2Oc1cc(OCc2cncc(C#N)c2)c(CNCC2CCC(=O)N2)cc1Cl] | +++ | 752.20 | 1.66 |
| 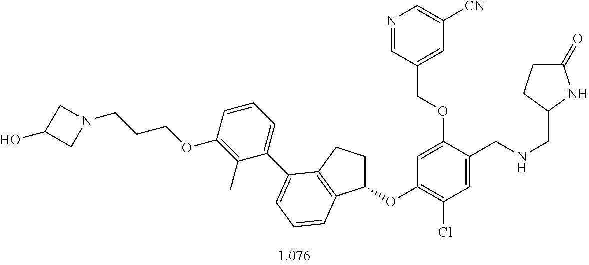 [SMILES: Cc1c(OCCCN2CC(O)C2)cccc1-c1cccc2c1CC[C@@H]2Oc1cc(OCc2cncc(C#N)c2)c(CNCC2CCC(=O)N2)cc1Cl] | +++ | 722.20 | 1.56 |
| 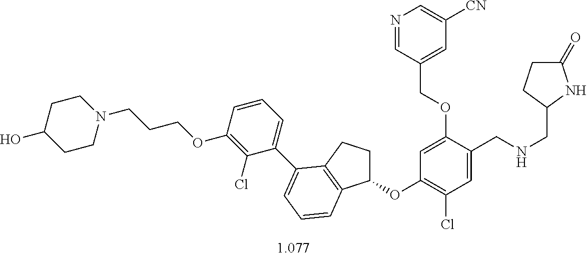 [SMILES: N#Cc1cncc(COc2cc(O[C@H]3CCc4c(-c5cccc(OCCCN6CCC(O)CC6)c5Cl)cccc43)c(Cl)cc2CNCC2CCC(=O)N2)c1] | +++ | 770.30 | 1.62 |
| 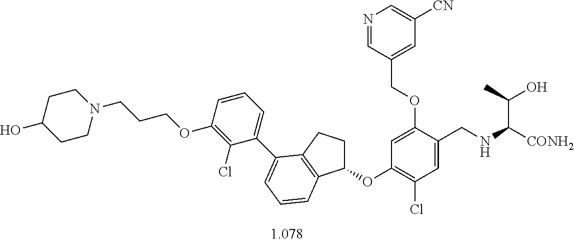 [SMILES: C[C@@H](O)[C@H](NCc1cc(Cl)c(O[C@H]2CCc3c(-c4cccc(OCCCN5CCC(O)CC5)c4Cl)cccc32)cc1OCc1cncc(C#N)c1)C(N)=O] | +++ | 774.20 | 1.62 |
| 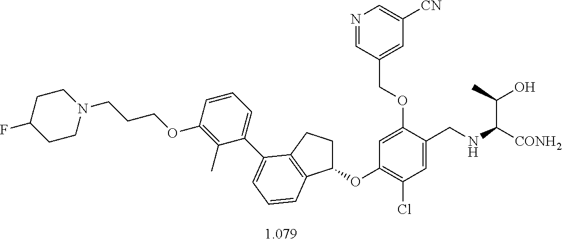 [SMILES: Cc1c(OCCCN2CCC(F)CC2)cccc1-c1cccc2c1CC[C@@H]2Oc1cc(OCc2cncc(C#N)c2)c(CN[C@H](C(N)=O)[C@@H](C)O)cc1Cl] | ++ | 756.20 | 1.68 |
| 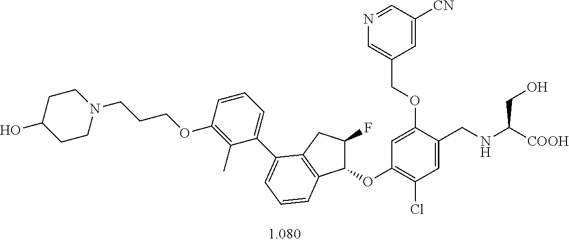 [SMILES: Cc1c(OCCCN2CCC(O)CC2)cccc1-c1cccc2c1C[C@@H](F)[C@@H]2Oc1cc(OCc2cncc(C#N)c2)c(CN[C@@H](CO)C(=O)O)cc1Cl] | +++ | 759.20 | 1.83 |
| 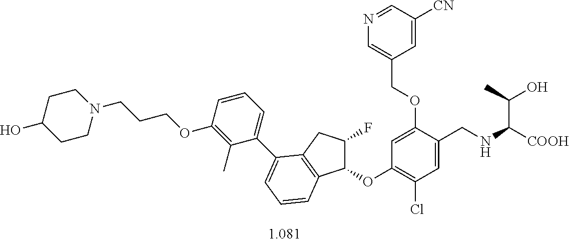 [SMILES: Cc1c(OCCCN2CCC(O)CC2)cccc1-c1cccc2c1C[C@H](F)[C@@H]2Oc1cc(OCc2cncc(C#N)c2)c(CN[C@H](C(=O)O)[C@@H](C)O)cc1Cl] | +++ | 773.20 | 1.74^{#} |
| 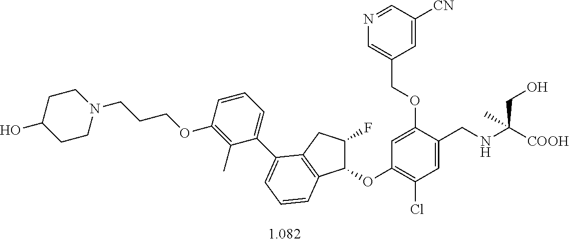 [SMILES: Cc1c(OCCCN2CCC(O)CC2)cccc1-c1cccc2c1C[C@H](F)[C@@H]2Oc1cc(OCc2cncc(C#N)c2)c(CN[C@@](C)(CO)C(=O)O)cc1Cl] | +++ | 773.20 | 1.76^{#} |
| 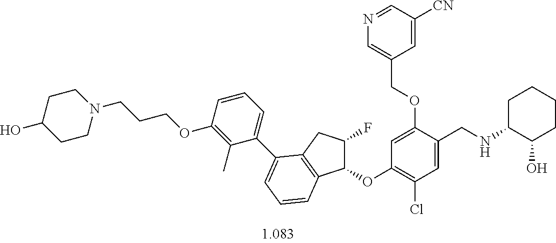 [SMILES: Cc1c(OCCCN2CCC(O)CC2)cccc1-c1cccc2c1C[C@H](F)[C@@H]2Oc1cc(OCc2cncc(C#N)c2)c(CN[C@@H]2CCCC[C@@H]2O)cc1Cl] | +++ | 769.20 | 1.61^{#} |
| 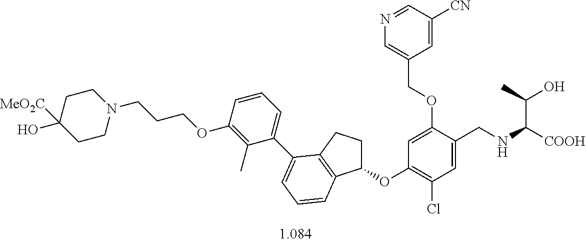 [SMILES: COC(=O)C1(O)CCN(CCCOc2cccc(-c3cccc4c3CC[C@@H]4Oc3cc(OCc4cncc(C#N)c4)c(CN[C@H](C(=O)O)[C@@H](C)O)cc3Cl)c2C)CC1] | +++ | 713.20 | 1.62 |
| 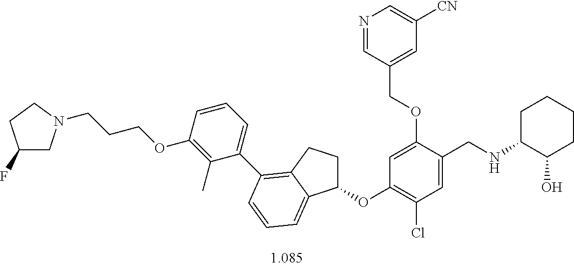 [SMILES: Cc1c(OCCCN2CC[C@H](F)C2)cccc1-c1cccc2c1CC[C@@H]2Oc1cc(OCc2cncc(C#N)c2)c(CN[C@@H]2CCCC[C@@H]2O)cc1Cl] | +++ | 739.20 | 2.20 |
| 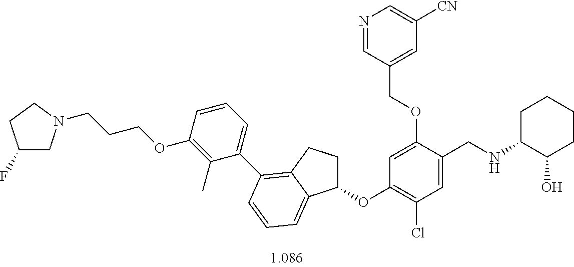 [SMILES: Cc1c(OCCCN2CC[C@@H](F)C2)cccc1-c1cccc2c1CC[C@@H]2Oc1cc(OCc2cncc(C#N)c2)c(CN[C@@H]2CCCC[C@@H]2O)cc1Cl] | +++ | 739.20 | 2.10 |
| 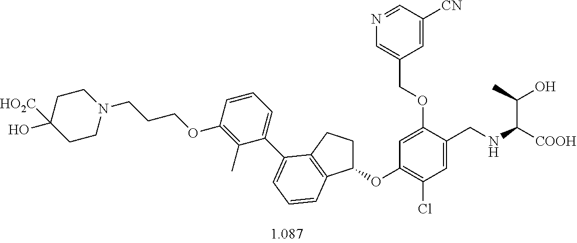 [SMILES: Cc1c(OCCCN2CCC(O)(C(=O)O)CC2)cccc1-c1cccc2c1CC[C@@H]2Oc1cc(OCc2cncc(C#N)c2)c(CN[C@H](C(=O)O)[C@@H](C)O)cc1Cl] | +++ | 799.20 | 1.79 |
| 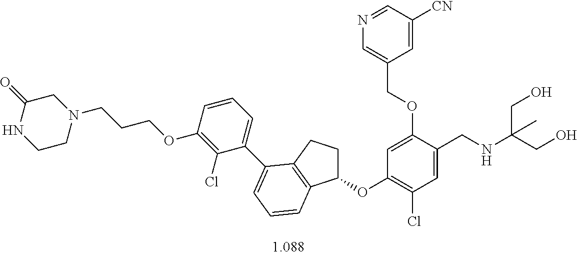 [SMILES: CC(CO)(CO)NCc1cc(Cl)c(O[C@H]2CCc3c(-c4cccc(OCCCN5CCNC(=O)C5)c4Cl)cccc32)cc1OCc1cncc(C#N)c1] | +++ | 760.70 | 2.26 |
| 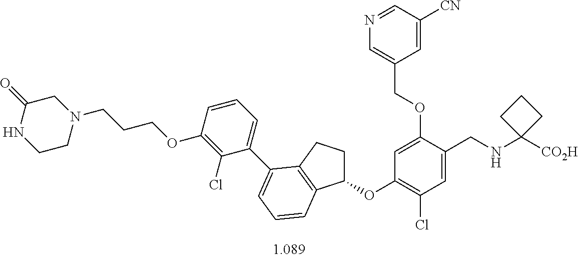 [SMILES: N#Cc1cncc(COc2cc(O[C@H]3CCc4c(-c5cccc(OCCCN6CCNC(=O)C6)c5Cl)cccc43)c(Cl)cc2CNC2(C(=O)O)CCC2)c1] | +++ | 770.70 | 2.35* |
| 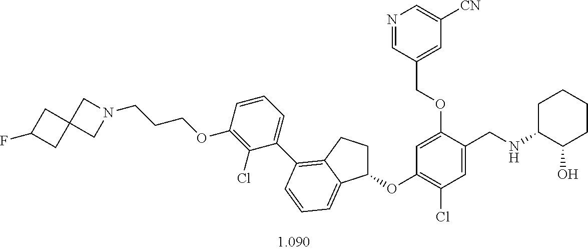 [SMILES: N#Cc1cncc(COc2cc(O[C@H]3CCc4c(-c5cccc(OCCCN6CC7(CC(F)C7)C6)c5Cl)cccc43)c(Cl)cc2CN[C@@H]2CCCC[C@@H]2O)c1] | +++ | 785.30 | 2.20 |
| 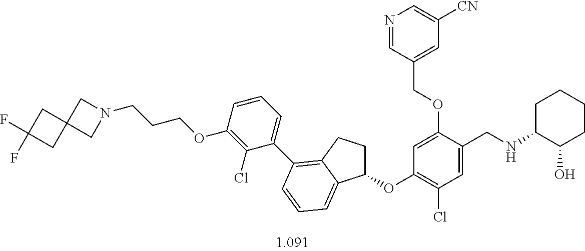 [SMILES: N#Cc1cncc(COc2cc(O[C@H]3CCc4c(-c5cccc(OCCCN6CC7(C6)CC(F)(F)C7)c5Cl)cccc43)c(Cl)cc2CN[C@@H]2CCCC[C@@H]2O)c1] | +++ | 803.20 | 2.70 |
| 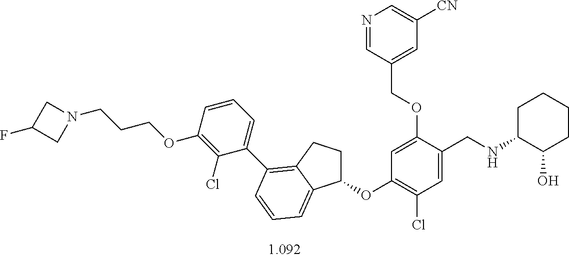 [SMILES: N#Cc1cncc(COc2cc(O[C@H]3CCc4c(-c5cccc(OCCCN6CC(F)C6)c5Cl)cccc43)c(Cl)cc2CN[C@@H]2CCCC[C@@H]2O)c1] | +++ | 746.80 | 2.30 |
| 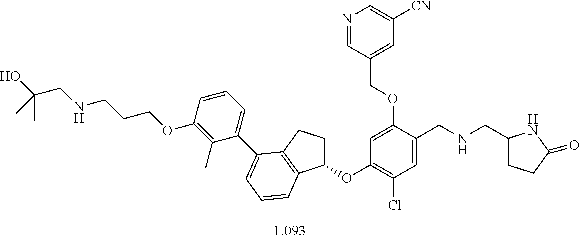 [SMILES: Cc1c(OCCCNCC(C)(C)O)cccc1-c1cccc2c1CC[C@@H]2Oc1cc(OCc2cncc(C#N)c2)c(CNCC2CCC(=O)N2)cc1Cl] | +++ | 738.60 | 1.86 |
| 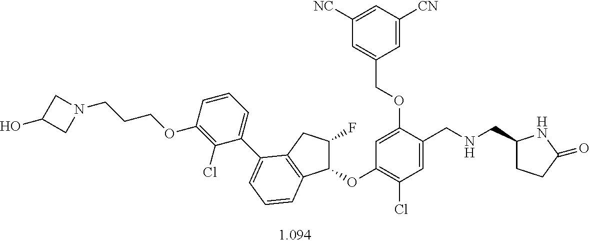 [SMILES: N#Cc1cc(C#N)cc(COc2cc(O[C@@H]3c4cccc(-c5cccc(OCCCN6CC(O)C6)c5Cl)c4C[C@@H]3F)c(Cl)cc2CNC[C@@H]2CCC(=O)N2)c1] | ++ | 784.70 | 1.73^{#} |
| 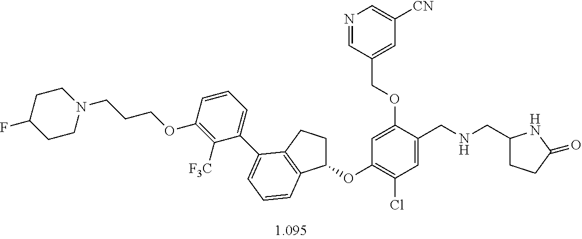 [SMILES: N#Cc1cncc(COc2cc(O[C@H]3CCc4c(-c5cccc(OCCCN6CCC(F)CC6)c5C(F)(F)F)cccc43)c(Cl)cc2CNCC2CCC(=O)N2)c1] | ++ | 806.70 | 1.93 |
| 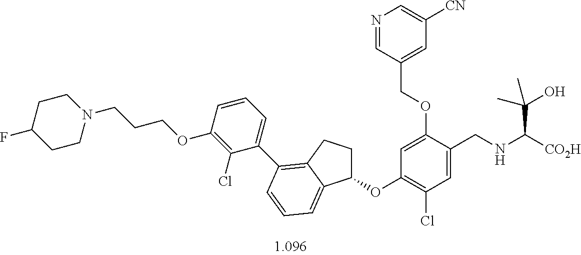 [SMILES: CC(C)(O)[C@H](NCc1cc(Cl)c(O[C@H]2CCc3c(-c4cccc(OCCCN5CCC(F)CC5)c4Cl)cccc32)cc1OCc1cncc(C#N)c1)C(=O)O] | +++ | 791.50 | 1.70 |
| 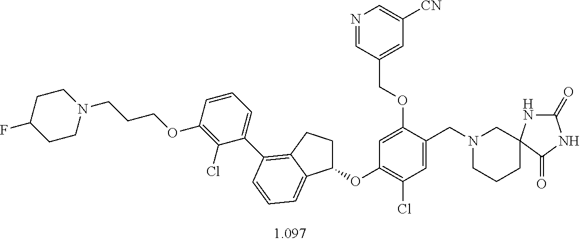 [SMILES: N#Cc1cncc(COc2cc(O[C@H]3CCc4c(-c5cccc(OCCCN6CCC(F)CC6)c5Cl)cccc43)c(Cl)cc2CN2CCCC3(C2)NC(=O)NC3=O)c1] | ++ | 827.50 | 1.80 |
| 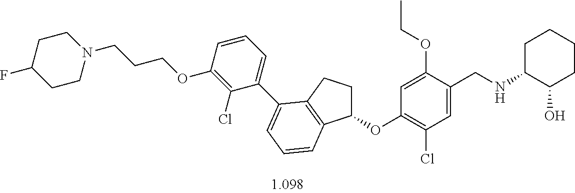 [SMILES: CCOc1cc(O[C@H]2CCc3c(-c4cccc(OCCCN5CCC(F)CC5)c4Cl)cccc32)c(Cl)cc1CN[C@@H]1CCCC[C@@H]1O] | ++ | 685.50 | 1.93 |
| 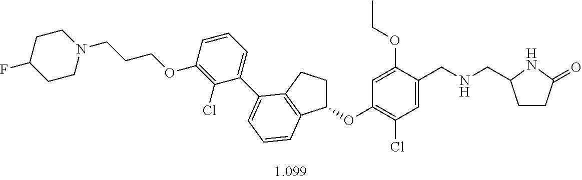 [SMILES: CCOc1cc(O[C@H]2CCc3c(-c4cccc(OCCCN5CCC(F)CC5)c4Cl)cccc32)c(Cl)cc1CNCC1CCC(=O)N1] | ++ | 684.50 | 1.79 |
| 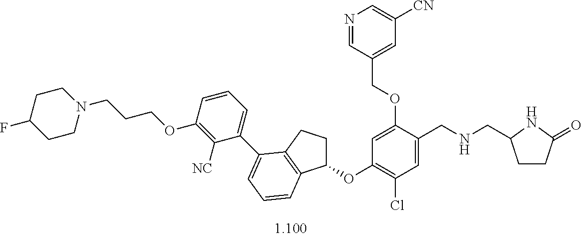 [SMILES: N#Cc1cncc(COc2cc(O[C@H]3CCc4c(-c5cccc(OCCCN6CCC(F)CC6)c5C#N)cccc43)c(Cl)cc2CNCC2CCC(=O)N2)c1] | ++ | 763.50 | 1.51 |
| 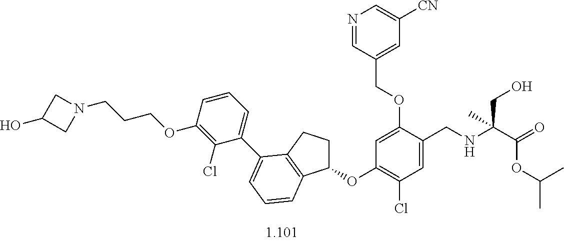 [SMILES: CC(C)OC(=O)[C@](C)(CO)NCc1cc(Cl)c(O[C@H]2CCc3c(-c4cccc(OCCCN5CC(O)C5)c4Cl)cccc32)cc1OCc1cncc(C#N)c1] | +++ | 789.50 | 2.02 |
| 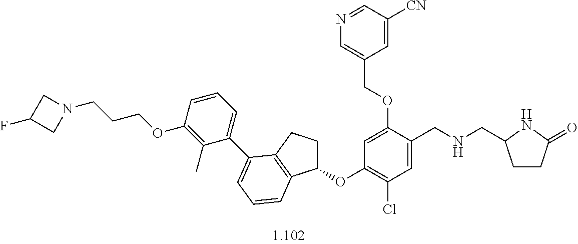 [SMILES: Cc1c(OCCCN2CC(F)C2)cccc1-c1cccc2c1CC[C@@H]2Oc1cc(OCc2cncc(C#N)c2)c(CNCC2CCC(=O)N2)cc1Cl] | +++ | 724.80 | 3.19 |
| 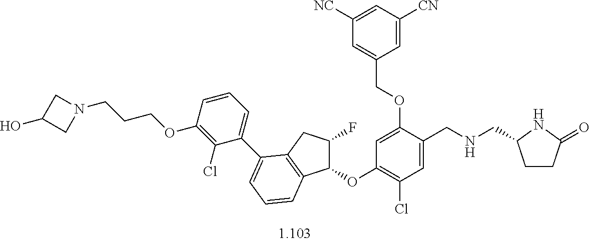 [SMILES: N#Cc1cc(C#N)cc(COc2cc(O[C@@H]3c4cccc(-c5cccc(OCCCN6CC(O)C6)c5Cl)c4C[C@@H]3F)c(Cl)cc2CNC[C@H]2CCC(=O)N2)c1] | +++ | 784.50 | 1.84^{#} |
| 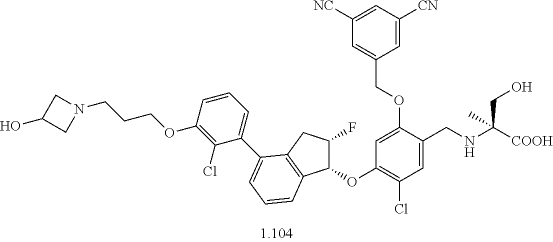 [SMILES: C[C@@](CO)(NCc1cc(Cl)c(O[C@@H]2c3cccc(-c4cccc(OCCCN5CC(O)C5)c4Cl)c3C[C@@H]2F)cc1OCc1cc(C#N)cc(C#N)c1)C(=O)O] | +++ | 789.50 | 1.78^{#} |
| 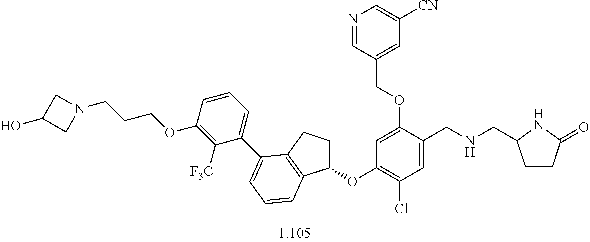 [SMILES: N#Cc1cncc(COc2cc(O[C@H]3CCc4c(-c5cccc(OCCCN6CC(O)C6)c5C(F)(F)F)cccc43)c(Cl)cc2CNCC2CCC(=O)N2)c1] | ++ | 776.50 | 1.67 |
| 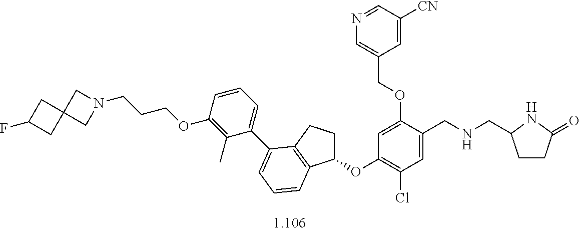 [SMILES: Cc1c(OCCCN2CC3(CC(F)C3)C2)cccc1-c1cccc2c1CC[C@@H]2Oc1cc(OCc2cncc(C#N)c2)c(CNCC2CCC(=O)N2)cc1Cl] | ++ | 764.80 | 3.83 |
| 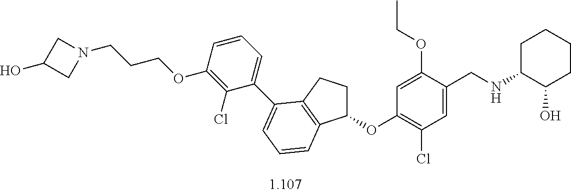 [SMILES: CCOc1cc(O[C@H]2CCc3c(-c4cccc(OCCCN5CC(O)C5)c4Cl)cccc32)c(Cl)cc1CN[C@@H]1CCCC[C@@H]1O] | ++ | 655.20 | 1.30 |
| 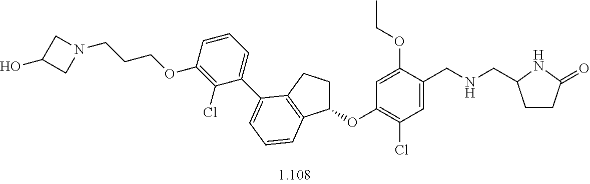 [SMILES: CCOc1cc(O[C@H]2CCc3c(-c4cccc(OCCCN5CC(O)C5)c4Cl)cccc32)c(Cl)cc1CNCC1CCC(=O)N1] | ++ | 654.20 | 1.14 |
| 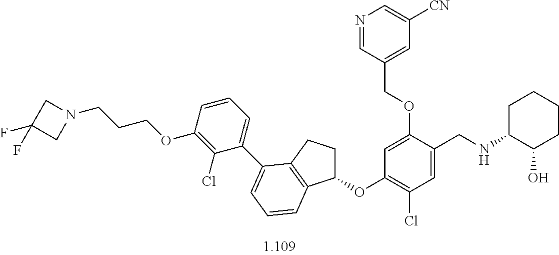 [SMILES: N#Cc1cncc(COc2cc(O[C@H]3CCc4c(-c5cccc(OCCCN6CC(F)(F)C6)c5Cl)cccc43)c(Cl)cc2CN[C@@H]2CCCC[C@@H]2O)c1] | ++ | 763.50 | 2.07 |
| 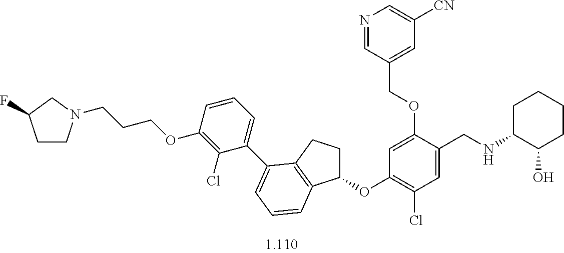 [SMILES: N#Cc1cncc(COc2cc(O[C@H]3CCc4c(-c5cccc(OCCCN6CC[C@@H](F)C6)c5Cl)cccc43)c(Cl)cc2CN[C@@H]2CCCC[C@@H]2O)c1] | +++ | 759.50 | 1.70 |
| 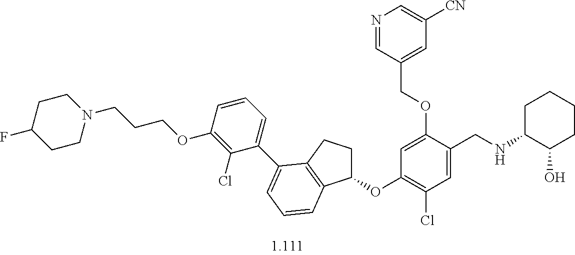 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCCC[C@@H]2O)c1] | +++ | 773.20 | 3.05* |
| 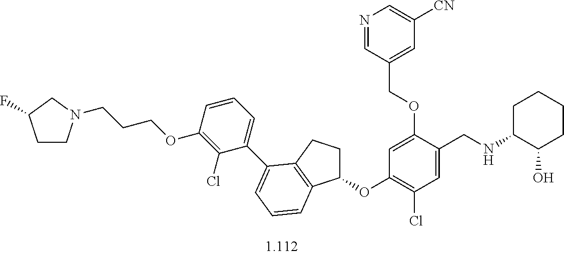 [SMILES: N#Cc1cncc(COc2cc(O[C@H]3CCc4c(-c5cccc(OCCCN6CC[C@H](F)C6)c5Cl)cccc43)c(Cl)cc2CN[C@@H]2CCCC[C@@H]2O)c1] | +++ | 759.50 | 1.90 |
| 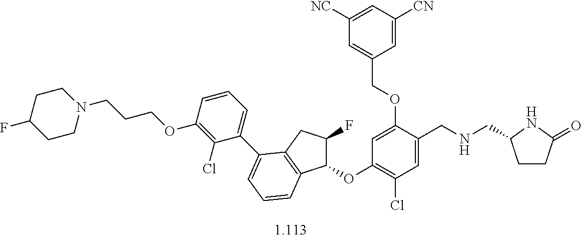 [SMILES: N#Cc1cc(C#N)cc(COc2cc(O[C@@H]3c4cccc(-c5cccc(OCCCN6CCC(F)CC6)c5Cl)c4C[C@H]3F)c(Cl)cc2CNC[C@H]2CCC(=O)N2)c1] | ++ | 814.50 | 1.83 |
| 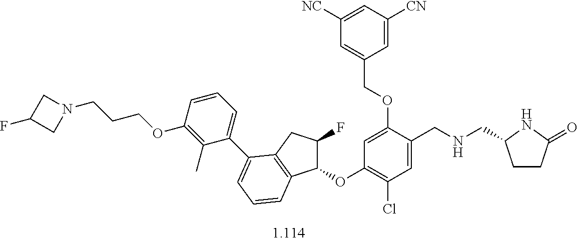 [SMILES: Cc1c(OCCCN2CC(F)C2)cccc1-c1cccc2c1C[C@@H](F)[C@@H]2Oc1cc(OCc2cc(C#N)cc(C#N)c2)c(CNC[C@H]2CCC(=O)N2)cc1Cl] | ++ | 786.30 | 1.69 |
| 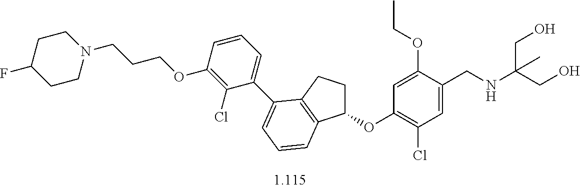 [SMILES: CCOc1cc(O[C@H]2CCc3c(-c4cccc(OCCCN5CCC(F)CC5)c4Cl)cccc32)c(Cl)cc1CNC(C)(CO)CO] | ++ | 675.60 | 1.67 |
| 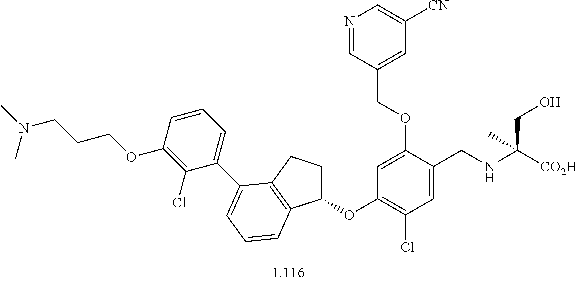 [SMILES: CN(C)CCCOc1cccc(-c2cccc3c2CC[C@@H]3Oc2cc(OCc3cncc(C#N)c3)c(CN[C@@](C)(CO)C(=O)O)cc2Cl)c1Cl] | +++ | 618.60 | 2.68 |
| 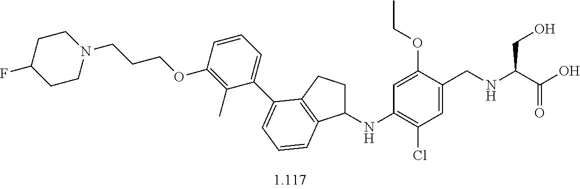 [SMILES: CCOc1cc(NC2CCc3c(-c4cccc(OCCCN5CCC(F)CC5)c4C)cccc32)c(Cl)cc1CN[C@@H](CO)C(=O)O] | ++ | 652.20 | 1.99 |
| 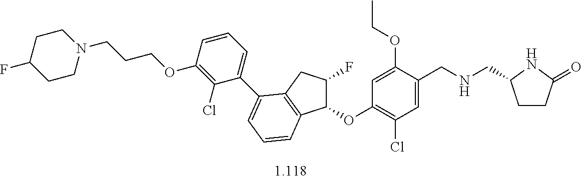 [SMILES: CCOc1cc(O[C@@H]2c3cccc(-c4cccc(OCCCN5CCC(F)CC5)c4Cl)c3C[C@@H]2F)c(Cl)cc1CNC[C@H]1CCC(=O)N1] | ++ | 702.30 | 1.64^{#} |
| 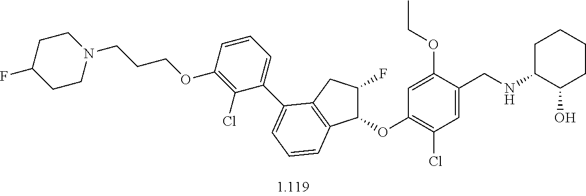 [SMILES: CCOc1cc(O[C@@H]2c3cccc(-c4cccc(OCCCN5CCC(F)CC5)c4Cl)c3C[C@@H]2F)c(Cl)cc1CN[C@@H]1CCCC[C@@H]1O] | ++ | 703.50 | 1.67^{#} |
| 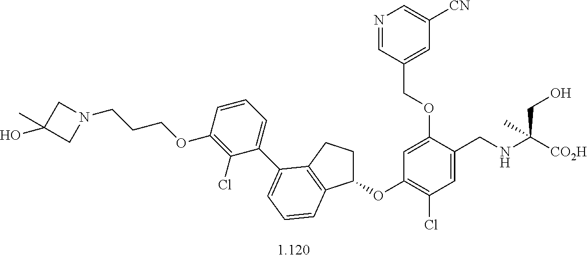 [SMILES: CC1(O)CN(CCCOc2cccc(-c3cccc4c3CC[C@@H]4Oc3cc(OCc4cncc(C#N)c4)c(CN[C@@](C)(CO)C(=O)O)cc3Cl)c2Cl)C1] | +++ | 761.70 | 3.45 |
| 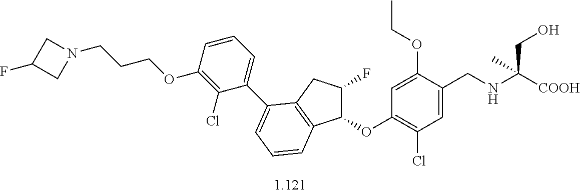 [SMILES: CCOc1cc(O[C@@H]2c3cccc(-c4cccc(OCCCN5CC(F)C5)c4Cl)c3C[C@@H]2F)c(Cl)cc1CN[C@@](C)(CO)C(=O)O] | ++ | 679.20 | 1.66^{#} |
| 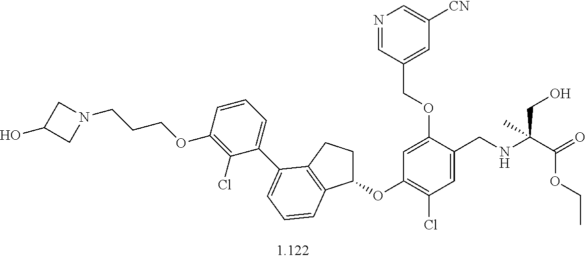 [SMILES: CCOC(=O)[C@](C)(CO)NCc1cc(Cl)c(O[C@H]2CCc3c(-c4cccc(OCCCN5CC(O)C5)c4Cl)cccc32)cc1OCc1cncc(C#N)c1] | +++ | 775.20 | 1.76 |
| 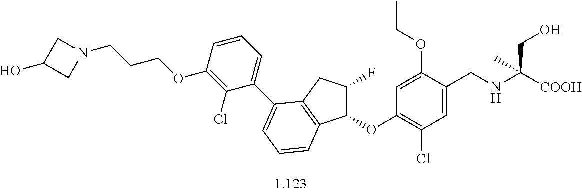 [SMILES: CCOc1cc(O[C@@H]2c3cccc(-c4cccc(OCCCN5CC(O)C5)c4Cl)c3C[C@@H]2F)c(Cl)cc1CN[C@@](C)(CO)C(=O)O] | ++ | 677.10 | 1.56^{#} |
| 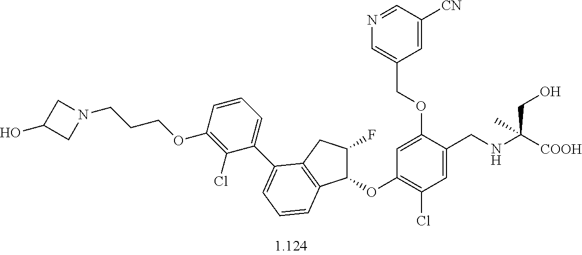 [SMILES: C[C@@](CO)(NCc1cc(Cl)c(O[C@@H]2c3cccc(-c4cccc(OCCCN5CC(O)C5)c4Cl)c3C[C@@H]2F)cc1OCc1cncc(C#N)c1)C(=O)O] | +++ | 765.20 | 1.83^{#} |
| 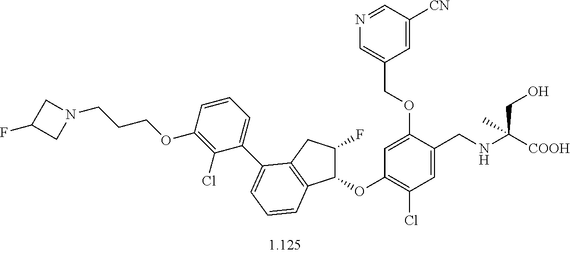 [SMILES: C[C@@](CO)(NCc1cc(Cl)c(O[C@@H]2c3cccc(-c4cccc(OCCCN5CC(F)C5)c4Cl)c3C[C@@H]2F)cc1OCc1cncc(C#N)c1)C(=O)O] | +++ | 767.10 | 1.76^{#} |
| 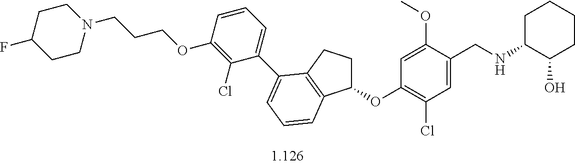 [SMILES: COc1cc(O[C@H]2CCc3c(-c4cccc(OCCCN5CCC(F)CC5)c4Cl)cccc32)c(Cl)cc1CN[C@@H]1CCCC[C@@H]1O] | ++ | 671.30 | 1.90 |
| 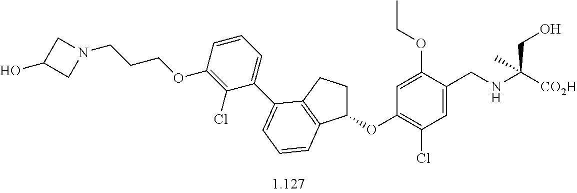 [SMILES: CCOc1cc(O[C@H]2CCc3c(-c4cccc(OCCCN5CC(O)C5)c4Cl)cccc32)c(Cl)cc1CN[C@@](C)(CO)C(=O)O] | +++ | 659.20 | 1.83 |
| 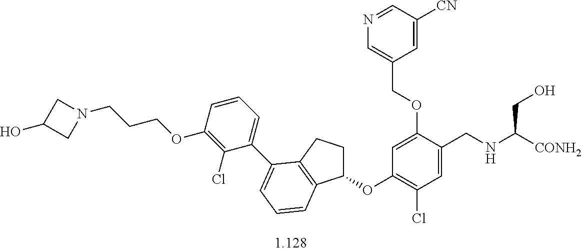 [SMILES: N#Cc1cncc(COc2cc(O[C@H]3CCc4c(-c5cccc(OCCCN6CC(O)C6)c5Cl)cccc43)c(Cl)cc2CN[C@@H](CO)C(N)=O)c1] | +++ | 746.20 | 0.40 |
| 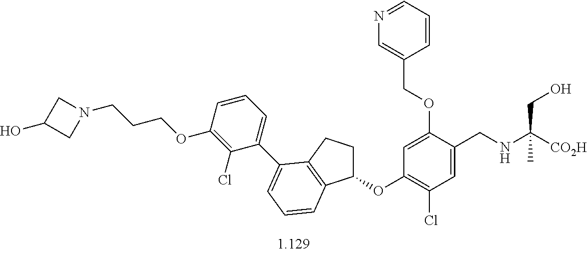 | +++ | 722.20 | 0.47 |
| 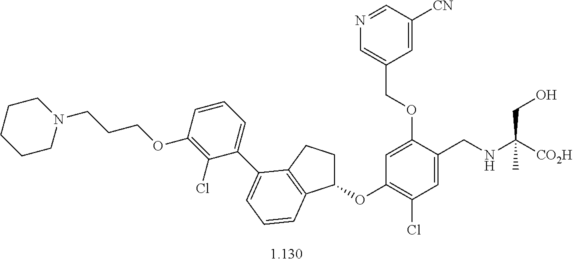 | +++ | 757.20 | 1.95 |
| 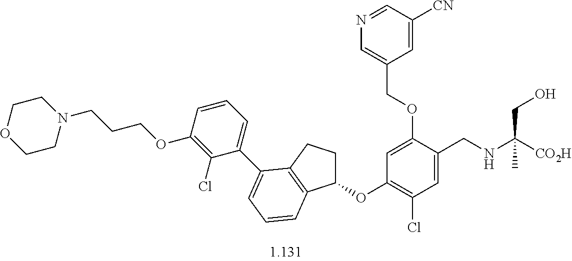 | +++ | 761.20 | 1.41 |
| 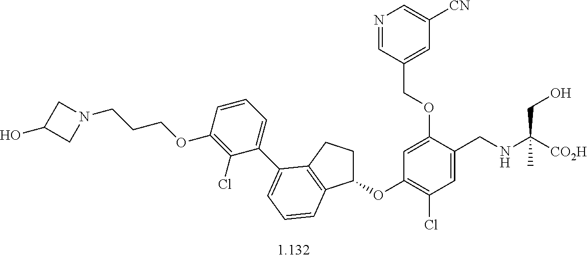 | +++ | 758.10 | 1.60 |
| 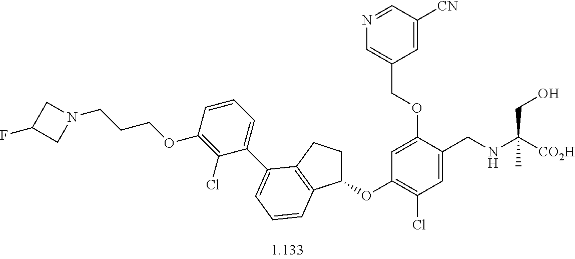 | +++ | 749.20 | 1.22 |
| 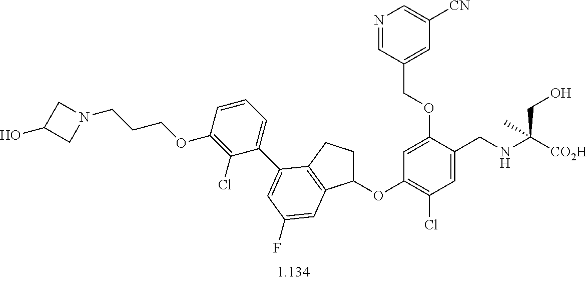 [SMILES: C[C@@](CO)(NCc1cc(Cl)c(OC2CCc3c(-c4cccc(OCCCN5CC(O)C5)c4Cl)cc(F)cc32)cc1OCc1cncc(C#N)c1)C(=O)O] | +++ | 765.20 | 1.55 |
| 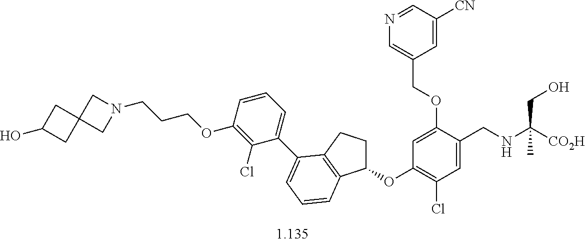 | +++ | 787.20 | 2.20 |
| 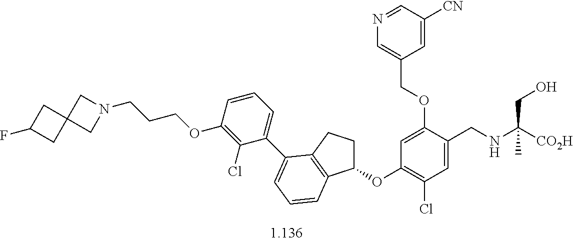 | +++ | 789.20 | 1.65 |
| 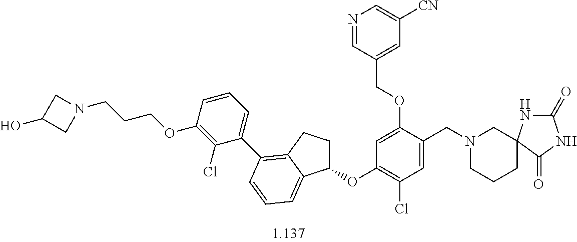 [SMILES: N#Cc1cncc(COc2cc(O[C@H]3CCc4c(-c5cccc(OCCCN6CC(O)C6)c5Cl)cccc43)c(Cl)cc2CN2CCCC3(C2)NC(=O)NC3=O)c1] | +++ | 798.70 | 1.80 |
| 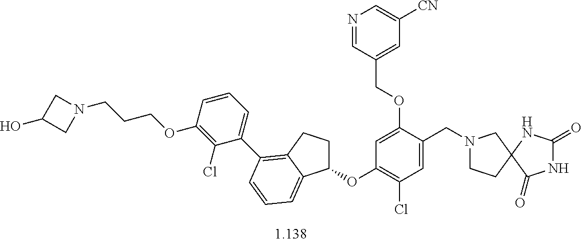 [SMILES: N#Cc1cncc(COc2cc(O[C@H]3CCc4c(-c5cccc(OCCCN6CC(O)C6)c5Cl)cccc43)c(Cl)cc2CN2CCC3(C2)NC(=O)NC3=O)c1] | ++ | 783.20 | 1.60 |
| 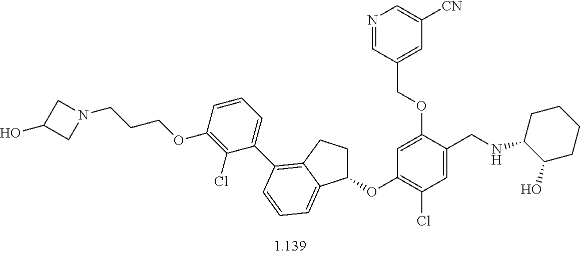 [SMILES: N#Cc1cncc(COc2cc(O[C@H]3CCc4c(-c5cccc(OCCCN6CC(O)C6)c5Cl)cccc43)c(Cl)cc2CN[C@@H]2CCCC[C@@H]2O)c1] | +++ | 743.20 | 1.80 |
| 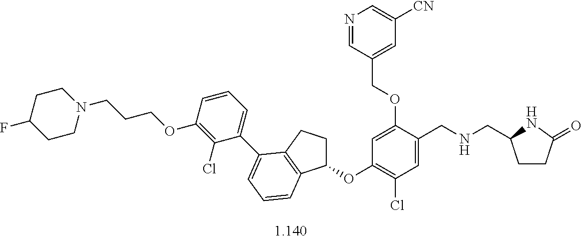 [SMILES: N#Cc1cncc(COc2cc(O[C@H]3CCc4c(-c5cccc(OCCCN6CCC(F)CC6)c5Cl)cccc43)c(Cl)cc2CNC[C@@H]2CCC(=O)N2)c1] | +++ | 772.20 | 1.81 |
| 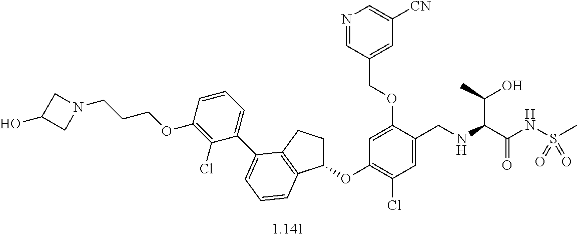 [SMILES: C[C@@H](O)[C@H](NCc1cc(Cl)c(O[C@H]2CCc3c(-c4cccc(OCCCN5CC(O)C5)c4Cl)cccc32)cc1OCc1cncc(C#N)c1)C(=O)NS(C)(=O)=O] | +++ | 824.10 | 1.85 |
| 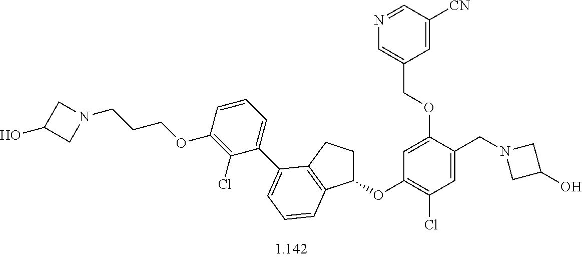 [SMILES: N#Cc1cncc(COc2cc(O[C@H]3CCc4c(-c5cccc(OCCCN6CC(O)C6)c5Cl)cccc43)c(Cl)cc2CN2CC(O)C2)c1] | +++ | 701.10 | 1.95 |
| 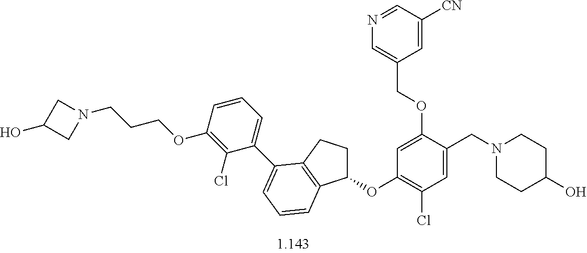 [SMILES: N#Cc1cncc(COc2cc(O[C@H]3CCc4c(-c5cccc(OCCCN6CC(O)C6)c5Cl)cccc43)c(Cl)cc2CN2CCC(O)CC2)c1] | +++ | 729.20 | 0.53 |
| 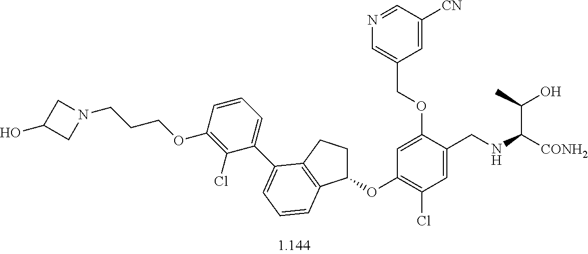 [SMILES: C[C@@H](O)[C@H](NCc1cc(Cl)c(O[C@H]2CCc3c(-c4cccc(OCCCN5CC(O)C5)c4Cl)cccc32)cc1OCc1cncc(C#N)c1)C(N)=O] | +++ | 746.20 | 0.40 |
| 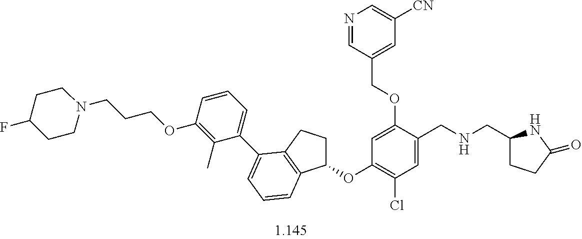 [SMILES: Cc1c(OCCCN2CCC(F)CC2)cccc1-c1cccc2c1CC[C@@H]2Oc1cc(OCc2cncc(C#N)c2)c(CNC[C@@H]2CCC(=O)N2)cc1Cl] | +++ | 752.20 | 1.90 |
| 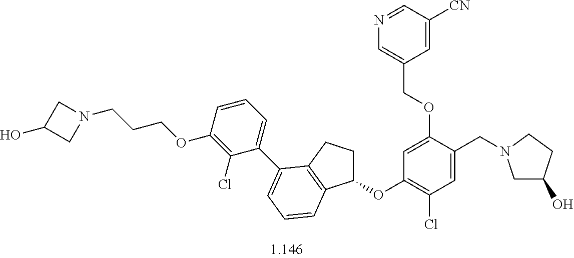 [SMILES: N#Cc1cncc(COc2cc(O[C@H]3CCc4c(-c5cccc(OCCCN6CC(O)C6)c5Cl)cccc43)c(Cl)cc2CN2CC[C@@H](O)C2)c1] | +++ | 715.10 | 1.94 |
| 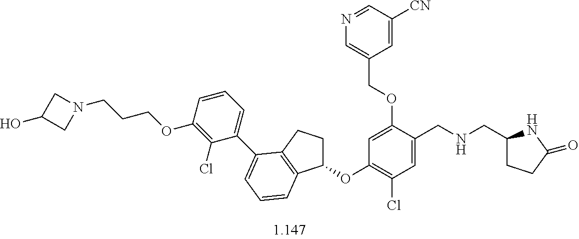 [SMILES: N#Cc1cncc(COc2cc(O[C@H]3CCc4c(-c5cccc(OCCCN6CC(O)C6)c5Cl)cccc43)c(Cl)cc2CNC[C@@H]2CCC(=O)N2)c1] | +++ | 742.00 | 1.79 |
| 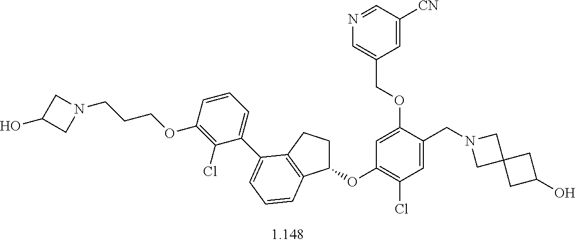 [SMILES: N#Cc1cncc(COc2cc(O[C@H]3CCc4c(-c5cccc(OCCCN6CC(O)C6)c5Cl)cccc43)c(Cl)cc2CN2CC3(CC(O)C3)C2)c1] | +++ | 741.00 | 2.03 |
| 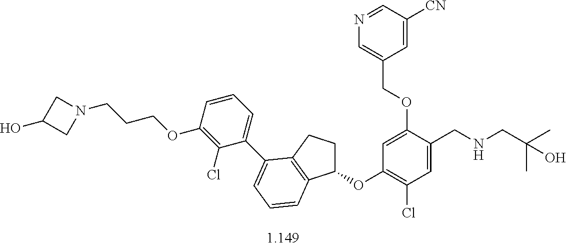 [SMILES: CC(C)(O)CNCc1cc(Cl)c(O[C@H]2CCc3c(-c4cccc(OCCCN5CC(O)C5)c4Cl)cccc32)cc1OCc1cncc(C#N)c1] | +++ | 717.00 | 2.03 |
| 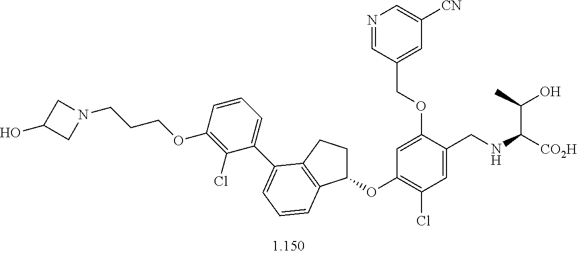 [SMILES: C[C@@H](O)[C@H](NCc1cc(Cl)c(O[C@H]2CCc3c(-c4cccc(OCCCN5CC(O)C5)c4Cl)cccc32)cc1OCc1cncc(C#N)c1)C(=O)O] | +++ | 747.20 | 1.82 |
| 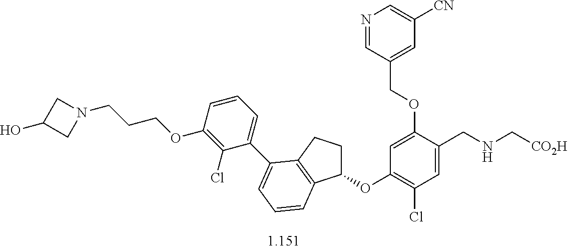 [SMILES: N#Cc1cncc(COc2cc(O[C@H]3CCc4c(-c5cccc(OCCCN6CC(O)C6)c5Cl)cccc43)c(Cl)cc2CNCC(=O)O)c1] | +++ | 703.20 | 1.87 |
| 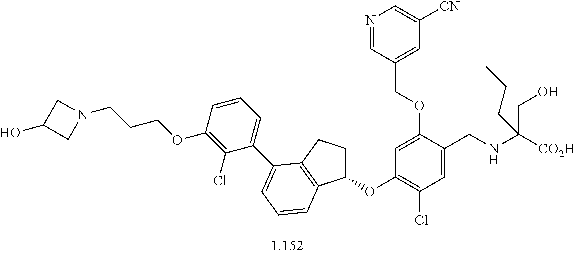 [SMILES: CCCC(CO)(NCc1cc(Cl)c(O[C@H]2CCc3c(-c4cccc(OCCCN5CC(O)C5)c4Cl)cccc32)cc1OCc1cncc(C#N)c1)C(=O)O] | +++ | 775.20 | 1.64 |
| 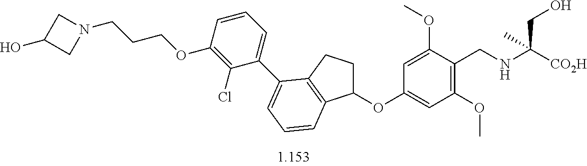 [SMILES: COc1cc(OC2CCc3c(-c4cccc(OCCCN5CC(O)C5)c4Cl)cccc32)cc(OC)c1CN[C@@](C)(CO)C(=O)O] | ++ | 641.20 | 2.68* |
| 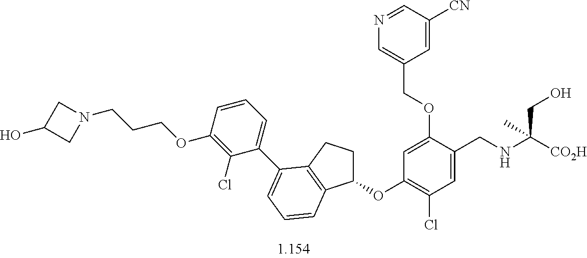 [SMILES: C[C@@](CO)(NCc1cc(Cl)c(O[C@H]2CCc3c(-c4cccc(OCCCN5CC(O)C5)c4Cl)cccc32)cc1OCc1cncc(C#N)c1)C(=O)O] | +++ | 717.20 | 1.80 |
| 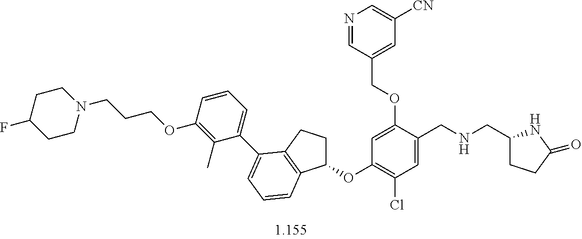 [SMILES: Cc1c(OCCCN2CCC(F)CC2)cccc1-c1cccc2c1CC[C@@H]2Oc1cc(OCc2cncc(C#N)c2)c(CNC[C@H]2CCC(=O)N2)cc1Cl] | +++ | 752.20 | 1.90 |
| 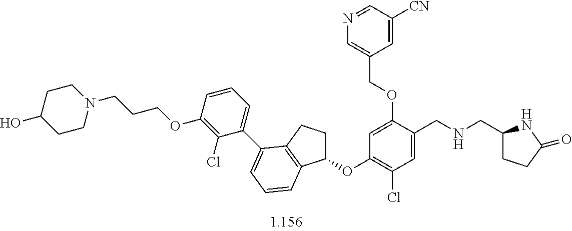 [SMILES: N#Cc1cncc(COc2cc(O[C@H]3CCc4c(-c5cccc(OCCCN6CCC(O)CC6)c5Cl)cccc43)c(Cl)cc2CNC[C@@H]2CCC(=O)N2)c1] | +++ | 772.20 | 1.45 |
| 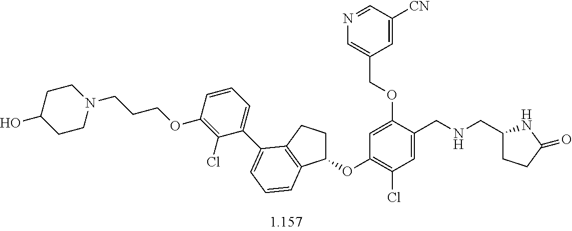 [SMILES: N#Cc1cncc(COc2cc(O[C@H]3CCc4c(-c5cccc(OCCCN6CCC(O)CC6)c5Cl)cccc43)c(Cl)cc2CNC[C@H]2CCC(=O)N2)c1] | +++ | 770.20 | 1.45 |
| 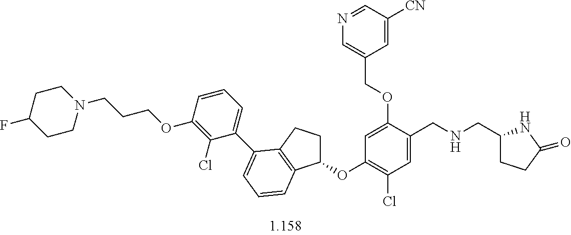 [SMILES: N#Cc1cncc(COc2cc(O[C@H]3CCc4c(-c5cccc(OCCCN6CCC(F)CC6)c5Cl)cccc43)c(Cl)cc2CNC[C@H]2CCC(=O)N2)c1] | +++ | 774.20 | 1.52 |
| 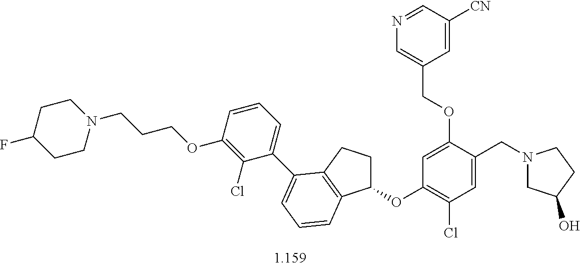 [SMILES: N#Cc1cncc(COc2cc(O[C@H]3CCc4c(-c5cccc(OCCCN6CCC(F)CC6)c5Cl)cccc43)c(Cl)cc2CN2CC[C@@H](O)C2)c1] | +++ | 745.20 | 1.57 |
| 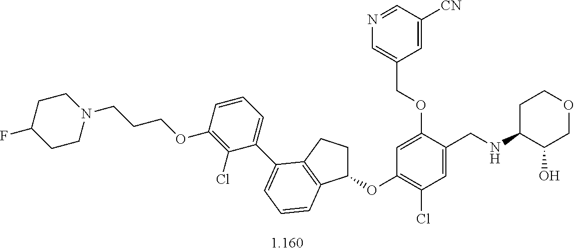 [SMILES: N#Cc1cncc(COc2cc(O[C@H]3CCc4c(-c5cccc(OCCCN6CCC(F)CC6)c5Cl)cccc43)c(Cl)cc2CN[C@H]2CCOC[C@@H]2O)c1] | +++ | 775.20 | 1.53 |
| 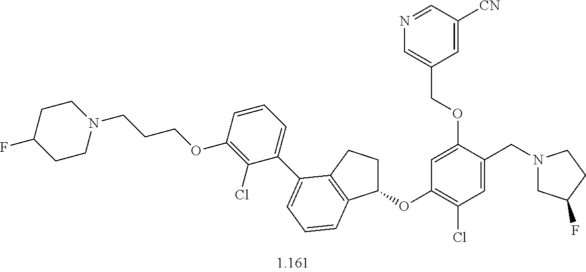 [SMILES: N#Cc1cncc(COc2cc(O[C@H]3CCc4c(-c5cccc(OCCCN6CCC(F)CC6)c5Cl)cccc43)c(Cl)cc2CN2CC[C@@H](F)C2)c1] | +++ | 747.10 | 2.27 |
| 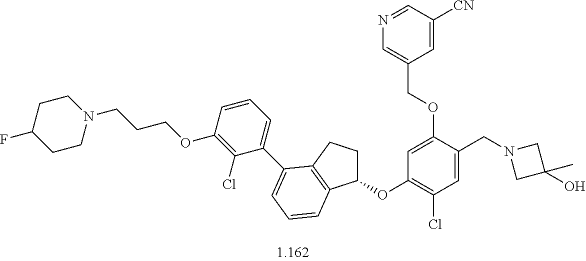 [SMILES: CC1(O)CN(Cc2cc(Cl)c(O[C@H]3CCc4c(-c5cccc(OCCCN6CCC(F)CC6)c5Cl)cccc43)cc2OCc2cncc(C#N)c2)C1] | +++ | 745.20 | 1.95 |
| 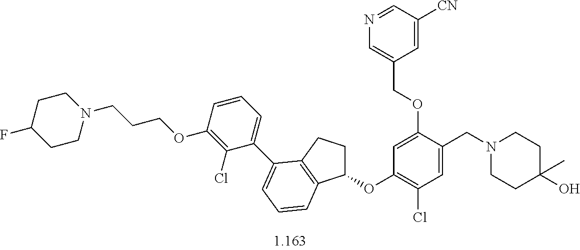 [SMILES: CC1(O)CCN(Cc2cc(Cl)c(O[C@H]3CCc4c(-c5cccc(OCCCN6CCC(F)CC6)c5Cl)cccc43)cc2OCc2cncc(C#N)c2)CC1] | +++ | 773.20 | 1.87 |
| 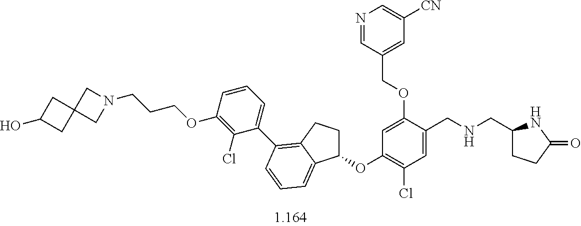 [SMILES: N#Cc1cncc(COc2cc(O[C@H]3CCc4c(-c5cccc(OCCCN6CC7(CC(O)C7)C6)c5Cl)cccc43)c(Cl)cc2CNC[C@@H]2CCC(=O)N2)c1] | +++ | 783.20 | 1.70 |
| 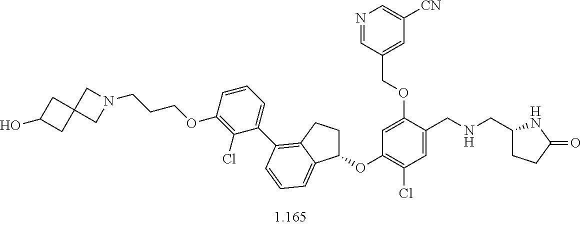 [SMILES: N#Cc1cncc(COc2cc(O[C@H]3CCc4c(-c5cccc(OCCCN6CC7(CC(O)C7)C6)c5Cl)cccc43)c(Cl)cc2CNC[C@H]2CCC(=O)N2)c1] | +++ | 783.20 | 1.70 |
| 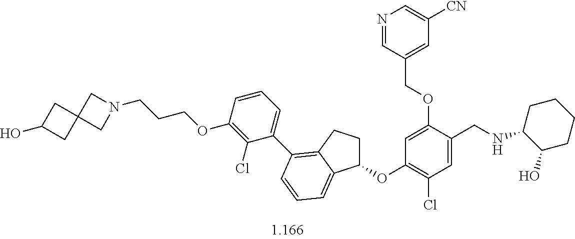 [SMILES: N#Cc1cncc(COc2cc(O[C@H]3CCc4c(-c5cccc(OCCCN6CC7(CC(O)C7)C6)c5Cl)cccc43)c(Cl)cc2CN[C@@H]2CCCC[C@@H]2O)c1] | +++ | 783.20 | 1.90 |
| 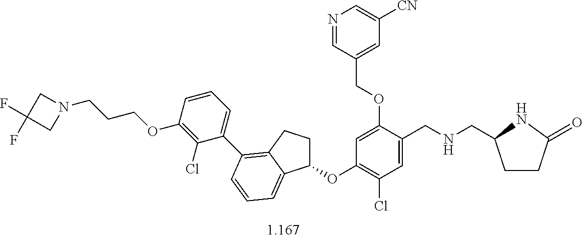 [SMILES: N#Cc1cncc(COc2cc(O[C@H]3CCc4c(-c5cccc(OCCCN6CC(F)(F)C6)c5Cl)cccc43)c(Cl)cc2CNC[C@@H]2CCC(=O)N2)c1] | ++ | 762.20 | 1.70 |
| 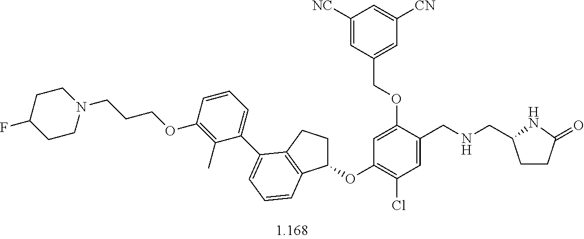 [SMILES: Cc1c(OCCCN2CCC(F)CC2)cccc1-c1cccc2c1CC[C@@H]2Oc1cc(OCc2cc(C#N)cc(C#N)c2)c(CNC[C@H]2CCC(=O)N2)cc1Cl] | ++ | 776.10 | 2.17 |
| 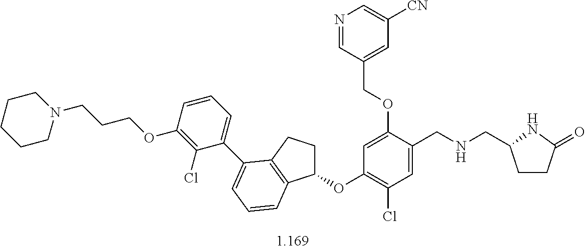 [SMILES: N#Cc1cncc(COc2cc(O[C@H]3CCc4c(-c5cccc(OCCCN6CCCCC6)c5Cl)cccc43)c(Cl)cc2CNC[C@H]2CCC(=O)N2)c1] | +++ | 754.10 | 1.87 |
| 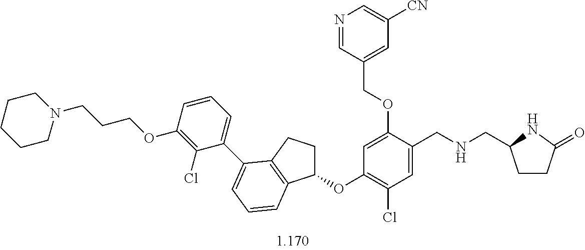 [SMILES: N#Cc1cncc(COc2cc(O[C@H]3CCc4c(-c5cccc(OCCCN6CCCCC6)c5Cl)cccc43)c(Cl)cc2CNC[C@@H]2CCC(=O)N2)c1] | +++ | 754.10 | 1.90 |
| 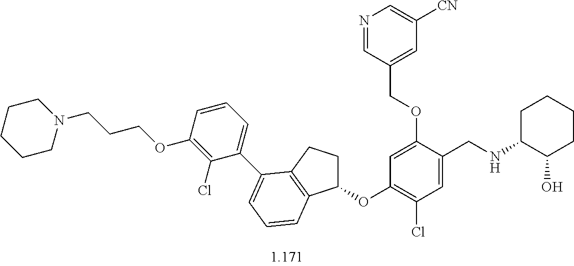 [SMILES: N#Cc1cncc(COc2cc(O[C@H]3CCc4c(-c5cccc(OCCCN6CCCCC6)c5Cl)cccc43)c(Cl)cc2CN[C@@H]2CCCC[C@@H]2O)c1] | +++ | 755.10 | 2.33 |
| 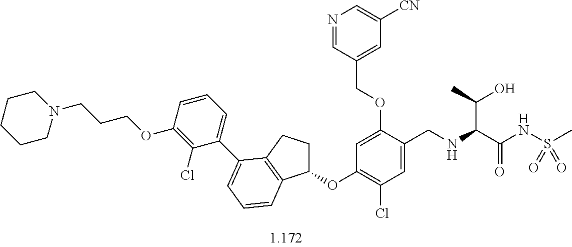 [SMILES: C[C@@H](O)[C@H](NCc1cc(Cl)c(O[C@H]2CCc3c(-c4cccc(OCCCN5CCCCC5)c4Cl)cccc32)cc1OCc1cncc(C#N)c1)C(=O)NS(C)(=O)=O] | +++ | 836.00 | 2.05 |
| 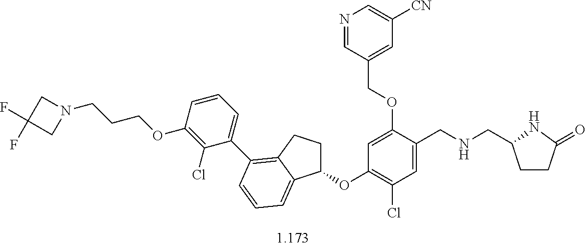 [SMILES: N#Cc1cncc(COc2cc(O[C@H]3CCc4c(-c5cccc(OCCCN6CC(F)(F)C6)c5Cl)cccc43)c(Cl)cc2CNC[C@H]2CCC(=O)N2)c1] | ++ | 762.20 | 1.70 |
| 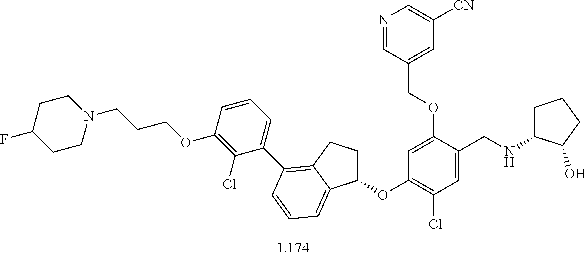 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCC[C@@H]2O)c1] | +++ | 759.20 | 1.80 |
| 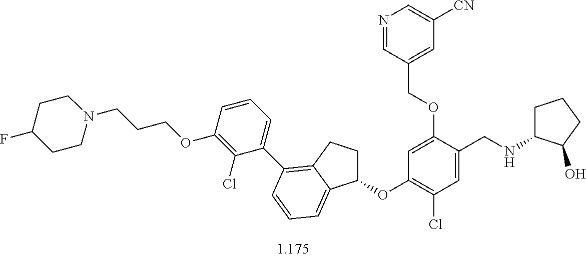 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCC[C@H]2O)c1] | +++ | 759.20 | 1.80 |
| 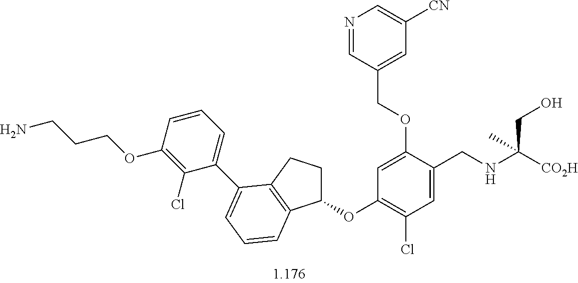 [SMILES: C[C@@](CO)(NCc1cc(Cl)c(O[C@H]2CCc3c(-c4cccc(OCCCN)c4Cl)cccc32)cc1OCc1cncc(C#N)c1)C(=O)O] | +++ | 691.00 | 1.60 |
| 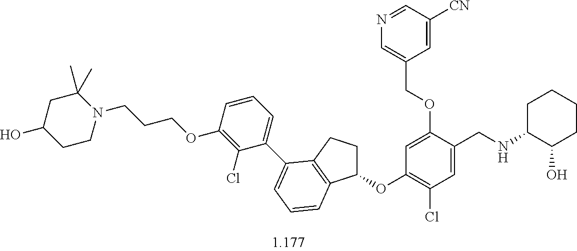 [SMILES: CC1(C)CC(O)CCN1CCCOc1cccc(-c2cccc3c2CC[C@@H]3Oc2cc(OCc3cncc(C#N)c3)c(CN[C@@H]3CCCC[C@@H]3O)cc2Cl)c1Cl] | +++ | 799.10 | 2.17 |
| 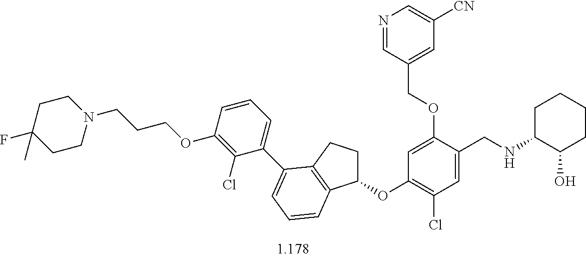 [SMILES: CC1(F)CCN(CCCOc2cccc(-c3cccc4c3CC[C@@H]4Oc3cc(OCc4cncc(C#N)c4)c(CN[C@@H]4CCCC[C@@H]4O)cc3Cl)c2Cl)CC1] | +++ | 787.20 | 2.08 |
| 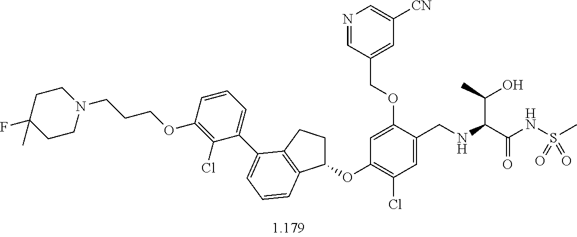 [SMILES: C[C@@H](O)[C@H](NCc1cc(Cl)c(O[C@H]2CCc3c(-c4cccc(OCCCN5CCC(C)(F)CC5)c4Cl)cccc32)cc1OCc1cncc(C#N)c1)C(=O)NS(C)(=O)=O] | +++ | 868.20 | 1.97 |
| 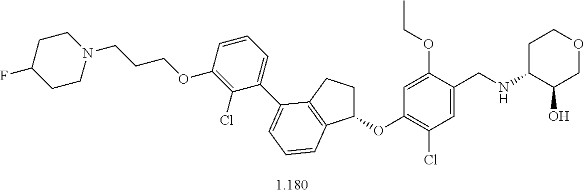 [SMILES: CCOc1cc(O[C@H]2CCc3c(-c4cccc(OCCCN5CCC(F)CC5)c4Cl)cccc32)c(Cl)cc1CN[C@@H]1CCOC[C@H]1O] | ++ | 687.20 | 1.69 |
| 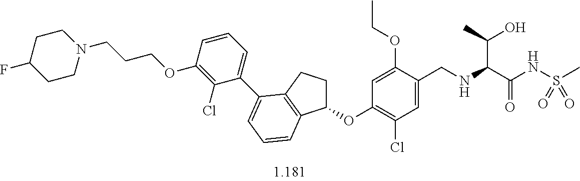 [SMILES: CCOc1cc(O[C@H]2CCc3c(-c4cccc(OCCCN5CCC(F)CC5)c4Cl)cccc32)c(Cl)cc1CN[C@H](C(=O)NS(C)(=O)=O)[C@@H](C)O] | ++ | 766.20 | 1.88 |
| 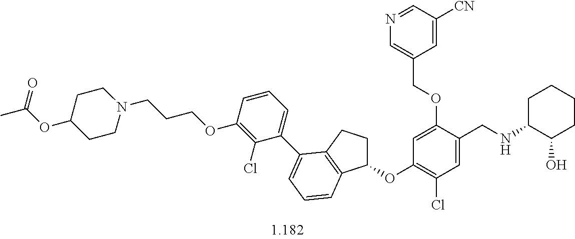 [SMILES: CC(=O)OC1CCN(CCCOc2cccc(-c3cccc4c3CC[C@@H]4Oc3cc(OCc4cncc(C#N)c4)c(CN[C@@H]4CCCC[C@@H]4O)cc3Cl)c2Cl)CC1] | +++ | 813.10 | 2.74 |
| 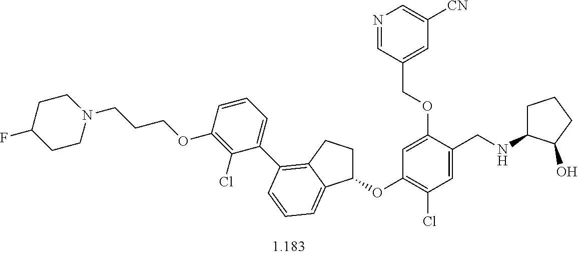 [SMILES: N#Cc1cncc(COc2cc(O[C@H]3CCc4c(-c5cccc(OCCCN6CCC(F)CC6)c5Cl)cccc43)c(Cl)cc2CN[C@H]2CCC[C@H]2O)c1] | +++ | 759.20 | 1.80 |
| 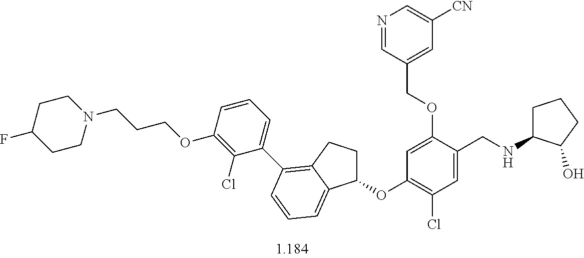 [SMILES: N#Cc1cncc(COc2cc(O[C@H]3CCc4c(-c5cccc(OCCCN6CCC(F)CC6)c5Cl)cccc43)c(Cl)cc2CN[C@H]2CCC[C@@H]2O)c1] | +++ | 759.20 | 1.70 |
| 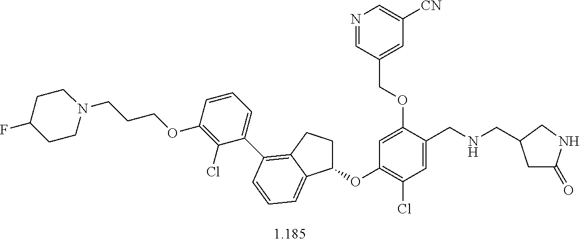 [SMILES: N#Cc1cncc(COc2cc(O[C@H]3CCc4c(-c5cccc(OCCCN6CCC(F)CC6)c5Cl)cccc43)c(Cl)cc2CNCC2CNC(=O)C2)c1] | +++ | 774.20 | 1.70 |
| 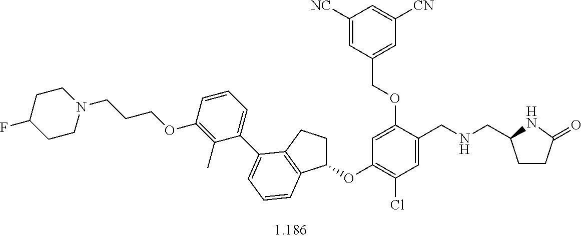 [SMILES: Cc1c(OCCCN2CCC(F)CC2)cccc1-c1cccc2c1CC[C@@H]2Oc1cc(OCc2cc(C#N)cc(C#N)c2)c(CNC[C@@H]2CCC(=O)N2)cc1Cl] | +++ | 776.20 | 1.92 |
| 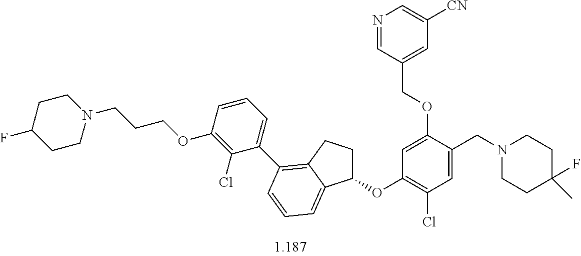 [SMILES: CC1(F)CCN(Cc2cc(Cl)c(O[C@H]3CCc4c(-c5cccc(OCCCN6CCC(F)CC6)c5Cl)cccc43)cc2OCc2cncc(C#N)c2)CC1] | ++ | 775.20 | 1.80 |
| 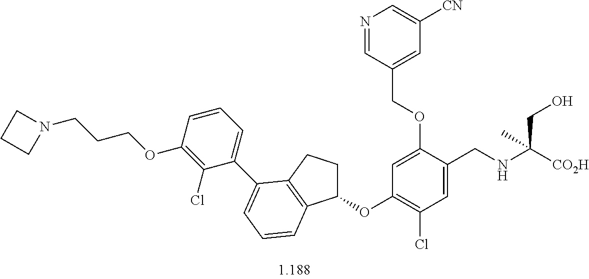 [SMILES: C[C@@](CO)(NCc1cc(Cl)c(O[C@H]2CCc3c(-c4cccc(OCCCN5CCC5)c4Cl)cccc32)cc1OCc1cncc(C#N)c1)C(=O)O] | +++ | 731.20 | 1.36 |
| 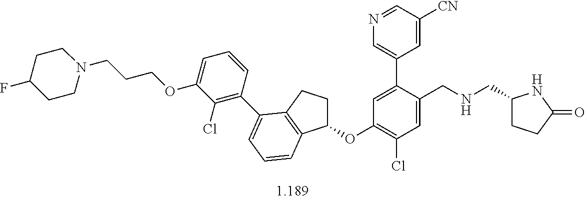 [SMILES: N#Cc1cncc(-c2cc(O[C@H]3CCc4c(-c5cccc(OCCCN6CCC(F)CC6)c5Cl)cccc43)c(Cl)cc2CNC[C@H]2CCC(=O)N2)c1] | + | 742.20 | 1.66 |
| 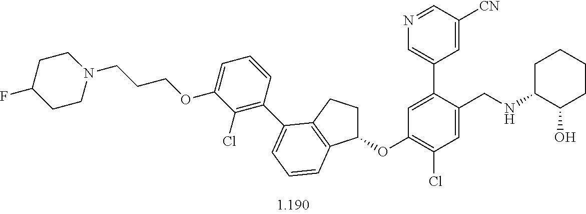 [SMILES: N#Cc1cncc(-c2cc(O[C@H]3CCc4c(-c5cccc(OCCCN6CCC(F)CC6)c5Cl)cccc43)c(Cl)cc2CN[C@@H]2CCCC[C@@H]2O)c1] | ++ | 743.20 | 2.02 |
| 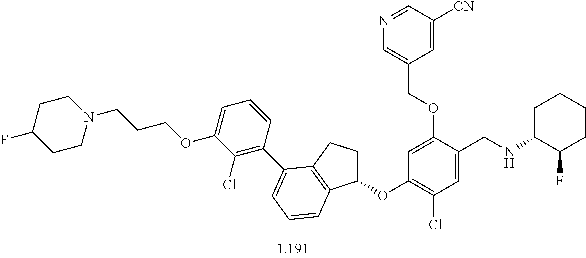 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCCC[C@H]2F)c1] | ++ | 775.20 | 3.26* |
| 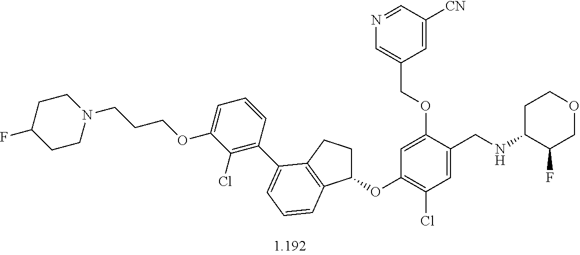 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCOC[C@H]2F)c1] | ++ | 777.20 | 3.06* |
| 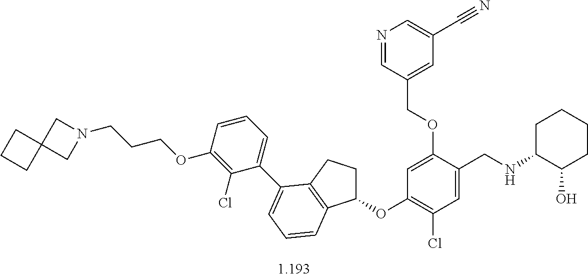 [SMILES: N#Cc1cncc(COc2cc(O[C@H]3CCc4c(-c5cccc(OCCCN6CC7(CCC7)C6)c5Cl)cccc43)c(Cl)cc2CN[C@@H]2CCCC[C@@H]2O)c1] | +++ | 652.20 | 2.51 |
| 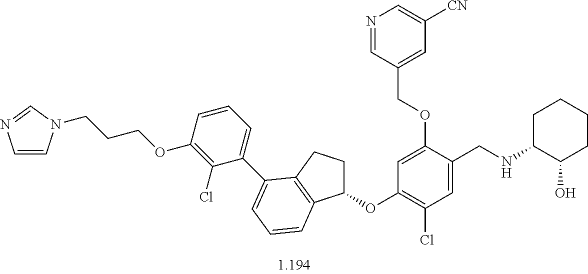 [SMILES: N#Cc1cncc(COc2cc(O[C@H]3CCc4c(-c5cccc(OCCCn6ccnc6)c5Cl)cccc43)c(Cl)cc2CN[C@@H]2CCCC[C@@H]2O)c1] | ++ | 738.20 | 2.27 |
| 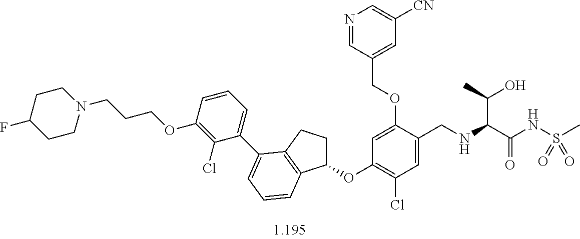 [SMILES: C[C@@H](O)[C@H](NCc1cc(Cl)c(O[C@H]2CCc3c(-c4cccc(OCCCN5CCC(F)CC5)c4Cl)cccc32)cc1OCc1cncc(C#N)c1)C(=O)NS(C)(=O)=O] | +++ | 854.10 | 2.09 |
| 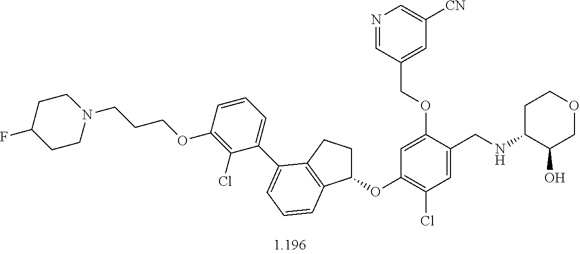 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCOC[C@H]2O)c1] | +++ | 775.20 | 2.06 |
| 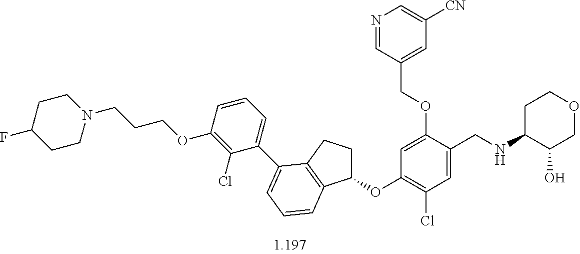 [SMILES: N#Cc1cncc(COc2cc(O[C@H]3CCc4c(-c5cccc(OCCCN6CCC(F)CC6)c5Cl)cccc43)c(Cl)cc2CN[C@H]2CCOC[C@@H]2O)c1] | +++ | 775.20 | 2.23 |
| 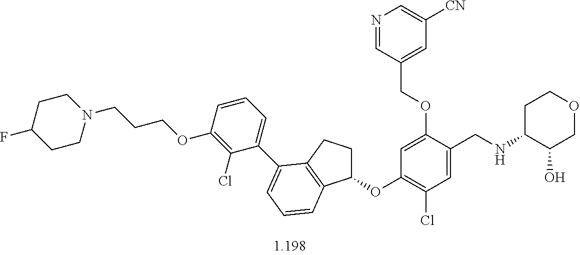 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCOC[C@@H]2O)c1] | +++ | 775.20 | 2.27 |
| 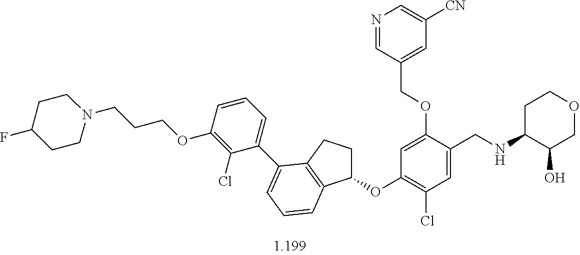 [SMILES: N#Cc1cncc(COc2cc(O[C@H]3CCc4c(-c5cccc(OCCCN6CCC(F)CC6)c5Cl)cccc43)c(Cl)cc2CN[C@H]2CCOC[C@H]2O)c1] | +++ | 775.20 | 2.15 |
| 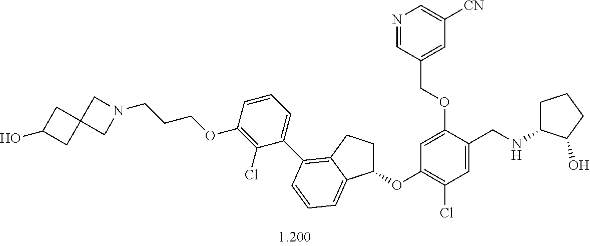 [SMILES: N#Cc1cncc(COc2cc(O[C@H]3CCc4c(-c5cccc(OCCCN6CC7(CC(O)C7)C6)c5Cl)cccc43)c(Cl)cc2CN[C@@H]2CCC[C@@H]2O)c1] | +++ | 769.20 | 1.90 |
| 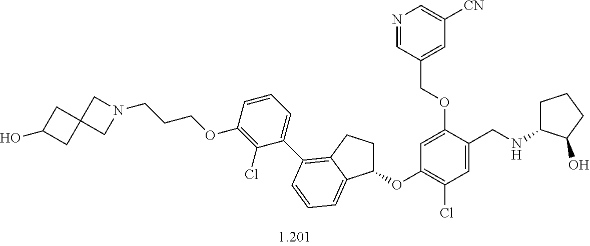 [SMILES: N#Cc1cncc(COc2cc(O[C@H]3CCc4c(-c5cccc(OCCCN6CC7(CC(O)C7)C6)c5Cl)cccc43)c(Cl)cc2CN[C@@H]2CCC[C@H]2O)c1] | +++ | 769.20 | 1.90 |
| 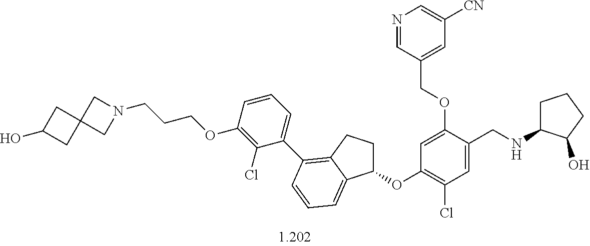 [SMILES: N#Cc1cncc(COc2cc(O[C@H]3CCc4c(-c5cccc(OCCCN6CC7(CC(O)C7)C6)c5Cl)cccc43)c(Cl)cc2CN[C@H]2CCC[C@H]2O)c1] | +++ | 769.20 | 1.80 |
| 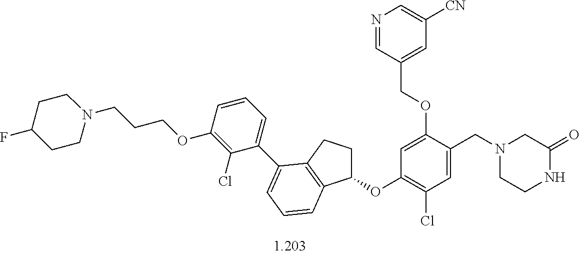 [SMILES: N#Cc1cncc(COc2cc(O[C@H]3CCc4c(-c5cccc(OCCCN6CCC(F)CC6)c5Cl)cccc43)c(Cl)cc2CN2CCNC(=O)C2)c1] | ++ | 758.20 | 2.30 |
| 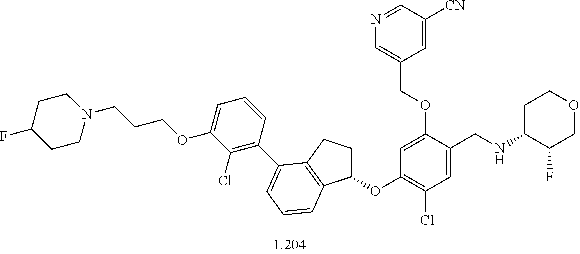 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCOC[C@@H]2F)c1] | ++ | 777.20 | 2.95* |
| 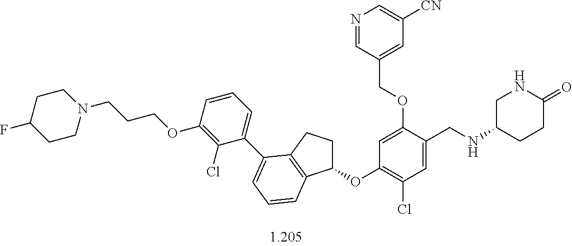 [SMILES: N#Cc1cncc(COc2cc(O[C@H]3CCc4c(-c5cccc(OCCCN6CCC(F)CC6)c5Cl)cccc43)c(Cl)cc2CN[C@H]2CCC(=O)NC2)c1] | +++ | 772.20 | 1.89 |
| 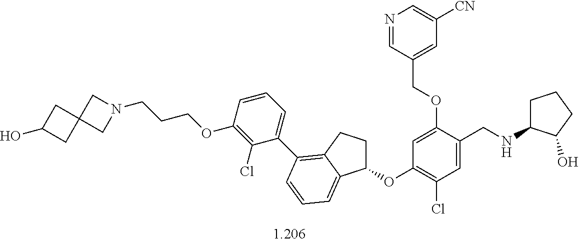 [SMILES: N#Cc1cncc(COc2cc(O[C@H]3CCc4c(-c5cccc(OCCCN6CC7(CC(O)C7)C6)c5Cl)cccc43)c(Cl)cc2CN[C@H]2CCC[C@@H]2O)c1] | +++ | 769.20 | 1.90 |
| 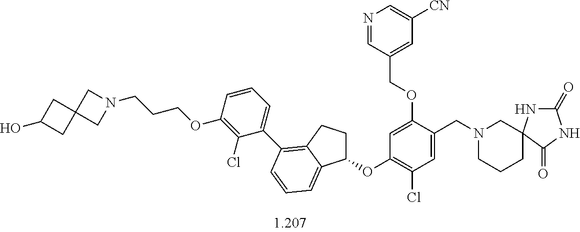 [SMILES: N#Cc1cncc(COc2cc(O[C@H]3CCc4c(-c5cccc(OCCCN6CC7(CC(O)C7)C6)c5Cl)cccc43)c(Cl)cc2CN2CCCC3(C2)NC(=O)NC3=O)c1] | ++ | 837.20 | 1.90 |
| 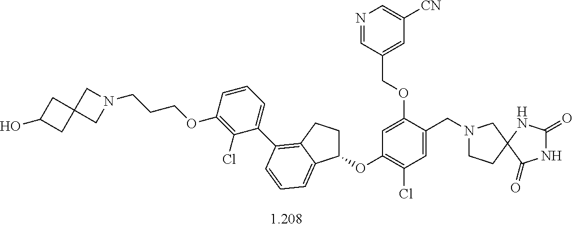 [SMILES: N#Cc1cncc(COc2cc(O[C@H]3CCc4c(-c5cccc(OCCCN6CC7(CC(O)C7)C6)c5Cl)cccc43)c(Cl)cc2CN2CCC3(C2)NC(=O)NC3=O)c1] | ++ | 823.20 | 1.80 |
| 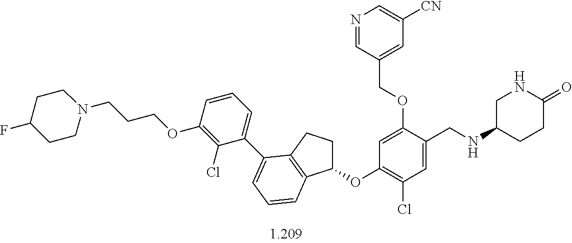 [SMILES: N#Cc1cncc(COc2cc(O[C@H]3CCc4c(-c5cccc(OCCCN6CCC(F)CC6)c5Cl)cccc43)c(Cl)cc2CN[C@@H]2CCC(=O)NC2)c1] | +++ | 772.20 | 2.12 |
| 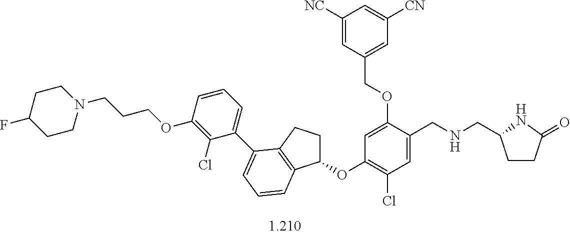 [SMILES: N#Cc1cc(C#N)cc(COc2cc(O[C@H]3CCc4c(-c5cccc(OCCCN6CCC(F)CC6)c5Cl)cccc43)c(Cl)cc2CNC[C@H]2CCC(=O)N2)c1] | +++ | 796.20 | 2.27 |
| 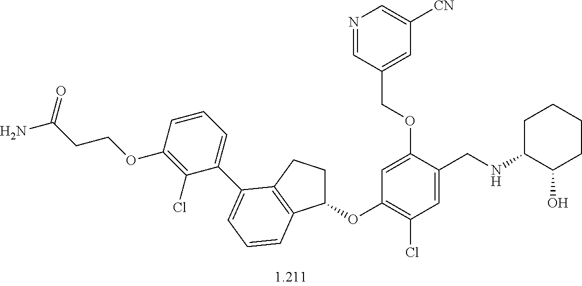 [SMILES: N#Cc1cncc(COc2cc(O[C@H]3CCc4c(-c5cccc(OCCC(N)=O)c5Cl)cccc43)c(Cl)cc2CN[C@@H]2CCCC[C@@H]2O)c1] | ++ | 701.20 | 1.53 |
| 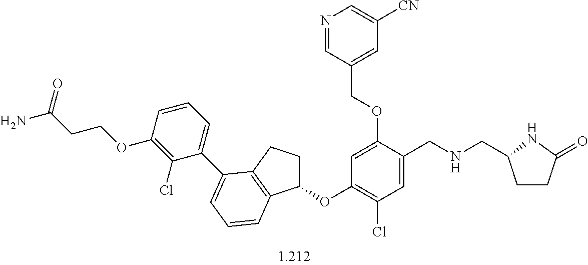 [SMILES: N#Cc1cncc(COc2cc(O[C@H]3CCc4c(-c5cccc(OCCC(N)=O)c5Cl)cccc43)c(Cl)cc2CNC[C@H]2CCC(=O)N2)c1] | ++ | 700.20 | 1.34 |
| 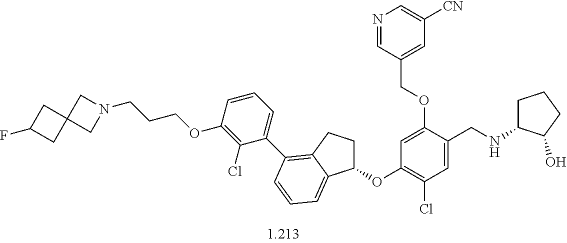 [SMILES: N#Cc1cncc(COc2cc(O[C@H]3CCc4c(-c5cccc(OCCCN6CC7(CC(F)C7)C6)c5Cl)cccc43)c(Cl)cc2CN[C@@H]2CCC[C@@H]2O)c1] | +++ | 771.20 | 2.00 |
| 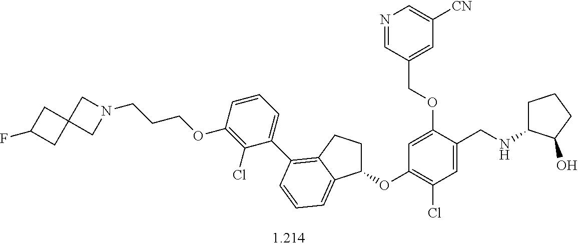 [SMILES: N#Cc1cncc(COc2cc(O[C@H]3CCc4c(-c5cccc(OCCCN6CC7(CC(F)C7)C6)c5Cl)cccc43)c(Cl)cc2CN[C@@H]2CCC[C@H]2O)c1] | +++ | 771.20 | 2.20 |
| 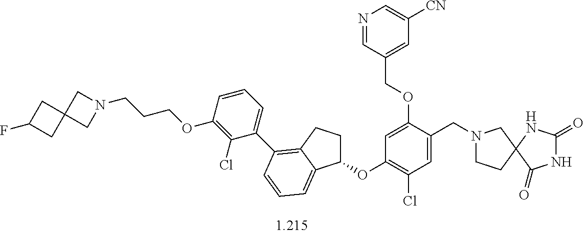 [SMILES: N#Cc1cncc(COc2cc(O[C@H]3CCc4c(-c5cccc(OCCCN6CC7(CC(F)C7)C6)c5Cl)cccc43)c(Cl)cc2CN2CCC3(C2)NC(=O)NC3=O)c1] | ++ | 825.20 | 2.10 |
| 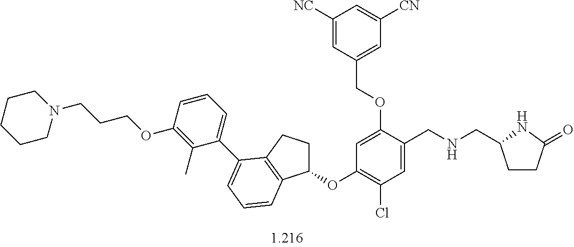 [SMILES: Cc1c(OCCCN2CCCCC2)cccc1-c1cccc2c1CC[C@@H]2Oc1cc(OCc2cc(C#N)cc(C#N)c2)c(CNC[C@H]2CCC(=O)N2)cc1Cl] | +++ | 758.30 | 2.23 |
| 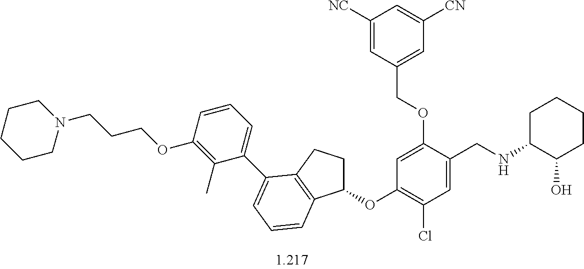 [SMILES: Cc1c(OCCCN2CCCCC2)cccc1-c1cccc2c1CC[C@@H]2Oc1cc(OCc2cc(C#N)cc(C#N)c2)c(CN[C@@H]2CCCC[C@@H]2O)cc1Cl] | +++ | 759.20 | 2.69 |
| 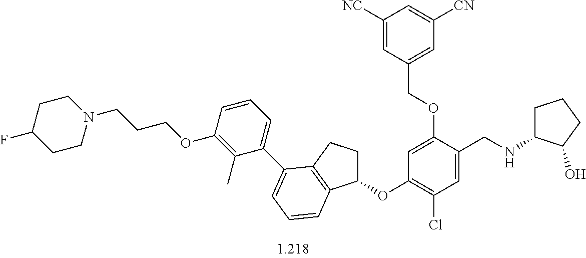 [SMILES: Cc1c(OCCCN2CCC(F)CC2)cccc1-c1cccc2c1CC[C@@H]2Oc1cc(OCc2cc(C#N)cc(C#N)c2)c(CN[C@@H]2CCC[C@@H]2O)cc1Cl] | +++ | 763.20 | 2.08 |
| 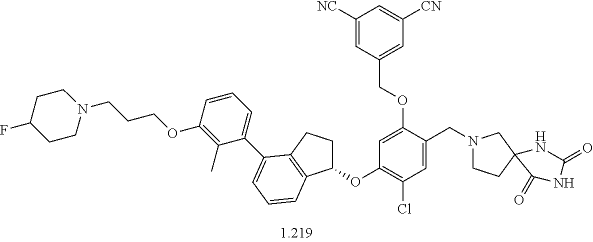 [SMILES: Cc1c(OCCCN2CCC(F)CC2)cccc1-c1cccc2c1CC[C@@H]2Oc1cc(OCc2cc(C#N)cc(C#N)c2)c(CN2CCC3(C2)NC(=O)NC3=O)cc1Cl] | ++ | 817.20 | 2.06 |
| 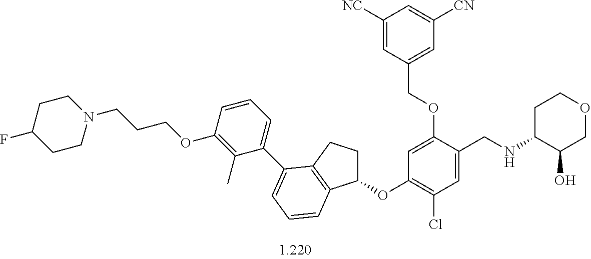 [SMILES: Cc1c(OCCCN2CCC(F)CC2)cccc1-c1cccc2c1CC[C@@H]2Oc1cc(OCc2cc(C#N)cc(C#N)c2)c(CN[C@@H]2CCOC[C@H]2O)cc1Cl] | +++ | 779.30 | 1.95 |
| 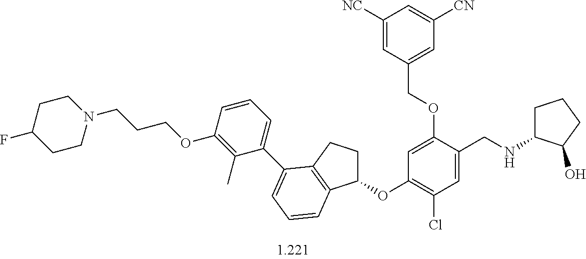 [SMILES: Cc1c(OCCCN2CCC(F)CC2)cccc1-c1cccc2c1CC[C@@H]2Oc1cc(OCc2cc(C#N)cc(C#N)c2)c(CN[C@@H]2CCC[C@H]2O)cc1Cl] | +++ | 763.20 | 2.14 |
| 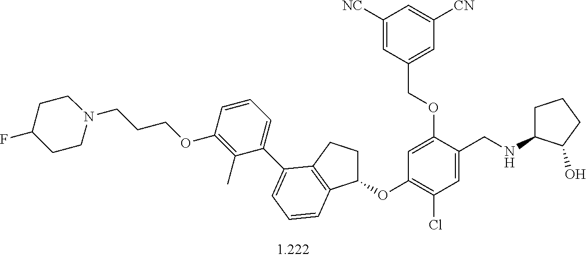 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC[C@@H]2O)cc1Cl] | +++ | 763.20 | 1.99 |
| 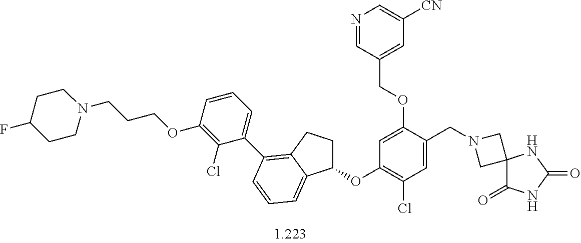 [SMILES: N#Cc1cncc(COc2cc(O[C@H]3CCc4c(-c5cccc(OCCCN6CCC(F)CC6)c5Cl)cccc43)c(Cl)cc2CN2CC3(C2)NC(=O)NC3=O)c1] | +++ | 799.20 | 2.00 |
| 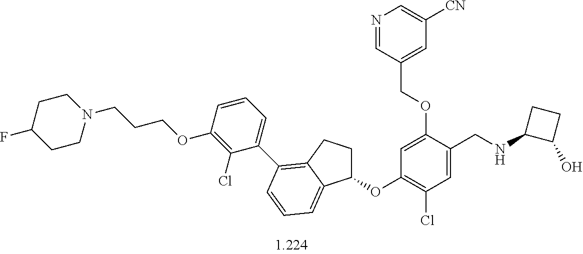 [SMILES: N#Cc1cncc(COc2cc(O[C@H]3CCc4c(-c5cccc(OCCCN6CCC(F)CC6)c5Cl)cccc43)c(Cl)cc2CN[C@H]2CC[C@@H]2O)c1] | +++ | 745.20 | 2.10 |
| 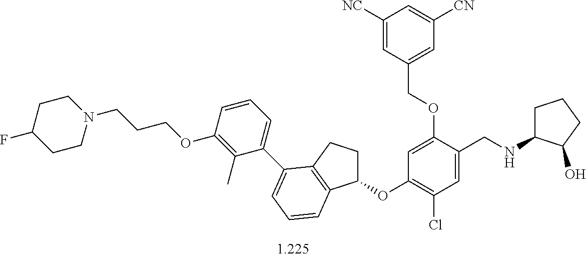 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC[C@H]2O)cc1Cl] | +++ | 763.20 | 2.42 |
| 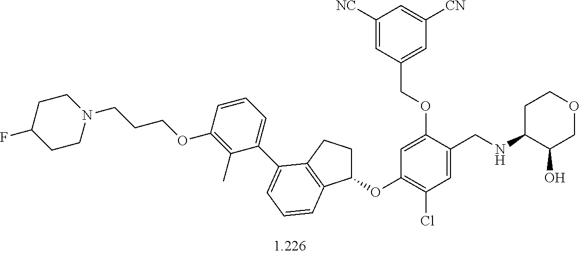 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCOC[C@H]2O)cc1Cl] | +++ | 779.30 | 2.07 |
| 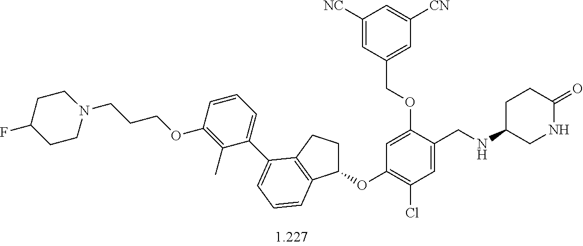 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC(=O)NC2)cc1Cl] | ++ | 776.20 | 2.29 |
| 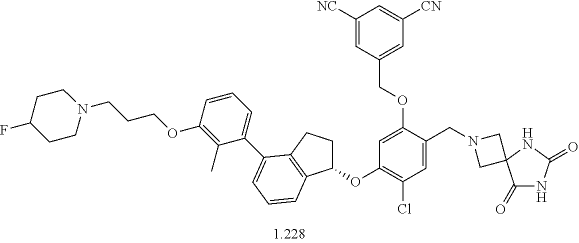 [SMILES: Cc1c(OCCCN2CCC(F)CC2)cccc1-c1cccc2c1CC[C@@H]2Oc1cc(OCc2cc(C#N)cc(C#N)c2)c(CN2CC3(C2)NC(=O)NC3=O)cc1Cl] | ++ | 803.20 | 2.20 |
| 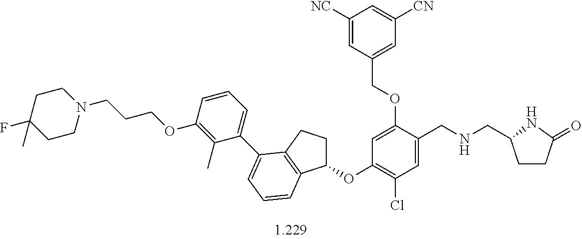 [SMILES: Cc1c(OCCCN2CCC(C)(F)CC2)cccc1-c1cccc2c1CC[C@@H]2Oc1cc(OCc2cc(C#N)cc(C#N)c2)c(CNC[C@H]2CCC(=O)N2)cc1Cl] | ++ | 790.20 | 2.38 |
| 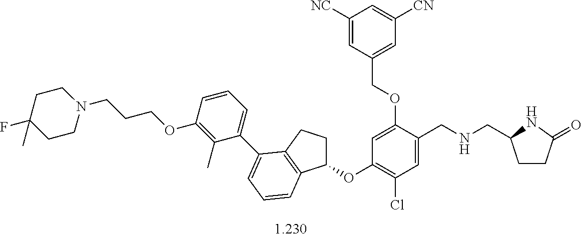 [SMILES: Cc1c(OCCCN2CCC(C)(F)CC2)cccc1-c1cccc2c1CC[C@@H]2Oc1cc(OCc2cc(C#N)cc(C#N)c2)c(CNC[C@@H]2CCC(=O)N2)cc1Cl] | ++ | 790.30 | 1.98 |
| 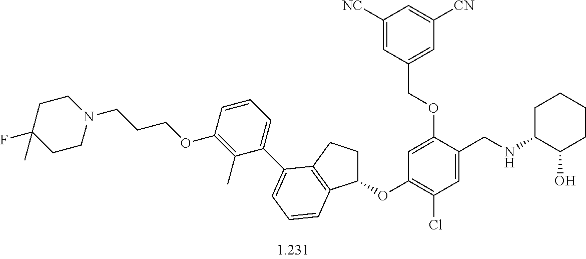 [SMILES: Cc1c(OCCCN2CCC(C)(F)CC2)cccc1-c1cccc2c1CC[C@@H]2Oc1cc(OCc2cc(C#N)cc(C#N)c2)c(CN[C@@H]2CCCC[C@@H]2O)cc1Cl] | ++ | 757.30 | 2.20 |
| 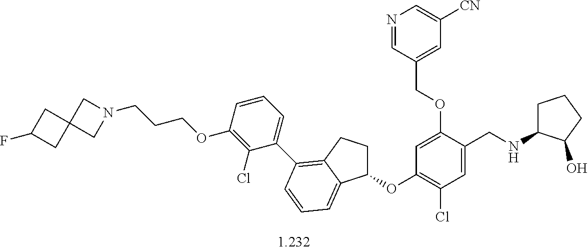 [SMILES: N#Cc1cncc(COc2cc(O[C@H]3CCc4c(-c5cccc(OCCCN6CC7(CC(F)C7)C6)c5Cl)cccc43)c(Cl)cc2CN[C@H]2CCC[C@H]2O)c1] | +++ | 771.20 | 2.20 |
| 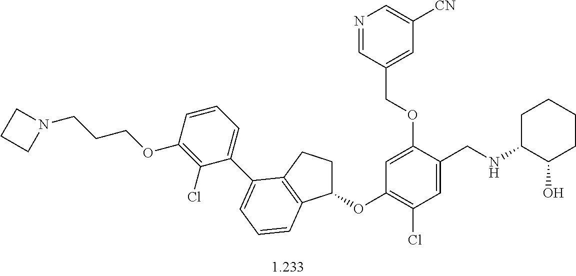 [SMILES: N#Cc1cncc(COc2cc(O[C@H]3CCc4c(-c5cccc(OCCCN6CCC6)c5Cl)cccc43)c(Cl)cc2CN[C@@H]2CCCC[C@@H]2O)c1] | +++ | 727.20 | 2.15 |
| 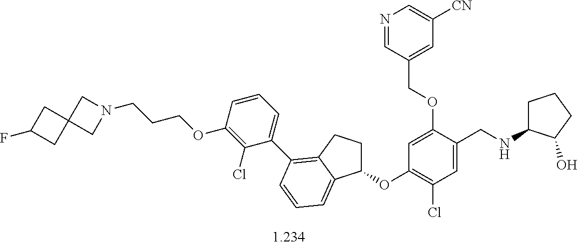 [SMILES: N#Cc1cncc(COc2cc(O[C@H]3CCc4c(-c5cccc(OCCCN6CC7(CC(F)C7)C6)c5Cl)cccc43)c(Cl)cc2CN[C@H]2CCC[C@@H]2O)c1] | +++ | 771.20 | 1.70 |
| 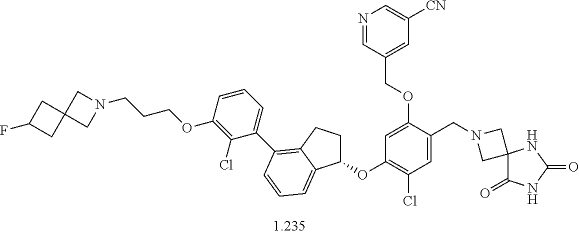 [SMILES: N#Cc1cncc(COc2cc(O[C@H]3CCc4c(-c5cccc(OCCCN6CC7(CC(F)C7)C6)c5Cl)cccc43)c(Cl)cc2CN2CC3(C2)NC(=O)NC3=O)c1] | ++ | 811.20 | 1.70 |
| 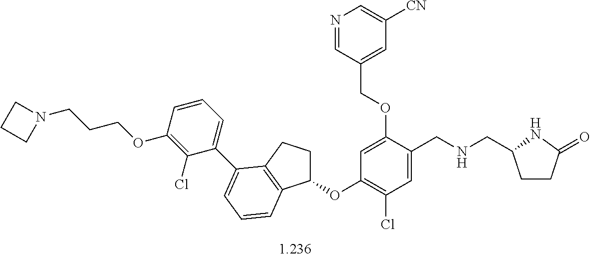 [SMILES: N#Cc1cncc(COc2cc(O[C@H]3CCc4c(-c5cccc(OCCCN6CCC6)c5Cl)cccc43)c(Cl)cc2CNC[C@H]2CCC(=O)N2)c1] | +++ | 726.20 | 1.95 |
| 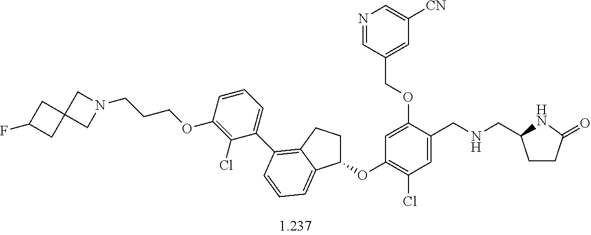 [SMILES: N#Cc1cncc(COc2cc(O[C@H]3CCc4c(-c5cccc(OCCCN6CC7(CC(F)C7)C6)c5Cl)cccc43)c(Cl)cc2CNC[C@@H]2CCC(=O)N2)c1] | +++ | 784.20 | 2.00 |
| 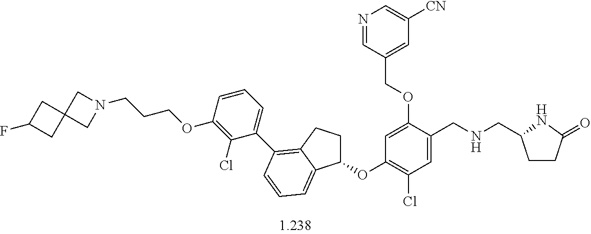 [SMILES: N#Cc1cncc(COc2cc(O[C@H]3CCc4c(-c5cccc(OCCCN6CC7(CC(F)C7)C6)c5Cl)cccc43)c(Cl)cc2CNC[C@H]2CCC(=O)N2)c1] | +++ | 784.20 | 2.00 |
| 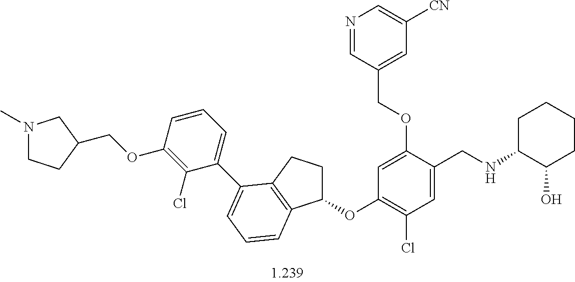 [SMILES: CN1CCC(COc2cccc(-c3cccc4c3CC[C@@H]4Oc3cc(OCc4cncc(C#N)c4)c(CN[C@@H]4CCCC[C@@H]4O)cc3Cl)c2Cl)C1] | +++ | 727.20 | 2.45 |
| 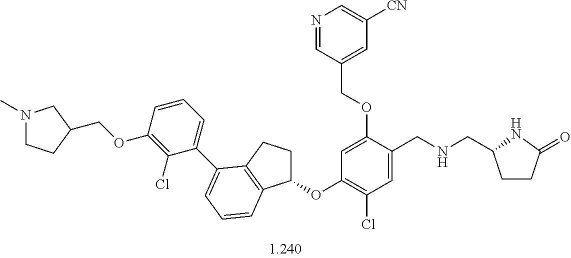 [SMILES: CN1CCC(COc2cccc(-c3cccc4c3CC[C@@H]4Oc3cc(OCc4cncc(C#N)c4)c(CNC[C@H]4CCC(=O)N4)cc3Cl)c2Cl)C1] | +++ | 726.20 | 2.01 |
| 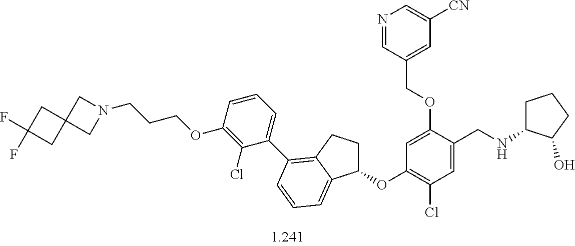 [SMILES: N#Cc1cncc(COc2cc(O[C@H]3CCc4c(-c5cccc(OCCCN6CC7(C6)CC(F)(F)C7)c5Cl)cccc43)c(Cl)cc2CN[C@@H]2CCC[C@@H]2O)c1] | +++ | 789.20 | 2.00 |
| 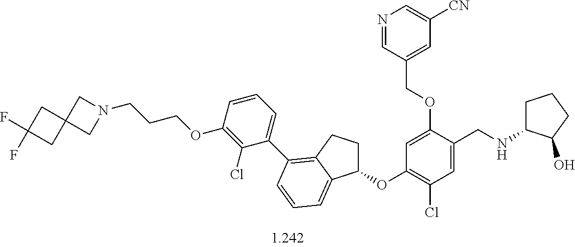 [SMILES: N#Cc1cncc(COc2cc(O[C@H]3CCc4c(-c5cccc(OCCCN6CC7(C6)CC(F)(F)C7)c5Cl)cccc43)c(Cl)cc2CN[C@@H]2CCC[C@H]2O)c1] | +++ | 789.20 | 1.90 |
| 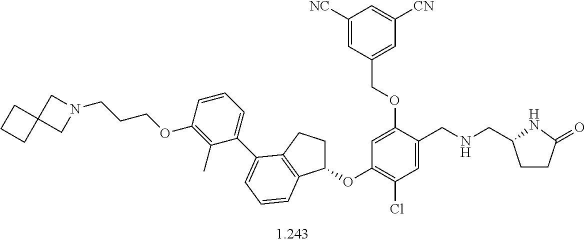 [SMILES: Cc1c(OCCCN2CC3(CCC3)C2)cccc1-c1cccc2c1CC[C@@H]2Oc1cc(OCc2cc(C#N)cc(C#N)c2)c(CNC[C@H]2CCC(=O)N2)cc1Cl] | ++ | 770.20 | 2.58 |
| 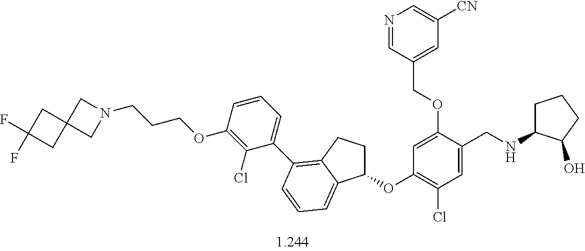 [SMILES: N#Cc1cncc(COc2cc(O[C@H]3CCc4c(-c5cccc(OCCCN6CC7(C6)CC(F)(F)C7)c5Cl)cccc43)c(Cl)cc2CN[C@H]2CCC[C@H]2O)c1] | +++ | 789.20 | 2.30 |
| 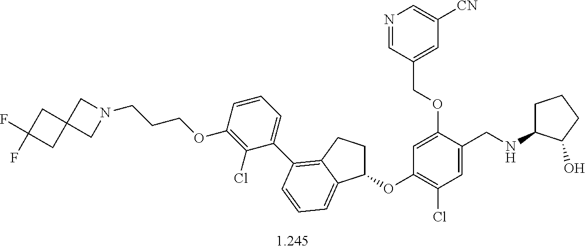 [SMILES: N#Cc1cncc(COc2cc(O[C@H]3CCc4c(-c5cccc(OCCCN6CC7(C6)CC(F)(F)C7)c5Cl)cccc43)c(Cl)cc2CN[C@H]2CCC[C@@H]2O)c1] | +++ | 789.20 | 1.80 |
| 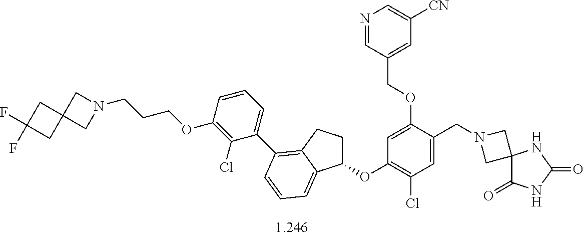 [SMILES: N#Cc1cncc(COc2cc(O[C@H]3CCc4c(-c5cccc(OCCCN6CC7(C6)CC(F)(F)C7)c5Cl)cccc43)c(Cl)cc2CN2CC3(C2)NC(=O)NC3=O)c1] | ++ | 829.00 | 2.00 |
| 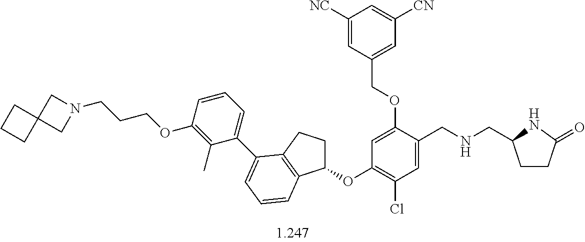 [SMILES: Cc1c(OCCCN2CC3(CCC3)C2)cccc1-c1cccc2c1CC[C@@H]2Oc1cc(OCc2cc(C#N)cc(C#N)c2)c(CNC[C@@H]2CCC(=O)N2)cc1Cl] | +++ | 770.10 | 2.62 |
| 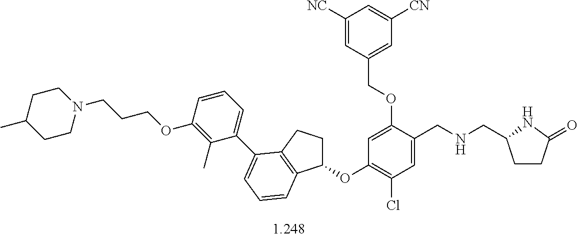 [SMILES: Cc1c(OCCCN2CCC(C)CC2)cccc1-c1cccc2c1CC[C@@H]2Oc1cc(OCc2cc(C#N)cc(C#N)c2)c(CNC[C@H]2CCC(=O)N2)cc1Cl] | +++ | 772.20 | 2.51 |
| 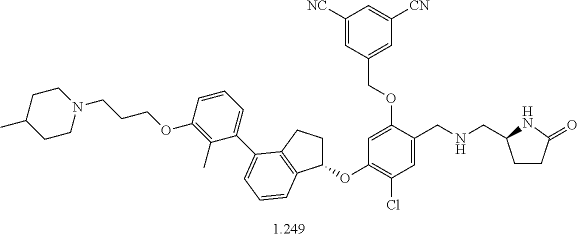 [SMILES: Cc1c(OCCCN2CCC(C)CC2)cccc1-c1cccc2c1CC[C@@H]2Oc1cc(OCc2cc(C#N)cc(C#N)c2)c(CNC[C@@H]2CCC(=O)N2)cc1Cl] | +++ | 772.20 | 2.50 |
| 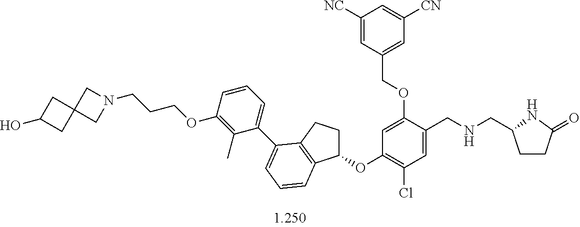 [SMILES: Cc1c(OCCCN2CC3(CC(O)C3)C2)cccc1-c1cccc2c1CC[C@@H]2Oc1cc(OCc2cc(C#N)cc(C#N)c2)c(CNC[C@H]2CCC(=O)N2)cc1Cl] | +++ | 786.20 | 2.08 |
| 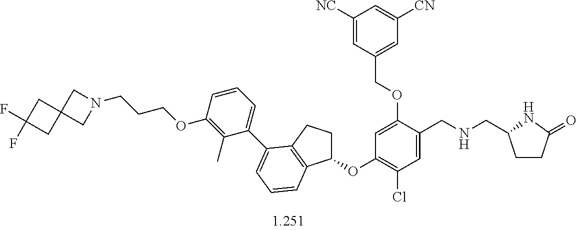 [SMILES: Cc1c(OCCCN2CC3(C2)CC(F)(F)C3)cccc1-c1cccc2c1CC[C@@H]2Oc1cc(OCc2cc(C#N)cc(C#N)c2)c(CNC[C@H]2CCC(=O)N2)cc1Cl] | ++ | 806.30 | 1.84 |
| 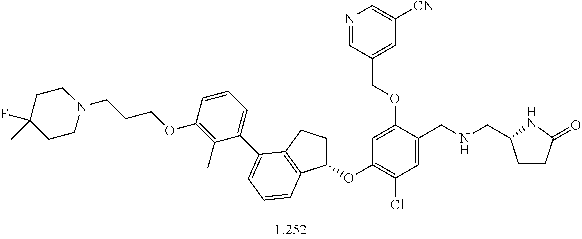 [SMILES: Cc1c(OCCCN2CCC(C)(F)CC2)cccc1-c1cccc2c1CC[C@@H]2Oc1cc(OCc2cncc(C#N)c2)c(CNC[C@H]2CCC(=O)N2)cc1Cl] | ++ | 766.20 | 3.32* |
| 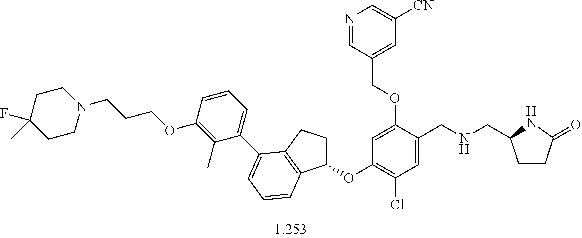 [SMILES: Cc1c(OCCCN2CCC(C)(F)CC2)cccc1-c1cccc2c1CC[C@@H]2Oc1cc(OCc2cncc(C#N)c2)c(CNC[C@@H]2CCC(=O)N2)cc1Cl] | +++ | 766.20 | 3.37* |
| 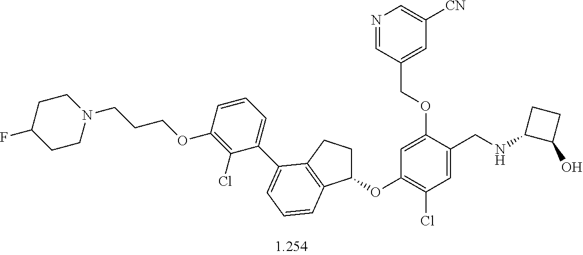 [SMILES: N#Cc1cncc(COc2cc(O[C@H]3CCc4c(-c5cccc(OCCCN6CCC(F)CC6)c5Cl)cccc43)c(Cl)cc2CN[C@@H]2CC[C@H]2O)c1] | +++ | 745.20 | 1.80 |
| 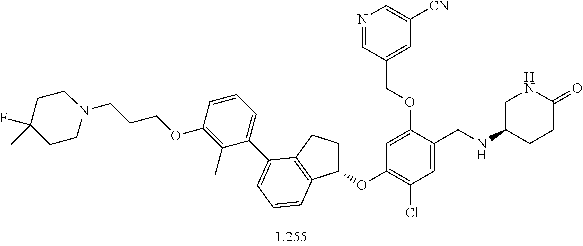 [SMILES: Cc1c(OCCCN2CCC(C)(F)CC2)cccc1-c1cccc2c1CC[C@@H]2Oc1cc(OCc2cncc(C#N)c2)c(CN[C@@H]2CCC(=O)NC2)cc1Cl] | +++ | 766.30 | 1.33 |
| 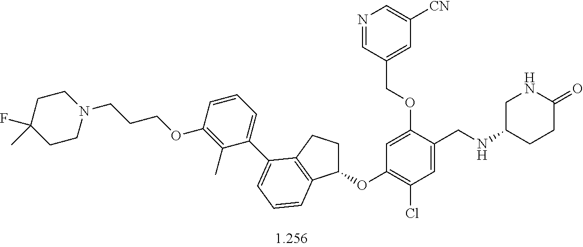 [SMILES: Cc1c(OCCCN2CCC(C)(F)CC2)cccc1-c1cccc2c1CC[C@@H]2Oc1cc(OCc2cncc(C#N)c2)c(CN[C@H]2CCC(=O)NC2)cc1Cl] | ++ | 766.20 | 1.30 |
| 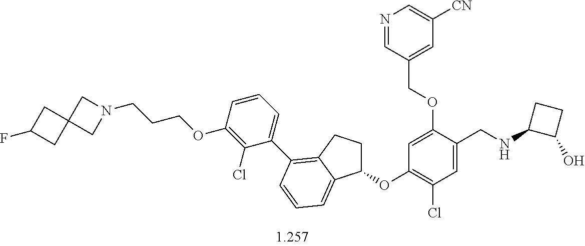 [SMILES: N#Cc1cncc(COc2cc(O[C@H]3CCc4c(-c5cccc(OCCCN6CC7(CC(F)C7)C6)c5Cl)cccc43)c(Cl)cc2CN[C@H]2CC[C@@H]2O)c1] | +++ | 757.20 | 2.00 |
| 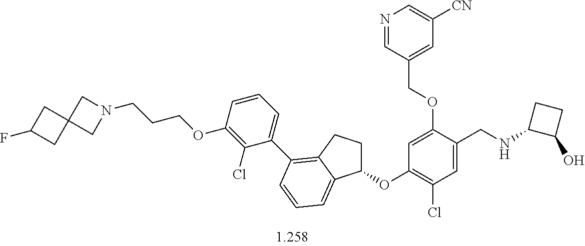 [SMILES: N#Cc1cncc(COc2cc(O[C@H]3CCc4c(-c5cccc(OCCCN6CC7(CC(F)C7)C6)c5Cl)cccc43)c(Cl)cc2CN[C@@H]2CC[C@H]2O)c1] | +++ | 757.20 | 1.80 |
| 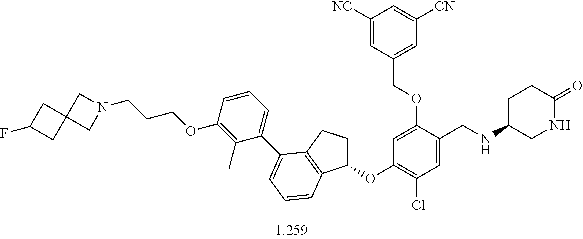 [SMILES: Cc1c(OCCCN2CC3(CC(F)C3)C2)cccc1-c1cccc2c1CC[C@@H]2Oc1cc(OCc2cc(C#N)cc(C#N)c2)c(CN[C@H]2CCC(=O)NC2)cc1Cl] | +++ | 788.20 | 2.44 |
| 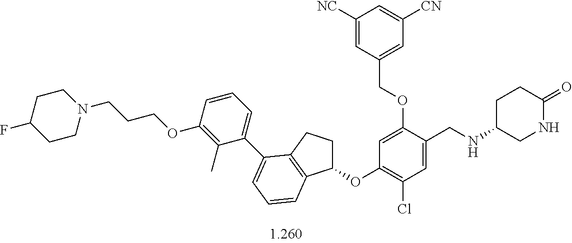 [SMILES: Cc1c(OCCCN2CCC(F)CC2)cccc1-c1cccc2c1CC[C@@H]2Oc1cc(OCc2cc(C#N)cc(C#N)c2)c(CN[C@@H]2CCC(=O)NC2)cc1Cl] | +++ | 776.20 | 1.88 |
| 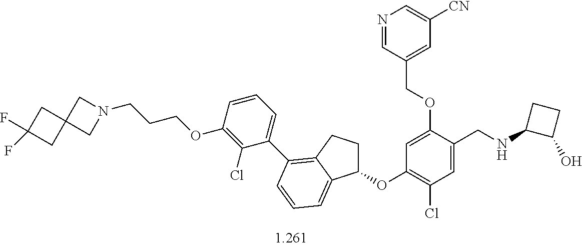 [SMILES: N#Cc1cncc(COc2cc(O[C@H]3CCc4c(-c5cccc(OCCCN6CC7(C6)CC(F)(F)C7)c5Cl)cccc43)c(Cl)cc2CN[C@H]2CC[C@@H]2O)c1] | ++ | 775.10 | 2.10 |
| 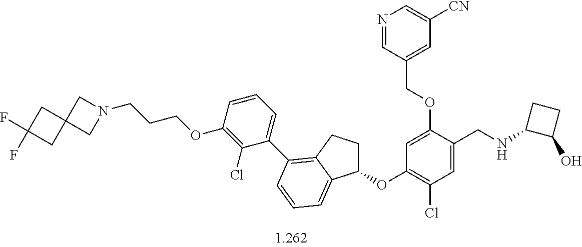 [SMILES: N#Cc1cncc(COc2cc(O[C@H]3CCc4c(-c5cccc(OCCCN6CC7(C6)CC(F)(F)C7)c5Cl)cccc43)c(Cl)cc2CN[C@@H]2CC[C@H]2O)c1] | +++ | 775.20 | 2.00 |
| 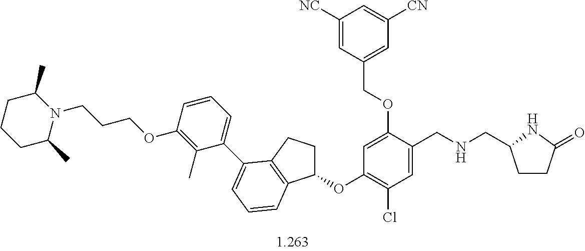 [SMILES: Cc1c(OCCCN2[C@H](C)CCC[C@@H]2C)cccc1-c1cccc2c1CC[C@@H]2Oc1cc(OCc2cc(C#N)cc(C#N)c2)c(CNC[C@H]2CCC(=O)N2)cc1Cl] | ++ | 786.30 | 2.43 |
| 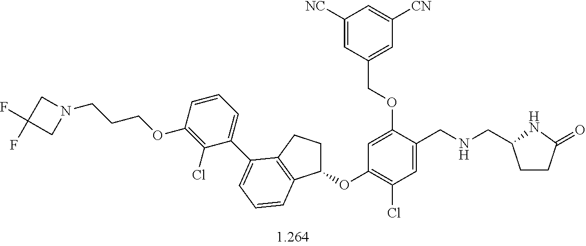 [SMILES: N#Cc1cc(C#N)cc(COc2cc(O[C@H]3CCc4c(-c5cccc(OCCCN6CC(F)(F)C6)c5Cl)cccc43)c(Cl)cc2CNC[C@H]2CCC(=O)N2)c1] | ++ | 766.20 | 2.19 |
| 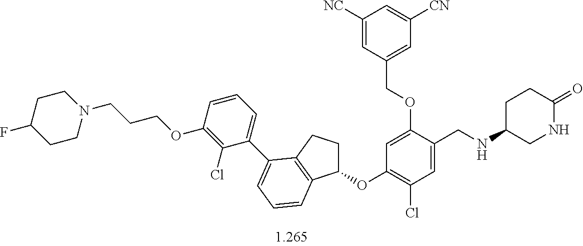 [SMILES: N#Cc1cc(C#N)cc(COc2cc(O[C@H]3CCc4c(-c5cccc(OCCCN6CCC(F)CC6)c5Cl)cccc43)c(Cl)cc2CN[C@H]2CCC(=O)NC2)c1] | ++ | 796.20 | 1.86 |
| 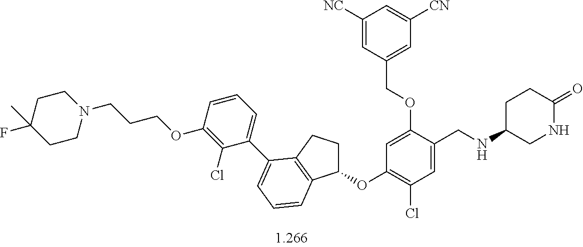 [SMILES: CC1(F)CCN(CCCOc2cccc(-c3cccc4c3CC[C@@H]4Oc3cc(OCc4cc(C#N)cc(C#N)c4)c(CN[C@H]4CCC(=O)NC4)cc3Cl)c2Cl)CC1] | ++ | 810.20 | 2.10 |
| 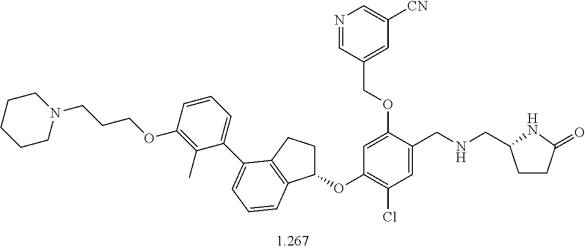 [SMILES: Cc1c(OCCCN2CCCCC2)cccc1-c1cccc2c1CC[C@@H]2Oc1cc(OCc2cncc(C#N)c2)c(CNC[C@H]2CCC(=O)N2)cc1Cl] | +++ | 734.20 | 3.12* |
| 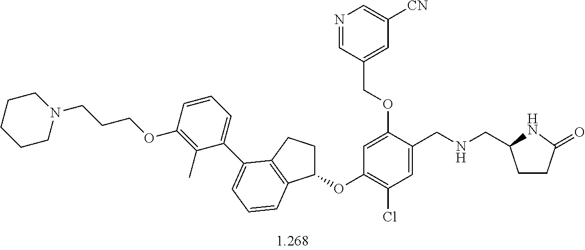 [SMILES: Cc1c(OCCCN2CCCCC2)cccc1-c1cccc2c1CC[C@@H]2Oc1cc(OCc2cncc(C#N)c2)c(CNC[C@@H]2CCC(=O)N2)cc1Cl] | +++ | 734.20 | 3.26* |
| 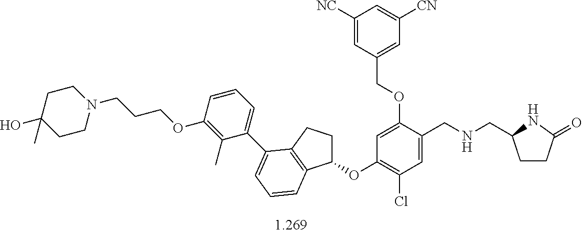 [SMILES: Cc1c(OCCCN2CCC(C)(O)CC2)cccc1-c1cccc2c1CC[C@@H]2Oc1cc(OCc2cc(C#N)cc(C#N)c2)c(CNC[C@@H]2CCC(=O)N2)cc1Cl] | +++ | 788.30 | 1.92* |
| 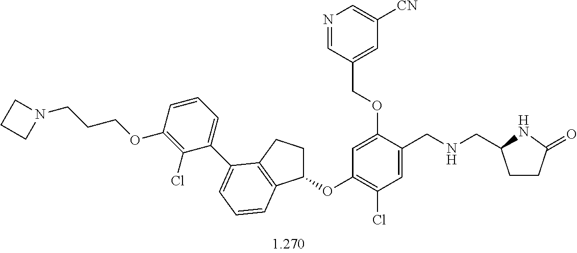 [SMILES: N#Cc1cncc(COc2cc(O[C@H]3CCc4c(-c5cccc(OCCCN6CCC6)c5Cl)cccc43)c(Cl)cc2CNC[C@@H]2CCC(=O)N2)c1] | +++ | 726.20 | 2.02 |
| 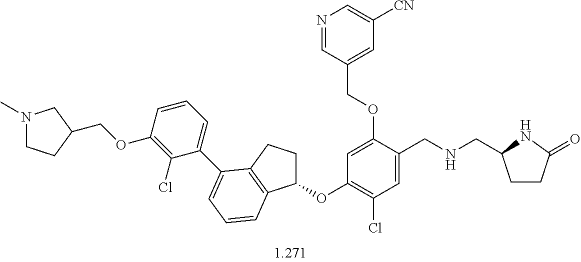 [SMILES: CN1CCC(COc2cccc(-c3cccc4c3CC[C@@H]4Oc3cc(OCc4cncc(C#N)c4)c(CNC[C@@H]4CCC(=O)N4)cc3Cl)c2Cl)C1] | +++ | 726.20 | 1.94 |
| 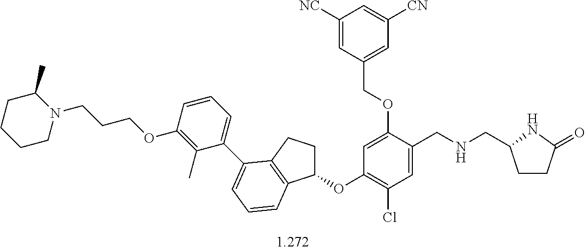 [SMILES: Cc1c(OCCCN2CCCC[C@H]2C)cccc1-c1cccc2c1CC[C@@H]2Oc1cc(OCc2cc(C#N)cc(C#N)c2)c(CNC[C@H]2CCC(=O)N2)cc1Cl] | +++ | 772.20 | 2.16 |
| 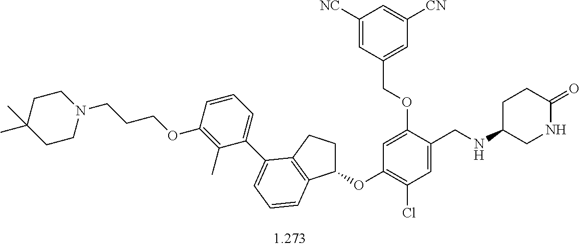 [SMILES: Cc1c(OCCCN2CCC(C)(C)CC2)cccc1-c1cccc2c1CC[C@@H]2Oc1cc(OCc2cc(C#N)cc(C#N)c2)c(CN[C@H]2CCC(=O)NC2)cc1Cl] | ++ | 786.30 | 2.11 |
| 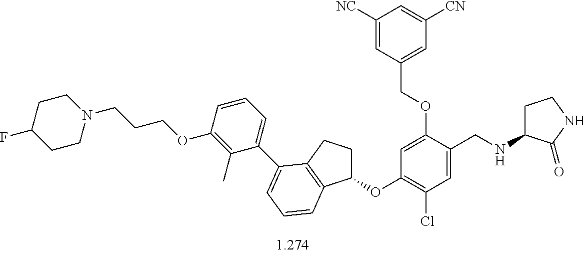 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCNC2=O)cc1Cl] | ++ | 762.20 | 1.92 |
| 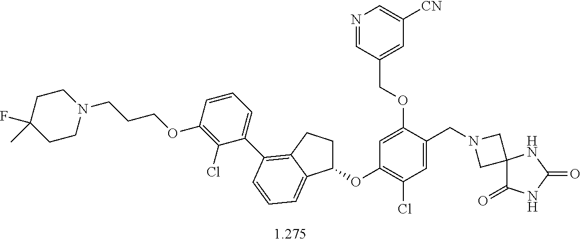 [SMILES: CC1(F)CCN(CCCOc2cccc(-c3cccc4c3CC[C@@H]4Oc3cc(OCc4cncc(C#N)c4)c(CN4CC5(C4)NC(=O)NC5=O)cc3Cl)c2Cl)CC1] | ++ | 813.00 | 2.20 |
| 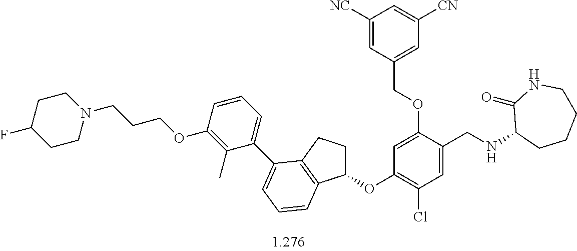 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCCCNC2=O)cc1Cl] | ++ | 790.20 | 2.21 |
| 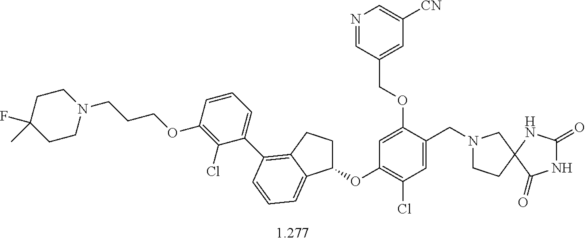 [SMILES: CC1(F)CCN(CCCOc2cccc(-c3cccc4c3CC[C@@H]4Oc3cc(OCc4cncc(C#N)c4)c(CN4CCC5(C4)NC(=O)NC5=O)cc3Cl)c2Cl)CC1] | ++ | 827.20 | 2.00 |
| 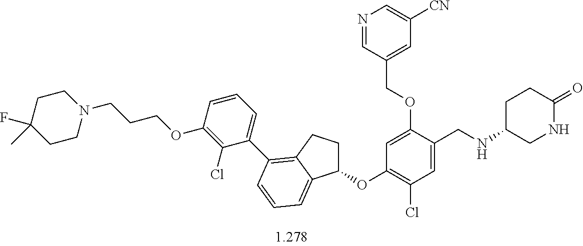 [SMILES: CC1(F)CCN(CCCOc2cccc(-c3cccc4c3CC[C@@H]4Oc3cc(OCc4cncc(C#N)c4)c(CN[C@@H]4CCC(=O)NC4)cc3Cl)c2Cl)CC1] | ++ | 786.20 | 1.70 |
| 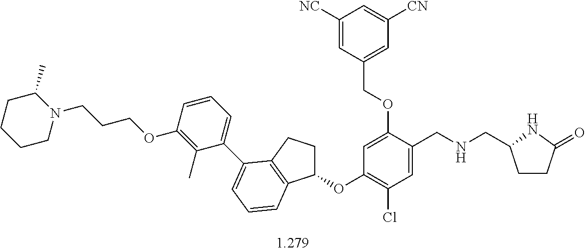 [SMILES: Cc1c(OCCCN2CCCC[C@@H]2C)cccc1-c1cccc2c1CC[C@@H]2Oc1cc(OCc2cc(C#N)cc(C#N)c2)c(CNC[C@H]2CCC(=O)N2)cc1Cl] | ++ | 772.20 | 2.38 |
| 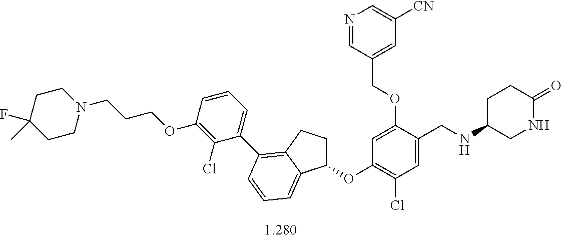 [SMILES: CC1(F)CCN(CCCOc2cccc(-c3cccc4c3CC[C@@H]4Oc3cc(OCc4cncc(C#N)c4)c(CN[C@H]4CCC(=O)NC4)cc3Cl)c2Cl)CC1] | ++ | 787.20 | 1.90 |
| 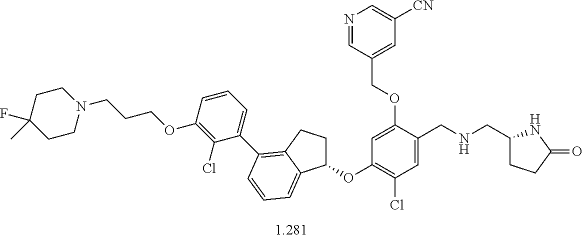 [SMILES: CC1(F)CCN(CCCOc2cccc(-c3cccc4c3CC[C@@H]4Oc3cc(OCc4cncc(C#N)c4)c(CNC[C@H]4CCC(=O)N4)cc3Cl)c2Cl)CC1] | ++ | 786.20 | 1.90 |
| 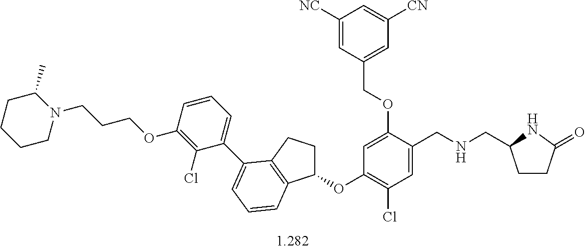 [SMILES: C[C@H]1CCCCN1CCCOc1cccc(-c2cccc3c2CC[C@@H]3Oc2cc(OCc3cc(C#N)cc(C#N)c3)c(CNC[C@@H]3CCC(=O)N3)cc2Cl)c1Cl] | +++ | 772.30 | 2.58 |
| 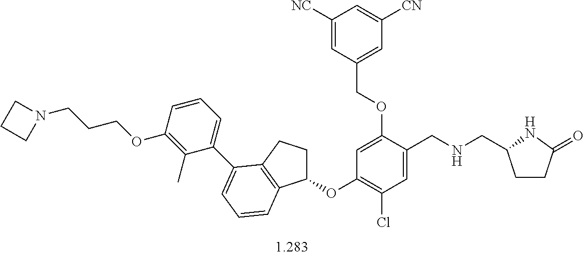 [SMILES: Cc1c(OCCCN2CCC2)cccc1-c1cccc2c1CC[C@@H]2Oc1cc(OCc2cc(C#N)cc(C#N)c2)c(CNC[C@H]2CCC(=O)N2)cc1Cl] | +++ | 730.20 | 2.15 |
| 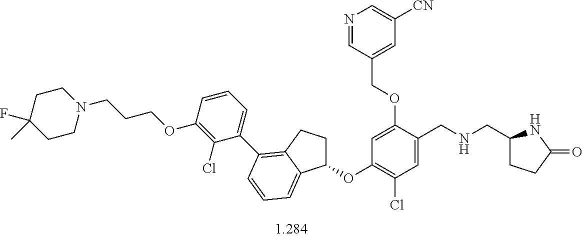 [SMILES: CC1(F)CCN(CCCOc2cccc(-c3cccc4c3CC[C@@H]4Oc3cc(OCc4cncc(C#N)c4)c(CNC[C@@H]4CCC(=O)N4)cc3Cl)c2Cl)CC1] | +++ | 786.10 | 2.20 |
| 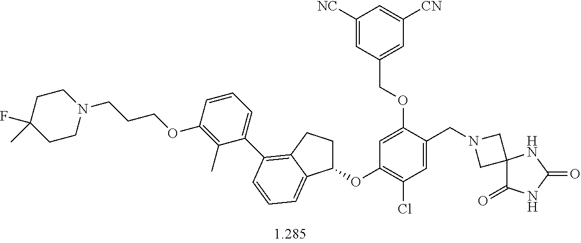 [SMILES: Cc1c(OCCCN2CCC(C)(F)CC2)cccc1-c1cccc2c1CC[C@@H]2Oc1cc(OCc2cc(C#N)cc(C#N)c2)c(CN2CC3(C2)NC(=O)NC3=O)cc1Cl] | + | 817.20 | 2.00 |
| 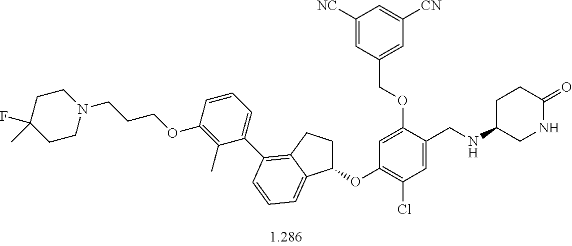 [SMILES: Cc1c(OCCCN2CCC(C)(F)CC2)cccc1-c1cccc2c1CC[C@@H]2Oc1cc(OCc2cc(C#N)cc(C#N)c2)c(CN[C@H]2CCC(=O)NC2)cc1Cl] | ++ | 790.20 | 2.00 |
| 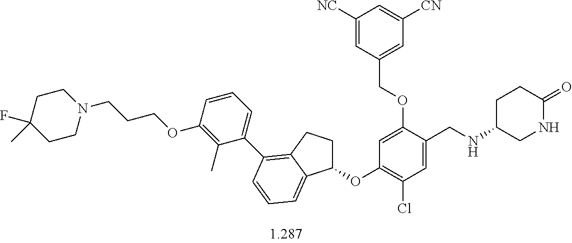 [SMILES: Cc1c(OCCCN2CCC(C)(F)CC2)cccc1-c1cccc2c1CC[C@@H]2Oc1cc(OCc2cc(C#N)cc(C#N)c2)c(CN[C@@H]2CCC(=O)NC2)cc1Cl] | ++ | 790.30 | 2.00 |
| 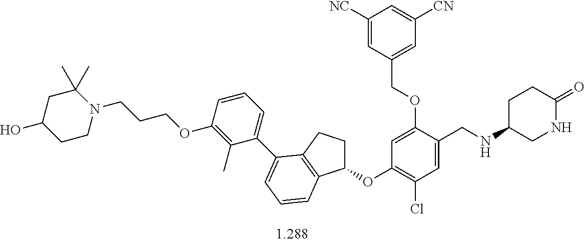 [SMILES: Cc1c(OCCCN2CCC(O)CC2(C)C)cccc1-c1cccc2c1CC[C@@H]2Oc1cc(OCc2cc(C#N)cc(C#N)c2)c(CN[C@H]2CCC(=O)NC2)cc1Cl] | +++ | 802.20 | 1.96 |
| 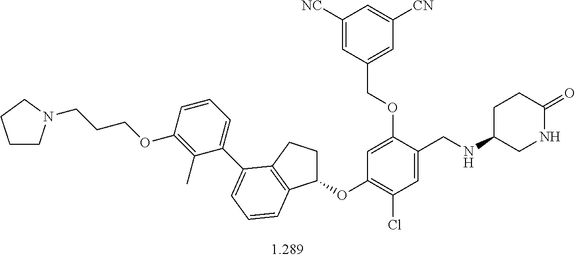 [SMILES: Cc1c(OCCCN2CCCC2)cccc1-c1cccc2c1CC[C@@H]2Oc1cc(OCc2cc(C#N)cc(C#N)c2)c(CN[C@H]2CCC(=O)NC2)cc1Cl] | +++ | 744.30 | 2.02 |
| 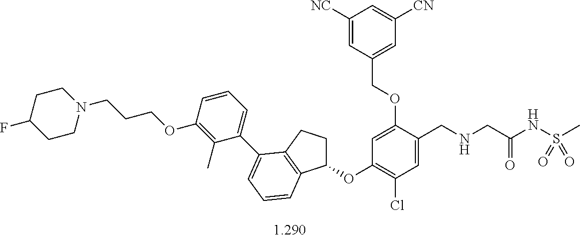 [SMILES: Cc1c(OCCCN2CCC(F)CC2)cccc1-c1cccc2c1CC[C@@H]2Oc1cc(OCc2cc(C#N)cc(C#N)c2)c(CNCC(=O)NS(C)(=O)=O)cc1Cl] | +++ | 814.20 | 2.20 |
| 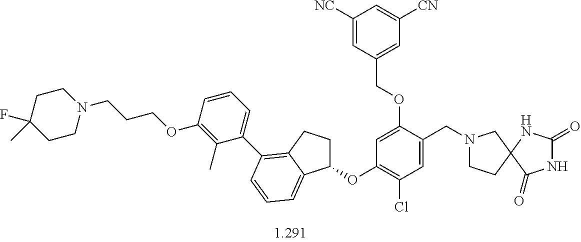 [SMILES: Cc1c(OCCCN2CCC(C)(F)CC2)cccc1-c1cccc2c1CC[C@@H]2Oc1cc(OCc2cc(C#N)cc(C#N)c2)c(CN2CCC3(C2)NC(=O)NC3=O)cc1Cl] | ++ | 831.30 | 2.20 |
| 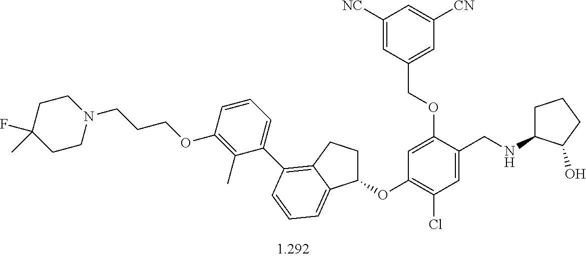 [SMILES: Cc1c(OCCCN2CCC(C)(F)CC2)cccc1-c1cccc2c1CC[C@@H]2Oc1cc(OCc2cc(C#N)cc(C#N)c2)c(CN[C@H]2CCC[C@@H]2O)cc1Cl] | ++ | 777.30 | 2.00 |
| 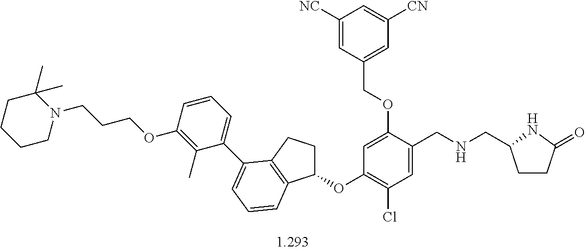 [SMILES: Cc1c(OCCCN2CCCCC2(C)C)cccc1-c1cccc2c1CC[C@@H]2Oc1cc(OCc2cc(C#N)cc(C#N)c2)c(CNC[C@H]2CCC(=O)N2)cc1Cl] | ++ | 786.30 | 2.25 |
| 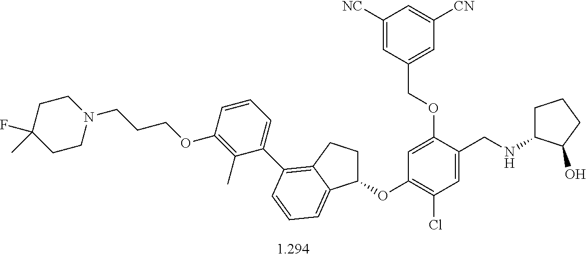 [SMILES: Cc1c(OCCCN2CCC(C)(F)CC2)cccc1-c1cccc2c1CC[C@@H]2Oc1cc(OCc2cc(C#N)cc(C#N)c2)c(CN[C@@H]2CCC[C@H]2O)cc1Cl] | ++ | 778.40 | 2.40 |
| 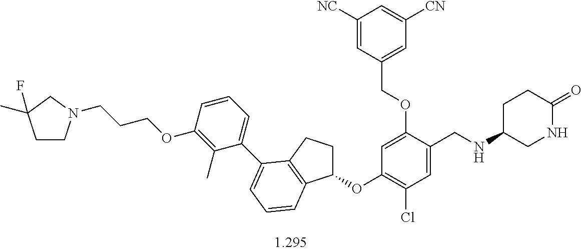 [SMILES: Cc1c(OCCCN2CCC(C)(F)C2)cccc1-c1cccc2c1CC[C@@H]2Oc1cc(OCc2cc(C#N)cc(C#N)c2)c(CN[C@H]2CCC(=O)NC2)cc1Cl] | ++ | 776.20 | 2.00 |
| 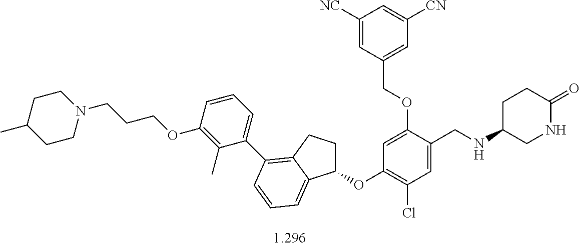 [SMILES: Cc1c(OCCCN2CCC(C)CC2)cccc1-c1cccc2c1CC[C@@H]2Oc1cc(OCc2cc(C#N)cc(C#N)c2)c(CN[C@H]2CCC(=O)NC2)cc1Cl] | ++ | 773.90 | 1.80 |
| 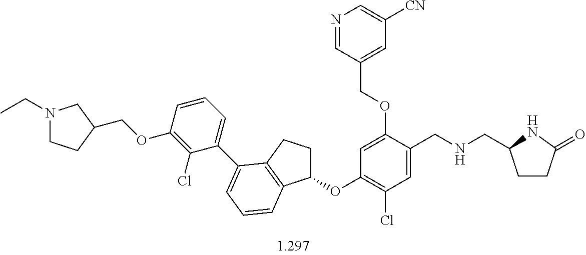 [SMILES: CCN1CCC(COc2cccc(-c3cccc4c3CC[C@@H]4Oc3cc(OCc4cncc(C#N)c4)c(CNC[C@@H]4CCC(=O)N4)cc3Cl)c2Cl)C1] | +++ | 739.80 | 1.77 |
| 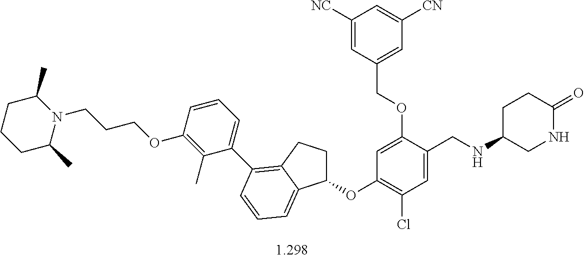 [SMILES: Cc1c(OCCCN2[C@H](C)CCC[C@@H]2C)cccc1-c1cccc2c1CC[C@@H]2Oc1cc(OCc2cc(C#N)cc(C#N)c2)c(CN[C@H]2CCC(=O)NC2)cc1Cl] | ++ | 787.90 | 1.80 |
| 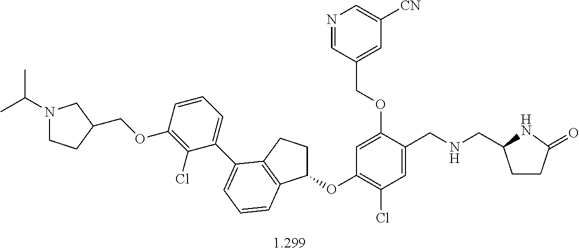 [SMILES: CC(C)N1CCC(COc2cccc(-c3cccc4c3CC[C@@H]4Oc3cc(OCc4cncc(C#N)c4)c(CNC[C@@H]4CCC(=O)N4)cc3Cl)c2Cl)C1] | +++ | 753.80 | 0.45 |
| 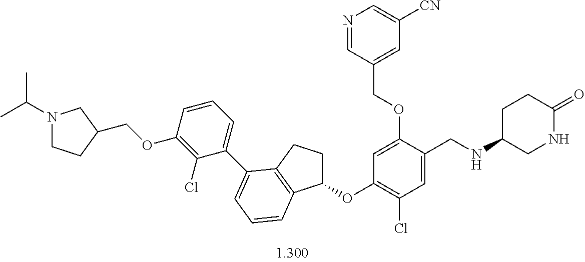 [SMILES: CC(C)N1CCC(COc2cccc(-c3cccc4c3CC[C@@H]4Oc3cc(OCc4cncc(C#N)c4)c(CN[C@H]4CCC(=O)NC4)cc3Cl)c2Cl)C1] | +++ | 754.20 | 2.15 |
| 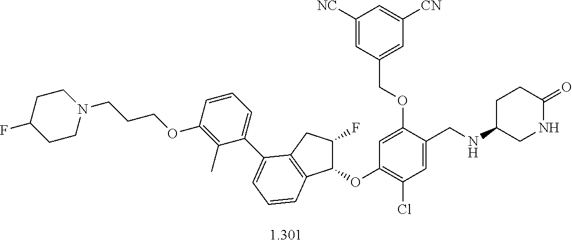 [SMILES: Cc1c(OCCCN2CCC(F)CC2)cccc1-c1cccc2c1C[C@H](F)[C@@H]2Oc1cc(OCc2cc(C#N)cc(C#N)c2)c(CN[C@H]2CCC(=O)NC2)cc1Cl] | ++ | 794.20 | 1.92^{#} |
| 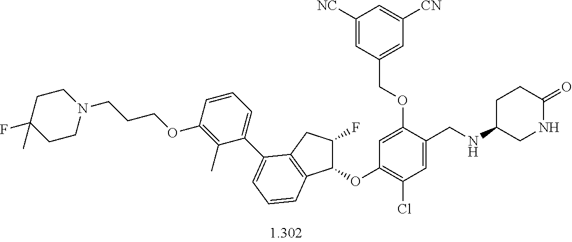 [SMILES: Cc1c(OCCCN2CCC(C)(F)CC2)cccc1-c1cccc2c1C[C@H](F)[C@@H]2Oc1cc(OCc2cc(C#N)cc(C#N)c2)c(CN[C@H]2CCC(=O)NC2)cc1Cl] | ++ | 808.20 | 1.99^{#} |
| 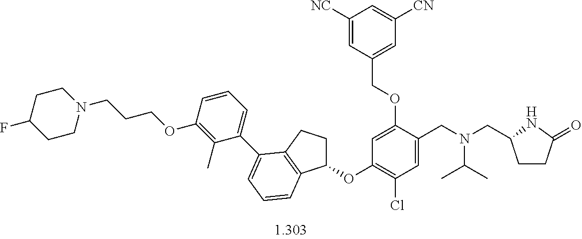 [SMILES: Cc1c(OCCCN2CCC(F)CC2)cccc1-c1cccc2c1CC[C@@H]2Oc1cc(OCc2cc(C#N)cc(C#N)c2)c(CN(C[C@H]2CCC(=O)N2)C(C)C)cc1Cl] | ++ | 878.20 | 2.95* |
| 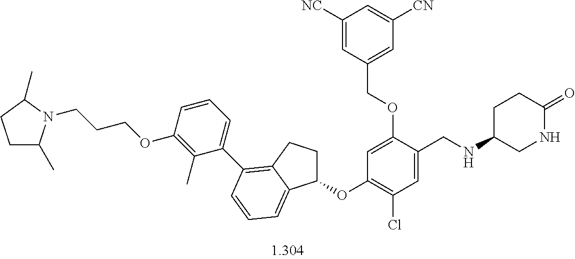 [SMILES: Cc1c(OCCCN2C(C)CCC2C)cccc1-c1cccc2c1CC[C@@H]2Oc1cc(OCc2cc(C#N)cc(C#N)c2)c(CN[C@H]2CCC(=O)NC2)cc1Cl] | +++ | 773.90 | 2.00 |
| 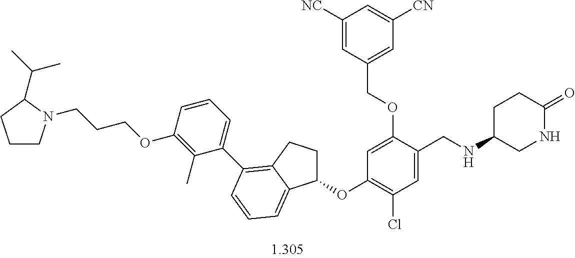 [SMILES: Cc1c(OCCCN2CCCC2C(C)C)cccc1-c1cccc2c1CC[C@@H]2Oc1cc(OCc2cc(C#N)cc(C#N)c2)c(CN[C@H]2CCC(=O)NC2)cc1Cl] | ++ | 787.90 | 1.70 |
| 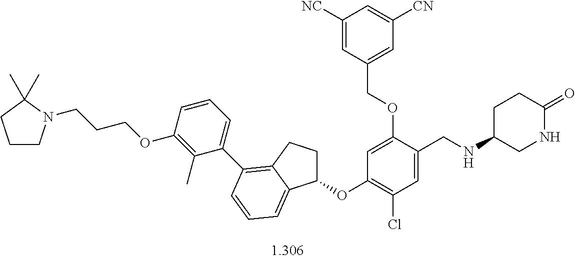 [SMILES: Cc1c(OCCCN2CCCC2(C)C)cccc1-c1cccc2c1CC[C@@H]2Oc1cc(OCc2cc(C#N)cc(C#N)c2)c(CN[C@H]2CCC(=O)NC2)cc1Cl] | +++ | 771.90 | 1.80 |
| 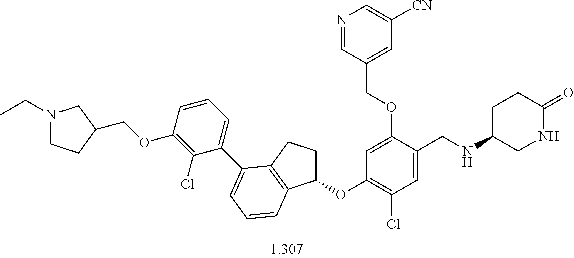 [SMILES: CCN1CCC(COc2cccc(-c3cccc4c3CC[C@@H]4Oc3cc(OCc4cncc(C#N)c4)c(CN[C@H]4CCC(=O)NC4)cc3Cl)c2Cl)C1] | +++ | 740.10 | 1.86 |
| 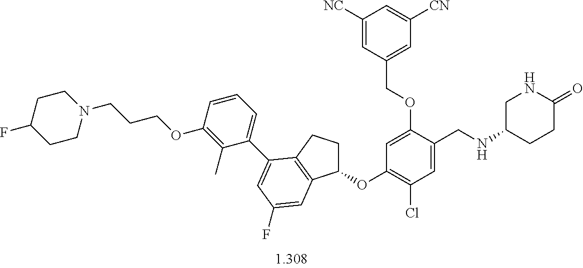 [SMILES: Cc1c(OCCCN2CCC(F)CC2)cccc1-c1cc(F)cc2c1CC[C@@H]2Oc1cc(OCc2cc(C#N)cc(C#N)c2)c(CN[C@H]2CCC(=O)NC2)cc1Cl] | ++ | 794.20 | 1.93 |
| 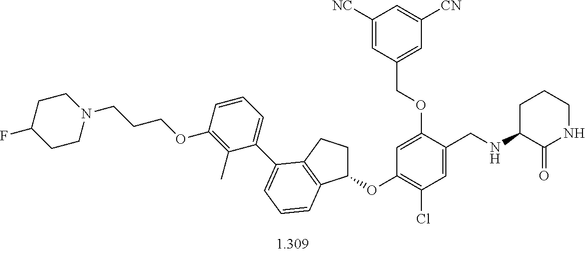 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCCNC2=O)cc1Cl] | ++ | 776.20 | 1.86 |
| 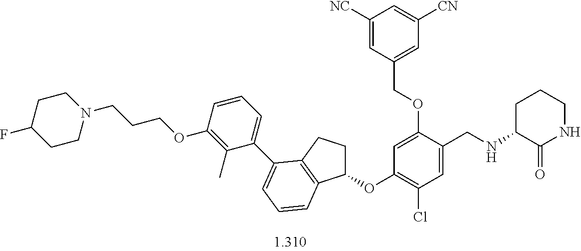 [SMILES: Cc1c(OCCCN2CCC(F)CC2)cccc1-c1cccc2c1CC[C@@H]2Oc1cc(OCc2cc(C#N)cc(C#N)c2)c(CN[C@@H]2CCCNC2=O)cc1Cl] | ++ | 776.20 | 1.88 |
| 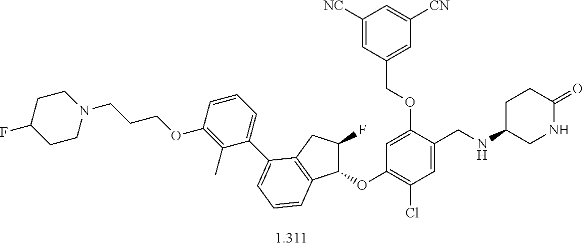 [SMILES: Cc1c(OCCCN2CCC(F)CC2)cccc1-c1cccc2c1C[C@@H](F)[C@@H]2Oc1cc(OCc2cc(C#N)cc(C#N)c2)c(CN[C@H]2CCC(=O)NC2)cc1Cl] | ++ | 793.80 | 1.88^{$} |
| 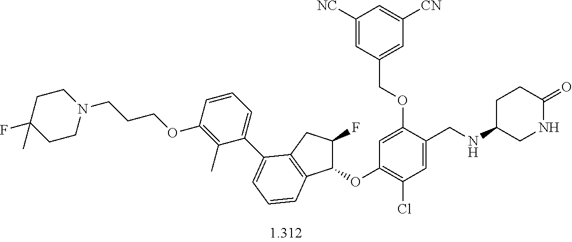 [SMILES: Cc1c(OCCCN2CCC(C)(F)CC2)cccc1-c1cccc2c1C[C@@H](F)[C@@H]2Oc1cc(OCc2cc(C#N)cc(C#N)c2)c(CN[C@H]2CCC(=O)NC2)cc1Cl] | ++ | 807.80 | 1.91^{$} |
| 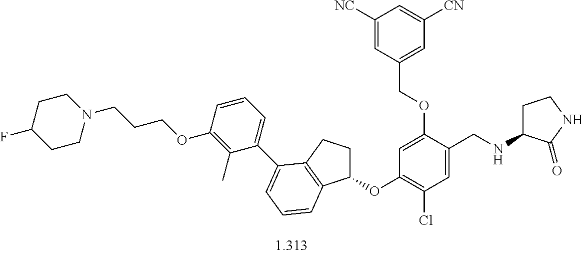 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCNC2=O)cc1Cl] | ++ | 762.20 | 1.75 |
| 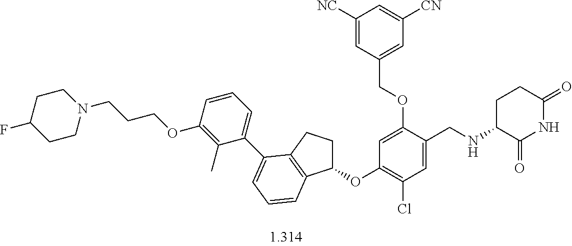 [SMILES: Cc1c(OCCCN2CCC(F)CC2)cccc1-c1cccc2c1CC[C@@H]2Oc1cc(OCc2cc(C#N)cc(C#N)c2)c(CN[C@@H]2CCC(=O)NC2=O)cc1Cl] | ++ | 790.20 | 1.76 |
| 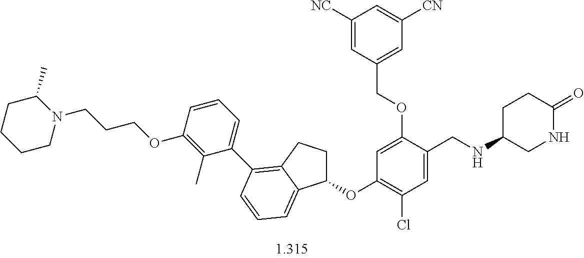 [SMILES: Cc1c(OCCCN2CCCC[C@@H]2C)cccc1-c1cccc2c1CC[C@@H]2Oc1cc(OCc2cc(C#N)cc(C#N)c2)c(CN[C@H]2CCC(=O)NC2)cc1Cl] | +++ | 773.90 | 1.90 |
| 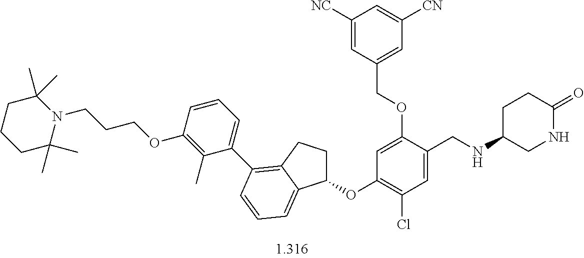 [SMILES: Cc1c(OCCCN2C(C)(C)CCCC2(C)C)cccc1-c1cccc2c1CC[C@@H]2Oc1cc(OCc2cc(C#N)cc(C#N)c2)c(CN[C@H]2CCC(=O)NC2)cc1Cl] | ++ | 813.90 | 2.10 |
| 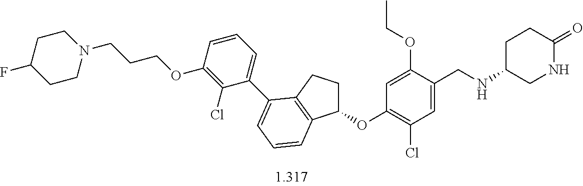 [SMILES: CCOc1cc(O[C@H]2CCc3c(-c4cccc(OCCCN5CCC(F)CC5)c4Cl)cccc32)c(Cl)cc1CN[C@@H]1CCC(=O)NC1] | ++ | 684.20 | 1.71 |
| 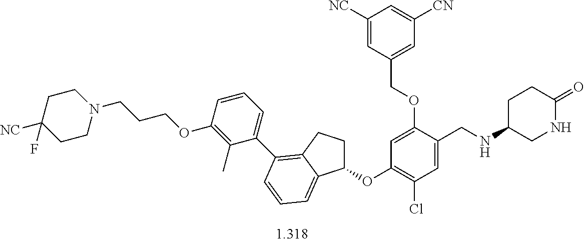 [SMILES: Cc1c(OCCCN2CCC(F)(C#N)CC2)cccc1-c1cccc2c1CC[C@@H]2Oc1cc(OCc2cc(C#N)cc(C#N)c2)c(CN[C@H]2CCC(=O)NC2)cc1Cl] | ++ | 800.80 | 1.90 |
| 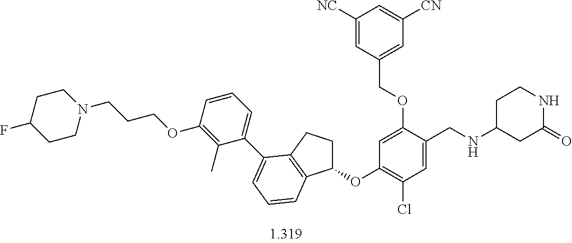 [SMILES: Cc1c(OCCCN2CCC(F)CC2)cccc1-c1cccc2c1CC[C@@H]2Oc1cc(OCc2cc(C#N)cc(C#N)c2)c(CNC2CCNC(=O)C2)cc1Cl] | +++ | 775.90 | 1.77 |
| 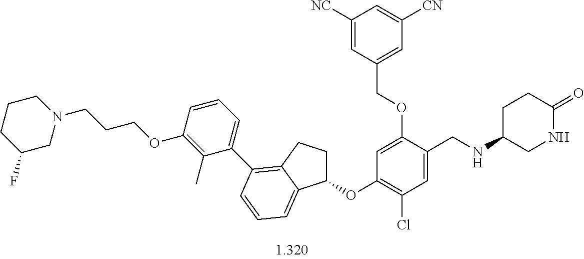 [SMILES: Cc1c(OCCCN2CCC[C@@H](F)C2)cccc1-c1cccc2c1CC[C@@H]2Oc1cc(OCc2cc(C#N)cc(C#N)c2)c(CN[C@H]2CCC(=O)NC2)cc1Cl] | ++ | 775.90 | 1.70 |
| 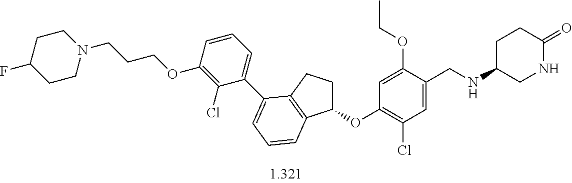 [SMILES: CCOc1cc(O[C@H]2CCc3c(-c4cccc(OCCCN5CCC(F)CC5)c4Cl)cccc32)c(Cl)cc1CN[C@H]1CCC(=O)NC1] | ++ | 684.20 | 1.77 |
| 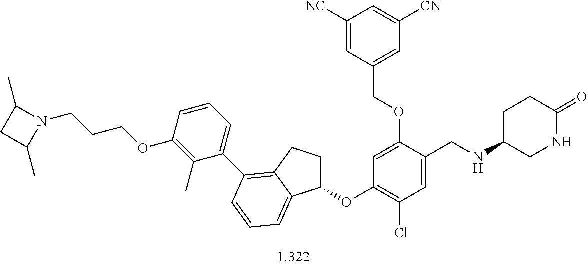 [SMILES: Cc1c(OCCCN2C(C)CC2C)cccc1-c1cccc2c1CC[C@@H]2Oc1cc(OCc2cc(C#N)cc(C#N)c2)c(CN[C@H]2CCC(=O)NC2)cc1Cl] | +++ | 757.90 | 2.00 |
| 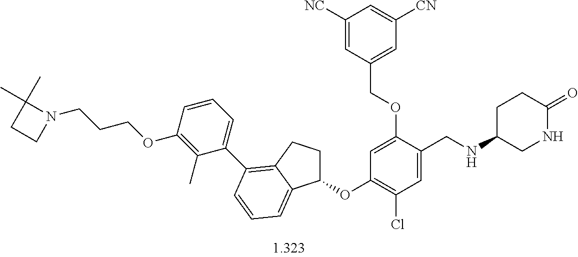 [SMILES: Cc1c(OCCCN2CCC2(C)C)cccc1-c1cccc2c1CC[C@@H]2Oc1cc(OCc2cc(C#N)cc(C#N)c2)c(CN[C@H]2CCC(=O)NC2)cc1Cl] | +++ | 759.90 | 1.90 |
| 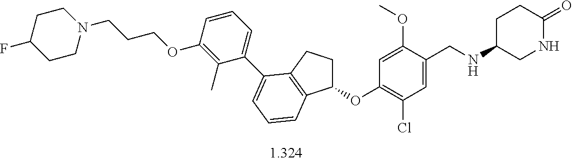 [SMILES: COc1cc(O[C@H]2CCc3c(-c4cccc(OCCCN5CCC(F)CC5)c4C)cccc32)c(Cl)cc1CN[C@H]1CCC(=O)NC1] | ++ | 650.30 | 1.71 |
| 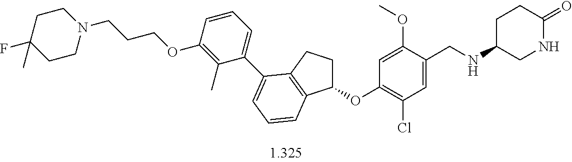 [SMILES: COc1cc(O[C@H]2CCc3c(-c4cccc(OCCCN5CCC(C)(F)CC5)c4C)cccc32)c(Cl)cc1CN[C@H]1CCC(=O)NC1] | ++ | 664.20 | 1.74 |
| 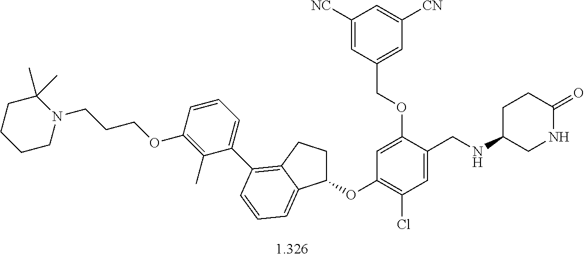 [SMILES: Cc1c(OCCCN2CCCCC2(C)C)cccc1-c1cccc2c1CC[C@@H]2Oc1cc(OCc2cc(C#N)cc(C#N)c2)c(CN[C@H]2CCC(=O)NC2)cc1Cl] | ++ | 785.90 | 1.90 |
| 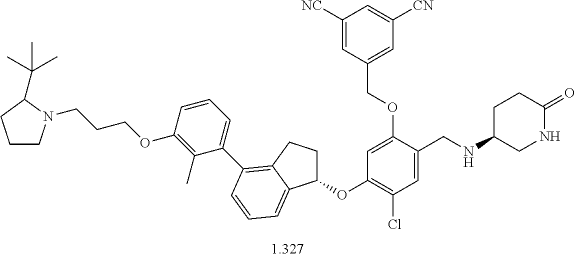 [SMILES: Cc1c(OCCCN2CCCC2C(C)(C)C)cccc1-c1cccc2c1CC[C@@H]2Oc1cc(OCc2cc(C#N)cc(C#N)c2)c(CN[C@H]2CCC(=O)NC2)cc1Cl] | ++ | 800.90 | 2.00 |
| 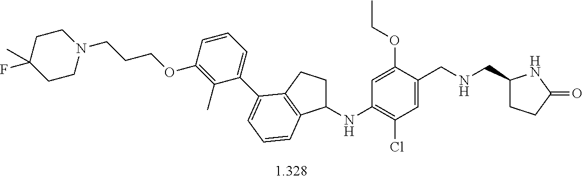 [SMILES: CCOc1cc(NC2CCc3c(-c4cccc(OCCCN5CCC(C)(F)CC5)c4C)cccc32)c(Cl)cc1CNC[C@@H]1CCC(=O)N1] | ++ | 677.20 | 1.79 |
| 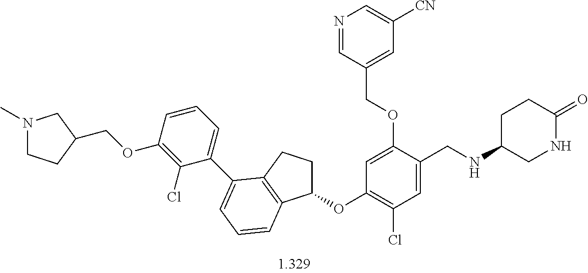 [SMILES: CN1CCC(COc2cccc(-c3cccc4c3CC[C@@H]4Oc3cc(OCc4cncc(C#N)c4)c(CN[C@H]4CCC(=O)NC4)cc3Cl)c2Cl)C1] | ++ | 726.20 | 1.75 |
| 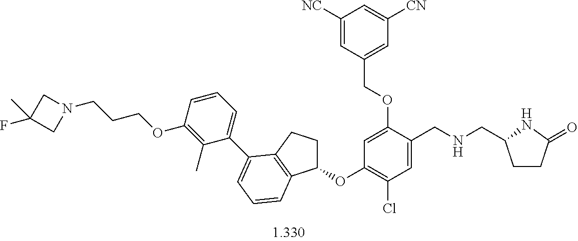 [SMILES: Cc1c(OCCCN2CC(C)(F)C2)cccc1-c1cccc2c1CC[C@@H]2Oc1cc(OCc2cc(C#N)cc(C#N)c2)c(CNC[C@H]2CCC(=O)N2)cc1Cl] | ++ | 762.20 | 1.90 |
| 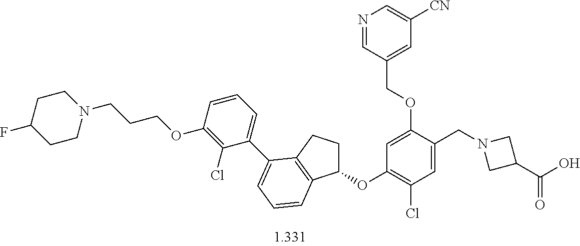 [SMILES: N#Cc1cncc(COc2cc(O[C@H]3CCc4c(-c5cccc(OCCCN6CCC(F)CC6)c5Cl)cccc43)c(Cl)cc2CN2CC(C(=O)O)C2)c1] | +++ | 759.10 | 2.33 |
| 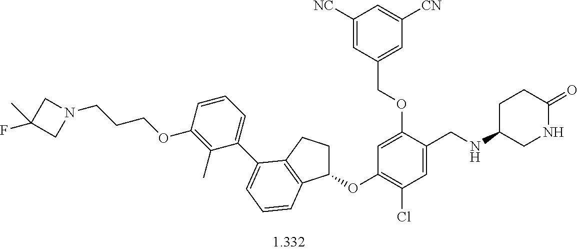 [SMILES: Cc1c(OCCCN2CC(C)(F)C2)cccc1-c1cccc2c1CC[C@@H]2Oc1cc(OCc2cc(C#N)cc(C#N)c2)c(CN[C@H]2CCC(=O)NC2)cc1Cl] | ++ | 762.20 | 1.91 |
| 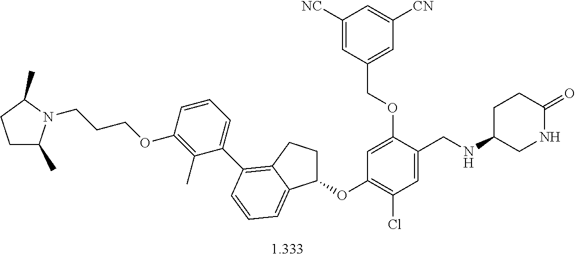 [SMILES: Cc1c(OCCCN2[C@H](C)CC[C@@H]2C)cccc1-c1cccc2c1CC[C@@H]2Oc1cc(OCc2cc(C#N)cc(C#N)c2)c(CN[C@H]2CCC(=O)NC2)cc1Cl] | +++ | 773.90 | 2.00 |
| 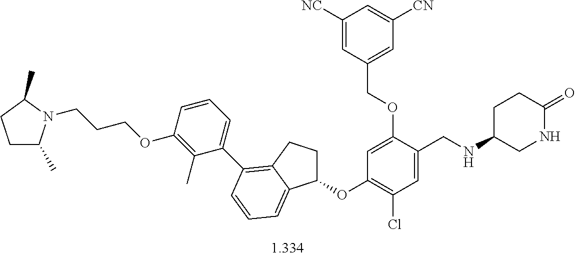 [SMILES: Cc1c(OCCCN2[C@H](C)CC[C@H]2C)cccc1-c1cccc2c1CC[C@@H]2Oc1cc(OCc2cc(C#N)cc(C#N)c2)c(CN[C@H]2CCC(=O)NC2)cc1Cl] | +++ | 773.90 | 2.00 |
| 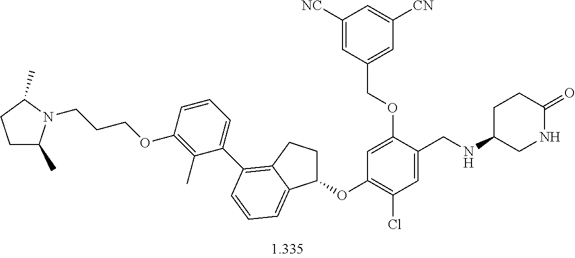 [SMILES: Cc1c(OCCCN2[C@@H](C)CC[C@@H]2C)cccc1-c1cccc2c1CC[C@@H]2Oc1cc(OCc2cc(C#N)cc(C#N)c2)c(CN[C@H]2CCC(=O)NC2)cc1Cl] | +++ | 773.90 | 2.00 |
| 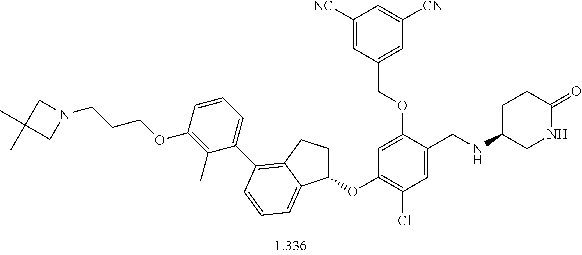 [SMILES: Cc1c(OCCCN2CC(C)(C)C2)cccc1-c1cccc2c1CC[C@@H]2Oc1cc(OCc2cc(C#N)cc(C#N)c2)c(CN[C@H]2CCC(=O)NC2)cc1Cl] | +++ | 757.90 | 1.90 |
| 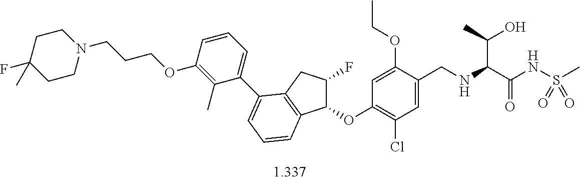 [SMILES: CCOc1cc(O[C@@H]2c3cccc(-c4cccc(OCCCN5CCC(C)(F)CC5)c4C)c3C[C@@H]2F)c(Cl)cc1CN[C@H](C(=O)NS(C)(=O)=O)[C@@H](C)O] | ++ | 778.20 | 1.94^{#} |
| 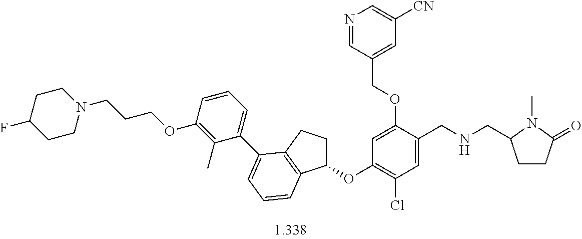 [SMILES: Cc1c(OCCCN2CCC(F)CC2)cccc1-c1cccc2c1CC[C@@H]2Oc1cc(OCc2cncc(C#N)c2)c(CNCC2CCC(=O)N2C)cc1Cl] | ++ | 766.20 | 1.87* |
| 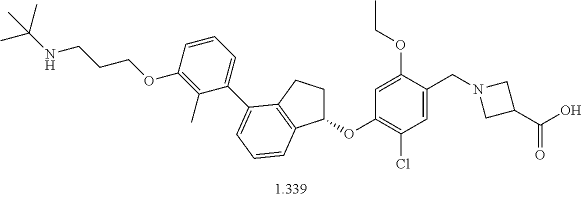 [SMILES: CCOc1cc(O[C@H]2CCc3c(-c4cccc(OCCCNC(C)(C)C)c4C)cccc32)c(Cl)cc1CN1CC(C(=O)O)C1] | ++ | 621.30 | 1.74* |
| 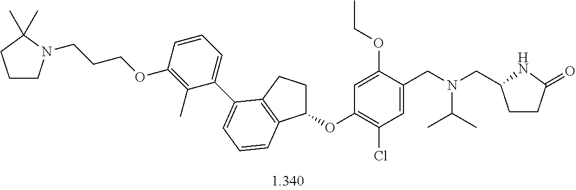 [SMILES: CCOc1cc(O[C@H]2CCc3c(-c4cccc(OCCCN5CCCC5(C)C)c4C)cccc32)c(Cl)cc1CN(C[C@H]1CCC(=O)N1)C(C)C] | + | 706.20 | 1.93 |
| 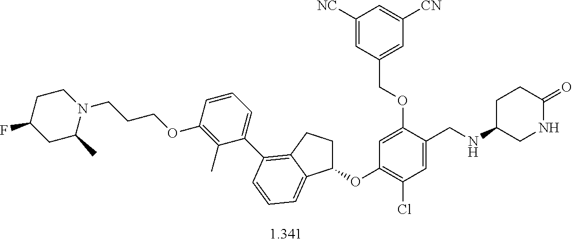 [SMILES: Cc1c(OCCCN2CC[C@H](F)C[C@@H]2C)cccc1-c1cccc2c1CC[C@@H]2Oc1cc(OCc2cc(C#N)cc(C#N)c2)c(CN[C@H]2CCC(=O)NC2)cc1Cl] | ++ | 789.80 | 1.90 |
| 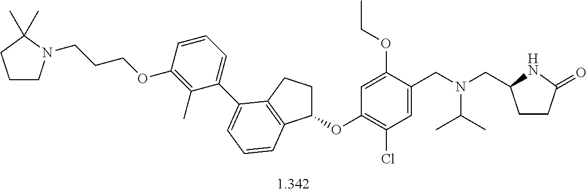 [SMILES: CCOc1cc(O[C@H]2CCc3c(-c4cccc(OCCCN5CCCC5(C)C)c4C)cccc32)c(Cl)cc1CN(C[C@@H]1CCC(=O)N1)C(C)C] | ++ | 702.30 | 1.68 |
| 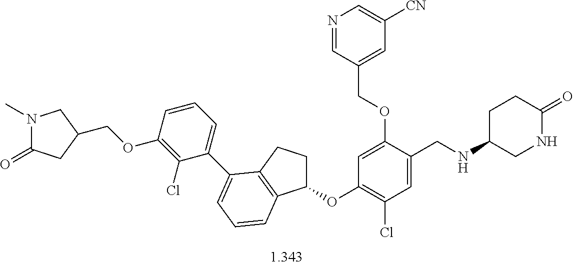 [SMILES: CN1CC(COc2cccc(-c3cccc4c3CC[C@@H]4Oc3cc(OCc4cncc(C#N)c4)c(CN[C@H]4CCC(=O)NC4)cc3Cl)c2Cl)CC1=O] | ++ | 740.10 | 2.05 |
| 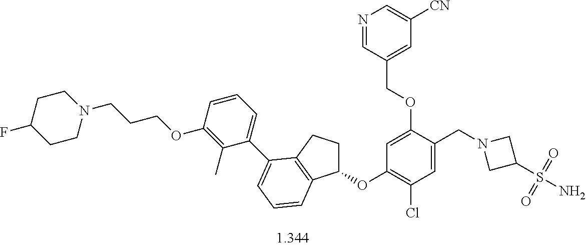 [SMILES: Cc1c(OCCCN2CCC(F)CC2)cccc1-c1cccc2c1CC[C@@H]2Oc1cc(OCc2cncc(C#N)c2)c(CN2CC(S(N)(=O)=O)C2)cc1Cl] | ++ | 774.20 | 2.13 |
| 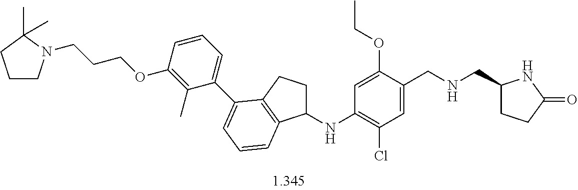 [SMILES: CCOc1cc(NC2CCc3c(-c4cccc(OCCCN5CCCC5(C)C)c4C)cccc32)c(Cl)cc1CNC[C@@H]1CCC(=O)N1] | ++ | 659.20 | 1.91 |
| 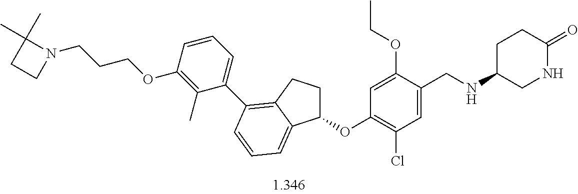 [SMILES: CCOc1cc(O[C@H]2CCc3c(-c4cccc(OCCCN5CCC5(C)C)c4C)cccc32)c(Cl)cc1CN[C@H]1CCC(=O)NC1] | ++ | 646.00 | 1.90 |
| 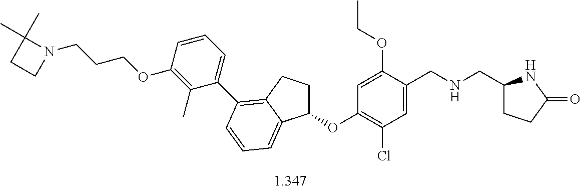 [SMILES: CCOc1cc(O[C@H]2CCc3c(-c4cccc(OCCCN5CCC5(C)C)c4C)cccc32)c(Cl)cc1CNC[C@@H]1CCC(=O)N1] | +++ | 646.00 | 1.80 |
| 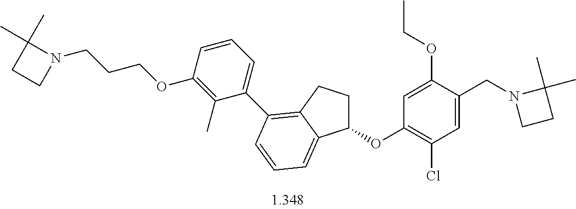 | ++ | 617.00 | 2.10 |
| 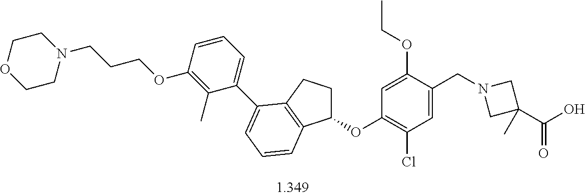 [SMILES: CCOc1cc(O[C@H]2CCc3c(-c4cccc(OCCCN5CCOCC5)c4C)cccc32)c(Cl)cc1CN1CC(C)(C(=O)O)C1] | ++ | 649.20 | 1.71 |
| 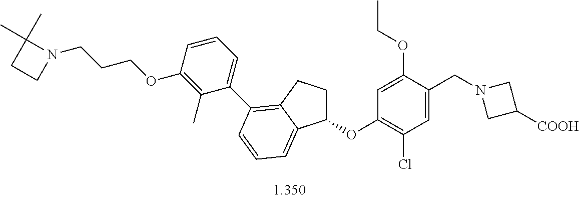 [SMILES: CCOc1cc(O[C@H]2CCc3c(-c4cccc(OCCCN5CCC5(C)C)c4C)cccc32)c(Cl)cc1CN1CC(C(=O)O)C1] | ++ | 633.20 | 1.82 |
| 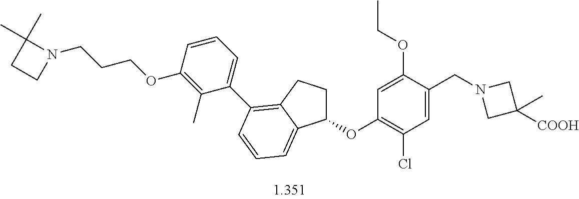 [SMILES: CCOc1cc(O[C@H]2CCc3c(-c4cccc(OCCCN5CCC5(C)C)c4C)cccc32)c(Cl)cc1CN1CC(C)(C(=O)O)C1] | ++ | 647.20 | 1.83 |
| 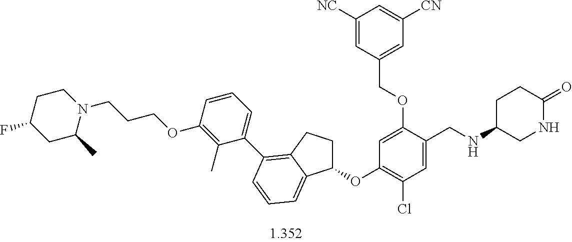 [SMILES: Cc1c(OCCCN2CC[C@@H](F)C[C@@H]2C)cccc1-c1cccc2c1CC[C@@H]2Oc1cc(OCc2cc(C#N)cc(C#N)c2)c(CN[C@H]2CCC(=O)NC2)cc1Cl] | ++ | 790.80 | 1.90 |
| 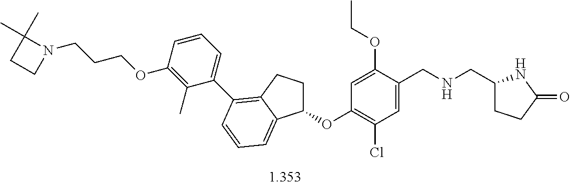 [SMILES: CCOc1cc(O[C@H]2CCc3c(-c4cccc(OCCCN5CCC5(C)C)c4C)cccc32)c(Cl)cc1CNC[C@H]1CCC(=O)N1] | ++ | 646.00 | 1.90 |
| 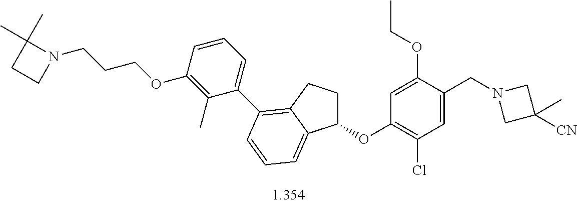 [SMILES: CCOc1cc(O[C@H]2CCc3c(-c4cccc(OCCCN5CCC5(C)C)c4C)cccc32)c(Cl)cc1CN1CC(C)(C#N)C1] | ++ | 628.20 | 1.97 |
| 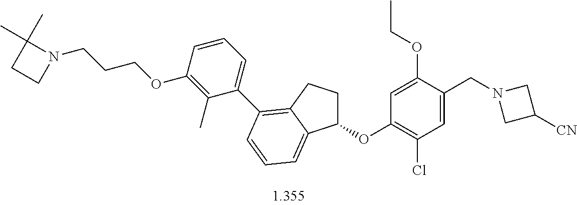 [SMILES: CCOc1cc(O[C@H]2CCc3c(-c4cccc(OCCCN5CCC5(C)C)c4C)cccc32)c(Cl)cc1CN1CC(C#N)C1] | ++ | 614.20 | 1.92 |
| 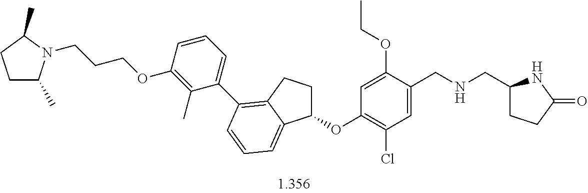 [SMILES: CCOc1cc(O[C@H]2CCc3c(-c4cccc(OCCCN5[C@H](C)CC[C@H]5C)c4C)cccc32)c(Cl)cc1CNC[C@@H]1CCC(=O)N1] | +++ | 660.00 | 1.90 |
| 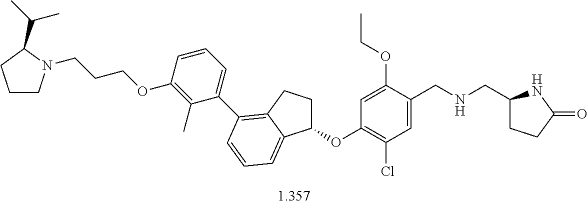 [SMILES: CCOc1cc(O[C@H]2CCc3c(-c4cccc(OCCCN5CCC[C@H]5C(C)C)c4C)cccc32)c(Cl)cc1CNC[C@@H]1CCC(=O)N1] | ++ | 674.00 | 1.90 |
| 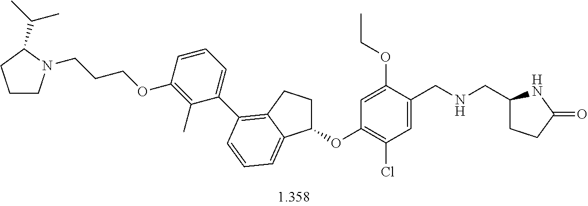 [SMILES: CCOc1cc(O[C@H]2CCc3c(-c4cccc(OCCCN5CCC[C@@H]5C(C)C)c4C)cccc32)c(Cl)cc1CNC[C@@H]1CCC(=O)N1] | +++ | 674.00 | 2.00 |
| 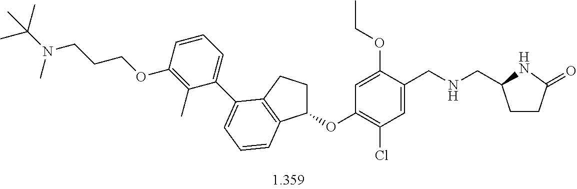 [SMILES: CCOc1cc(O[C@H]2CCc3c(-c4cccc(OCCCN(C)C(C)(C)C)c4C)cccc32)c(Cl)cc1CNC[C@@H]1CCC(=O)N1] | +++ | 648.30 | 1.82 |
| 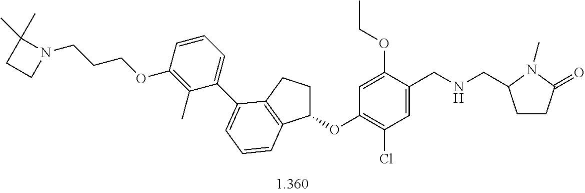 [SMILES: CCOc1cc(O[C@H]2CCc3c(-c4cccc(OCCCN5CCC5(C)C)c4C)cccc32)c(Cl)cc1CNCC1CCC(=O)N1C] | ++ | 660.30 | 1.89 |
| 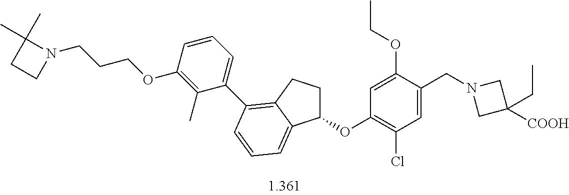 [SMILES: CCOc1cc(O[C@H]2CCc3c(-c4cccc(OCCCN5CCC5(C)C)c4C)cccc32)c(Cl)cc1CN1CC(CC)(C(=O)O)C1] | ++ | 661.20 | 1.95 |
| 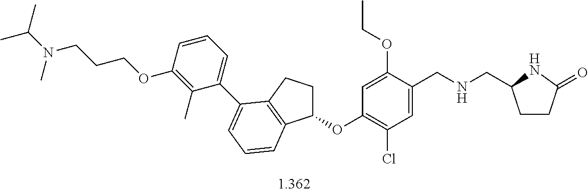 [SMILES: CCOc1cc(O[C@H]2CCc3c(-c4cccc(OCCCN(C)C(C)C)c4C)cccc32)c(Cl)cc1CNC[C@@H]1CCC(=O)N1] | +++ | 634.20 | 1.81 |
| 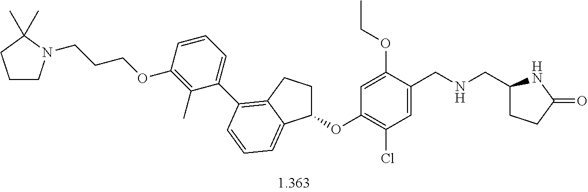 [SMILES: CCOc1cc(O[C@H]2CCc3c(-c4cccc(OCCCN5CCCC5(C)C)c4C)cccc32)c(Cl)cc1CNC[C@@H]1CCC(=O)N1] | +++ | 660.00 | 1.80 |
| 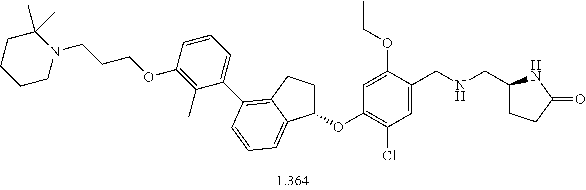 [SMILES: CCOc1cc(O[C@H]2CCc3c(-c4cccc(OCCCN5CCCCC5(C)C)c4C)cccc32)c(Cl)cc1CNC[C@@H]1CCC(=O)N1] | ++ | 674.00 | 1.90 |
| 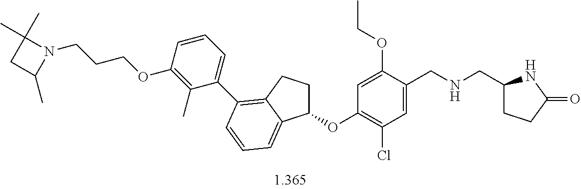 [SMILES: CCOc1cc(O[C@H]2CCc3c(-c4cccc(OCCCN5C(C)CC5(C)C)c4C)cccc32)c(Cl)cc1CNC[C@@H]1CCC(=O)N1] | +++ | 660.00 | 1.70 |
| 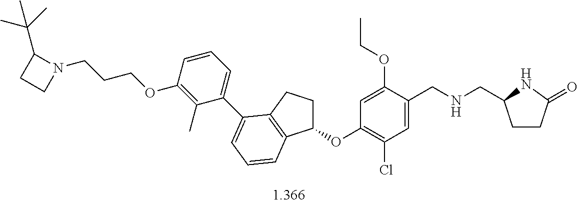 [SMILES: CCOc1cc(O[C@H]2CCc3c(-c4cccc(OCCCN5CCC5C(C)(C)C)c4C)cccc32)c(Cl)cc1CNC[C@@H]1CCC(=O)N1] | ++ | 674.30 | 2.01 |
| 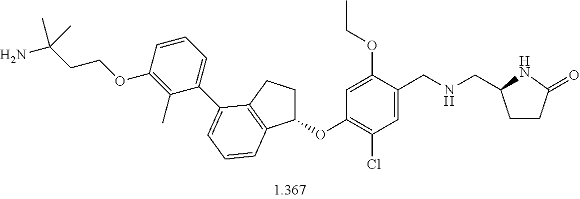 [SMILES: CCOc1cc(O[C@H]2CCc3c(-c4cccc(OCCC(C)(C)N)c4C)cccc32)c(Cl)cc1CNC[C@@H]1CCC(=O)N1] | ++ | 606.20 | 1.56 |
| 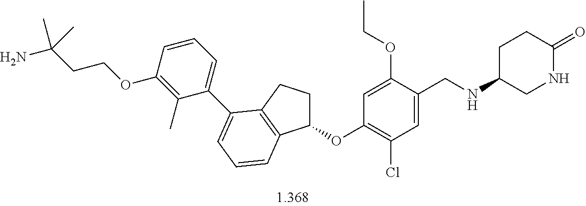 [SMILES: CCOc1cc(O[C@H]2CCc3c(-c4cccc(OCCC(C)(C)N)c4C)cccc32)c(Cl)cc1CN[C@H]1CCC(=O)NC1] | ++ | 606.20 | 1.66 |
| 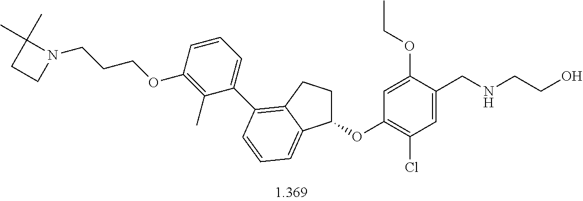 [SMILES: CCOc1cc(O[C@H]2CCc3c(-c4cccc(OCCCN5CCC5(C)C)c4C)cccc32)c(Cl)cc1CNCCO] | +++ | 593.00 | 1.90 |
| 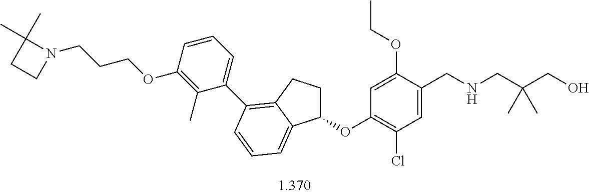 [SMILES: CCOc1cc(O[C@H]2CCc3c(-c4cccc(OCCCN5CCC5(C)C)c4C)cccc32)c(Cl)cc1CNCC(C)(C)CO] | ++ | 635.00 | 2.00 |
| 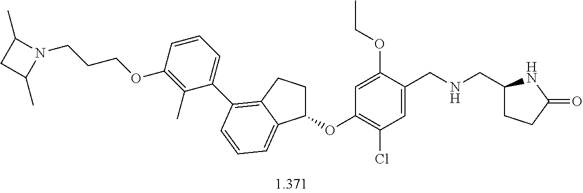 [SMILES: CCOc1cc(O[C@H]2CCc3c(-c4cccc(OCCCN5C(C)CC5C)c4C)cccc32)c(Cl)cc1CNC[C@@H]1CCC(=O)N1] | +++ | 646.00 | 1.90 |
| 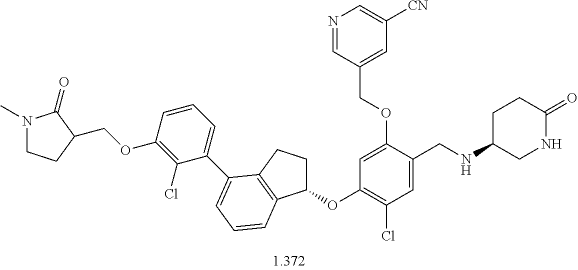 [SMILES: CN1CCC(COc2cccc(-c3cccc4c3CC[C@@H]4Oc3cc(OCc4cncc(C#N)c4)c(CN[C@H]4CCC(=O)NC4)cc3Cl)c2Cl)C1=O] | ++ | 740.10 | 2.13 |
| 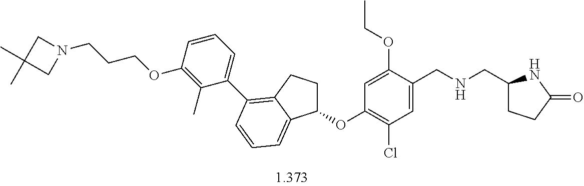 [SMILES: CCOc1cc(O[C@H]2CCc3c(-c4cccc(OCCCN5CC(C)(C)C5)c4C)cccc32)c(Cl)cc1CNC[C@@H]1CCC(=O)N1] | ++ | 646.00 | 1.80 |
| 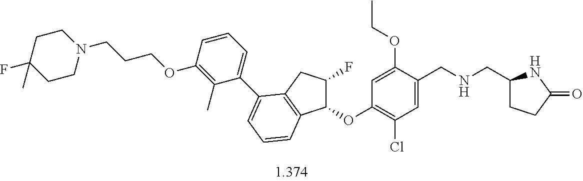 [SMILES: CCOc1cc(O[C@@H]2c3cccc(-c4cccc(OCCCN5CCC(C)(F)CC5)c4C)c3C[C@@H]2F)c(Cl)cc1CNC[C@@H]1CCC(=O)N1] | ++ | 696.20 | 2.04^{#} |
| 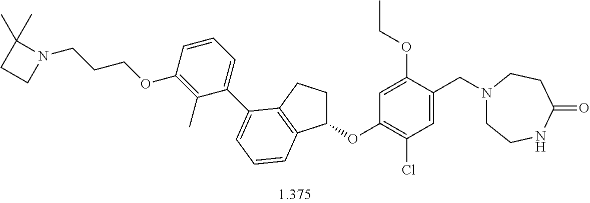 [SMILES: CCOc1cc(O[C@H]2CCc3c(-c4cccc(OCCCN5CCC5(C)C)c4C)cccc32)c(Cl)cc1CN1CCNC(=O)CC1] | ++ | 646.20 | 2.00 |
| 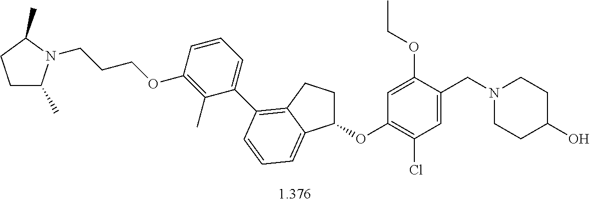 [SMILES: CCOc1cc(O[C@H]2CCc3c(-c4cccc(OCCCN5[C@H](C)CC[C@H]5C)c4C)cccc32)c(Cl)cc1CN1CCC(O)CC1] | ++ | 647.00 | 1.90 |
| 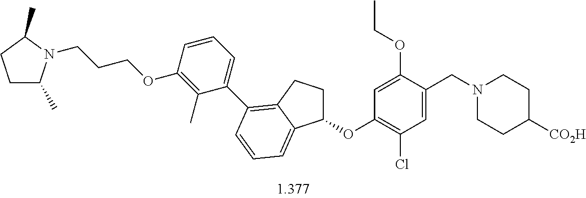 [SMILES: CCOc1cc(O[C@H]2CCc3c(-c4cccc(OCCCN5[C@H](C)CC[C@H]5C)c4C)cccc32)c(Cl)cc1CN1CCC(C(=O)O)CC1] | ++ | 675.00 | 2.20 |
| 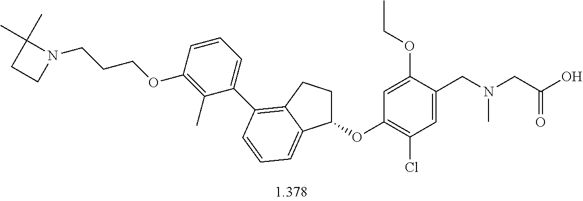 [SMILES: CCOc1cc(O[C@H]2CCc3c(-c4cccc(OCCCN5CCC5(C)C)c4C)cccc32)c(Cl)cc1CN(C)CC(=O)O] | ++ | 622.00 | 1.80 |
| 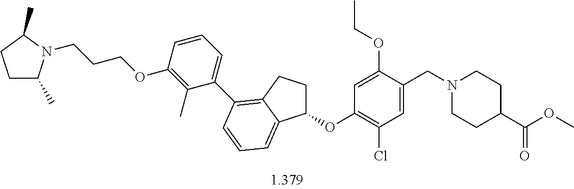 [SMILES: CCOc1cc(O[C@H]2CCc3c(-c4cccc(OCCCN5[C@H](C)CC[C@H]5C)c4C)cccc32)c(Cl)cc1CN1CCC(C(=O)OC)CC1] | ++ | 689.00 | 2.10 |
| 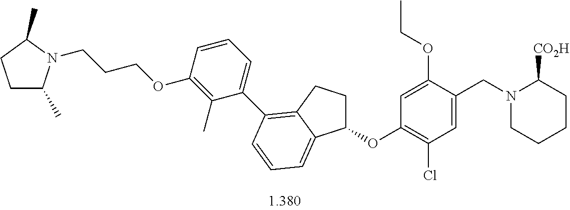 [SMILES: CCOc1cc(O[C@H]2CCc3c(-c4cccc(OCCCN5[C@H](C)CC[C@H]5C)c4C)cccc32)c(Cl)cc1CN1CCCC[C@@H]1C(=O)O] | ++ | 677.30 | 2.00 |
| 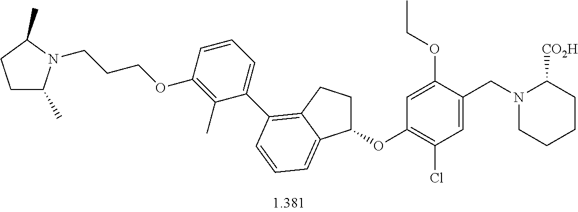 [SMILES: CCOc1cc(O[C@H]2CCc3c(-c4cccc(OCCCN5[C@H](C)CC[C@H]5C)c4C)cccc32)c(Cl)cc1CN1CCCC[C@H]1C(=O)O] | ++ | 676.30 | 2.30 |
| 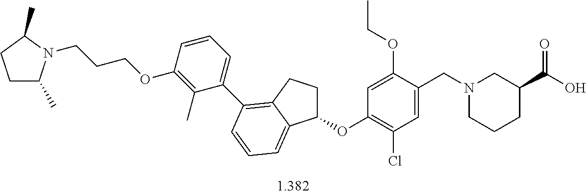 [SMILES: CCOc1cc(O[C@H]2CCc3c(-c4cccc(OCCCN5[C@H](C)CC[C@H]5C)c4C)cccc32)c(Cl)cc1CN1CCC[C@H](C(=O)O)C1] | ++ | 675.30 | 2.00 |
| 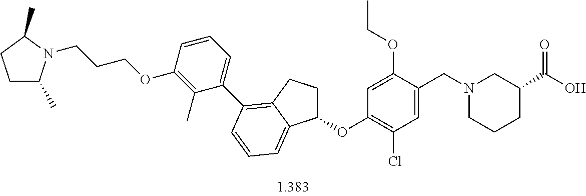 [SMILES: CCOc1cc(O[C@H]2CCc3c(-c4cccc(OCCCN5[C@H](C)CC[C@H]5C)c4C)cccc32)c(Cl)cc1CN1CCC[C@@H](C(=O)O)C1] | ++ | 675.30 | 2.10 |
| 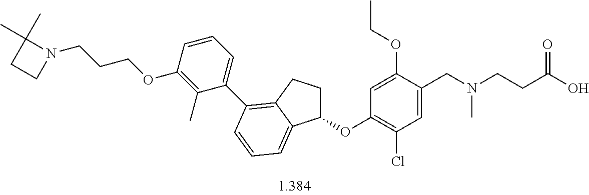 [SMILES: CCOc1cc(O[C@H]2CCc3c(-c4cccc(OCCCN5CCC5(C)C)c4C)cccc32)c(Cl)cc1CN(C)CCC(=O)O] | ++ | 635.30 | 1.70 |
| 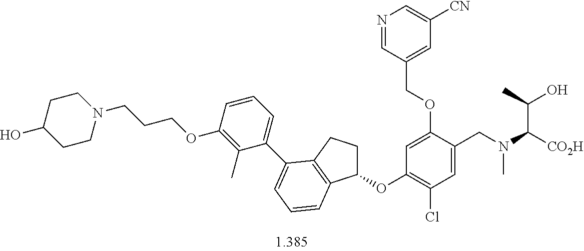 [SMILES: Cc1c(OCCCN2CCC(O)CC2)cccc1-c1cccc2c1CC[C@@H]2Oc1cc(OCc2cncc(C#N)c2)c(CN(C)[C@H](C(=O)O)[C@@H](C)O)cc1Cl] | +++ | 769.20 | 2.17 |
| 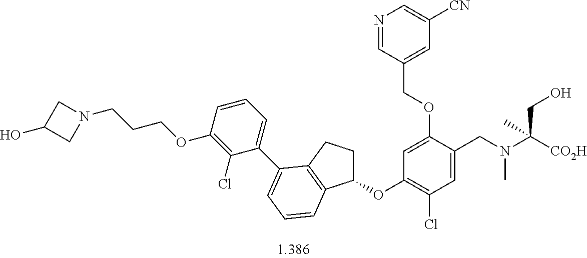 [SMILES: CN(Cc1cc(Cl)c(O[C@H]2CCc3c(-c4cccc(OCCCN5CC(O)C5)c4Cl)cccc32)cc1OCc1cncc(C#N)c1)[C@@](C)(CO)C(=O)O] | +++ | 761.10 | 2.08 |
| 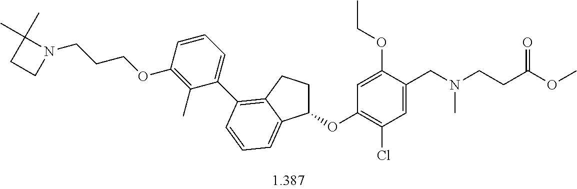 | ++ | 651.40 | 1.70 |
| 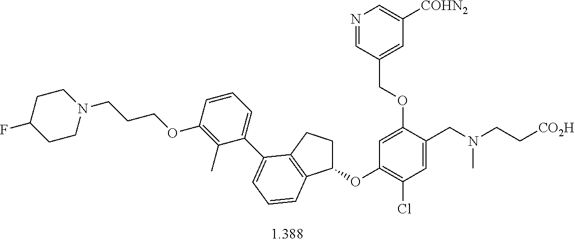 | +++ | 759.30 | 2.15 |
| 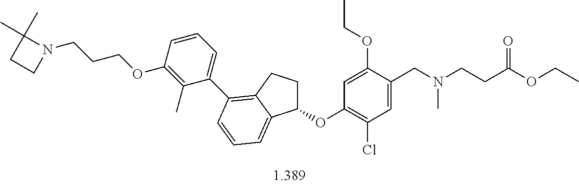 [SMILES: CCOC(=O)CCN(C)Cc1cc(Cl)c(O[C@H]2CCc3c(-c4cccc(OCCCN5CCC5(C)C)c4C)cccc32)cc1OCC] | ++ | 665.40 | 1.90 |
| 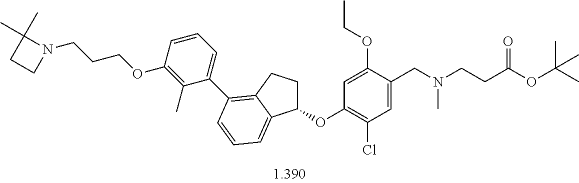 [SMILES: CCOc1cc(O[C@H]2CCc3c(-c4cccc(OCCCN5CCC5(C)C)c4C)cccc32)c(Cl)cc1CN(C)CCC(=O)OC(C)(C)C] | + | 693.40 | 2.00 |
| 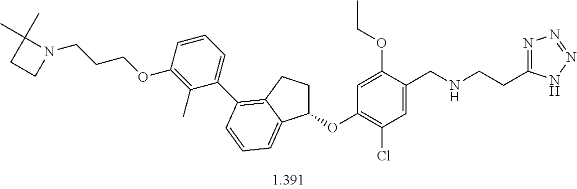 [SMILES: CCOc1cc(O[C@H]2CCc3c(-c4cccc(OCCCN5CCC5(C)C)c4C)cccc32)c(Cl)cc1CNCCc1nnn[nH]1] | ++ | 645.40 | 2.37 |
| 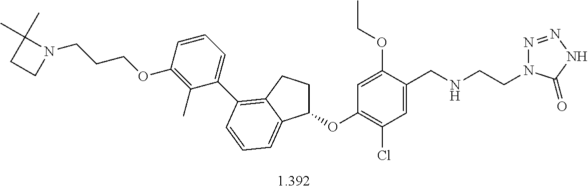 [SMILES: CCOc1cc(O[C@H]2CCc3c(-c4cccc(OCCCN5CCC5(C)C)c4C)cccc32)c(Cl)cc1CNCCn1nn[nH]c1=O] | ++ | 661.40 | 2.35* |
| 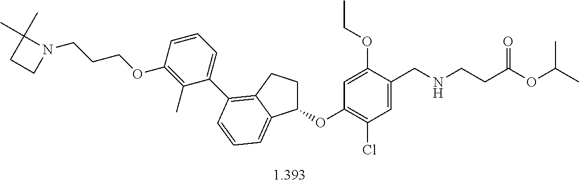 [SMILES: CCOc1cc(O[C@H]2CCc3c(-c4cccc(OCCCN5CCC5(C)C)c4C)cccc32)c(Cl)cc1CNCCC(=O)OC(C)C] | ++ | 664.50 | 2.10 |
| 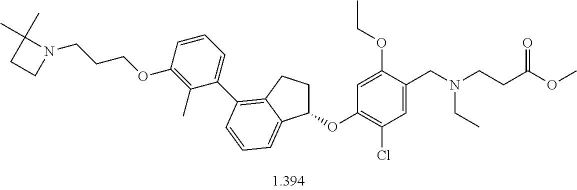 | ++ | 663.40 | 1.90 |
| 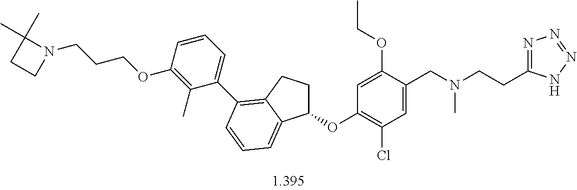 [SMILES: CCOc1cc(O[C@H]2CCc3c(-c4cccc(OCCCN5CCC5(C)C)c4C)cccc32)c(Cl)cc1CN(C)CCc1nnn[nH]1] | ++ | 659.20 | 1.70 |
| 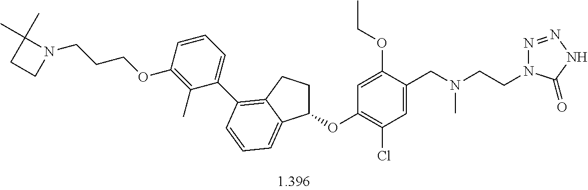 [SMILES: CCOc1cc(O[C@H]2CCc3c(-c4cccc(OCCCN5CCC5(C)C)c4C)cccc32)c(Cl)cc1CN(C)CCn1nn[nH]c1=O] | ++ | 675.30 | 1.70 |
| 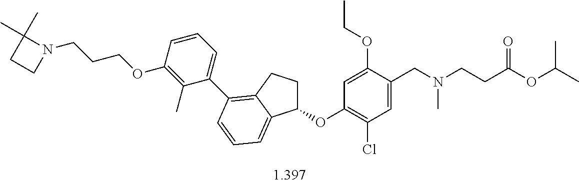 [SMILES: CCOc1cc(O[C@H]2CCc3c(-c4cccc(OCCCN5CCC5(C)C)c4C)cccc32)c(Cl)cc1CN(C)CCC(=O)OC(C)C] | ++ | 677.50 | 1.70 |
| 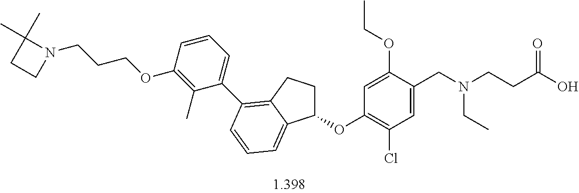 [SMILES: CCOc1cc(O[C@H]2CCc3c(-c4cccc(OCCCN5CCC5(C)C)c4C)cccc32)c(Cl)cc1CN(CC)CCC(=O)O] | ++ | 649.50 | 1.80 |
| 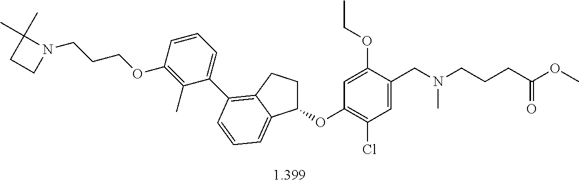 | ++ | 663.40 | 1.70 |
| 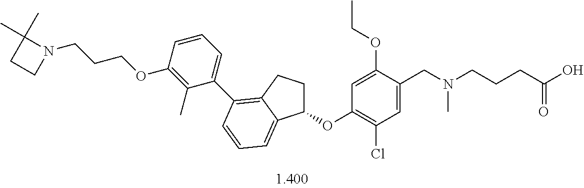 [SMILES: CCOc1cc(O[C@H]2CCc3c(-c4cccc(OCCCN5CCC5(C)C)c4C)cccc32)c(Cl)cc1CN(C)CCCC(=O)O] | +++ | 651.40 | 1.80 |
| 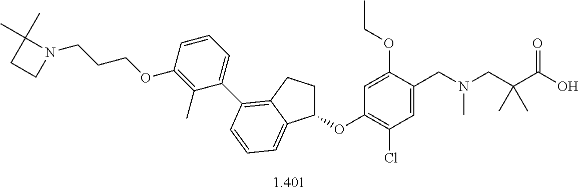 [SMILES: CCOc1cc(O[C@H]2CCc3c(-c4cccc(OCCCN5CCC5(C)C)c4C)cccc32)c(Cl)cc1CN(C)CC(C)(C)C(=O)O] | ++ | 665.50 | 1.80 |
| 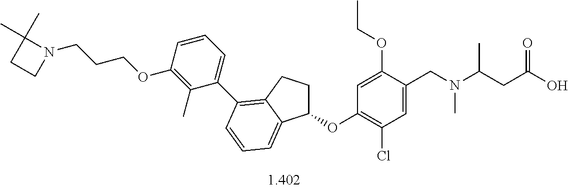 [SMILES: CCOc1cc(O[C@H]2CCc3c(-c4cccc(OCCCN5CCC5(C)C)c4C)cccc32)c(Cl)cc1CN(C)C(C)CC(=O)O] | ++ | 649.40 | 1.80 |
| 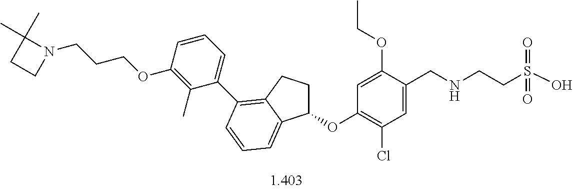 [SMILES: CCOc1cc(O[C@H]2CCc3c(-c4cccc(OCCCN5CCC5(C)C)c4C)cccc32)c(Cl)cc1CNCCS(=O)(=O)O] | ++ | 657.30 | 2.45* |
| 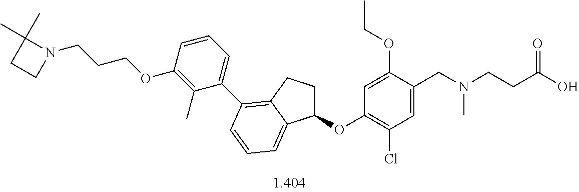 [SMILES: CCOc1cc(O[C@@H]2CCc3c(-c4cccc(OCCCN5CCC5(C)C)c4C)cccc32)c(Cl)cc1CN(C)CCC(=O)O] | + | 635.40 | 1.70 |
| 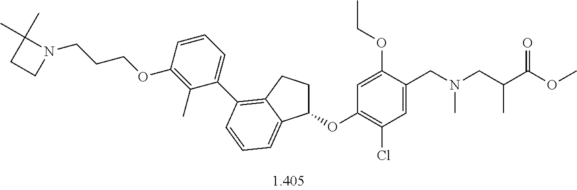 | + | 663.40 | 1.80 |
| 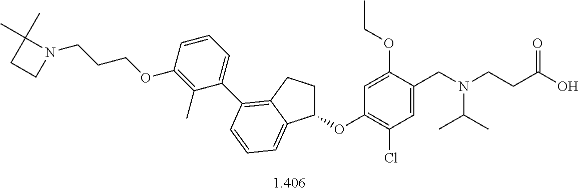 [SMILES: CCOc1cc(O[C@H]2CCc3c(-c4cccc(OCCCN5CCC5(C)C)c4C)cccc32)c(Cl)cc1CN(CCC(=O)O)C(C)C] | ++ | 663.30 | 1.80 |
| 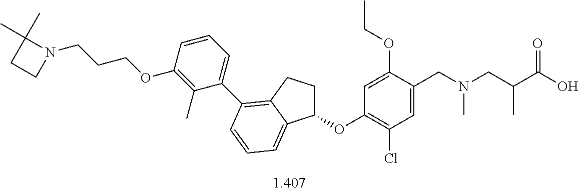 [SMILES: CCOc1cc(O[C@H]2CCc3c(-c4cccc(OCCCN5CCC5(C)C)c4C)cccc32)c(Cl)cc1CN(C)CC(C)C(=O)O] | ++ | 649.40 | 1.80 |
| 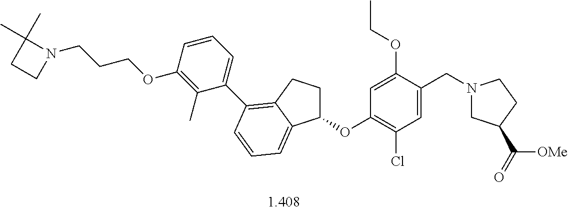 [SMILES: CCOc1cc(O[C@H]2CCc3c(-c4cccc(OCCCN5CCC5(C)C)c4C)cccc32)c(Cl)cc1CN1CC[C@@H](C(=O)OC)C1] | ++ | 661.40 | 1.70 |
| 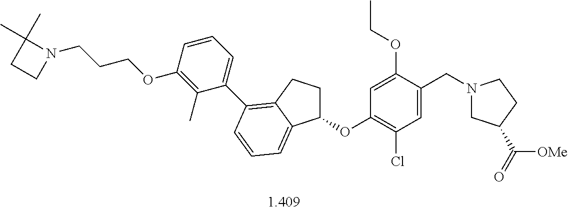 [SMILES: CCOc1cc(O[C@H]2CCc3c(-c4cccc(OCCCN5CCC5(C)C)c4C)cccc32)c(Cl)cc1CN1CC[C@H](C(=O)OC)C1] | ++ | 661.40 | 1.60 |
| 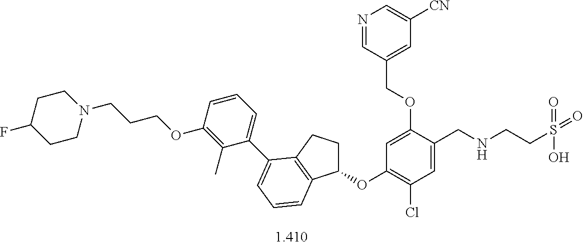 [SMILES: Cc1c(OCCCN2CCC(F)CC2)cccc1-c1cccc2c1CC[C@@H]2Oc1cc(OCc2cncc(C#N)c2)c(CNCCS(=O)(=O)O)cc1Cl] | +++ | 763.20 | 2.46* |
| 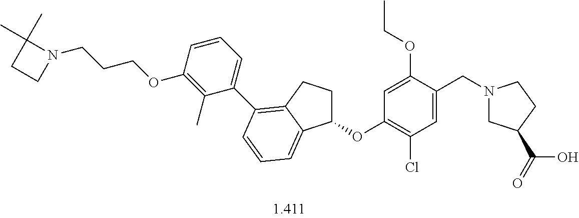 [SMILES: CCOc1cc(O[C@H]2CCc3c(-c4cccc(OCCCN5CCC5(C)C)c4C)cccc32)c(Cl)cc1CN1CC[C@@H](C(=O)O)C1] | ++ | 647.50 | 1.70 |
| 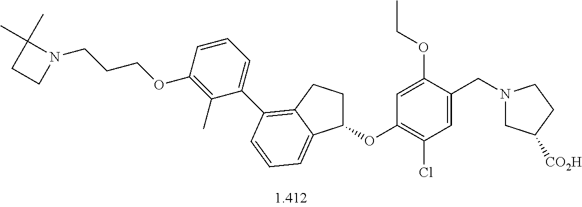 [SMILES: CCOc1cc(O[C@H]2CCc3c(-c4cccc(OCCCN5CCC5(C)C)c4C)cccc32)c(Cl)cc1CN1CC[C@H](C(=O)O)C1] | ++ | 648.40 | 1.80 |
| 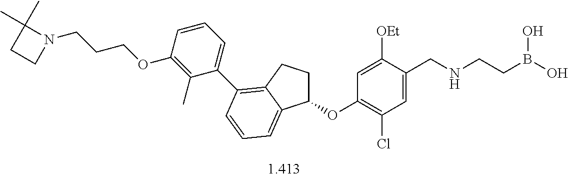 [SMILES: CCOc1cc(O[C@H]2CCc3c(-c4cccc(OCCCN5CCC5(C)C)c4C)cccc32)c(Cl)cc1CNCCB(O)O] | ++ | 621.30 | 2.36* |
| 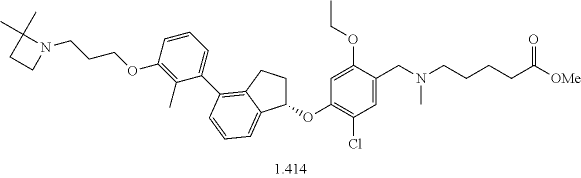 [SMILES: CCOc1cc(O[C@H]2CCc3c(-c4cccc(OCCCN5CCC5(C)C)c4C)cccc32)c(Cl)cc1CN(C)CCCCC(=O)OC] | ++ | 677.20 | 1.70 |
| 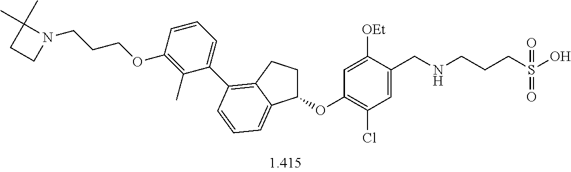 [SMILES: CCOc1cc(O[C@H]2CCc3c(-c4cccc(OCCCN5CCC5(C)C)c4C)cccc32)c(Cl)cc1CNCCCS(=O)(=O)O] | ++ | 671.30 | 2.49* |
| 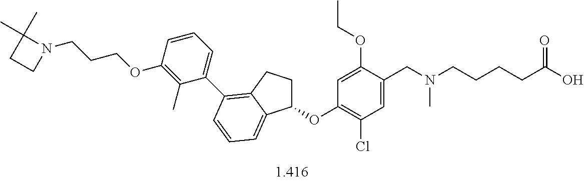 [SMILES: CCOc1cc(O[C@H]2CCc3c(-c4cccc(OCCCN5CCC5(C)C)c4C)cccc32)c(Cl)cc1CN(C)CCCCC(=O)O] | ++ | 663.30 | 1.80 |

^{#}Relative cis isomer corresponding to F-indane ether
^{$}Relative trans isomer corresponding to F-indane ether

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of preventing and/or treating cancer in an individual in need thereof, said method comprising administering an effective amount of a small molecule programmed death ligand 1 (PD-L1) inhibitor.

2. The method of claim 1, wherein said small molecule PD-L1 inhibitor is a compound of Formula (I). 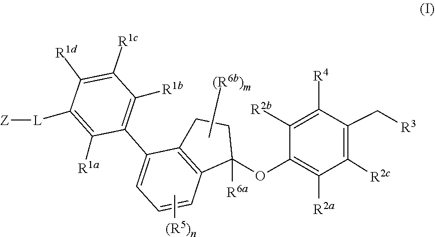
or a pharmaceutically acceptable salt thereof, or a prodrug or bioisostere thereof;
wherein:
each of R^{1a}, R^{1b} R^{1c} and R^{1d} is independently selected from the group consisting of H, halogen, CF₃, CN, C₁₋₄alkyl and —O—C₁₋₄ alkyl, wherein the C₁₋₄ alkyl and —O—C₁₋₄ alkyl are optionally further substituted with halogen, hydroxyl, methoxy or ethoxy;
L is a linking group selected from the group consisting of: 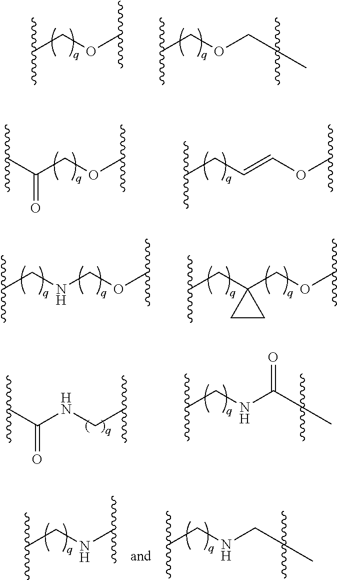
wherein each of the subscripts q is independently 1, 2, 3 or 4, and L is optionally further substituted with one or two members selected from the group consisting of halogen, hydroxy, C₁₋₃ alkyl, —O—C₁₋₃ alkyl, C₁₋₃ hydroxyalkyl, C₁₋₃ haloalkyl and —CO₂H;
Z is selected from the group consisting of azetidinyl, pyrollidinyl, piperidinyl, piperazinyl, morpholinyl, pyridyl, pyrimidinyl, imidazolyl, guanidinyl, quinuclidine, 2-azaspiro[3.3]heptane and 8-azabicyclo[3.2.1]octane, each of which is optionally substituted with from 1 to 4 groups independently selected from halogen, CN, hydroxy, oxo, C₁₋₄ alkyl, —NH₂, —NHC₁₋₃alkyl, —N(C₁₋₃alkyl)₂, —O—C₁₋₃ alkyl, C₁₋₃ hydroxyalkyl, C₁₋₃ haloalkyl, —OC(O)(C₁₋₄ alkyl), —CO₂(C₁₋₄ alkyl) and —CO₂H;
or
Z is selected from the group consisting of —CO₂R^{a} and —NR^{a}R^{b}; wherein R^{a} is selected from the group consisting of H, C₁₋₈ alkyl, C₁₋₈ haloalkyl and C₁₋₈ hydroxyalkyl; and R^{b} is selected from H, —C₁₋₈ alkyl, C₁₋₈ haloalkyl, C₁₋₈alkyl-COOH, C₁₋₈ alkyl-OH, C₁₋₈alkyl-CONH₂, C₁₋₈alkyl-SO₂NH₂, C₁₋₈ alkyl-PO₃H₂, C₁₋₈ alkyl-C(O)NHOH, —C(O)—C₁₋₈alkyl-OH, —C(O)—C₁₋₈alkyl-COOH, C₃₋₁₀ cycloalkyl, —C₃₋₁₀ cycloalkyl-COOH, —C₃₋₁₀ cycloalkyl-OH,—C₄₋₈ heterocyclyl, —C₄₋₈ heterocyclyl-COOH, —C₄₋₈heterocyclyl-OH, —C₁₋₈ alkyl-C₄₋₈ heterocyclyl, —C₁₋₈ alkyl-C₃₋₁₀ cycloalkyl, C₅₋₁₀ heteroaryl and —C₁₋₈alkyl-C₅₋₁₀heteroaryl;
each R^{2a}, R^{2b} and R^{2c} a is independently selected from the group consisting of H, halogen, —CN, —R^{d}, —CO₂R^{e}, —CONRₑR^{f}, —OC(O)NR^{e}R^{f}, —NR^{f}C(O)R^{d}, —NR^{e}—C(O)₂R^{d}, —NR^{e}—C(O)NR^{e}R^{f}, —NR^{e}R^{f}, —OR³, —X²—OR^{e}, —X²—NR^{e}R^{f}, —X²—CO₂R^{e}, —SF₅, and —S(O)₂NR^{e}R^{f}, wherein each X² is a C₁₋₄ alkylene; each R^{e} and R^{f} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O and S, and optionally substituted with oxo; each R^{d} is independently selected from the group consisting of C₁₋₈ alkyl, C₂₋₈ alkenyl, and C₁₋₈ haloalkyl;
R³ is selected from the group consisting of —NR^{g}R^{h} and C₄₋₁₂ heterocyclyl, wherein the C₄₋₁₂ heterocyclyl is optionally substituted with 1 to 6 R^{3a};
each R^{3a} is independently selected from the group consisting of halogen, —CN, oxo, —Rⁱ, —CO₂R^{j}, —CONR^{j}R^{k}, —CONHC₁₋₆ alkyl-OH, —C(O)R^{j}, —OC(O)NR^{j}R^{k}, —NR^{j}C(O)R^{k}, —NR^{j}C(O)₂R^{k}, —CONHOH, —PO₃H₂, —NR^{j}—-X³—C(O)₂R^{k}, —NR^{j}C(O)NR^{j}R^{k}, -NR¹R^{k}, —OR^{j}, —S(O)₂NR^{j}R^{k}, —O—X³—OR^{j}, —O—X³—NR^{j}R^{k}, —O—X³—CO₂R^{j}, —O—X³—CONR^{j}R^{k}, —X³—NR^{j} R^{k}, —X³—CO₂R^{j}, —X³—CONR^{j}R^{k}, —X³—CONHSO₂R^{j} and SF₅; wherein X³ is C₁₋₆ alkylene and is optionally further substituted with OH, SO₂NH₂, CONH₂, C(O)NHOH, PO₃H₂, COO-C₁₋₈alkyl or CO₂H, wherein each R^{j} and R^{k} is independently selected from hydrogen, C₁₋₈ alkyl optionally substituted with 1 to 2 substituents selected from OH, SO₂NH₂, CONH₂, C(O)NHOH, PO₃H₂, COO—C₁₋₈alkyl or CO₂H, and C₁₋₈ haloalkyl optionally substituted with 1 to 2 substituents selected from OH, SO₂NH₂, CONH₂, C(O)NHOH, PO₃H₂, COO—C₁₋₈alkyl or CO₂H, or when attached to the same nitrogen atom R^{j} and R^{k} can be combined with the nitrogen atom to form a five or six-membered ring having from 0 to 2 additional heteroatoms as ring members selected from N, O or S, and optionally substituted with oxo; each R′ is independently selected from the group consisting of —OH, C₁₋₈ alkyl, C₂₋₈ alkenyl, and C₁₋₈ haloalkyl each of which may be optionally substituted with OH, SO₂NH₂, CONH₂, C(O)NHOH, PO₃H₂, COO—C₁₋₈alkyl or CO₂H;
R^{g} is selected from the group consisting of H, C₁₋₈ haloalkyl and C₁₋₈ alkyl;
R^{h} is selected from —C₁₋₈ alkyl, C₁₋₈ haloalkyl, C₁₋₈ hydroxyalkyl, C₁₋₈alkyl-CO₂R^{j}, C₁₋₈alkyl-CONRJR^{j}R^{k}, and C₁₋₈alkyl-CONHSO₂R^{j}, C₁₋₈ alkyl-SO₂NR^{h1}R^{k}, C₁₋₈ alkyl-SO₃R^{j}, C₁₋₈ alkyl-B(OH)₂, C₁₋₈alkyl-PO₃H₂, C₁₋₈ alkyl-C(O)NHOH, C₁₋₈ alkyl-NR^{h1}R^{h2}, —C(O)R^{j}, C₃₋₁₀ cycloalkyl,-C₃₋₁₀ cycloalkyl-COOR^{j}, —C₃₋₁₀ cycloalkyl-OR^{j}, C₄₋₈heterocyclyl, —C₄₋₈ heterocyclyl-COOR^{j}, —C₄₋₈ heterocyclyl-OR^{j}, —C₁₋₈ alkyl-C₄₋₈heterocyclyl, —C(═O)OC₁₋₈ alkyl-C₄₋₈heterocyclyl, —C₁₋₈ alkyl-C₃₋₁₀ cycloalkyl, C₅₋₁₀ heteroaryl, —C₁₋₈alkyl-C₅₋₁₀ heteroaryl, —C₁₋₈ alkyl-C₆₋₁₀ aryl,—C₁₋₈ alkyl-(C═O)—C₆₋₁₀ aryl, —CO₂—C₁₋₈ alkyl-O₂C—C₁₋₈ alkyl, —C₁₋₈ alkyl-NH(C═O)—C₂₋₈ alkenyl , —C₁₋₈ alkyl-NH(C═O)—C₁₋₈ alkyl, —C₁₋₈ alkyl-NH(C═O)-13 C₂₋₈ alkynyl, —C₁₋₈ alkyl—(C═O)—NH—C₁₋₈ alkyl-COOR^{j}, and —C₁₋₈ alkyl-(C═O)—NH-C₁₋₈ alkyl-OR^{j} optionally substituted with CO₂H; or
R^{h} combined with the N to which it is attached is a mono-, di- or tri-peptide comprising 1-3 natural amino acids and 0-2 non-natural amino acids, wherein
the non-natural aminoacids have an alpha carbon substituent selected from the group consisting of C₂₋₄ hydroxyalkyl, C₁₋₃ alkyl-guanidinyl, and C₁₋₄ alkyl-heteroaryl,
the alpha carbon of each natural or non-natural amino acids are optionally further substituted with a methyl group, and
the terminal moiety of the mono-, di-, or tri-peptide is selected from the group consisting of C(O)OH, C(O)O—C₁₋₆ alkyl, and PO₃H₂, wherein
R^{h1} and R^{h2} are each independently selected from the group consisting of H, C₁₋₆ alkyl, and C₁₋₄hydroxyalkyl;
the C₁₋₈ alkyl portions of R^{h} are optionally further substituted with from 1 to 3 substituents independently selected from OH, COOH, SO₂NH₂, CONH₂, C(O)NHOH, COO—C₁₋₈ alkyl, PO₃H₂ and C₅₋₆ heteroaryl optionally substituted with 1 to 2 C₁₋₃ alkyl substituents,
the C₅₋₁₀ heteroaryl and the C₆₋₁₀ aryl portions of R^{h} are optionally substituted with 1 to 3 substituents independently selected from OH, B(OH)₂, COOH, SO₂NH₂, CONH₂, C(O)NHOH, PO₃H₂, COO—C₁₋₈alkyl, C₁₋₄alkyl, C₁₋₄alkyl-OH, C₁₋₄alkyl-SO₂NH₂, C₁₋₄alkyl CONH₂, C₁₋₄alkyl-C(O)NHOH, C₁₋₄alkyl- PO₃H₂, C₁₋₄alkyl-COOH, and phenyl and the C₄₋₈heterocyclyl and C₃₋₁₀ cycloalkyl portions of R^{h} are optionally substituted with 1 to 4 R^{w} substituents;
each R^{w} substituent is independently selected from C₁₋₄ alkyl, C₁₋₄ alkyl-OH, C₁₋₄ alkyl-COOH, C₁₋₄ alkyl-SO₂NH₂, C₁₋₄ alkyl CONH₂, C₁₋₄ alkyl-C(O)NHOH, C₁₋₄ alkyl-PO₃H, OH, COO-C₁₋₈ alkyl, COOH, SO₂NH₂, CONH₂, C(O)NHOH, PO₃H₂ and oxo;
R⁴ is selected from the group consisting of O—C₁₋₈ alkyl, O—C₁₋₈haloalkyl, C₆₋₁₀aryl, C₅₋₁₀ heteroaryl , —O—C₁₋₄ alkyl-C₄₋₇ heterocycloalkyl, —O—C₁₋₄ alkyl-C₆₋₁₀aryl and —O—-C₁₋₄ alkyl-C₅₋₁₀ heteroaryl, each of which is optionally substituted with 1 to 5 R⁴a;
each R^{4a} is independently selected from the group consisting of halogen, —CN, —R^{m}, —CO₂Rⁿ, —CONRⁿR^{p}, —C(O)Rⁿ, —OC(O)NRⁿR^{p}, —NRⁿC(O)R^{p}, —NRⁿC(O)₂R^{m}, —NRⁿ—C(O)NRⁿR^{p}, —NRⁿR^{p}, —ORⁿ, —O—X⁴—ORⁿ, —O—X⁴—NRⁿR^{p}, —O—X⁴—CO₂Rⁿ, —O—X⁴-CONRⁿR^{p}, —X⁴—ORⁿ, —X⁴—NRⁿR^{p}, —X⁴—CO₂Rⁿ, —X⁴-CONRⁿR^{p}, —SF₅, —S(O)₂RⁿR^{p}, —S(O)₂NRⁿR^{p}, C₃₋₇ cycloalkyl and C₄₋₇ heterocycloalkyl, wherein the cycloalkyl and heterocycloalkyl rings are optionally substituted with 1 to 5 R^{t}, wherein each R^{t} is independently selected from the group consisting of C₁₋₈ alkyl, C₁₋₈haloalkyl, —CO₂Rⁿ, —CONRⁿR^{p}, —C(O)Rⁿ, —OC(O)NRⁿR^{p}, —NRⁿC(O)R^{p}, —NRⁿC(O)₂R^{m}, —NRⁿ—C(O)NRⁿR^{p}, —NRⁿR^{p}, —ORⁿ, —O—X⁴—-ORⁿ, —O—X⁴-NRⁿR^{p},—O—X⁴—CO²Rⁿ, —O—X⁴—CONRⁿR^{p}, —X⁴—ORⁿ, —X⁴—NRⁿR^{p}, —X⁴—CO2Rⁿ, —X⁴—CONRⁿR^{p}, —SF₅, and —S(O)₂NRⁿR^{p};
wherein each X⁴ is a C₁₋₆ alkylene; each Rⁿ and R^{p} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each R^{m} is independently selected from the group consisting of C₁₋₈ alkyl, C₂₋₈ alkenyl, and C₁₋₈haloalkyl; and optionally when two R^{4a} substituents are on adjacent atoms, they are combined to form a fused five or six-membered carbocyclic or heterocyclic ring optionally substituted with oxo;
n is 0, 1, 2 or 3;
each R⁵ is independently selected from the group consisting of halogen, —CN, —R^{q}, —CO₂R^{r}, —CONR^{r}R^{s}, —C(O)R^{r}, —OC(O)NR^{r}R^{s}, —NR^{r}C(O)R^{s}, —NR^{r}C(O)²R^{q}, —NR^{r}—C(O)NR^{r}R^{s}, —NR^{r}R^{s}, —OR^{r}, —O—X⁵—OR^{r}, —O—X⁵, —NR^{r}R^{s}, —O—X⁵-—CO²R^{r}, —O—X⁵—CONR ^{r}R^{s}, —X⁵—OR^{r}, —X⁵—NR^{r}R^{s}, —X⁵—CO₂R^{r}, —X⁵-CONR^{r}R^{s}, —SF⁵, —S(O)₂NR^{r}R^{s},wherein each X⁵ is a C₁₋₄ alkylene; each R^{r} and R^{s} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each R^{q} is independently selected from the group consisting of C₁₋₈ alkyl, and C₁₋₄ shaloalkyl;
R^{6a} is selected from the group consisting of H, C₁₋₄ alkyl and C₁₋₄haloalkyl;
m is 0, 1, 2, 3 or 4;
each R^{6b} is independently selected from the group consisting of F, C₁₋₄ alkyl, O—R^{u}, C₁₋₄ haloalkyl, NR^{u}R^{v}, wherein each R^{u} and R^{v} is independently selected from hydrogen, C₁₋₈ alkyl, and C₁₋₈ -ghaloalkyl, or when attached to the same nitrogen atom can be combined with the nitrogen atom to form a five or six-membered ring having from 0 to 2 additional heteroatoms as ring members selected from N, O or S, and optionally substituted with oxo.

3. The method of claim 2, wherein the small molecule PD-L1 inhibitors has the formula (Ia) 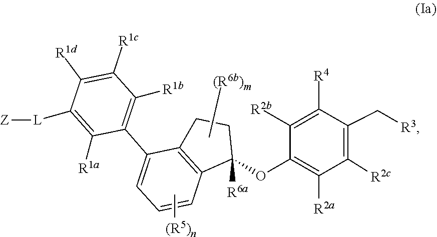
or a pharmaceutically acceptable salt thereof.

4. The method of claim 2, wherein the small molecule PD-L1 inhibitor has the formula (Ib) 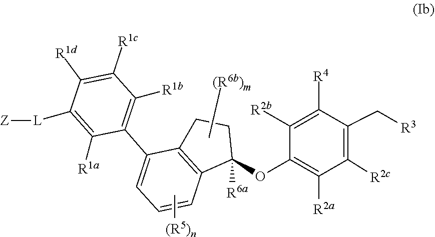
or a pharmaceutically acceptable salt thereof.

5. The method of claim 2, wherein the PD-L1 inhibitor has the formula (Ia1) or (Ia2): 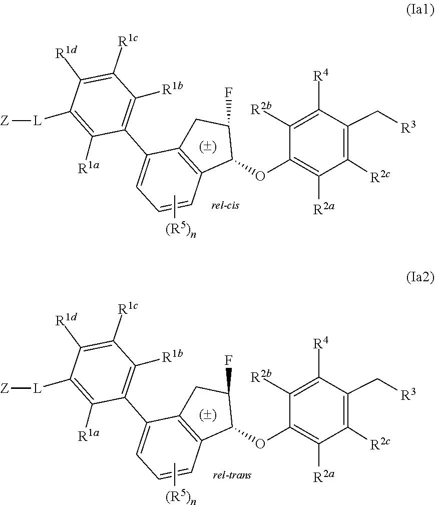
or a pharmaceutically acceptable salt thereof.

6. The method of any one of claims 2 to 4, wherein each of R^{2a}, R^{2b} and R_{2c} is independently selected from the group consisting of hydrogen, halogen, CN, C₁₋₄ alkyl, and C₁₋₄ haloalkyl.

7. The method of any one of claims 2 to 4, wherein R³ is —NR^{g}R^{h}.

8. The method of any one of claims 2 to 4, wherein R³ is C₄₋₁₂ heterocyclyl, wherein the C₄₋₁₂ heterocyclyl is optionally substituted with 1 to 6 R^{3a}.

9. The method of claim 8, wherein the C₄₋₁₂ heterocyclyl a C₇₋₁₁ spiroheterocyclyl and is optionally substituted with 1 to 6 R^{3a}

10. The method of any one of claims 2 to 4, wherein R³ is selected from the group consisting of:

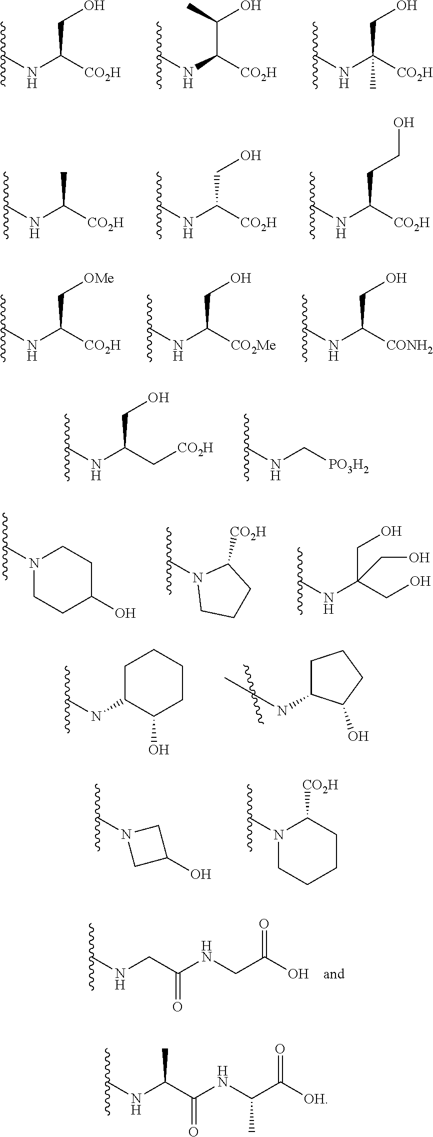

11. The method of any one of claims 2 to 4, wherein R³ is selected from the group consisting of:

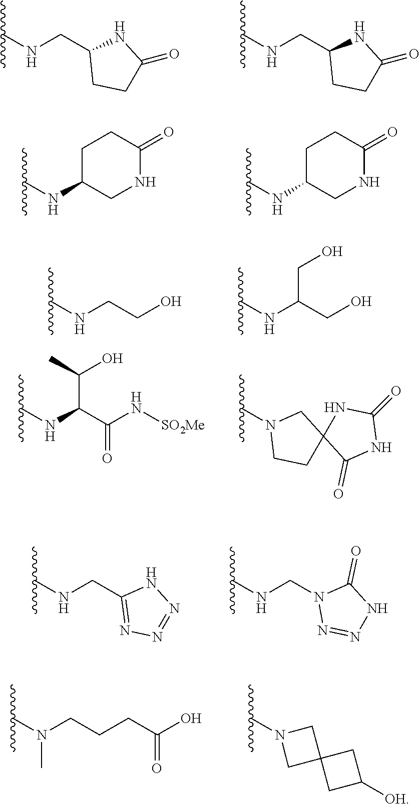

12. The method of any one of claims 2 to 4, wherein R⁴ is selected from the group consisting of:

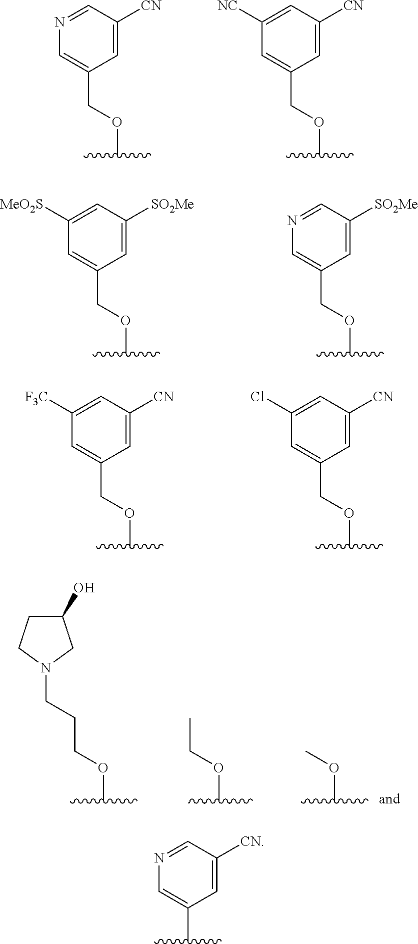

13. The method of any one of claims 2 to 12, wherein n is 0.

14. The method of any one of claims 2 to 13, wherein R^{6a} and R^{6b} are each independently selected from the group consisting of hydrogen, halogen, C₁₋₄ alkyl and C₁₋₄ haloalkyl.

15. The method of any one of claims 2 to 13, wherein the group Z-L- is selected from the group consisting of:

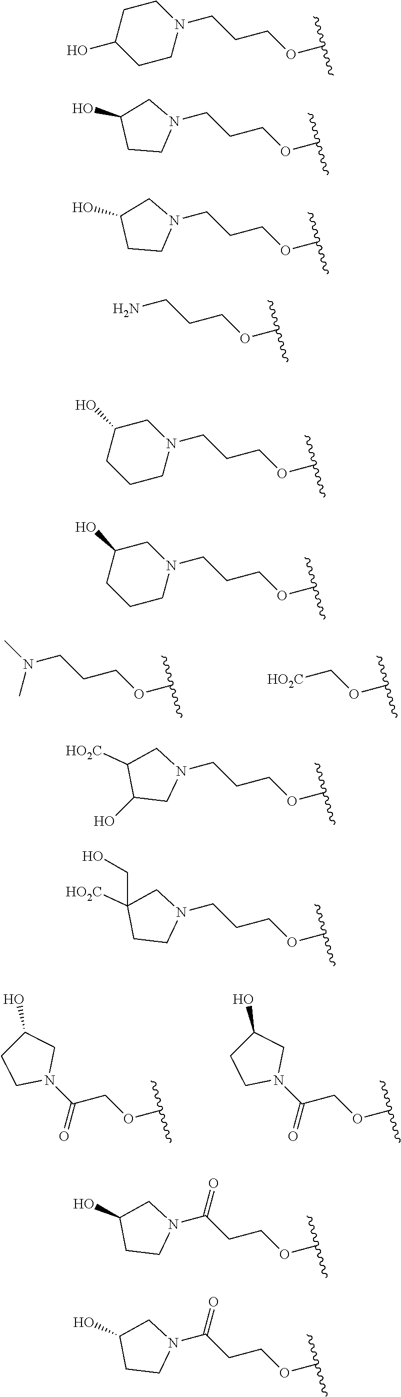

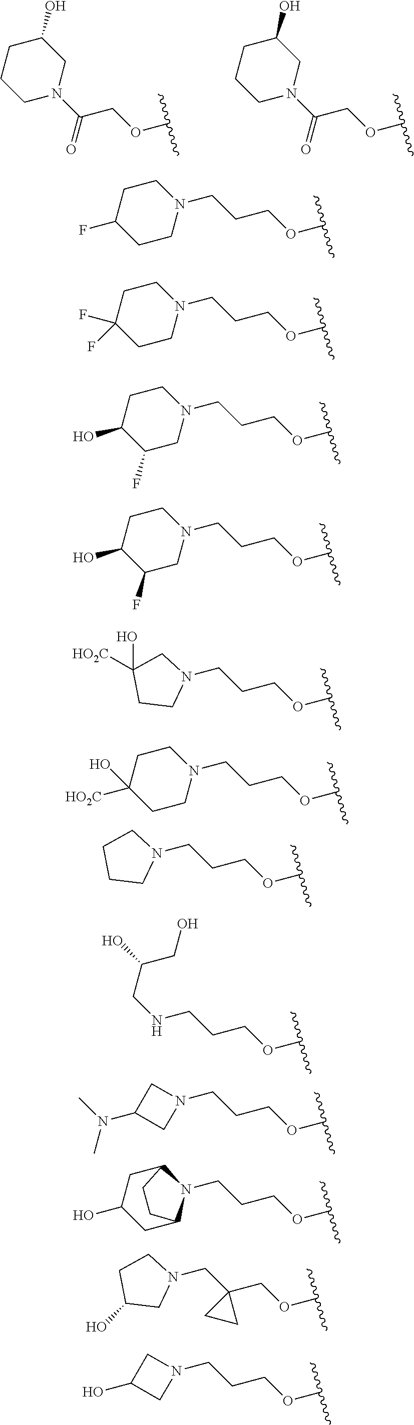

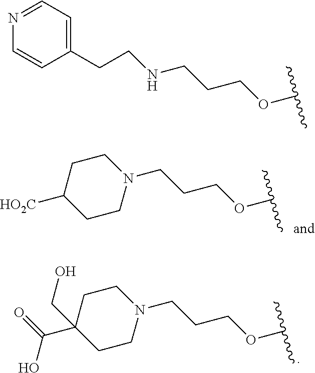 [SMILES: *OCCCNCCc1ccncc1 ; *OCCCN1CCC(C(=O)O)CC1 ; *OCCCN1CCC(CO)(C(=O)O)CC1]

16. The method of any one of claims 2 to 13, wherein the group Z-L- is selected from the group consisting of:

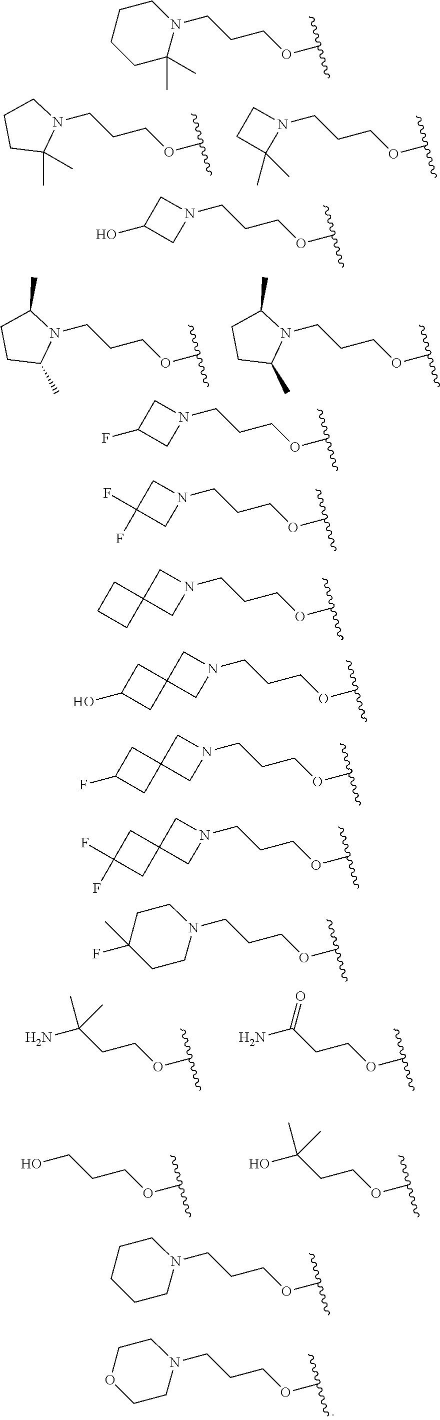

17. The method of any one of claims 2 to 13, wherein R⁴ is selected from the group consisting of:

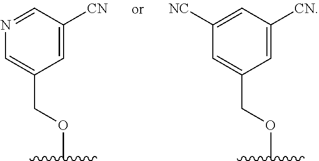 [SMILES: *OCc1cncc(C#N)c1 ; *OCc1cc(C#N)cc(C#N)c1]

18. The method of any one of claims 2 to 4, wherein R^{2b} and R^{2c} are both H and R^{2a} is selected from the group consisting of halogen, C₁₋₄ alkyl, C₂₋₄ alkenyl, C₁₋₃ haloalkyl, —CN, —OMe and OEt.

19. The method of any one of claims 2 to 4, wherein R^{2b} and R^{2c} are both H and R^{2a} is halogen.

20. The method of any one of claims 2 to 4, wherein R^{2b} and R^{2c} are both H and R^{2a} is Cl.

21. The method of any one of claims 2 to 4, wherein R^{6a} is H.

22. The method of any one of claims 2 to 4, wherein m is 0.

23. The method of any one of claims 2 to 4, wherein m is 1 and R^{6b} is selected from the group consisting of F, C₁₋₄ alkyl, O-13 R^{u}, C₁₋₄ haloalkyl and NR^{u}R^{v}, wherein each R^{u} and R^{v} is independently selected from hydrogen, C₁₋₈ alkyl, and C₁₋₈ haloalkyl.

24. The method of any one of claims 2 to 4, wherein m is 1 and R^{6b} is F.

25. The method of claim 1, wherein said small molecule PD-L1 inhibitor is a compound in Table 1.

26. The method of claim 1, wherein said small molecule PD-L1 inhibitor has the formula of Compound 1.004 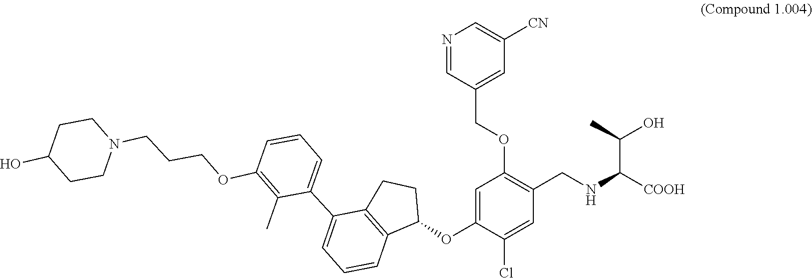 [SMILES: Cc1c(OCCCN2CCC(O)CC2)cccc1-c1cccc2c1CC[C@@H]2Oc1cc(OCc2cncc(C#N)c2)c(CN[C@H](C(=O)O)[C@@H](C)O)cc1Cl]
or a pharmaceutically acceptable salt thereof.

27. The method of claim 1, wherein said small molecule PD-L1 inhibitor has the formula of Compound 1.041 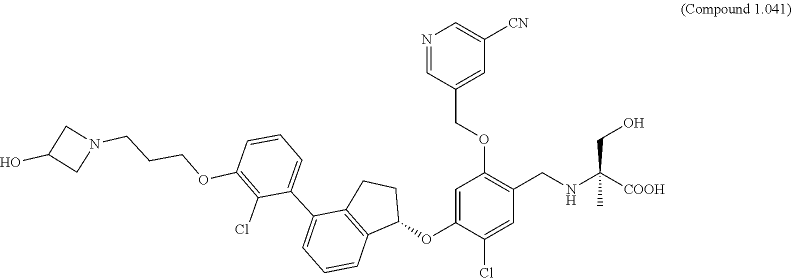
or a pharmaceutically acceptable salt thereof.

28. The method of claim 1, wherein said small molecule PD-L1 inhibitor has the formula of Compound 1.227 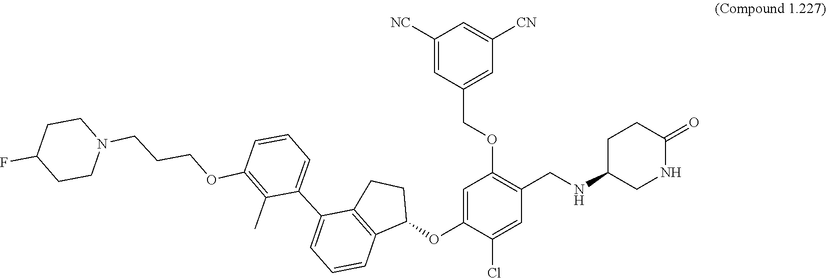 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC(=O)NC2)cc1Cl]
or a pharmaceutically acceptable salt thereof.

29. The method of claim 1, wherein said small molecule PD-L1 inhibitor has the formula of Compound 1.347 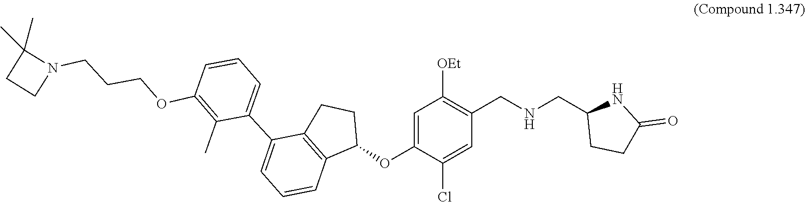 [SMILES: CCOc1cc(O[C@H]2CCc3c(-c4cccc(OCCCN5CCC5(C)C)c4C)cccc32)c(Cl)cc1CNC[C@@H]1CCC(=O)N1]
or a pharmaceutically acceptable salt thereof.

30. The method of any one of claims 1-29, wherein said cancer is a solid tumor.

31. The method of any one of claims 1-29, wherein said cancer is melanoma.

32. The method of any one of claims 1 to 31, wherein said treatment provides tumor size reduction as compared to an individual who was not administered a PD-L1 inhibitor.

33. The method of any one of claims 1 to 31, wherein said treatment reduces tumor growth.

34. The method of any one of claims 1 to 31, wherein said treatment destroys the cancer.

35. The method of any one of claims 1 to 34, wherein said small molecule PD-L1 inhibitor is administered orally.

36. A method of preventing and/or treating melanoma in an individual in need thereof, said method comprising administering an effective amount of a small molecule programmed death ligand 1 (PD-L1) inhibitor selected from the group consisting of 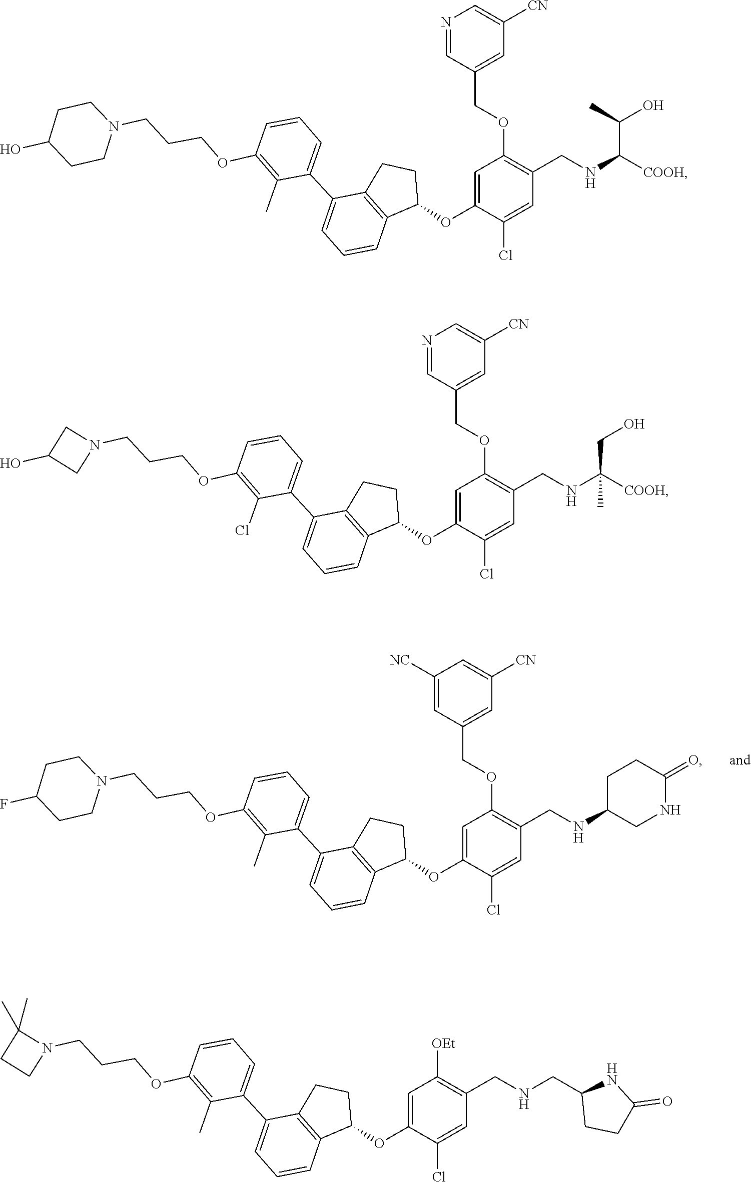
or a pharmaceutically acceptable salt thereof.

37. The method of claim 36, wherein said small molecule PD-L1 inhibitor has the formula of Compound 1.004 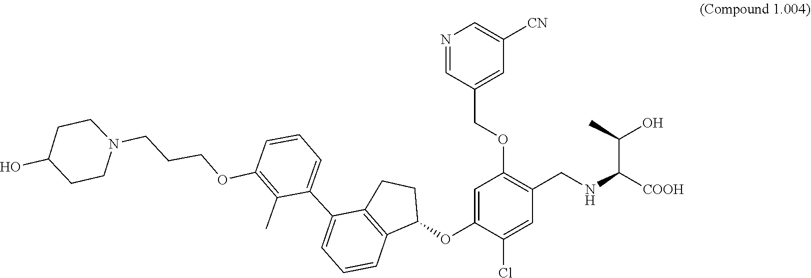 [SMILES: Cc1c(OCCCN2CCC(O)CC2)cccc1-c1cccc2c1CC[C@@H]2Oc1cc(OCc2cncc(C#N)c2)c(CN[C@H](C(=O)O)[C@@H](C)O)cc1Cl]
or a pharmaceutically acceptable salt thereof.

38. The method of claim 36, wherein said small molecule PD-L1 inhibitor has the formula of Compound 1.041 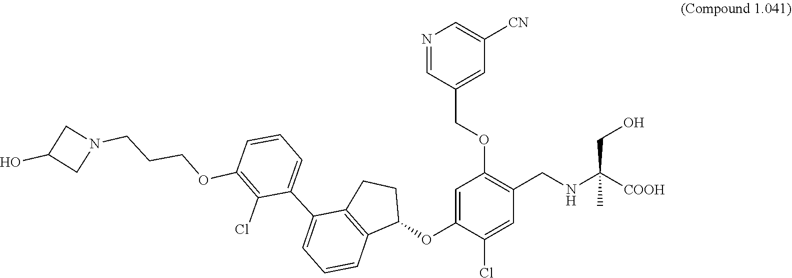
or a pharmaceutically acceptable salt thereof.

39. The method of claim 36, wherein said small molecule PD-L1 inhibitor has the formula of Compound 1.227 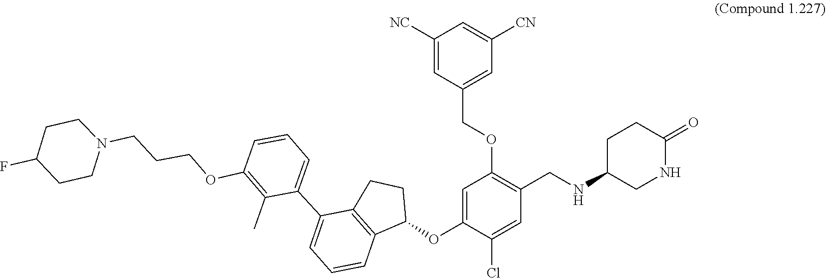 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC(=O)NC2)cc1Cl]
or a pharmaceutically acceptable salt thereof.

40. The method of claim 36, wherein said small molecule PD-L1 inhibitor has the formula of Compound 1.347 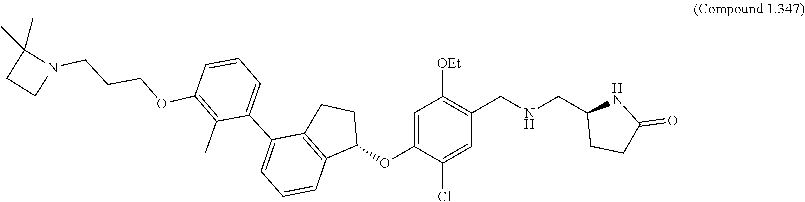 [SMILES: CCOc1cc(O[C@H]2CCc3c(-c4cccc(OCCCN5CCC5(C)C)c4C)cccc32)c(Cl)cc1CNC[C@@H]1CCC(=O)N1]
or a pharmaceutically acceptable salt thereof.

41. A method of increasing the CD8+ T cell/CD4+ T cell ratio in a solid tumor microenvironment, said method comprising administering an effective amount of a small molecule programmed death ligand 1 (PD-L1) inhibitor of Formula (I) 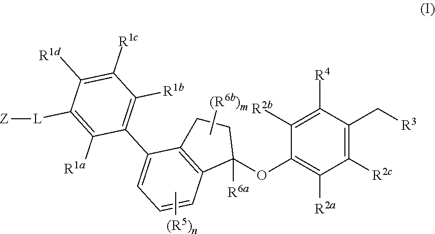
or a pharmaceutically acceptable salt thereof, or a prodrug or bioisostere thereof;
wherein:
each of R^{1a}, R^{1b}, R^{1c} and R^{1d}is independently selected from the group consisting of H, halogen, CF₃, CN, C₁₋₄ alkyl and —O—C₁₋₄ alkyl, wherein the C₁₋₄ alkyl and —O—C₁₋₄ alkyl are optionally further substituted with halogen, hydroxyl, methoxy or ethoxy;
L is a linking group selected from the group consisting of: 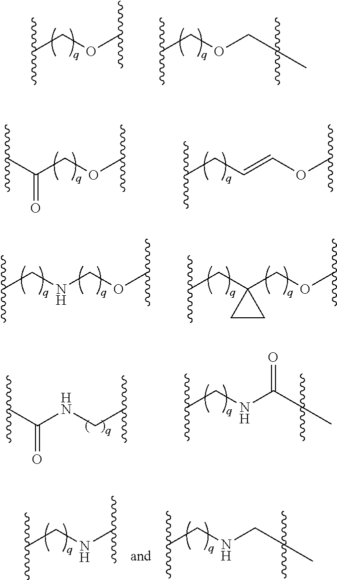
wherein each of the subscripts q is independently 1, 2, 3 or 4, and L is optionally further substituted with one or two members selected from the group consisting of halogen, hydroxy, C₁₋₃ alkyl, —O—C₁₋₃ alkyl, C₁₋₃ hydroxyalkyl, C₁₋₃ haloalkyl and —CO₂H;
Z is selected from the group consisting of azetidinyl, pyrollidinyl, piperidinyl, piperazinyl, morpholinyl, pyridyl, pyrimidinyl, imidazolyl, guanidinyl, quinuclidine, 2-azaspiro[3.3]heptane and 8-azabicyclo[3.2.1]octane, each of which is optionally substituted with from 1 to 4 groups independently selected from halogen, CN, hydroxy, oxo, C₁₋₄ alkyl, —NH₂, —NHC₁₋₃alkyl, —N(C₁₋₃alkyl)₂, —O—C₁₋₃ alkyl, C₁₋₃ hydroxyalkyl, C₁₋₃ haloalkyl, —OC(O)(C₁₋₄ alkyl), —CO₂(C₁₋₄ alkyl) and —CO₂H;
or
Z is selected from the group consisting of —CO₂Rₐ and —NR^{a}R^{b}; wherein R^{a} is selected from the group consisting of H, C₁₋₈ alkyl, C₁₋₈haloalkyl and C₁₋₈hydroxyalkyl; and R^{b} is selected from H, —C₁₋₈ alkyl, C₁₋₈haloalkyl, C₁₋₈ alkyl-COOH, C₁₋₈ alkyl-OH, C₁₋₈alkyl-CONH₂, C₁₋₈ alkyl-SO₂NH₂ C₁₋₈ alkyl-PO₃H₂, C₁₋₈ alkyl-C(O)NHOH, —C(O)—C₁₋₈alkyl-OH, —C(O)—C₁₋₈alkyl-COOH, C₃₋₁₀ cycloalkyl, —C₃₋₁₀ cycloalkyl-COOH, —C₃₋₁₀cycloalkyl-OH, C₄₋₈ heterocyclyl, —C₄₋₈ heterocyclyl-COOH, —C₄₋₈ heterocyclyl-OH, —C₁₋₈ alkyl-C₄₋₈ heterocyclyl, —C₁₋₈ alkyl-C₃₋₁₀ cycloalkyl, C₅₋₁₀ heteroaryl and —C₁₋₈alkyl-C₅₋₁₀ heteroaryl;
each R^{2a}, R^{2b} and R^{2c} is independently selected from the group consisting of H, halogen, —CN, —R^{d}, —CO₂R^{e}, —CONR^{e}R^{f}—OC(O)NR^{e}R^{f}, —NR^{f}C(O)R^{e}, —NR^{f}C(O)₂R^{d}, —NR^{e}—C(O)NR^{e}R^{f}, —NR^{e}R^{f}, —OR^{e}, —X²—NR^{e}R^{f}, —X²—CO₂R^{e}, —SF₅, and —S(O)₂NR^{e}R^{f}, wherein each X² is a C₁₋₄ alkylene; each R^{e} and R^{f} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O and S, and optionally substituted with oxo; each R^{d} is independently selected from the group consisting of C₁₋₈ alkyl, C₂₋₈ alkenyl, and C₁₋₈haloalkyl;
R³ is selected from the group consisting of —NR^{g}R^{h} and C₄₋₁₂ heterocyclyl, wherein the C₄₋₁₂ heterocyclyl is optionally substituted with 1 to 6 R^{3a};
each R^{3a} is independently selected from the group consisting of halogen, —CN, oxo, —Rⁱ, —CO₂R^{j}, —CONR^{j}R^{k}, —CONHC₁₋₆alkyl-OH, —C(O)R^{j}, —OC(O)NR^{j}R^{k}, —NR^{j}C(O)R^{k}, -NR^{j}C(O)₂R^{k}, —CONHOH, —PO₃H₂, —NR^{j}—X³—C(O)₂R^{k}, —NRC(O)NR^{j}R^{k}, —NR^{j}R^{k}, —OR^{j}, —S(O)₂NR^{j}R^{k}, —O—X³—O^{j}, —O—X³—NR^{j}R^{k}, —O—X³—CO₂R^{j}, —O—X³—CONR^{j}R^{k}, —X³—OR^{j}, —X³—NR^{j}R^{k}, —X³—CO₂R, —X³—CONR^{j}R^{k}, −X³—CONHSO₂R^{j} and SF₅; wherein X³ is C₁₋₆ alkylene and is optionally further substituted with OH, SO₂NH₂, CONH₂, C(O)NHOH, PO₃H₂, COO—C₁₋₈alkyl or CO₂H, wherein each R^{j} and R^{k} is independently selected from hydrogen, C₁₋₈ alkyl optionally substituted with 1 to 2 substituents selected from OH, SO₂NH₂, CONH₂, C(O)NHOH, PO₃H₂, COO—C₁₋₈alkyl or CO₂H, and C₁₋₈haloalkyl optionally substituted with 1 to 2 substituents selected from OH, SO₂NH₂, CONH₂, C(O)NHOH, PO₃H₂, COO—C₁₋₈alkyl or CO₂H, or when attached to the same nitrogen atom R^{j} and R^{k} can be combined with the nitrogen atom to form a five or six-membered ring having from 0 to 2 additional heteroatoms as ring members selected from N, O or S, and optionally substituted with oxo; each Rⁱ s independently selected from the group consisting of —OH, C₁₋₈ alkyl, C₂₋₈ alkenyl, and C₁₋₈ haloalkyl each of which may be optionally substituted with OH, SO₂NH₂, CONH₂, C(O)NHOH, PO₃H₂, COO—C₁₋₈alkyl or CO₂H;
R^{g} is selected from the group consisting of H, C₁₋₈haloalkyl and C₁₋₈ alkyl;
R^{h} is selected from —C₁₋₈alkyl, C₁₋₈haloalkyl, C₁₋₈hydroxyalkyl, C₁₋₈alkyl—CO₂R^{j}, C₁₋₈alkyl-CONR^{j}R^{k} , and C₁₋₈alkyl-CONHSO₂R^{j}, C₁₋₈alkyl-SO₂NR^{j}R^{k}, C₁₋₈alkyl-SO₃R^{j}, C₁₋₈ alkyl-B(OH)₂, C₁₋₈alkyl-PO₃H₂, C₁₋₈alkyl-C(O)NHOH, C₁₋₈ alkyl-NR^{h1}R^{h2}, —C(O)R^{j}, C₃₋₁₀ cycloalkyl,-C₃₋₁₀ cycloalkyl-COOR^{j}, —C₃₋₁₀ cycloalkyl-OR^{j}, C₄₋₈ heterocyclyl, —C₄₋₈ heterocyclyl-COOR^{j}, —C₄₋₈ heterocyclyl-OR^{j}, —C₁₋₈ alkyl-C₄₋₈ heterocyclyl, —C(═O)OC₁₋₈ alkyl-C₄₋₈ heterocyclyl, —C₁₋₈ alkyl-C₃₋₁₀ cycloalkyl, C₅₋₁₀heteroaryl, —C₁₋₈alkyl-C₅₋₁₀ heteroaryl, -C₁₋₈ alkyl-C₆₋₁₀ aryl, —C₁₋₈ alkyl-(C═O)—C₆₋₁₀ aryl, —CO₂—C₁₋₈alkyl-O₂C—C₁₋₈ alkyl, —C₁₋₈ alkyl-NH(C═O)—C₂₋₈ alkenyl , —C₁₋₈ alkyl-NH(C═O)—C₁₋₈ alkyl, —C₁₋₈ alkyl-NH(C═O)—C₂₋₈ alkynyl, —C₁₋₈ alkyl-(C═O)—NH—C₁₋₈ alkyl-COOR^{j}, and —C₁₋₈ alkyl-(C═O)—NH—C₁₋₈ alkyl-OR^{j} optionally substituted with CO₂H; or
R^{h} combined with the N to which it is attached is a mono-, di- or tri-peptide comprising 1-3 natural amino acids and 0-2 non-natural amino acids, wherein
the non-natural aminoacids have an alpha carbon substituent selected from the group consisting of C₂₋₄ hydroxyalkyl, C₁₋₃ alkyl-guanidinyl, and C₁₋₄ alkyl-heteroaryl,
the alpha carbon of each natural or non-natural amino acids are optionally further substituted with a methyl group, and
the terminal moiety of the mono-, di-, or tri-peptide is selected from the group consisting of C(O)OH, C(O)O—C₁₋₆ alkyl, and PO₃H₂, wherein
R^{h1} and R^{h2} are each independently selected from the group consisting of H, C₁₋₆ alkyl, and C₁₋₄ hydroxyalkyl;
the C₁₋₈ alkyl portions of R^{h} are optionally further substituted with from 1 to 3 substituents independently selected from OH, COOH, SO₂NH₂, CONH₂, C(O)NHOH, COO-C₁₋₈ alkyl, PO₃H₂ and C₅₋₆ heteroaryl optionally substituted with 1 to 2 C₁₋₃ alkyl substituents,
the C₅₋₁₀ heteroaryl and the C₆₋₁₀ aryl portions of R^{h} are optionally substituted with 1 to 3 substituents independently selected from OH, B(OH)₂, COOH, SO₂NH₂, CONH₂, C(O)NHOH, PO₃H₂, COO-C₁₋₈alkyl, C₁₋₄alkyl, C₁₋₄alkyl-OH, C₁₋₄alkyl-SO₂NH₂, C₁₋₄alkyl CONH₂, C₁₋₄alkyl-C(O)NHOH, C₁₋₄alkyl- PO₃H₂, C₁₋₄alkyl-COOH, and phenyl and the C₄₋₈ heterocyclyl and C₃₋₁₀ cycloalkyl portions of R^{h} are optionally substituted with 1 to 4 R^{w} substituents;
each R^{w} substituent is independently selected from C₁₋₄ alkyl, C₁₋₄ alkyl-OH, C₁₋₄ alkyl-COOH, C₁₋₄ alkyl-SO₂NH₂, C₁₋₄ alkyl CONH₂, C₁₋₄ alkyl-C(O)NHOH, C₁₋₄ alkyl-PO₃H, OH, COO-C₁₋₈ alkyl, COOH, SO₂NH₂, CONH₂, C(O)NHOH, PO₃H₂ and oxo;
R⁴ is selected from the group consisting of O—C₁₋₈ alkyl, O—C₁₋₈ haloalkyl, C₆₋₁₀ aryl, C₅₋₁₀ heteroaryl , —O—C₁₋₄ alkyl-C₄₋₇heterocycloalkyl, —O—C₁₋₄ alkyl-C₆₋₁₀aryl and —O—C₁₋₄ alkyl-C₅₋₁₀ heteroaryl, each of which is optionally substituted with 1 to 5 R^{4a};
each R^{4a} is independently selected from the group consisting of halogen, —CN, —R^{m}, —CO₂Rⁿ, —CONRⁿR^{p}, —C(O)Rⁿ, —OC(O)NRⁿR^{p}, —NRⁿC(O)R^{p}, —NRⁿC(O)₂R^{m}, —NRⁿ—C(O)NRⁿR^{p}, —NRⁿR^{p}, —ORⁿ, —O—X⁴—ORⁿ, —O—X ⁴—CO₂Rⁿ,—O—X⁴—CONRⁿR^{p}, —X⁴—ORⁿ, —X⁴—NRⁿR^{p}, —X⁴—CO₂Rⁿ, —X⁴—CONRⁿR^{p}, —SF₅, —S(O)₂RⁿR^{p}, —S(O)₂NRⁿR^{p}, C₃₋₇ cycloalkyl and C₄₋₇ heterocycloalkyl, wherein the cycloalkyl and heterocycloalkyl rings are optionally substituted with 1 to 5 R^{t}, wherein each R^{t} is independently selected from the group consisting of C₁₋₈ alkyl, C₁₋₈haloalkyl, —CO₂Rⁿ, —CONRⁿR^{p}, —C(O)Rⁿ, —OC(O)NRⁿR^{p}, —NRⁿC(O)R^{p}, —NRⁿC(O)₂R^{m}, —NRⁿ—C(O)NRⁿR^{p}, —NRⁿR^{p}, —ORⁿ, —O—X⁴—ORⁿ, —O—X⁴—CO²Rⁿ, —O—X⁴-CONRⁿR^{p}, —X⁴—ORⁿ, —X⁴—NRⁿR^{p}, —X⁴-CO²Rⁿ, —X⁴—CONRⁿRP, —SF₅, and —S(O)₂NRⁿR^{p};
wherein each X⁴ is a C₁₋₆ alkylene; each Rⁿ and R^{p} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each Rm is independently selected from the group consisting of C₁₋₈alkyl, C₂₋₈ alkenyl, and C₁₋₈ haloalkyl; and optionally when two R^{4a} substituents are on adjacent atoms, they are combined to form a fused five or six-membered carbocyclic or heterocyclic ring optionally substituted with oxo;
n is 0, 1, 2 or 3;
each R⁵ is independently selected from the group consisting of halogen, —CN, —R^{q}, —CO₂R^{r}, —CONR^{r}R^{s}, —C(O)R^{r}, —OC(O)N^{r}R^{s}, —NR^{r}C(O)R^{s}, —NR^{r}C(O)₂R^{q}, —NR^{r}—C(O)NR^{r}R^{s}, —NR^{r}R^{s}, —OR^{r}, —O—X⁵—OR^{r}, —O—X⁵—NR^{r}R^{s}, —O—X⁵—CO ₂R^{r}, —O—X⁵-CONR^{r}R^{s}, —X⁵—OR^{r}, —X⁵—NR^{r}R^{s}, —X⁵—CO₂R^{r}, —X⁵—CONR^{r}R^{s}, —SF₅, —S(O)₂NR^{r}R^{s}, wherein each X⁵ is a C₁₋₄ alkylene; each R^{r} and R^{s} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S, and optionally substituted with oxo; each Rq is independently selected from the group consisting of C₁₋₈ alkyl, and C₁₋₈ haloalkyl;
R^{6a} is selected from the group consisting of H, C₁₋₄ alkyl and C₁₋₄ haloalkyl;
m is 0, 1, 2, 3 or 4;
each R^{6b} is independently selected from the group consisting of F, C₁₋₄ alkyl, O—R^{u}, C₁₋₄haloalkyl, NR^{u}R^{v}, wherein each R^{u} and R^{v} is independently selected from hydrogen, C₁₋₈ alkyl, and C₁₋₈haloalkyl, or when attached to the same nitrogen atom can be combined with the nitrogen atom to form a five or six-membered ring having from 0 to 2 additional heteroatoms as ring members selected from N, O or S, and optionally substituted with oxo..

42. The method of claim 41, wherein said small molecule PD-L1 inhibitor of Formula (I) has the formula of Compound 1.004 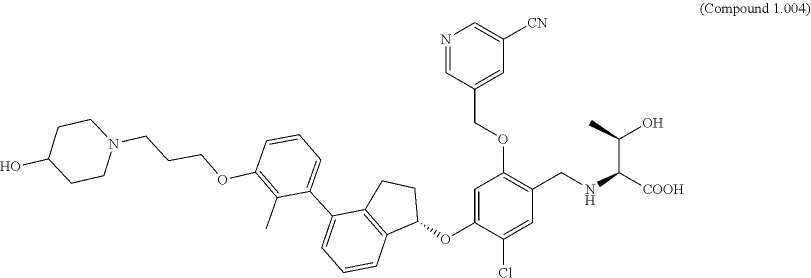 [SMILES: Cc1c(OCCCN2CCC(O)CC2)cccc1-c1cccc2c1CC[C@@H]2Oc1cc(OCc2cncc(C#N)c2)c(CN[C@H](C(=O)O)[C@@H](C)O)cc1Cl]
or a pharmaceutically acceptable salt thereof.

43. The method of claim 41, wherein said small molecule PD-L1 inhibitor of Formula (I) has the formula of Compound 1.041 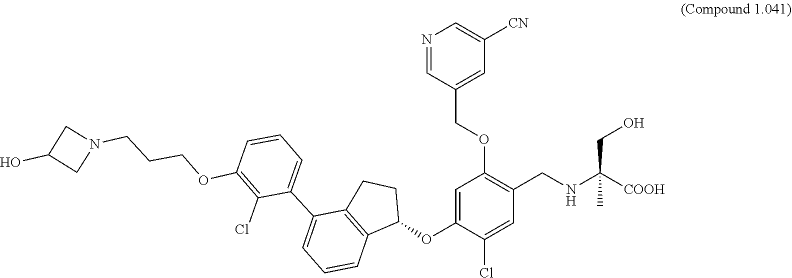
or a pharmaceutically acceptable salt thereof.

44. The method of claim 41, wherein said small molecule PD-L1 inhibitor of Formula (I) has the formula of Compound 1.227 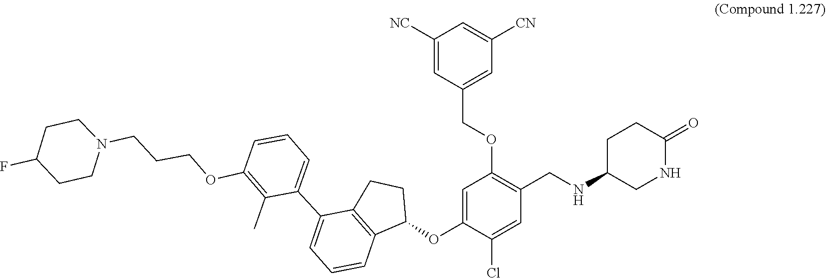 [SMILES: Cc1c(OCCCN2CCC(F)CC2)cccc1-c1cccc2c1CC[C@@H]2Oc1cc(OCc2cc(C#N)cc(C#N)c2)c(CN[C@H]2CCC(=O)NC2)cc1Cl]
or a pharmaceutically acceptable salt thereof.

45. The method of claim 41, wherein said small molecule PD-L1 inhibitor of Formula (I) has the formula of Compound 1.347 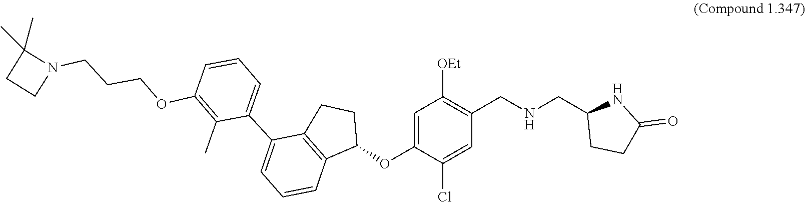 [SMILES: CCOc1cc(O[C@H]2CCc3c(-c4cccc(OCCCN5CCC5(C)C)c4C)cccc32)c(Cl)cc1CNC[C@@H]1CCC(=O)N1]
or a pharmaceutically acceptable salt thereof.
